(19) United States
(12) Patent Application Publication
(10) Pub. No.: US 2025/0135031 A1
(43) Pub. Date: May 1, 2025

(54) SINGLE-VECTOR GENE CONSTRUCT COMPRISING INSULIN AND GLUCOKINASE GENES

(71) Applicant: Universitat Autònoma de Barcelona, Barcelona (ES)

(72) Inventors: Fatima BOSCH TUBERT, Cerdanyola del Valles (ES); Miquel GARCIA MARTINEZ, Cerdanyola del Valles (ES); Veronica JIMÉNEZ CENZANO, Cerdanyola del Valles (ES); Virginia HAURIGOT MENDONÇA, Cerdanyola del Valles (ES)

(21) Appl. No.: 18/741,296

(22) Filed: Jun. 12, 2024

Related U.S. Application Data
(63) Continuation of application No. 16/801,262, filed on Feb. 26, 2020, now abandoned.
Application No. 16/801,262 is a division of application No. 15/541,679, filed on Jul. 5, 2017, now Pat. No. 11,033,638.

(30) Foreign Application Priority Data
Jan. 7, 2015 (EP) 15150376.0

(51) Int. Cl. A61K 48/00; A61K 38/28; A61K 38/45; C07K 14/62; C12N 9/12; C12N 15/86

(52) U.S. Cl. CPC A61K 48/005; A61K 38/28; A61K 38/45; C07K 14/62; C12N 9/1205; C12N 15/86; C12Y 207/01002; C12N 2710/16143; C12N 2750/14143; C12N 2830/60

(57) ABSTRACT

The invention relates to a viral expression construct and related viral vector and composition and to their use wherein said construct and vector comprise elements a) and b):
a) a nucleotide sequence encoding an insulin operably linked to a first promoter,
b) a nucleotide sequence encoding a glucokinase operably linked to a second promoter and said viral expression construct and related viral vector comprise at least one of elements c), d) and e):
c) the first and the second promoters are positioned in reverse orientation within the expression construct,
d) the first and the second promoters are positioned in reverse orientation within the expression construct and are located adjacent to each other and
e) the first promoter is a CMV promoter, preferably a mini CMV promoter.

### FIELD OF THE INVENTION

The invention pertains to the medical field, comprising gene therapy compositions for use in the treatment of Diabetes Type 1 (T1D), Diabetes Type 2 (T2D) and/or Monogenic Diabetes, either in higher mammals, particularly pets and more particularly dogs; or in human beings.

### BACKGROUND OF THE INVENTION

The two main forms of diabetes mellitus are type 1 (T1D) and type 2 (T2D) (Diabetes care, 1997, 20-1183-1197). TID is characterized by a severe lack of insulin production due to specific destruction of the pancreatic β-cells. β-cell loss in TID is the result of an autoimmune mediated process, where a chronic inflammation called insulitis causes β-cell destruction (Eizirik D. L. et al, 2001, Diabetologia, 44:2115-2133 and Mathis D et al, 2001, Nature, 414: 792-798).

TID is one of the most common endocrine and metabolic conditions in childhood; incidence is rapidly increasing, especially among young children. TID is diagnosed when the autoimmune-mediated β-cell destruction is almost complete and patients need insulin-replacement therapy to survive. TID in an adult may present itself as T2D, with a slow deterioration in metabolic control, and subsequent progression to insulin dependency. This form is called latent autoimmune diabetes mellitus in adults (LADA) (Diabetes Atlas 4^{th} edition, 2009, International Diabetes Federation).

Lifelong insulin treatment is the therapy of choice for TID. While lifelong treatment with exogenous insulin successfully manages diabetes, correct maintenance of a normoglycemic state can be challenging, Chronic hyperglycemia leads to severe microvascular (retinopathy and nephropathy), macrovascular (stroke, myocardial infarction), and neurological complications. These devastating complications can be prevented by normalization of blood glucose levels. Brittle diabetes is one example of a difficult-to-manage disease. Additionally, in many underdeveloped countries, especially in less privileged families, access to self-care tools and also to insulin is limited and this may lead to severe handicap and early death in diabetic children (Diabetes Atlas 4^{th} edition, 2009, International Diabetes Federation, Beran D. et al 2006, Lancet, 368: 1689-1695, and Gale E. A., et al, 2006, Lancet, 368: 1626-1628). The most common cause of death in a child with diabetes, from a global perspective, is lack of access to insulin; thus the availability of a one-time gene therapy approach could make a difference in terms of prognosis when access to insulin is limited (Greenwood H. L. et al, 2006, PLoS Med 3.e381).

The reduction of hyperglycemia and maintenance of normoglycemia is a goal of any therapeutic approach to TID. The current therapy for most diabetic patients is based on regular subcutaneous injections of mixtures of soluble (short-acting) insulin and lente (long-acting) insulin preparations. Other therapeutical approaches include gene therapy, which would offer the potential advantage of an administration of a viral vector, which could ideally provide the necessary insulin through the lifetime of the diabetic subject.

WO 2012/007458 discloses the generation of two viral vectors, one expressing the insulin gene and one expressing the glucokinase gene as a treatment of diabetes.

However, there is still a need for an improved diabetes treatment wherein a lower dose of vector could be used, wherein a concomitant expression of each gene is provided in each transfected cell, wherein an attractive yield of the virus could be obtained and/or wherein potential induced side effects due to immunological properties of the capsid are lowered.

Therefore there is still a need for designing new treatments for diabetes which do not have all the drawbacks of existing treatments.

### DESCRIPTION OF THE INVENTION

The inventors designed improved gene therapy strategies based on adeno-associated viral (AAV) vector-mediated insulin/glucokinase muscle gene transfer to counteract diabetic hyperglycemia, dual-gene viral constructs encoding insulin and glucokinase were generated to ensure concomitant expression of both transgenes in transduced muscle cells.

Generation of dual-gene vectors will also allow decreasing vector dose, which in turn, should result in reduced risk of capsid-triggered immunity or other toxicities. From a regulatory point of view, the use of a dual vector will greatly facilitate the development of the treatment. Moreover, the use of a dual vector will allow for a dramatic reduction in the cost of manufacturing of AAV vectors. However, the skilled person knows that such a dual vector due to its size may not always be produced in sufficient yields to be used in a therapeutic setting and may not always be found to ensure acceptable expression levels of both transgenes. All dual vectors tested in the experimental part could be produced at acceptable titers and were found to be able to ensure acceptable expression levels of both transgenes.

Therefore the generation of such AAV dual vectors that contain both the insulin and glucokinase transgenes and potentially have improved therapeutic efficacy is not routine for a person skilled in the art, as demonstrated in the experimental part.

### Viral Expression Construct

In a first aspect there is provided a viral expression construct comprising the elements a) and b):
a) a nucleotide sequence encoding an insulin operably linked to a first promoter,
b) a nucleotide sequence encoding a glucokinase operably linked to a second promoter, and said viral expression construct comprises at least one of elements c), d) and e)
c) the first and the second promoters are positioned in reverse orientation within the expression construct,
d) the first and the second promoters are positioned in reverse orientation within the expression construct and are located adjacent to each other and
e) the first promoter is a CMV promoter, preferably a mini CMV promoter.

The definition of “viral expression construct”, “promoter”, “operatively linked” has been provided in the part of the description entitled “general definitions”. Within the context of the invention, elements a) and b) define the expression cassette of a viral expression construct of the invention as further explained in the part of the description entitled “general definitions”.

In the context of the invention, a nucleotide sequence encoding an insulin could be replaced by:
i. a nucleotide sequence comprising a nucleotide sequence that has at least 60% sequence identity or similarity with SEQ ID NO: 1;
ii. a nucleotide sequence the complementary strand of which hybridizes to a nucleic acid molecule of sequence of (i);
iii. a nucleotide sequence the sequence of which differs from the sequence of a nucleic acid molecule of (i) or (ii) due to the degeneracy of the genetic code; or,
iv. a nucleotide sequence that encodes an amino acid sequence that has at least 60% amino acid identity or similarity with an amino acid sequence encoded by a nucleotide sequence SEQ ID NO: 1.

A preferred nucleotide sequence encoding an insulin has at least 60%, 65%, 70%, 75%, 80%, 85%, 90%, 95%, 99%, 100% identity with SEQ ID NO:1. Identity may be assessed over the whole SEQ ID NO or over part thereof as explained in the part of the description entitled “general definitions”. SEQ ID NO:1 is a nucleotide sequence encoding human insulin. The nucleotide sequence encoding an insulin may be derived from any insulin gene, preferably from dog, human or rat; or a mutated insulin gene, or a codon optimized insulin gene, preferably from human, dog or rat as for example disclosed in WO 2012/007458 which is incorporated by reference in its entirety.

An insulin as used herein exerts at least a detectable level of an activity of an insulin as known to the skilled person. An activity of an insulin is the regulation of hyperglycemia.

This could be assessed using any technique known to the skilled person or as was done in the experimental part.

In the context of the invention, a nucleotide sequence encoding a glucokinase could be replaced by:
i. a nucleotide sequence comprising a nucleotide sequence that has at least 60% sequence identity or similarity with SEQ ID NO: 2;
ii. a nucleotide sequences the complementary strand of which hybridizes to a nucleic acid molecule of sequence of (i);
iii. a nucleotide sequence the sequence of which differs from the sequence of a nucleic acid molecule of (i) or (ii) due to the degeneracy of the genetic code; or,
iv. a nucleotide sequence that encodes an amino acid sequence that has at least 60% amino acid identity or similarity with an amino acid sequence encoded by a nucleotide sequence SEQ ID NO: 2.

A preferred nucleotide sequence encoding an insulin has at least 60%, 65%, 70%, 75%, 80%, 85%, 90%, 95%, 99%, 100% identity with SEQ ID NO:2. Identity may be assessed over the whole SEQ ID NO or over part thereof as explained in the part of the description entitled “general definitions”. SEQ ID NO:2 is a nucleotide sequence encoding human glucokinase. The nucleotide sequence encoding a glucokinase may be derived from any glucokinase gene, preferably from human or rat; or a mutated glucokinase gene, or a codon optimized glucokinase gene, preferably from human or rat as for example disclosed in WO 2012/007458 which is incorporated by reference in its entirety.

A glucokinase as used herein exerts at least a detectable level of an activity of a glucokinase as known to the skilled person. An activity of a glucokinase is to phosphorylate glucose. This activity could be assessed using assays known to the skilled person.

In the context of the invention, a first promoter is a promoter which is operatively linked to the insulin nucleotide sequence defined above and a second promoter is a promoter which is operatively linked to the glucokinase nucleotide sequence defined above.

In one embodiment, the first and second promoters are different. It is therefore not excluded that the first and second promoters are identical. In one embodiment, both promoters are cell-specific and/or tissue-specific, preferably both promoters are specific for skeletal muscle.

A preferred first promoter is a CMV promoter (element e).

In the context of the invention, a nucleotide sequence of a CMV promoter could be replaced by a nucleotide sequence comprising a nucleotide sequence that has at least 60% sequence identity or similarity with SEQ ID NO: 3. A preferred nucleotide sequence has at least 60%, 65%, 70%, 75%, 80%, 85%, 90%, 95%, 99%, 100% identity with SEQ ID NO:3. Identity may be assessed over the whole SEQ ID NO or over part thereof as explained in the part of the description entitled “general definitions”.

A first promoter as used herein (especially when his sequence is defined has having a minimal identity percentage with a given SEQ ID NO) should exert at least an activity of a promoter as known to the skilled person. Please be referred to the part of the description entitled “general definitions” for a definition of such activity. Preferably a first promoter defined has having a minimal identity percentage with a given SEQ ID NO should control transcription of the nucleotide sequence it is operably linked thereto (i.e. a nucleotide sequence encoding an insulin for the first promoter) as assessed in an assay known to the skilled person. The same holds for a second promoter with a nucleotide sequence encoding a glucokinase).

Preferably said CMV promoter is used together with an intronic sequence. In this context an intronic sequence may be replaced by a nucleotide sequence comprising a nucleotide sequence that has at least 60% sequence identity or similarity with SEQ ID NO: 4. A preferred nucleotide sequence has at least 60%, 65%, 70%, 75%, 80%, 85%, 90%, 95%, 99%, 100% identity with SEQ ID NO:4. Identity may be assessed over the whole SEQ ID NO or over part thereof as explained in the part of the description entitled “general definitions”.

In a more preferred embodiment, a CMV promoter is a mini CMV promoter. In the context of the invention, a nucleotide sequence of a mini CMV promoter could be replaced by a nucleotide sequence comprising a nucleotide sequence that has at least 60% sequence identity or similarity with SEQ ID NO: 5. A preferred nucleotide sequence has at least 60%, 65%, 70%, 75%, 80%, 85%, 90%, 95%, 99%, 100% identity with SEQ ID NO:5. Identity may be assessed over the whole SEQ ID NO or over part thereof as explained in the part of the description entitled “general definitions”.

In an even more preferred embodiment, a nucleotide sequence of a mini CMV promoter comprising a nucleotide sequence that has at least 60% sequence identity or similarity with SEQ ID NO: 24. A preferred nucleotide sequence has at least 60%, 65%, 70%, 75%, 80%, 85%, 90%, 95%, 99%, 100% identity with SEQ ID NO:24. Even more preferably, a nucleotide sequence a mini CMV promoter has at least 60% sequence identity or similarity with SEQ ID NO: 24 or at least 60%, 65%, 70%, 75%, 80%, 85%, 90%, 95%, 99%, 100% identity with SEQ ID NO:24 and has a length of 93, 94, 95, 96, 97, 98, 99, 100, 101, 102, 103, 104, 105, 106, 107, 109, 110 nucleotides.

Identity may be assessed over the whole SEQ ID NO or over part thereof as explained in the part of the description entitled “general definitions”.

In an embodiment, said mini CMV promoter may be used together with the intronic sequence defined above.

A preferred second promoter is a RSV promoter.

In the context of the invention, a nucleotide sequence of a RSV promoter could be replaced by a nucleotide sequence comprising a nucleotide sequence that has at least 60% sequence identity or similarity with SEQ ID NO: 6. A preferred nucleotide sequence has at least 60%, 65%, 70%, 75%, 80%, 85%, 90%, 95%, 99%, 100% identity with SEQ ID NO:6. Identity may be assessed over the whole SEQ ID NO or over part thereof as explained in the part of the description entitled “general definitions”.

Preferably said RSV promoter is used together with an intronic sequence. In this context an intronic sequence may be replaced by a nucleotide sequence comprising a nucleotide sequence that has at least 60% sequence identity or similarity with SEQ ID NO: 23. A preferred nucleotide sequence has at least 60%, 65%, 70%, 75%, 80%, 85%, 90%, 95%, 99%, 100% identity with SEQ ID NO:23. Identity may be assessed over the whole SEQ ID NO or over part thereof as explained in the part of the description entitled “general definitions”.

In a preferred embodiment, the first and the second promoters are positioned in reverse orientation within the viral expression construct (element c). In this embodiment, it implies that the insulin and the glucokinase nucleotide sequences are read in opposite directions. More preferably, in this configuration, the first and the second promoters are adjacent to each other (element d). In this context, “adjacent” means that 0, 2, 5, 10, 20, 30, 50, 100, 200, 300, 400, 500, 600, 700 bases are present between the first and the second promoters.

Several viral expression constructs are therefore encompassed by the present invention:
A viral expression construct comprising elements a), b) and c),
A viral expression construct comprising elements a), b) and d),
A viral expression construct comprising elements a), b) and e),
A viral expression construct comprising elements a), b) and e), wherein the CMV promoter is a mini CMV promoter,
A viral expression construct comprising elements a), b), d) and e),
A viral expression construct comprising elements a), b), d) and e), wherein the CMV promoter is a mini CMV promoter.

For each of these preferred viral expression constructs defined above, the second promoter is preferably a RSV promoter as defined herein.

In an embodiment, a viral expression construct is encompassed comprising elements a) and b) and at least one of elements c), d) and e), wherein the first promoter is a CMV promoter, preferably a mini CMV promoter and/or wherein the second promoter is a RSV promoter.

Additional sequences may be present in the viral expression construct of the invention as explained in detail in the part of the description entitled “general definitions”. Preferred additional sequences include ITRs, SV40 (i.e. which means SV40 polyadenylation signal) (SEQ ID NO: 22), bGH (i.e. which means bGH polyadenylation signal) (SEQ ID NO:7), SV40 polyadenylation signal and enhancer sequence (SEQ ID NO:30), SV40 enhancer sequence (SEQ ID NO: 33). Within the context of the invention, “ITRs” is intended to encompass one 5′ITR and one 3′ITR, each being derived from the genome of a AAV. Preferred ITRs are from AAV2 and are represented by SEQ ID NO: 31 (5′ ITR) and SEQ ID NO: 32 (3′ ITR). Within the context of the invention, it is encompassed to use the SV40 enhancer sequence either included in the SV40 polyadenylation signal (as SEQ ID NO:30) or as a separate sequence (as SEQ ID NO:33). It is also encompassed to use the SV40 polyadenylation signal and the SV40 enhancer sequence as two separate sequences (SEQ ID NO:22 and SEQ ID NO: 33) or as a single sequence (SEQ ID NO:30).

Each of these additional sequences may be present in the viral expression construct of the invention. (see for example as depicted in FIGS. 1, 2, 4, 7, 13, 16).

In an embodiment, the viral expression construct comprising elements a) and b), and at least one of elements c), d) and e) as earlier defined and further comprises:
ITRs that flank the expression cassette of said construct,
SV40 or bGH polyadenylation signals that are located at the 3′ of the nucleotide sequence encoding the glucokinase or insulin and/or
SV40 polyadenylation signals and enhancer sequence that is located at the 3′ of the nucleotide sequence encoding the glucokinase or insulin and/or
SV40 enhancer sequence that is located at the 5′ of the nucleotide sequence encoding the glucokinase or insulin.

In a preferred embodiment, the viral expression construct comprising elements a) and b), and at least one of elements c), d) and e) as earlier defined and further comprises ITRs that flank the expression cassette of said construct and optionally
SV40 or bGH polyadenylation signals that are located at the 3′ of the nucleotide sequence encoding the glucokinase or insulin and/or
SV40 polyadenylation signals and enhancer sequence that is located at the 3′ of the nucleotide sequence encoding the glucokinase or insulin and/or
SV40 enhancer sequence that is located at the 5′ of the nucleotide sequence encoding the glucokinase or insulin.

If the SV40 enhancer sequence is not included in the SV40 polyadenylation signal, the SV40 enhancer sequence is preferably located 5′ of the nucleotide sequence encoding the glucokinase or insulin.

These sequences were used in the experimental part in some of the constructs identified herein.

Therefore in one embodiment, for each of these preferred viral expression constructs defined above an additional sequence may be present selected from the group consisting of ITRs, SV40 polyadenylation signal, bGH polyadenylation signal, SV40 polyadenylation signal and enhancer sequence, SV40 enhancer sequence.

In a preferred embodiment, a viral expression construct is encompassed comprising elements a) and b) and at least one of elements c), d) and e),
wherein the first promoter is a CMV promoter, preferably a mini CMV promoter and/or
wherein the second promoter is a RSV promoter and/or
wherein an additional sequence is present which is selected from the group consisting of: ITRs, SV40 polyadenylation signal, bGH polyadenylation signal, SV40 polyadenylation signal and enhancer sequence, SV40 enhancer sequence.

Preferred ITRs are those of AAV2 which are represented by SEQ ID NO: 31 (5′ ITR) and SEQ ID NO: 32 (3′ ITR).

Preferred viral expression constructs comprise elements a) and b) and at least one of elements c), d) and e) and are such that the expression cassette as defined by elements a), b) and at least one of elements c), d), e) is flanked by a 5′ITR and a 3′ITR.

Other preferred viral expression constructs comprise elements a) and b) and at least one of elements c), d) and e) and are such that the expression cassette as defined by elements a), b) and at least one of c), d), e) is flanked by a 5′ITR and a 3′ITR. In addition, SV40 polyadenylation signals are present.

Other preferred viral expression constructs comprise elements a) and b) and at least one of elements c), d) and e) and are such that the expression cassette as defined by elements a), b) and at least one of c), d), e) is flanked by a 5′ITR and a 3′ITR. In addition, SV40 and bGH polyadenylation signals are present.

Other preferred viral expression constructs comprise elements a) and b) and at least one of elements c), d) and e) and are such that the expression cassette as defined by elements a), b) and at least one of c), d), e) is flanked by a 5′ITR and a 3′ITR. In addition, SV40 enhancer sequence is present. Other preferred viral expression constructs comprise elements a) and b) and at least one of elements c), d) and e) and are such that the expression cassette as defined by elements a), b) and at least one of c), d), e) is flanked by a 5′ITR and a 3′ITR. In addition, SV40 enhancer sequence and SV40 polyadenylation signals are present as two separate sequences.

Other preferred viral expression constructs comprise elements a) and b) and at least one of elements c), d) and e) and are such that the expression cassette as defined by elements a), b) and at least one of c), d), e) is flanked by a 5′ITR and a 3′ITR. In addition, SV40 enhancer sequence and SV40 polyadenylation signals are present as two separate sequences. In this embodiment, bGH polyadenylation signals are also present.

Other preferred viral expression constructs comprise elements a) and b) and at least one of elements c), d) and e) and are such that the expression cassette as defined by elements a), b) and at least one of c), d), e) is flanked by a 5′ITR and a 3′ITR. In addition, SV40 polyadenylation signals and enhancer sequence are present together with bGH polyadenylation signals are present.

Most preferred designed viral expression constructs include:
Construct A (represented by a nucleotide sequence comprising SEQ ID NO: 8),
Construct D (represented by a nucleotide sequence comprising SEQ ID NO: 9),
Construct E (represented by a nucleotide sequence comprising SEQ ID NO: 10),
Construct F (represented by a nucleotide sequence comprising SEQ ID NO: 11),
Construct G (represented by a nucleotide sequence comprising SEQ ID NO: 12),
Construct J (represented by a nucleotide sequence comprising SEQ ID NO: 13),
Construct K (represented by a nucleotide sequence comprising SEQ ID NO: 14),
Construct L (represented by a nucleotide sequence comprising SEQ ID NO: 15),
Construct M (represented by a nucleotide sequence comprising SEQ ID NO: 16).
Construct Q (represented by a nucleotide sequence comprising SEQ ID NO: 27).
Construct S (represented by a nucleotide sequence comprising SEQ ID NO: 29).

As the skilled person will understand, each of these viral expression constructs already comprise two ITRs from AAV2 (i.e. SEQ ID NO: 31 (5′ ITR) and SEQ ID NO: 32 (3′ ITR)).

Best results were obtained with constructs F (SEQ ID NO: 11), construct J (SEQ ID NO: 13), construct K (SEQ ID NO: 14), construct L (SEQ ID NO: 15), construct M (SEQ ID NO: 16), construct Q (SEQ ID NO: 27) and construct S (SEQ ID NO: 29).

Constructs L and Q comprise both bGH polyadenylation signal and SV40 polyadenylation signal sequences, the order of each of these 3′untranslated sequences being interchanged (see FIGS. 7 and 13).

Construct S comprises both bGH polyadenylation signal and SV40 polyadenylation signal and enhancer sequences (see FIG. 16).

As explained in the general part entitled “general definitions”, throughout this application, each time one refers to a specific nucleotide sequence SEQ ID NO (take SEQ ID NO: 8, 9, 10, 11, 12, 13, 14, 15, 16, 27, 29) representing the preferred constructs designed herein, one may replace it by:
i. a nucleotide sequence comprising a nucleotide sequence that has at least 60% sequence identity or similarity with SEQ ID NO: 8, 9, 10, 11, 12, 13, 14, 15, 16, 27 or 29;
ii. a nucleotide sequences the complementary strand of which hybridizes to a nucleic acid molecule of sequence of (i);
iii. a nucleotide sequence the sequence of which differs from the sequence of a nucleic acid molecule of (i) or (ii) due to the degeneracy of the genetic code.

Each nucleotide sequence described herein by virtue of its identity percentage (at least 60%) with a given nucleotide sequence respectively has in a further preferred embodiment an identity of at least 65%, 70%, 75%, 80%, 85%, 90%, 95%, 97%, 98%, 99% or more identity with the given nucleotide respectively. In a preferred embodiment, sequence identity is determined by comparing the whole length of the sequences as identified herein. Unless otherwise indicated herein, identity with a given SEQ ID NO means identity or similarity based on the full length of said sequence (i.e. over its whole length or as a whole).

A construct defined by its minimum identity (i.e. at least 60%) to a given SEQ ID NO as identified above is encompassed within the scope of the invention when this construct or a viral expression construct or a viral vector comprising this construct or a composition comprising this construct or vector is able to induce the expression of insulin and glucokinase in a cell, preferably in a muscle cell. The expression of both genes could be assessed using techniques known to the skilled person. In a preferred embodiment, said expression is assessed as carried out in the experimental part.

In a preferred embodiment, a viral expression construct is such that the construct is represented by a nucleotide sequence comprising SEQ ID NO: 8, 9, 10, 11, 12, 13, 14, 15, 16, 27 or 29 or a sequence having at least 60% identity with SEQ ID NO: 8, 9, 10, 11, 12, 13, 14, 15, 16, 27 or 29.

### Viral Vector

In a further aspect, there is provided a viral vector. A viral vector comprises a viral expression construct as defined above. A viral vector is further defined in the part of the description entitled “general definitions”. Preferably a viral vector is a retrovirus vector, an adenovirus vector, an adeno-associated virus vector, a herpesvirus vector, a polyoma virus vector or a vaccinia virus vector. More detail is also provided in the part of the description entitled “general definitions”.

In an embodiment, an adeno-associated viral vector is used comprising each of the elements defined earlier herein and a rAAV based genome comprising inverted terminal repeats (ITR) or a part thereof. Preferred ITRs are those of AAV2 which are represented by SEQ ID NO: 31 (5′ ITR) and SEQ ID NO: 32 (3′ ITR).

Preferably, said adeno-associated viral vector is an adeno-associated virus vector, more preferably an AAV1 vector.

A viral expression construct and a viral vector of the invention are preferably for use as a medicament. The medicament is preferably for preventing, delaying, curing, reverting and/or treating a diabetes. Diabetes may be Diabetes Type 1, Diabetes Type 2 or Monogenic Diabetes. The subject treated may be a higher mammal, e.g. cats, rodent, (preferably mice, rats, gerbils and guinea pigs, and more preferably mice and rats), or dogs, or in human beings.

### Composition

In a further aspect there is provided a composition comprising a viral expression construct or a viral vector as defined earlier herein. This composition is preferably called a gene therapy composition. Preferably, the composition is a pharmaceutical composition said pharmaceutical composition comprising a pharmaceutically acceptable carrier, adjuvant, diluents, solubilizer, filler, preservative and/or excipient.

Such pharmaceutically acceptable carrier, filler, preservative, solubilizer, diluent and/or excipient may for instance be found in Remington: The Science and Practice of Pharmacy, 20th Edition. Baltimore, MD: Lippincott Williams & Wilkins, 2000.

In a preferred embodiment, said composition is for use as a medicament, preferably for preventing, delaying, curing, reverting and/or treating a diabetes. Diabetes may be Diabetes Type 1, Diabetes Type 2 or Monogenic Diabetes. The subject treated may be a higher mammal, e.g. cats, rodent, (preferably mice, rats, gerbils and guinea pigs, and more preferably mice and rats), or dogs, or in human beings.

Said viral expression construct, viral vector and/or composition are preferably said to be able to be used for preventing, delaying, reverting, curing and/or treating a diabetes, when said viral expression construct, viral vector and/or composition are able to exhibit an anti-diabetes effect. An anti-diabetes effect may be reached when glucose disposal in blood is increased and/or when glucose tolerance is improved. This could be assessed using techniques known to the skilled person or as done in the experimental part. In this context, “increase” (respectively “improvement”) means at least a detectable increase (respectively a detectable improvement) using an assay known to the skilled person or using assays as carried out in the experimental part.

An anti-diabetes effect may also be observed when the progression of a typical symptom (i.e. insulitis, beta cell loss, . . . ) has been slowed down as assessed by a physician. A decrease of a typical symptom may mean a slow down in progression of symptom development or a complete disappearance of symptoms. Symptoms, and thus also a decrease in symptoms, can be assessed using a variety of methods, to a large extent the same methods as used in diagnosis of diabetes, including clinical examination and routine laboratory tests. Such methods include both macroscopic and microscopic methods, as well as molecular methods, X-rays, biochemical, immunohistochemical and others.

A medicament as defined herein (viral expression construct, viral vector, composition) is preferably able to alleviate one symptom or one characteristic of a patient or of a cell, tissue or organ of said patient if after at least one week, one month, six month, one year or more of treatment using a viral expression vector or a composition of the invention, said symptom or characteristic is no longer detectable.

A viral expression construct or a viral vector or a composition as defined herein for use according to the invention may be suitable for administration to a cell, tissue and/or an organ in vivo of individuals affected by or at risk of developing a diabetes, and may be administered in vivo, ex vivo or in vitro. Said combination and/or composition may be directly or indirectly administrated to a cell, tissue and/or an organ in vivo of an individual affected by or at risk of developing a diabetes, and may be administered directly or indirectly in vivo, ex vivo or in vitro. A preferred administration mode is intramuscular.

A viral expression construct or a viral vector or a composition of the invention may be directly or indirectly administered using suitable means known in the art. Improvements in means for providing an individual or a cell, tissue, organ of said individual with a viral expression construct or a viral vector or a composition of the invention are anticipated, considering the progress that has already thus far been achieved. Such future improvements may of course be incorporated to achieve the mentioned effect of the invention. A viral expression construct or a viral vector or a composition can be delivered as is to an individual, a cell, tissue or organ of said individual. Depending on the disease or condition, a cell, tissue or organ of said individual may be as earlier defined herein. When administering a viral expression construct or a viral vector or a composition of the invention, it is preferred that such viral expression construct or vector or composition is dissolved in a solution that is compatible with the delivery method. For intravenous, subcutaneous, intramuscular, intrathecal, intraarticular and/or intraventricular administration it is preferred that the solution is a physiological salt solution. Intramuscular administration is a preferred administration mode. More preferably intramuscular administration is carried out using a multineedle.

As encompassed herein, a therapeutically effective dose of a viral expression construct, vector or composition as mentioned above is preferably administered in a single and unique dose hence avoiding repeated periodical administration. More preferably, the single dose is administered to muscle tissue, and even more preferably by means of a unique multi-needle injection.

A further compound may be present in a composition of the invention. Said compound may help in delivery of the composition. Below is provided a list of suitable compounds: compounds capable of forming complexes, nanoparticles, micelles and/or liposomes that deliver each constituent as defined herein, complexed or trapped in a vesicle or liposome through a cell membrane. Many of these compounds are known in the art. Suitable compounds comprise polyethylenimine (PEI), or similar cationic polymers, including polypropyleneimine or polyethylenimine copolymers (PECs) and derivatives, synthetic amphiphiles (SAINT-18), Lipofectin™, DOTAP.

Depending on their identity, the skilled person will know which type of formulation is the most appropriate for the composition as defined herein.

In this context a further compound may be insulin that could be regularly injected.

### Method/Use

In a further aspect there is provided a method for preventing, delaying, reverting, curing and/or treating a diabetes wherein a viral expression construct or viral vector or composition as defined herein as defined herein is being used.

Such a method is preferably for alleviating one or more symptom(s) of diabetes in an individual, in a cell, tissue or organ of said individual or alleviate one or more characteristic(s) or symptom(s) of a cell, tissue or organ of said individual, the method comprising administering to said individual a viral expression construct or viral vector or a composition as defined herein.

In a further aspect there is provided a use of a viral expression construct or viral vector or a composition as defined herein for the manufacture of a medicament for preventing, delaying, reverting, curing and/or treating a diabetes.

Diabetes and the type of subject treated have been earlier defined herein.

In one embodiment said method or use is performed in vitro, for instance using a cell culture. Preferably, said method or use is in vivo. Each feature of these methods/uses has already been defined herein. In a method of the invention, a viral expression construct or vector and/or a composition may be combined with an additional compound known to be used for treating diabetes in an individual.

In a preferred embodiment, a treatment in a use or in a method according to the invention does not have to be repeated. Alternatively in a use or a method according to the invention said administration of the viral expression construct or of said composition may be repeated each year or each 2, 3, 4, 5, 6 years.

### General Definitions

### Identity/Similarity

In the context of the invention, a protein or a protein fragment as insulin or glucokinase is represented by an amino acid sequence.

In the context of the invention, a nucleic acid molecule as a nucleic acid molecule encoding an insulin or a nucleic acid molecule encoding a glucokinase is represented by a nucleic acid or nucleotide sequence which encodes a protein or a polypeptide or a protein fragment or a peptide or a derived peptide. A nucleic acid molecule may comprise a regulatory region.

It is to be understood that each nucleic acid molecule or protein or protein fragment or peptide or derived peptide or polypeptide or construct as identified herein by a given Sequence Identity Number (SEQ ID NO) is not limited to this specific sequence as disclosed. Each gene sequence or nucleotide sequence or nucleic acid sequence as identified herein encoding a given protein or polypeptide or construct or protein fragment or peptide or derived peptide or is itself a protein or a protein fragment or polypeptide or construct or peptide or derived peptide. Throughout this application, each time one refers to a specific nucleotide sequence SEQ ID NO (take SEQ ID NO: X as example) encoding a given polypeptide, one may replace it by:
i. a nucleotide sequence comprising a nucleotide sequence that has at least 60% sequence identity or similarity with SEQ ID NO: X;
ii. a nucleotide sequences the complementary strand of which hybridizes to a nucleic acid molecule of sequence of (i);
iii. a nucleotide sequence the sequence of which differs from the sequence of a nucleic acid molecule of (i) or (ii) due to the degeneracy of the genetic code; or,
iv. a nucleotide sequence that encodes an amino acid sequence that has at least 60% amino acid identity or similarity with an amino acid sequence encoded by a nucleotide sequence SEQ ID NO: X.

Throughout this application, each time one refers to a specific amino acid sequence SEQ ID NO (take SEQ ID NO: Y as example), one may replace it by: a polypeptide comprising an amino acid sequence that has at least 60% sequence identity or similarity with amino acid sequence SEQ ID NO: Y.

Each nucleotide sequence or amino acid sequence described herein by virtue of its identity or similarity percentage (at least 60%) with a given nucleotide sequence or amino acid sequence respectively has in a further preferred embodiment an identity or a similarity of at least 65%, 70%, 75%, 80%, 85%, 90%, 95%, 97%, 98%, 99% or more identity or similarity with the given nucleotide or amino acid sequence respectively. In a preferred embodiment, sequence identity or similarity is determined by comparing the whole length of the sequences as identified herein. Unless otherwise indicated herein, identity or similarity with a given SEQ ID NO means identity or similarity based on the full length of said sequence (i.e. over its whole length or as a whole).

Each non-coding nucleotide sequence (i.e. of a promoter or of another regulatory region) could be replaced by a nucleotide sequence comprising a nucleotide sequence that has at least 60% sequence identity or similarity with SEQ ID NO: A. A preferred nucleotide sequence has at least 60%, 65%, 70%, 75%, 80%, 85%, 90%, 95%, 99%, 100% identity with SEQ ID NO:A. Identity may be assessed over the whole SEQ ID NO or over part thereof as explained herein. In a preferred embodiment, such non-coding nucleotide sequence such as a promoter exhibits or exerts at least an activity of such a non-coding nucleotide sequence such as an activity of a promoter as known to the skilled person.

“Sequence identity” is herein defined as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO or on part thereof. Part thereof preferably means at least 10%, 20%, 30%, 40%, 50%, 60%, 70%, 80%, 90%, or 100% of both SEQ ID NO. In the art, “identity” also means the degree of sequence relatedness between amino acid or nucleic acid sequences, as the case may be, as determined by the match between strings of such sequences.

“Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, MD 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, WI. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

### Gene or Coding Sequence

“Gene” or “coding sequence” or “nucleic acid” or “nucleic” refers to a DNA or RNA region (the transcribed region) which “encodes” a particular protein such as an insulin or a glucokinase. A coding sequence is transcribed (DNA) and translated (RNA) into a polypeptide when placed under the control of an appropriate regulatory region, such as a promoter. A gene may comprise several operably linked fragments, such as a promoter, a 5′ leader sequence, an intron, a coding sequence and a 3′nontranslated sequence, comprising a polyadenylation site or a signal sequence. A chimeric or recombinant gene (such as a chimeric insulin gene or a chimeric glucokinase gene) is a gene not normally found in nature, such as a gene in which for example the promoter is not associated in nature with part or all of the transcribed DNA region. “Expression of a gene” refers to the process wherein a gene is transcribed into an RNA and/or translated into an active protein.

### Promoter

As used herein, the term “promoter” refers to a nucleic acid fragment that functions to control the transcription of one or more genes (or coding sequence), located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is preferentially active in specific types of differentiated cells/tissues, such as preferably a muscle cell or tissue derived therefrom.

### Operably Linked

“Operably linked” is defined herein as a configuration in which a control sequence such as a promoter sequence or regulating sequence is appropriately placed at a position relative to the nucleotide sequence of interest, preferably coding for an insulin or a glucokinase such that the promoter or control or regulating sequence directs or affects the transcription and/or production or expression of the nucleotide sequence of interest, preferably encoding an insulin or a glucokinase in a cell and/or in a subject. For instance, a promoter is operably linked to a coding sequence if the promoter is able to initiate or regulate the transcription or expression of a coding sequence, in which case the coding sequence should be understood as being “under the control of” the promoter. When one or more nucleotide sequences and/or elements comprised within a construct are defined herein to be “configured to be operably linked to an optional nucleotide sequence of interest”, said nucleotide sequences and/or elements are understood to be configured within said construct in such a way that these nucleotide sequences and/or elements are all operably linked to said nucleotide sequence of interest once said nucleotide sequence of interest is present in said construct.

### Viral Expression Construct

An expression construct carries a genome that is able to stabilize and remain episomal in a cell. Within the context of the invention, a cell may mean to encompass a cell used to make the construct or a cell wherein the construct will be administered. Alternatively a construct is capable of integrating into a cell's genome, e.g. through homologous recombination or otherwise. A particularly preferred expression construct is one wherein a nucleotide sequence encoding an insulin and a glucokinase as defined herein, is operably linked to a first and a second promoters as defined herein wherein said promoters are capable of directing expression of said nucleotide sequences (i.e. coding sequences) in a cell. Such a preferred expression construct is said to comprise an expression cassette. An expression cassette as used herein comprises or consists of a nucleotide sequence encoding an insulin and an nucleotide sequence encoding a glucokinase, each of them being operably linked to a promoter (i.e. a first and a second promoter) wherein said promoters are capable of directing expression of said nucleotide sequences. A viral expression construct is an expression construct which is intended to be used in gene therapy. It is designed to comprise part of a viral genome as later defined herein.

Expression constructs disclosed herein could be prepared using recombinant techniques in which nucleotide sequences encoding said insulin and glucokinased are expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34: 315 (describing cassette mutagenesis).

Typically, a nucleic acid or nucleotide sequence encoding an insulin and a glucokinase are used in an expression construct or expression vector. The phrase “expression vector” generally refers to a nucleotide sequence that is capable of effecting expression of a gene in a host compatible with such sequences. These expression vectors typically include at least suitable promoter sequences and optionally, transcription termination signals. An additional factor necessary or helpful in effecting expression can also be used as described herein. A nucleic acid or DNA or nucleotide sequence encoding an insulin and a glucokinase is incorporated into a DNA construct capable of introduction into and expression in an in vitro cell culture. Specifically, a DNA construct is suitable for replication in a prokaryotic host, such as bacteria, e.g., E. coli, or can be introduced into a cultured mammalian, plant, insect, (e.g., Sf9), yeast, fungi or other eukaryotic cell lines.

A DNA construct prepared for introduction into a particular host may include a replication system recognized by the host, an intended DNA segment encoding a desired polypeptide, and transcriptional and translational initiation and termination regulatory sequences operably linked to the polypeptide-encoding segment. The term “operably linked” has already been defined herein. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis.

The selection of an appropriate promoter sequence generally depends upon the host cell selected for the expression of a DNA segment. Examples of suitable promoter sequences include prokaryotic, and eukaryotic promoters well known in the art (see, e.g. Sambrook and Russell, 2001, supra). A transcriptional regulatory sequence typically includes a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). An expression vector includes the replication system and transcriptional and translational regulatory sequences together with the insertion site for the polypeptide encoding segment can be employed. In most cases, the replication system is only functional in the cell that is used to make the vector (bacterial cell as E. Coli). Most plasmids and vectors do not replicate in the cells infected with the vector. Examples of workable combinations of cell lines and expression vectors are described in Sambrook and Russell (2001, supra) and in Metzger et al. (1988) Nature 334: 31-36. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media.

Alternatively, a host cell is a cell that is part of a multicellular organism such as a transgenic plant or animal.

### Viral Vector

A viral vector or a gene therapy vector is a vector that comprises a viral expression construct as defined above.

A viral vector or a gene therapy vector is a vector that is suitable for gene therapy. Vectors that are suitable for gene therapy are described in Anderson 1998, Nature 392: 25-30; Walther and Stein, 2000, Drugs 60: 249-71; Kay et al., 2001, Nat. Med. 7: 33-40; Russell, 2000, J. Gen. Virol. 81: 2573-604; Amado and Chen, 1999, Science 285: 674-6; Federico, 1999, Curr. Opin. Biotechnol. 10: 448-53; Vigna and Naldini, 2000, J. Gene Med. 2: 308-16; Marin et al., 1997, Mol. Med. Today 3: 396-403; Peng and Russell, 1999, Curr. Opin. Biotechnol. 10: 454-7; Sommerfelt, 1999, J. Gen. Virol. 80: 3049-64; Reiser, 2000, Gene Ther. 7: 910-3; and references cited therein.

A particularly suitable gene therapy vector includes an Adenoviral and Adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications. (Russell, 2000, J. Gen. Virol. 81: 2573-2604; Goncalves, 2005, Virol J. 2(1):43) as indicated above. AAV vectors are even more preferred since they are known to result in very stable long term expression of transgene expression (up to 9 years in dog (Niemeyer et al, Blood. 2009 Jan. 22; 113(4):797-806) and ˜2 years in human (Nathwani et al, N Engl J Med. 2011 Dec. 22; 365(25):2357-65, Simonelli et al, Mol Ther. 2010 March; 18(3):643-50. Epub 2009 Dec. 1.)). Preferred adenoviral vectors are modified to reduce the host response as reviewed by Russell (2000, supra). Method for gene therapy using AAV vectors are described by Wang et al., 2005, J Gene Med. March 9 (Epub ahead of print), Mandel et al., 2004, Curr Opin Mol Ther. 6(5):482-90, and Martin et al., 2004, Eye 18(11):1049-55, Nathwani et al, N Engl J Med. 2011 Dec. 22; 365(25):2357-65, Apparailly et al, Hum Gene Ther. 2005 April; 16(4):426-34.

Another suitable gene therapy vector includes a retroviral vector. A preferred retroviral vector for application in the present invention is a lentiviral based expression construct. Lentiviral vectors have the ability to infect and to stably integrate into the genome of dividing and non-dividing cells (Amado and Chen, 1999 Science 285: 674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10: 448-53) and Vigna et al. (2000, J Gene Med 2000; 2: 308-16).

Other suitable gene therapy vectors include a herpes virus vector, a polyoma virus vector or a vaccinia virus vector.

A gene therapy vector comprises a nucleotide sequence encoding an insulin and a glucokinase to be expressed, whereby each of said nucleotide sequence is operably linked to the appropriate regulatory sequences. Such regulatory sequence will at least comprise a promoter sequence. Suitable promoters for expression of a nucleotide sequence encoding an insulin and a glycokinase from gene therapy vectors include e.g. cytomegalovirus (CMV) intermediate early promoter, viral long terminal repeat promoters (LTRs), such as those from murine moloney leukaemia virus (MMLV) rous sarcoma virus, or HTLV-1, the simian virus 40 (SV 40) early promoter and the herpes simplex virus thymidine kinase promoter. Suitable promoters are described below.

Several inducible promoter systems have been described that may be induced by the administration of small organic or inorganic compounds. Such inducible promoters include those controlled by heavy metals, such as the metallothionine promoter (Brinster et al. 1982 Nature 296: 39-42; Mayo et al. 1982 Cell 29: 99-108), RU-486 (a progesterone antagonist) (Wang et al. 1994 Proc. Natl. Acad. Sci. USA 91: 8180-8184), steroids (Mader and White, 1993 Proc. Natl. Acad. Sci. USA 90: 5603-5607), tetracycline (Gossen and Bujard 1992 Proc. Natl. Acad. Sci. USA 89: 5547-5551; U.S. Pat. No. 5,464,758; Furth et al. 1994 Proc. Natl. Acad. Sci. USA 91: 9302-9306; Howe et al. 1995 J. Biol. Chem. 270: 14168-14174; Resnitzky et al. 1994 Mol. Cell. Biol. 14: 1669-1679; Shockett et al. 1995 Proc. Natl. Acad. Sci. USA 92: 6522-6526) and the tTAER system that is based on the multi-chimeric transactivator composed of a tetR polypeptide, as activation domain of VP16, and a ligand binding domain of an estrogen receptor (Yee et al., 2002, U.S. Pat. No. 6,432,705).

A gene therapy vector may optionally comprise a further nucleotide sequence coding for a further polypeptide. A further polypeptide may be a (selectable) marker polypeptide that allows for the identification, selection and/or screening for cells containing the expression construct. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel (2001) “Molecular Cloning: A Laboratory Manual (3^{rd} edition), Cold Spring Harbor Laboratory, Cold Spring Harbor Laboratory Press, New York.

A gene therapy vector is preferably formulated in a pharmaceutical composition as defined herein. In this context, a pharmaceutical composition may comprise a suitable pharmaceutical carrier as earlier defined herein.

### Adeno-Associated Virus Vector (AAV Vector)

A preferred viral vector or a preferred gene therapy vector is an AAV vector. An AAV vector as used herein preferably comprises a recombinant AAV vector (rAAV). A “rAAV vector” as used herein refers to a recombinant vector comprising part of an AAV genome encapsidated in a protein shell of capsid protein derived from an AAV serotype as explained herein. Part of an AAV genome may contain the inverted terminal repeats (ITR) derived from an adeno-associated virus serotype, such as AAV1, AAV2, AAV3, AAV4, AAV5 and others. Preferred ITRs are those of AAV2 which are represented by SEQ ID NO: 31 (5′ ITR) and SEQ ID NO: 32 (3′ ITR).

Protein shell comprised of capsid protein may be derived from an AAV serotype such as AAV1, 2, 3, 4, 5 and others. A preferred AAV capsid is a AAV1 capsid. A preferred ITR is from the AAV2. A protein shell may also be named a capsid protein shell. rAAV vector may have one or preferably all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the present invention a capsid protein shell may be of a different serotype than the rAAV vector genome ITR.

A nucleic acid molecule represented by a nucleic acid sequence of choice is preferably inserted between the rAAV genome or ITR sequences as identified above, for example an expression construct comprising an expression regulatory element operably linked to a coding sequence and a 3′ termination sequence. Said nucleic acid molecule may also be called a transgene.

“AAV helper functions” generally refers to the corresponding AAV functions required for rAAV replication and packaging supplied to the rAAV vector in trans. AAV helper functions complement the AAV functions which are missing in the rAAV vector, but they lack AAV ITRs (which are provided by the rAAV vector genome). AAV helper functions include the two major ORFs of AAV, namely the rep coding region and the cap coding region or functional substantially identical sequences thereof. Rep and Cap regions are well known in the art, see e.g. Chiorini et al. (1999, J. of Virology, Vol 73(2): 1309-1319) or U.S. Pat. No. 5,139,941, incorporated herein by reference. The AAV helper functions can be supplied on a AAV helper construct. Introduction of the helper construct into the host cell can occur e.g. by transformation, transfection, or transduction prior to or concurrently with the introduction of the rAAV genome present in the rAAV vector as identified herein. The AAV helper constructs of the invention may thus be chosen such that they produce the desired combination of serotypes for the rAAV vector's capsid protein shell on the one hand and for the rAAV genome present in said rAAV vector replication and packaging on the other hand.

“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456 incorporated herein by reference.

A “transgene” is herein defined as a gene or a nucleic acid molecule (i.e. a molecule encoding an insulin and a molecule encoding a glucokinase) that has been newly introduced into a cell, i.e. a gene that may be present but may normally not be expressed or expressed at an insufficient level in a cell. In this context, “insufficient” means that although said insulin and glucokinase is expressed in a cell, a condition and/or disease as defined herein could still be developed. In this case, the invention allows the over-expression of an insulin and a glucokinase. The transgene may comprise sequences that are native to the cell, sequences that naturally do not occur in the cell and it may comprise combinations of both. A transgene may contain sequences coding for an insulin and a glucokinase and/or additional proteins as earlier identified herein that may be operably linked to appropriate regulatory sequences for expression of the sequences coding for an insulin and a glucokinase in the cell. Preferably, the transgene is not integrated into the host cell's genome.

“Transduction” refers to the delivery of an insulin and a glucokinase into a recipient host cell by a viral vector. For example, transduction of a target cell by a rAAV vector of the invention leads to transfer of the rAAV genome contained in that vector into the transduced cell. “Host cell” or “target cell” refers to the cell into which the DNA delivery takes place, such as the muscle cells of a subject. AAV vectors are able to transduce both dividing and non-dividing cells.

### Production of an AAV Vector

The recombinant AAV vector, including all combinations of AAV serotype capsid and AAV genome ITRs, is produced using methods known in the art, as described in Pan et al. (J. of Virology 1999, Vol 73(4):3410-3417) and Clark et al. (Human Gene Therapy, 1999, 10:1031-1039), incorporated herein by reference. In short, the methods generally involve (a) the introduction of the rAAV genome into a host cell, (b) the introduction of an AAV helper construct into the host cell, wherein the helper construct comprises the viral functions missing from the rAAV genome and (c) introducing a helper virus into the host cell. All functions for rAAV vector replication and packaging need to be present, to achieve replication and packaging of the rAAV genome into rAAV vectors. The introduction into the host cell can be carried out using standard virological techniques and can be simultaneously or sequentially. Finally, the host cells are cultured to produce rAAV vectors and are purified using standard techniques such as CsCl gradients (Xiao et al. 1996, J. Virol. 70: 8098-8108). Residual helper virus activity can be inactivated using known methods, such as for example heat inactivation. The purified rAAV vector is then ready for use in the methods. High titres of more than 10¹² particles per ml and high purity (free of detectable helper and wild type viruses) can be achieved (Clark et al. supra and Flotte et al. 1995, Gene Ther. 2: 29-37).

The rAAV genome present in a rAAV vector comprises at least the nucleotide sequences of the inverted terminal repeat regions (ITR) of one of the AAV serotypes (preferably the ones of serotype AAV2 as disclosed earlier herein), or nucleotide sequences substantially identical thereto or nucleotide sequences having at least 60% identity thereto, and nucleotide sequence encoding an insulin and a glucokinase (under control of a suitable regulatory element) inserted between the two ITRs. A vector genome requires the use of flanking 5′ and a 3′ ITR sequences to allow for efficient packaging of the vector genome into the rAAV capsid.

The complete genome of several AAV serotypes and corresponding ITR has been sequenced (Chiorini et al. 1999, J. of Virology Vol. 73, No. 2, p 1309-1319). They can be either cloned or made by chemical synthesis as known in the art, using for example an oligonucleotide synthesizer as supplied e.g. by Applied Biosystems Inc. (Fosters, CA, USA) or by standard molecular biology techniques. The ITRs can be cloned from the AAV viral genome or excised from a vector comprising the AAV ITRs. The ITR nucleotide sequences can be either ligated at either end to the nucleotide sequence encoding one or more therapeutic proteins using standard molecular biology techniques, or the wild type AAV sequence between the ITRs can be replaced with the desired nucleotide sequence.

Preferably, the rAAV genome as present in a rAAV vector does not comprise any nucleotide sequences encoding viral proteins, such as the rep (replication) or cap (capsid) genes of AAV. This rAAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

The rAAV genome as present in said rAAV vector further comprises a promoter sequence operably linked to the nucleotide sequence encoding an insulin and a glucokinased. Preferred promoter sequences are promoters which confer expression in muscle cells and/or muscle tissues. Examples of such promoters include a CMV and a RSV promoters as earlier defined herein.

A suitable 3′ untranslated sequence may also be operably linked to the nucleotide sequence encoding an insulin and a glucokinase. Suitable 3′ untranslated regions may be those naturally associated with the nucleotide sequence or may be derived from different genes, such as for example the bovine growth hormone 3′ untranslated region (bGH polyadenylation signal (SEQ ID NO:7), SV40 polyadenylation signal (SEQ ID NO:22), SV40 polyadenylation signal and enhancer sequence (SEQ ID NO: 30).

Within the context of the invention, when one refers to “SV40”, it means SV40 polyadenylation signal. When one refers to “SV40 enhancer sequence”, it means SV40 polyadenylation signal and enhancer sequence. However, the invention also encompasses the use of SV40 polyadenylation signal (SEQ ID NO:22) and SV40 enhancer sequence (SEQ ID NO:33) as two separate sequences.

These sequences were used in the preferred constructs prepared in the experimental part. Constructs L and Q comprise both bGH polyA and SV40 polyadenylation signal sequences, the order of each of these 3′untranslated sequences being interchanged (see FIGS. 7 and 13).

Construct S comprises both bGH polyA and SV40 polyadenylation signal and enhancer sequences (see FIG. 16).

Optionally, additional nucleotide sequences may be operably linked to the nucleotide sequence(s) encoding an insulin and a glucokinase, such as nucleotide sequences encoding signal sequences, nuclear localization signals, expression enhancers, and the like.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a viral expression construct, viral vector, composition, gene therapy composition, as defined herein may comprise additional component(s) than the ones specifically identified, said additional component(s) not altering the unique characteristic of the invention.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “approximately” or “about” when used in association with a numerical value (approximately 10, about 10) preferably means that the value may be the given value of 10 more or less 1% of the value.

All patent and literature references cited in the present specification are hereby incorporated by reference in their entirety. In this context WO 2012/007458 is incorporated by reference in its entirety. Each embodiment as identified herein may be combined together unless otherwise indicated.

The invention is further explained in the following examples. These examples do not limit the scope of the invention, but merely serve to clarify the invention.

### FIGURE LEGENDS

FIG. 1. Schematic representation of the dual-gene RSV-rGck-CMV-hIns AAV construct described in A.2. ITR: Inverted Terminal Repeat; RSV: Rous Sarcoma Virus promoter; rGck: rat glucokinase cDNA; SV40: simian virus 40 polyadenylation signal; CMV: cytomegalovirus promoter; hINS: human insulin cDNA.

Construct A: RSV-rGck-CMV-hIns (size: 4.9 kb) (SEQ ID NO: 8) is depicted in FIG. 1.

FIG. 2. Schematic representation of the single-gene AAV constructs described in A.2. ITR: Inverted Terminal Repeat; CMV: cytomegalovirus promoter; hINS: human insulin cDNA; SV40: simian virus 40 polyadenylation signal; RSV: Rous Sarcoma Virus promoter; rGck: rat glucokinase cDNA.

Construct B is depicted in FIG. 2: CMV-hIns (SEQ ID NO: 17).

Construct C is depicted in FIG. 2: RSV-rGck (SEQ ID NO: 18).

FIG. 3. Expression of insulin and glucokinase in HEK293 cells. The left histogram represents the expression of insulin in cells transfected with CMV-hIns (B) or RSV-rGck-CMV-hIns (A) plasmids. The right histogram represents the expression of glucokinase in cells transfected with RSVr-Gck (C) or RSV-rGck-CMV-hIns (A).

FIG. 4. Schematic representation of the dual-gene AAV constructs described in A.3. ITR: Inverted Terminal Repeat; CMV: cytomegalovirus promoter; hINS: human insulin cDNA; SV40: simian virus 40 polyadenylation signal; RSV: Rous Sarcoma Virus promoter; hGck: human glucokinase cDNA; bGH: bovine growth hormone polyadenylation signal.

Construct D is depicted in FIG. 4: CMV-hIns-RSV-hGck (size: 4.7 kb) (SEQ ID NO:9).

Construct E is depicted in FIG. 4: RSV-hGck-CMV-hIns (size: 4.7 kb) (SEQ ID NO:10).

Construct F is depicted in FIG. 4: CMV-hIns(rev)-RSV-hGck (size: 4.7 kb) (SEQ ID NO: 11).

Construct G is depicted in FIG. 4: RSV-hGck-CMV-hlns(rev) (size: 4.7 kb) (SEQ ID NO: 12).

FIG. 5. Schematic representation of the single-gene AAV constructs described in A.3. ITR: Inverted Terminal Repeat; CMV: cytomegalovirus promoter; hINS: human insulin cDNA; SV40: simian virus 40 polyadenylation signal; RSV: Rous Sarcoma Virus promoter; hGck: human glucokinase cDNA; bGH: bovine growth hormone polyadenylation signal.

Construct H is depicted in FIG. 5: CMV-hIns (SEQ ID NO:19).

Construct I is depicted in FIG. 5: RSV-hGck (SEQ ID NO: 20).

FIG. 6. Expression of insulin and glucokinase in HEK293 cells. The left histogram represents the expression of human insulin in cells transfected with CMV-hIns (construct H), CMV-hlns-RSV-hGck (construct D), RSV-hGck-CMV-hlns (construct E), CMV-hlns(rev)-RSV-hGck (construct F) or RSV-hGck-CMV-hlns(rev) (construct G) plasmids. The right histogram represents the expression of human glucokinase in cells transfected with RSV-hGck (construct I), CMV-hlns-RSV-hGck (construct D), RSV-hGck-CMV-hlns (construct E), CMV-hlns(rev)-RSV-hGck (construct F) or RSVh-Gck-CMV-hIns(rev) (construct G).

FIG. 7. Schematic representation of the dual-gene AAV constructs described in A.4. ITR: Inverted Terminal Repeat; MiniCMV: minicytomegalovirus promoter; hINS: human insulin cDNA; SV40: simian virus 40 polyadenylation signal; RSV: Rous Sarcoma Virus promoter; hGck: human glucokinase cDNA; bGH: bovine growth hormone polyadenylation signal.

Construct J is depicted in FIG. 7: miniCMV-hlns-RSV-hGck (size: 4 kb) (SEQ ID NO:13).

Construct K is depicted in FIG. 7: RSV-hGck-miniCMV-hlns (size: 4 kb) (SEQ ID NO:14).

Construct L is depicted in FIG. 7: miniCMV-hlns(rev)-RSV-hGck (size: 4 kb) (SEQ ID NO:15).

Construct M is depicted in FIG. 7: RSV-hGck-miniCMV-hlns(rev) (size: 4 kb) (SEQ ID NO:16).

FIG. 8. Schematic representation of the single-gene AAV described in A.4. ITR: Inverted Terminal Repeat; MiniCMV: minicytomegalovirus promoter; INS: human insulin cDNA; SV40: simian virus 40 polyadenylation signal; RSV: Rous Sarcoma Virus promoter; Gck: human glucokinase cDNA; bGH: bovine growth hormone polyadenylation signal.

Construct N is depicted in FIG. 8: miniCMV-hIns (SEQ ID NO:21).

Construct I is depicted in FIG. 8: RSV-hGcK-bGH (SEQ ID NO:20).

FIG. 9. Expression levels of insulin and glucokinase in HEK293 cells. The left histogram represents the expression of human insulin in cells transfected with miniCMV-Ins (construct N), miniCMV-hlns-RSV-Gck (construct J), RSV-hGck-miniCMV-hlns (construct K), miniCMV-hlns(rev)-RSV-hGck (construct L) or RSV-hGck-miniCMV-hIns(rev) (construct M) plasmids. The right histogram represents the expression of human glucokinase in cells transfected with RSV-hGck (construct I), miniCMV-hIns-RSV-hGck (construct J), RSV-hGck-miniCMV-hlns (construct K), miniCMV-hlns(rev)-RSV-hGck (construct L) or RSV-hGck-miniCMV-hlns(rev) (construct M) plasmids.

FIG. 10. AAV-mediated expression levels of insulin and glucokinase in the skeletal muscle of wild-type animals. Three weeks after vector administration, insulin (A) and glucokinase (B) expression was analysed by quantitative real time PCR in tibialis and gastrocnemius of control uninjected mice (CT), or in mice injected with the combination of the single vectors AAV1-miniCMV-hINS and AAV1-RSV-hGck (constructs N+I) or with the dual vector AAV1-miniCMV-hINS-rev-RSV-hGck (construct L). The amount of insulin and glucokinase was normalized to 36B4 expression. N.D., non detected, a.u. arbitrary units.

FIG. 11. Comparison of the ability to dispose of glucose after a load in animals injected with either a combination of single vectors or a dual-gene AAV vector. (A) Control mice (CT), mice injected with the combination of single vectors AAV1-miniCMV-hINS and AAV1-RSV-hGck (constructs N+I) and mice injected with the dual viral vector AAV1-miniCMV-hINS-rev-RSV-hGck (construct L) were given an intraperitoneal injection of 2 g glucose/kg body weight. Blood samples were taken from the tail of the animals at indicated time points and glucose concentration was determined. (B) The area under the curve (AUC) of the glucose tolerance test was calculated. a.u. arbitrary units. *p<0.05 vs N+I.

FIG. 12. Comparison of the ability to dispose of glucose after a load in diabetic animals injected with either a combination of single vectors or a dual-gene AAV vector. Healthy mice (No STZ), diabetic control mice (CT), diabetic mice injected with the combination of single vectors AAV1-miniCMV-hINS and AAV1-RSV-hGck (constructs N+I), and diabetic mice injected with the dual viral vector AAV1-miniCMV-hINS-rev-RSV-hGck (construct L) were given an intraperitoneal injection of 1 g glucose/kg body weight. (A) Fasting glucose levels. (B) Blood samples were taken from the tail of the animals at indicated time points and glucose concentration was determined. (C) The area under the curve (AUC) of the glucose tolerance test was calculated. a.u., arbitrary units. * p<0.05 vs N+I

FIG. 13. Schematic representation of the dual-gene and single-gene AAV described in A.5. ITR: Inverted Terminal Repeat; MiniCMV: minicytomegalovirus promoter; INS: human insulin cDNA; SV40: simian virus 40 polyadenylation signal; RSV: Rous Sarcoma Virus promoter; Gck: human glucokinase cDNA; bGH: bovine growth hormone polyadenyilation signal.

Construct O is depicted in FIG. 13: miniCMV-hIns-bGH (size: 1.4 kb) (SEQ ID NO:25).

Construct P is depicted in FIG. 13: RSV-hGck-SV40 (size: 2.9 kb) (SEQ ID NO:26).

Construct Q is depicted in FIG. 13: miniCMV-hIns-bGH(rev)-RSV-hGck-SV40 (size: 4 kb) (SEQ ID NO:27).

FIG. 14. AAV-mediated expression levels of insulin and glucokinase in the skeletal muscle of wild-type animals. Three weeks after vector administration, insulin (A) and glucokinase (B) expression was analysed by quantitative real time PCR in tibialis and gastrocnemius of control uninjected mice (CT), or in mice injected with the combination of the single vectors AAV1-miniCMV-hIns-bGH and AAV1-RSV-hGck-SV40 (constructs O+P) or with the dual vector AAV1-miniCMV-Insulin-bGH(rev)-RSV-Glucokinase-SV40 (construct Q). The amount of insulin and glucokinase was normalized to 36B4 expression. N.D., non detected. a.u., arbitrary units. * p<0.05 vs O+P

FIG. 15. Comparison of the ability to dispose of glucose after a load in animals injected with either a combination of single vectors or a dual-gene AAV vector. (A) Control mice (CT), mice injected with the combination of single vectors AAV1-miniCMV-hIns-bGH and AAV1-RSV-hGck-SV40 (constructs O+P) and mice injected with the dual viral vector AAV1-miniCMV-Insulin-bGH(rev)-RSV-Glucokinase-SV40 (construct Q) were given an intraperitoneal injection of 2 g glucose/kg body weight. Blood samples were taken from the tail of the animals at indicated time points and glucose concentration was determined. (B) The area under the curve (AUC) of the glucose tolerance test was calculated. a.u., arbitrary units. * p<0.05 vs O+P

FIG. 16. Schematic representation of the dual-gene and single-gene AAV described in A.6. ITR: Inverted Terminal Repeat; MiniCMV: minicytomegalovirus promoter; INS: human insulin cDNA; SV40 enhancer: SV40 enhancer and simian virus 40 polyadenylation signal; RSV: Rous Sarcoma Virus promoter; Gck: human glucokinase cDNA; bGH: bovine growth hormone polyadenylation signal.

Construct R is depicted in FIG. 16: miniCMV-hIns-SV40enhancer (size: 1.6 kb) (SEQ ID NO:28).

Construct S is depicted in FIG. 16: miniCMV-hIns-SV40enhancer(rev)-RSV-hGck-bGH (size: 4.2 kb) (SEQ ID NO:29).

FIG. 17. AAV-mediated expression levels of insulin and glucokinase in the skeletal muscle of wild-type animals. Three weeks after vector administration, insulin (A) and glucokinase (B) expression was analysed by quantitative real time PCR in tibialis and gastrocnemius of control uninjected mice (CT), or in mice injected with the combination of the single vectors AAV1-miniCMV-hIns-SV40enhancer and AAV1-RSV-hGck (constructs R+I) or with the dual vector AAV1-miniCMV-hIns-SV40enhancer(rev)-RSV-hGck-bGH (construct S). The amount of insulin and glucokinase was normalized to 36B4 expression. N.D., non detected. a.u., arbitrary units. *p<0.05 vs R+I.

FIG. 18. Comparison of the ability to dispose of glucose after a load in animals injected with either a combination of single vectors or a dual-gene AAV vector. (A) Control mice (CT), mice injected with the combination of single vectors AAV1-miniCMV-hIns-SV40enhancer and AAV1-RSV-hGck (R+I) and mice injected with the dual viral vector AAV1-miniCMV-hIns-SV40enhancer(rev)-RSV-hGck-bGH (S) were given an intraperitoneal injection of 2 g glucose/kg body weight. Blood samples were taken from the tail of the animals at indicated time points and glucose concentration was determined. (B) The area under the curve (AUC) of the glucose tolerance test was calculated. a.u., arbitrary units. * p<0.05 vs R+I.

### EXAMPLES

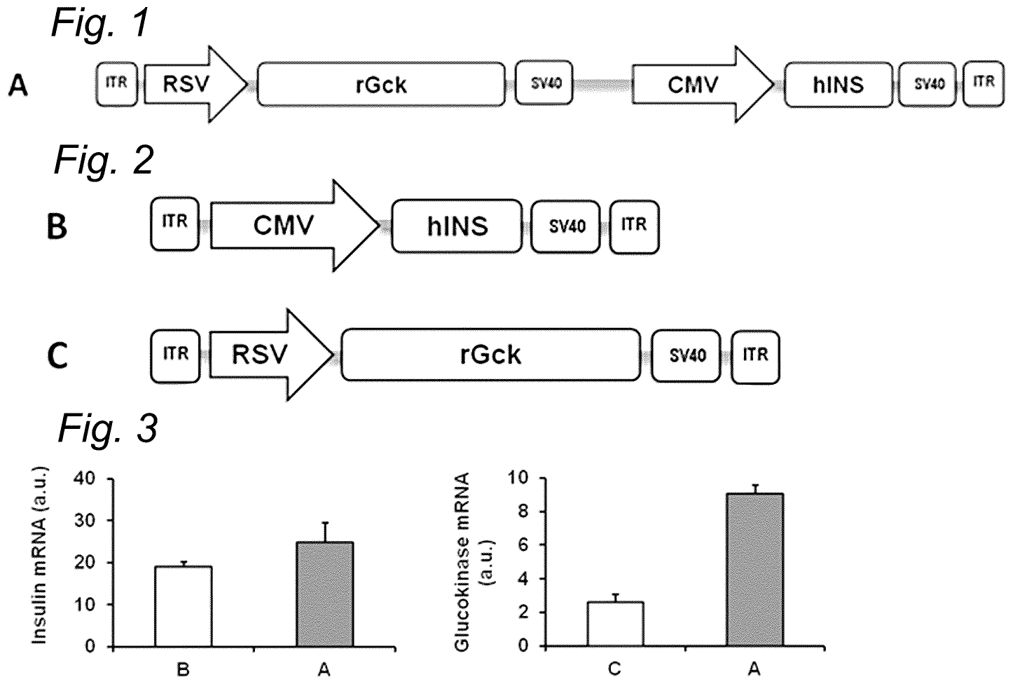

Throughout the application, one refers to constructs or vectors based on/comprising constructs A to S. The letter identifies the type of construct used and the same letter could be used to refer to a vector based on/derived from and/or comprising said construct. This is the reason why the ITRs are present in each of the FIG. 1, 2, 4, 5, 7, 8, 13 or 16 depicting each of the AAV viral vectors comprising said construct.

### A. Generation of Dual-Gene Adeno-Associated Viral (AAV) Vector Constructs for the Concomitant Expression of Insulin and Glucokinase

In order to develop more effective gene therapy strategies based on adeno-associated viral vector-mediated insulin/glucokinase muscle gene transfer to counteract diabetic hyperglycemia, dual-gene viral constructs encoding insulin and glucokinase were generated to ensure concomitant expression of both transgenes in transduced muscle cells.

Generation of dual-gene AAV1-Ins+Gck vectors will also allow decreasing vector dose, which in turn, should result in reduced risk of capsid-triggered immunity or other toxicities. From a regulatory point of view, the use of a dual vector will greatly facilitate the development of the treatment. Moreover, the use of a dual vector will allow for a dramatic reduction in the cost of manufacturing of AAV vectors.

The generation of such AAV dual vectors that contain both the insulin and glucokinase transgenes and potentially have improved therapeutic efficacy is not, however, entirely routine for a person skilled in the art, as demonstrated below.

In the experimental part, the nucleotide sequence encoding insulin was SEQ ID NO:1, the nucleotide sequence encoding glucokinase was SEQ ID NO:2. The nucleotide sequence of the CMV promoter was SEQ ID NO: 3 used with associated intronic sequence SEQ ID NO:4. The nucleotide sequence of the RSV promoter was SEQ ID NO: 6 with associated intronic sequence SEQ ID NO:23. The nucleotide sequence of the mini CMV promoter was SEQ ID NO:5. The nucleotide sequence of the bGH regulatory region was SEQ ID NO: 7. The nucleotide sequence of the SV40 was SEQ ID NO: 22.

### A.1. Dual-Gene AAV-CMV-Insulin-CMV-Glucokinase Construct

In the therapeutic approach that utilized 2 different AAV1 vectors to mediate the gene transfer to the skeletal muscle of the insulin and glucokinase genes when administered to mice and dogs (Mas, A. et al., Diabetes (2006) 55:1546-1553, Callejas, D. et al. Diabetes (2013) 62:1718-1729), the expression of both transgenes was driven by the CMV promoter. Therefore, the most obvious option to be considered while generating the dual-gene AAV constructs would have been to use CMV-Insulin and CMV-Glucokinase expression cassettes within the same vector. However, this option was discarded because the presence of the same promoter in 2 regions within the same construct increases dramatically the high risk of intramolecular recombination events that are sometimes observed during AAV production due to the presence of repeated sequences.

### A.2. Dual-Gene CMV-Insulin-RSV-Glucokinase AAV Constructs

Taking into account the restrictions on the use of promoters discussed above, the ubiquitous Rous Sarcoma Virus (RSV) promoter was chosen to drive expression of glucokinase in the dual-gene AAV construct. This promoter was selected because, similar to the CMV promoter, it has been reported to mediate strong transgene expression in muscle cells (Yue Y. et al, 2002, Biotechniques, 33:672, p 676 Development of Multiple Cloning Site cis-Vectors for Recombinant Adeno-Associated Virus Production). Additionally, its small size is convenient given the limited cloning capacity of AAV vectors.

A dual-gene AAV1-Ins+Gck construct bearing the human insulin coding sequence driven by the CMV promoter and the rat glucokinase coding sequence driven by the RSV promoter (FIG. 1) was generated. In this dual-gene construct the SV40 polyA sequence was cloned after the insulin and glucokinase genes:
Construct A: RSV-rGck-CMV-hIns (size: 4.9 kb) (SEQ ID NO: 8) is depicted in FIG. 1.

In addition to the previously described dual-gene AAV1-Ins+Gck construct, two additional single-gene plasmids encoding either human insulin or rat glucokinase were generated, using the same AAV backbone (FIG. 2), for comparison with the dual-gene AAV1-Ins+Gck construct:
Construct B is depicted in FIG. 2: CMV-hIns (SEQ ID NO: 17).
Construct C is depicted in FIG. 2: C: RSV-rGck (SEQ ID NO: 18).

The function of the dual-gene plasmid RSV-rGck-CMV-hIns (construct A) was assessed in vitro before AAV production and insulin and glucokinase were expressed at very high levels (FIG. 3).

Having verified the functionality of the RSV-rGck-CMV-hIns (construct A) in vitro, the plasmid was used to produce the corresponding dual-gene AAV1 vector in HEK293 cells. The yield of the vector batch was, however, low. The first production of AAV1-RSV-rGck-CMV-hIns rendered no AAV vectors and the yield of the second production run was 4E11 viral genomes (vg)/roller bottle (RB), considerably lower than our in house average yield for AAV1 production (expected yield: 2E12 vg/RB). The final size of the AAV constructs was close to the limit of encapsidation capacity of the AAV1, and the observation of low yields could be consistent with the low efficiency of encapsidation of oversized genomes. Nevertheless, this result was not foreseeable because in some cases AAV constructs of approximately 5 kb have been successfully produced by our lab.

### A.3. Optimized CMV-Insulin-RSV-Glucokinase Dual-Gene AAV Constructs

Given the relative low yield of the AAV batches produced with the previous dual-gene AAV constructs, we decided to completely remake the dual insulin and glucokinase expression cassettes. To this end, we designed a novel modular system that allowed us the test different combinations of coding sequences (optimized or not, and from different species) and cis-acting sequences (promoters, polyAs) at minimum effort and within optimal size for encapsidation. This new approached greatly simplified vector design.

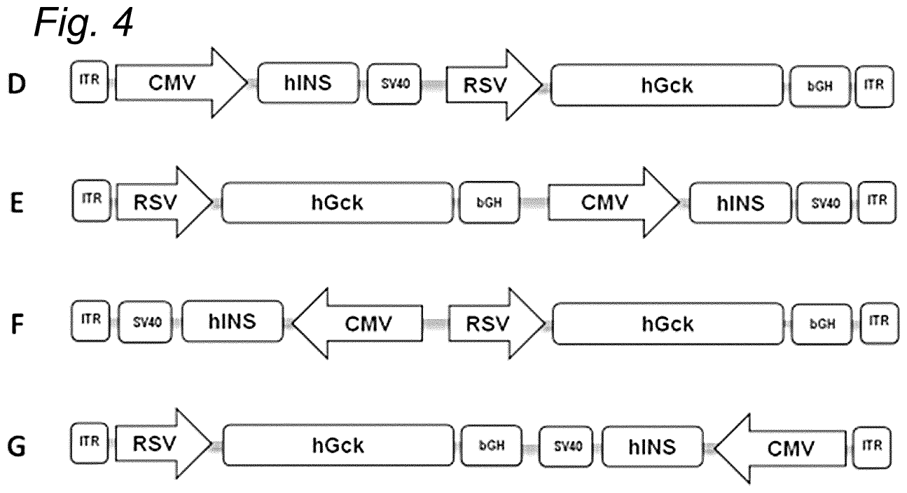

First, we generated 4 additional dual-gene constructs containing the human insulin coding sequence under the control of the CMV promoter and the human glucokinase coding sequence driven by the RSV promoter. We tested the effect of positioning the insulin expression cassette upstream of the glucokinase expression cassette and viceversa, and also in reverse orientation (FIG. 4).

In addition, in this new set of constructs, the CMV-hInsulin cassette included the SV40 polyA sequence whereas the bovine growth hormone polyA sequence was cloned in the RSV-hGlucokinase cassette, as the latter is shorter and mediates higher transgene expression than the SV40 polyA (Azzoni A R, J Gene Med. 2007: The impact of polyadenylation signals on plasmid nuclease-resistance and transgene expression). The new constructs are:
Construct D is depicted in FIG. 4: CMV-hIns-RSV-hGck (size: 4.7 kb) (SEQ ID NO:9).
Construct E is depicted in FIG. 4: RSV-hGck-CMV-hIns (size: 4.7 kb) (SEQ ID NO:10).
Construct F is depicted in FIG. 4: CMV-hIns(rev)-RSV-hGck (size: 4.7 kb) (SEQ ID NO: 11).
Construct G is depicted in FIG. 4: RSV-hGck-CMV-hIns(rev) (size: 4.7 kb) (SEQ ID NO: 12).

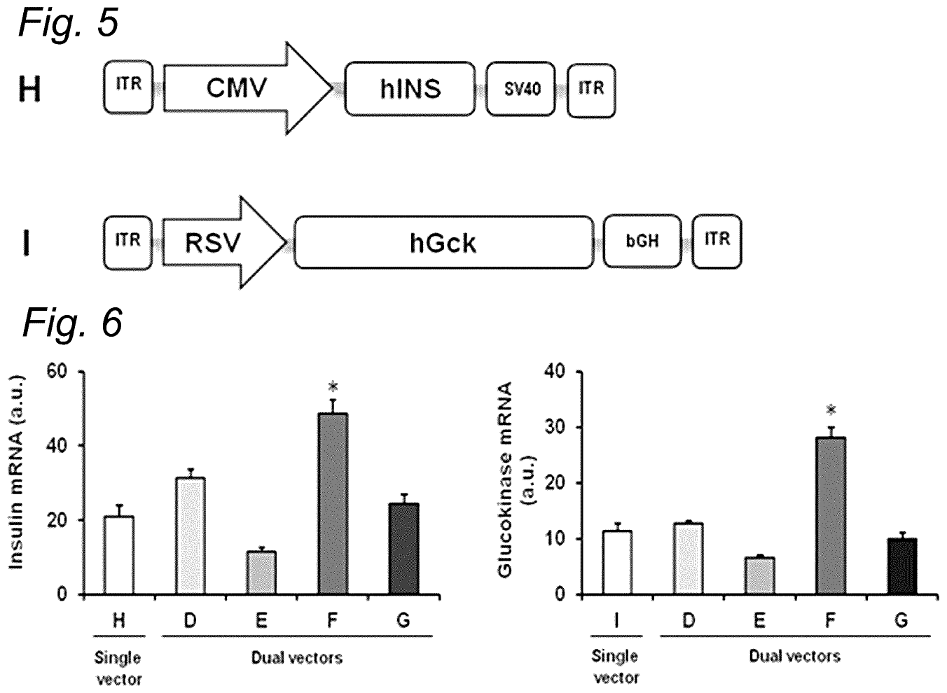

In addition to the aforementioned 4 dual-gene AAV1-Ins+Gck constructs (constructs D, E, F and G)), two additional single-gene plasmids encoding either insulin or glucokinase were generated using the same AAV backbone (FIG. 5) for comparison with the four new dual-gene AAV1-Ins+Gck constructs:
Construct H is depicted in FIG. 5: CMV-hIns (SEQ ID NO: 19).
Construct I is depicted in FIG. 5: RSV-hGck (SEQ ID NO: 20).

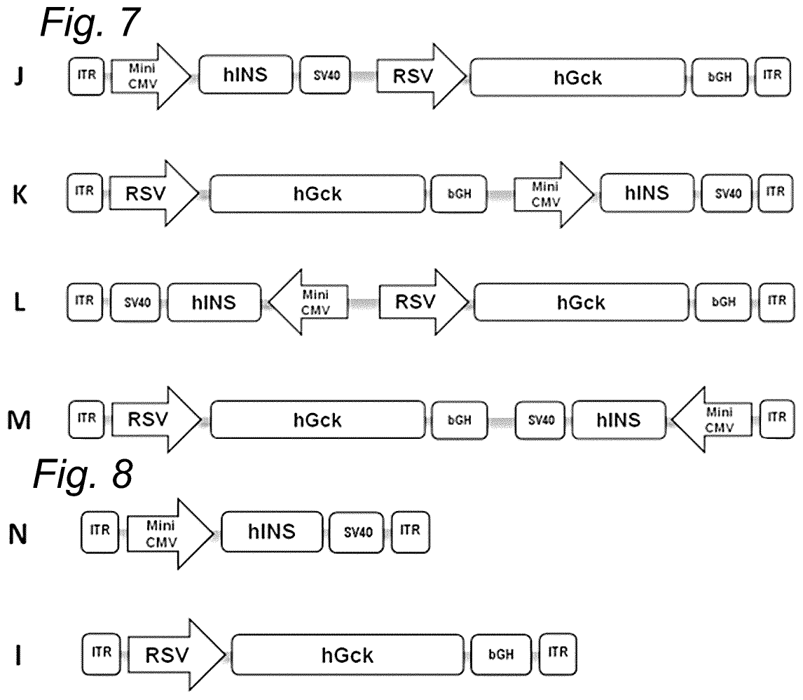

We assessed the function of the dual-gene constructs D, E, F and G plasmids in vitro in HEK293 cells and the F construct (CMV-hIns(rev)-RSV-rGck) mediated the highest insulin and glucokinase expression (FIG. 6). Therefore, said plasmid was used to produce the corresponding dual-gene AAV1 vector in HEK293 cells. Although the size of the CMV-hIns(rev)-RSV-rGck (construct F) genome construct was within optimal AAV encapsidation capacity, a vector batch of low yield was obtained again (5.5E11 vg/RB). Based on previous observations with other AAV constructs manufactured in our lab, we postulate that, in addition to the size of the vector genome, the conformation of the DNA may also impacts encapsidation efficiency, which could potentially explain the relative low manufacturing yield of this new dual construct.

A.4. Optimized miniCMV-Insulin-RSV-Glucokinase Dual-Gene AAV Constructs

Given that the AAV1-CMV-hlns(rev)-RSV-hGck production rendered a relative low yield, we decided to further decrease the size of the dual-gene construct replacing the CMV promoter by a short version of such promoter, named mini CMV promoter.

We generated 4 new dual-gene constructs bearing the human insulin coding sequence under the control of the mini CMV promoter and the human glucokinase coding sequence driven by the RSV promoter. The SV40 and the bGH polyA were used as polyA sequences, respectively. Again, we tested the effect of positioning the insulin expression cassette upstream of the glucokinase expression cassette or viceversa, and also the effect of positioning it the glucokinase expression cassette in reverse orientation (FIG. 7). The new constructs are:
Construct J is depicted in FIG. 7: miniCMV-hlns-RSV-hGck (size: 4 kb) (SEQ ID NO:13).
Construct K is depicted in FIG. 7: RSV-hGck-miniCMV-hlns (size: 4 kb) (SEQ ID NO:14).
Construct L is depicted in FIG. 7: miniCMV-hlns(rev)-RSV-hGck (size: 4 kb) (SEQ ID NO:15).
Construct M is depicted in FIG. 7: RSV-hGck-miniCMV-hlns(rev) (size: 4 kb) (SEQ ID NO:16).

In addition to these 4 new dual-gene AAV1-Ins+Gck constructs (J, K, L and M), an additional single-gene plasmid encoding insulin was generated using the same AAV backbone for comparison with the 4 new dual-gene AAV1-Ins+Gck constructs. The single-gene plasmid encoding Gck was the previously mentioned RSV-hGCK (construct I) (FIG. 8).
Construct N is depicted in FIG. 8: miniCMV-hlns (SEQ ID NO:21).
Construct I is depicted in FIG. 8: RSV-hGCK-bGH (SEQ ID NO:20).

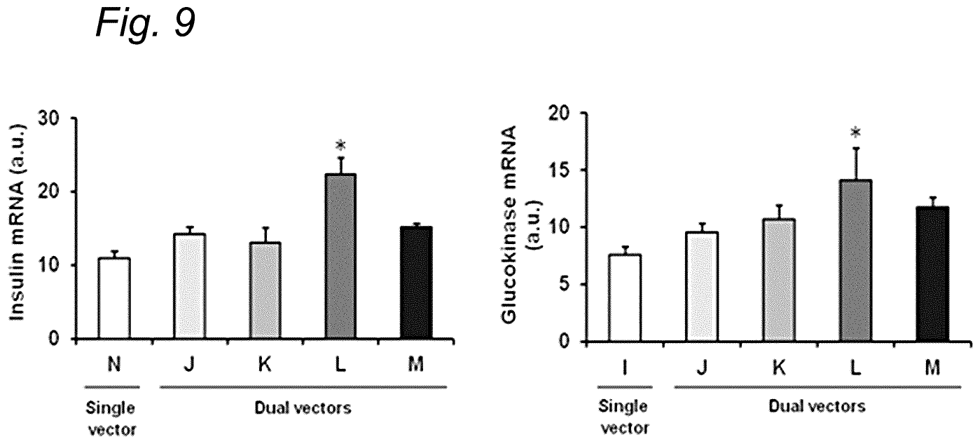

We assessed the function of constructs J, K, L and M dual-gene plasmids in vitro in HEK293 cells and the (L) construct, miniCMV-hIns(rev)-RSV-hGck, mediated the highest expression of insulin and glucokinase (FIG. 9).

This (L) construct (miniCMV-hIns(rev)-RSV-hGck) and the same construct (J) but in sense orientation (miniCMV-hIns-RSV-hGck dual-promoter) were used to produce the corresponding dual-gene AAV1 vectors in HEK293 cells.

In these cases, AAV production yields were within the expected value, being 2.1E12 vg/RB for AAV1-miniCMV-hIns(rev)-RSV-hGck (construct L) and 1.9E12 vg/RB for AAV1-miniCMV-hIns-RSV-hGck (construct J).

### B. Increased Transgene Expression and Efficacy of Dual-Gene AAV1-miniCMV-hIns(Rev)-RSV-hGck Vectors

### B.1. Increased Transgene Expression In Vivo

To verify if the administration of the double-gene AAV1-Ins+Gck vectors was superior than the co-delivery of two single-gene AAV vectors in mediating the expression of insulin and/or glucokinase and/or in the ability to improve glucose disposal in response to a glucose overload, an in vivo experiment was performed in mice.

Two groups of wild type mice were treated with either the 2 single vectors together (constructs N+I) (AAV1-miniCMV-hINS and AAV1-RSV-hGck) or with the dual gene (construct L) (AAV1-miniCMV-hINS-rev-RSV-hGck). Vectors were administered intramuscularly into tibialis and gastrocnemius muscles of both hindlimbs at a dose of 5E10 vg/muscle of each vector (constructs N and I or L).

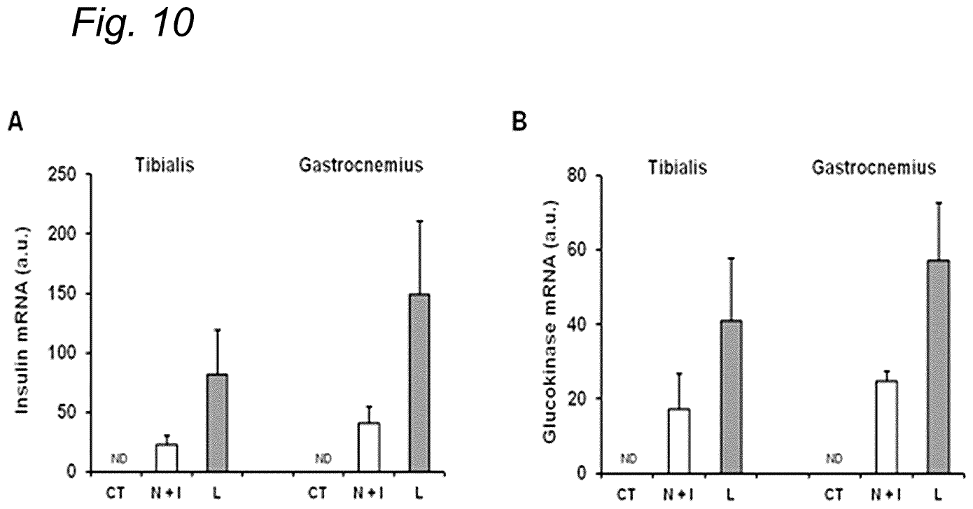

Three weeks after vector administration, animals were sacrificed and the expression of both transgenes (insulin and glucokinase) was analysed by real time quantitative PCR in the different experimental groups. We observed that the expression of both Insulin (FIG. 10A) and Glucokinase (FIG. 10B) was higher in the muscles obtained from the animals that received the double-gene vector (construct L), in comparison to the combination of the two single vectors (constructs N+I).

### B.2. Increased Efficacy In Vivo

To demonstrate the efficacy of the newly designed dual-gene constructs, the ability of the vector to enhance glucose disposal in vivo was assessed in the previous described experimental groups. To this end, a glucose tolerance test was performed in which all groups of mice were injected intraperitoneally with 2 g glucose/kg body weight, and blood glucose levels were determined at different time points.

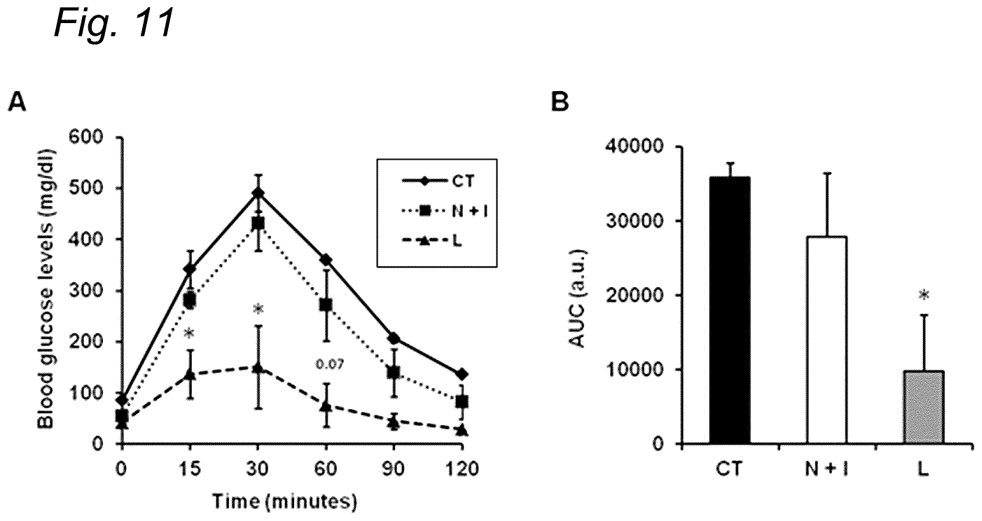

As observed in FIG. 11, animals injected with the L dual vector showed higher glucose tolerance than animals injected with the combination of the two single vectors.

### B.3. Increased Efficacy In Vivo in Diabetic Mice

In order to assess efficacy of the dual-gene (construct L) vector (AAV1-miniCMV-hIns(rev)-RSV-hGck) in diabetic animals, a dose of 5E10 vg/muscle was administered intramuscularly into tibialis and gastrocnemius muscles of both hindlimbs of mice treated with streptozotocin (STZ) to trigger the diabetic process. As control, the 2 single vectors were administered together (construct N+I) (AAV1-miniCMV-hINS and AAV1-RSV-hGck).

Eight weeks post-AAV administration, a glucose tolerance test was performed in which all groups of mice were injected intraperitoneally with 1 g glucose/kg body weight, and blood glucose levels were determined at different time points.

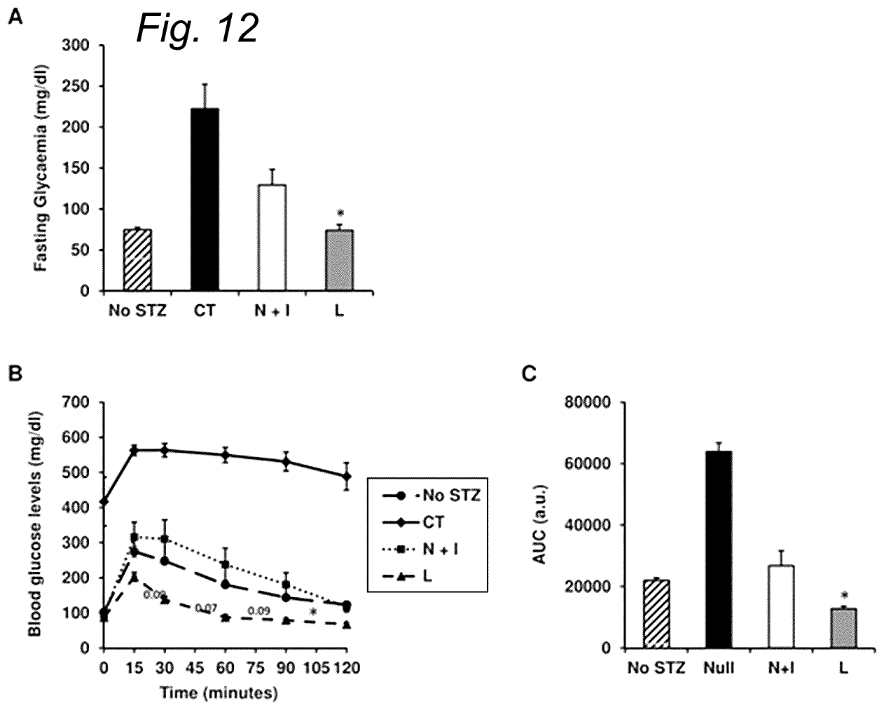

As observed in FIG. 12A, diabetic animals injected with the L dual vector showed decreased levels of glycaemia in fasted conditions in comparison with animals treated with the combination of the N+I single vectors. Noticeably, glucose levels displayed by animals treated with the L dual-gene vector were similar to those of non-diabetic healthy mice (FIG. 12A). Moreover, diabetic animals injected with the L dual vector showed higher glucose tolerance than animals injected with the combination of the two single vectors (N+I) (FIG. 12B-C).

### C. Increased Transgene Expression and Efficacy of Dual-Gene AAV1-miniCMV-Insulin-bGH(Rev)-RSV-Glucokinase-SV40

C1. Generation of Optimized miniCMV-Insulin-bGH(Rev)-RSV-Glucokinase-SV40 Dual-Gene AAV Constructs

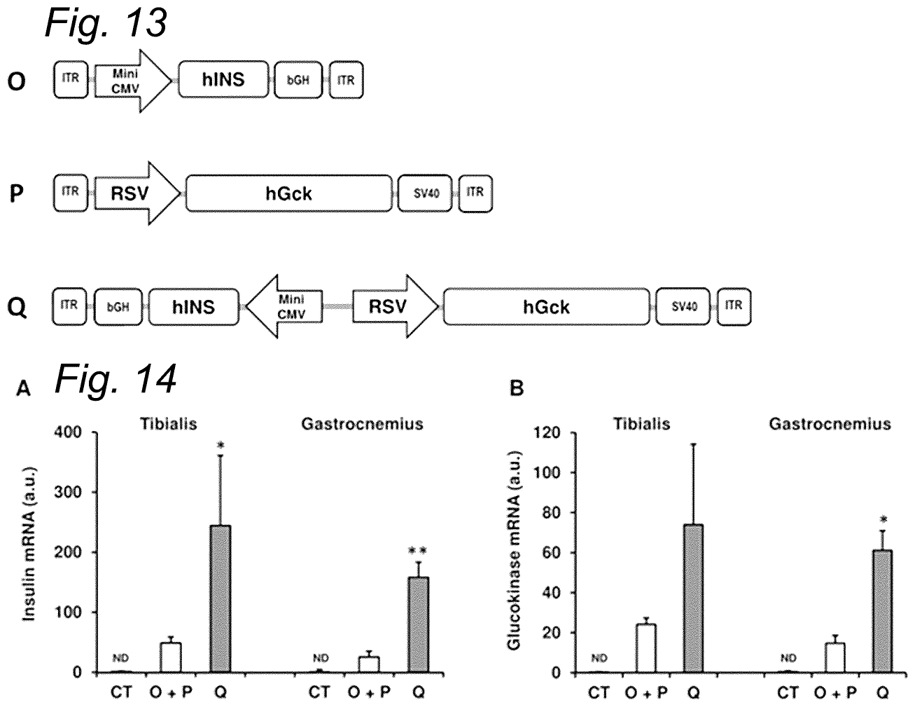

Given that polyadenylation signals have been reported to influence transgene expression (Azzoni et al., J Gene Med 2007; 9: 392-40.), we generated a new dual-gene construct bearing the human insulin coding sequence under the control of the mini CMV promoter and the bGH polyA (expression cassette in reverse orientation) and the human glucokinase coding sequence driven by the RSV promoter and SV40 polyA (construct Q; same construct as L but with polyA signals interchanged). Two additional single-gene plasmids encoding insulin and glucokinase (constructs O and P, respectively) were generated using the same AAV backbone for comparison with the new dual-gene AAV1-Ins+Gck (Q) construct (FIG. 13). The new constructs are:
Construct O is depicted in FIG. 13: miniCMV-hIns-bGH (size: 1.4 kb) (SEQ ID NO:25).
Construct P is depicted in FIG. 13: RSV-hGck-SV40 (size: 2.9 kb) (SEQ ID NO:26).
Construct Q is depicted in FIG. 13: miniCMV-hIns-bGH(rev)-RSV-hGck-SV40 (size: 4 kb) (SEQ ID NO:27).

### C.2. Increased Transgene Expression In Vivo

Two groups of wild type mice were treated with either the 2 single vectors together (constructs O+P) (AAV1-miniCMV-hIns-bGH and AAV1-RSV-hGck-SV40) or with the dual gene (construct Q) (AAV1-miniCMV-Insulin-bGH(rev)-RSV-Glucokinase-SV40). Vectors were administered intramuscularly into tibialis and gastrocnemius muscles of both hindlimbs at a dose of 5E10 vg/muscle of each vector (constructs O and P or Q).

Three weeks after vector administration, animals were sacrificed and the expression of both transgenes (insulin and glucokinase) was analysed by real time quantitative PCR in the different experimental groups. We observed that the expression of both Insulin (FIG. 14A) and Glucokinase (FIG. 14B) was higher in the muscles obtained from the animals that received the double-gene vector (construct Q), in comparison to the combination of the two single vectors (constructs O+P).

### C.3. Increased Efficacy In Vivo

To demonstrate the efficacy of the newly designed Q dual-gene construct (AAV1-miniCMV-Insulin-bGH(rev)-RSV-Glucokinase-SV40), the ability of the vector to enhance glucose disposal in vivo was assessed in the experimental groups previously described in section C.2. To this end, a glucose tolerance test was performed in which all groups of mice were injected intraperitoneally with 2 g glucose/kg body weight, and blood glucose levels were determined at different time points.

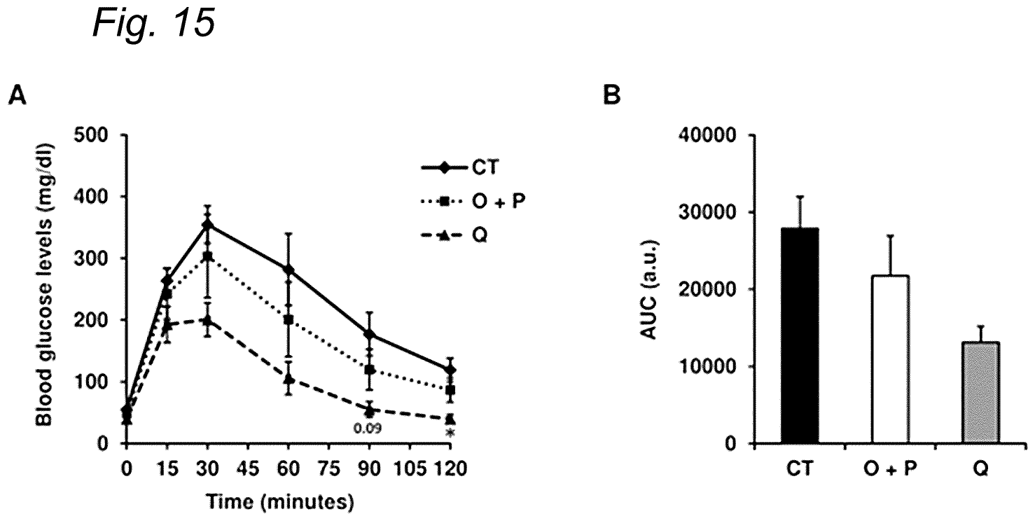

As observed in FIG. 15, animals injected with the Q dual vector showed higher glucose tolerance than animals injected with the combination of the two single vectors (O+P).

D. Increased Transgene Expression and Efficacy of Dual-Gene AAV1-miniCMV-hIns-SV40enhancer(Rev)-RSV-hGck-bGH
D.1. Generation of Optimized miniCMV-Insulin-SV40enhancer-RSV-Glucokinase-bGH Dual-Gene AAV Constructs

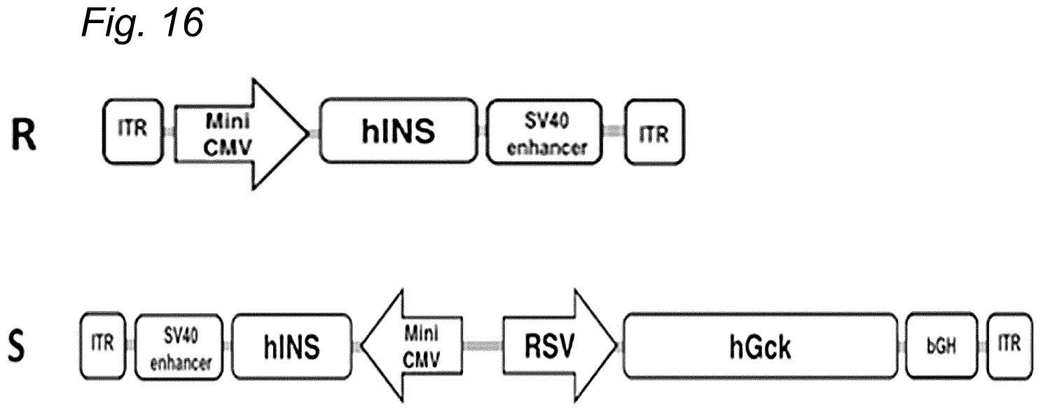

In order to increase the expression levels of insulin, the enhancer of the SV40 was incorporated at the 3′ end of the polyA. A new dual-gene construct bearing the human insulin coding sequence under the control of the mini CMV promoter and the SV40 enhancer at the 3′ end of the SV40 polyA (expression cassette in reverse orientation) and the human glucokinase coding sequence driven by the RSV promoter and the bGH polyA (construct S) was generated (FIG. 16). As control, a single-gene plasmid encoding insulin under the control of the mini CMV promoter and the SV40 enhancer at the 3′ end of the SV40 polyA (construct R) was generated (FIG. 16). The single-gene plasmid encoding Gck was the previously mentioned RSV-hGCK (construct I) (FIG. 8). The new constructs are:
Construct R is depicted in FIG. 16: miniCMV-hIns-SV40enhancer (size: 1.6 kb) (SEQ ID NO: 28).
Construct S is depicted in FIG. 16: miniCMV-hIns-SV40enhancer(rev)-RSV-hGck-bGH (size: 4.2 kb) (SEQ ID NO: 29).

### D.2. Increased Transgene Expression In Vivo

Two groups of wild type mice were treated with either the 2 single vectors together (constructs R+I) (AAV1-miniCMV-hIns-SV40enhancer and AAV1-RSV-hGck) or with the dual gene (construct S) (AAV1-miniCMV-hIns-SV40enhancer(rev)-RSV-hGck-bGH). Vectors were administered intramuscularly into tibialis and gastrocnemius muscles of both hindlimbs at a dose of 5E10 vg/muscle of each vector (constructs R and I or S).

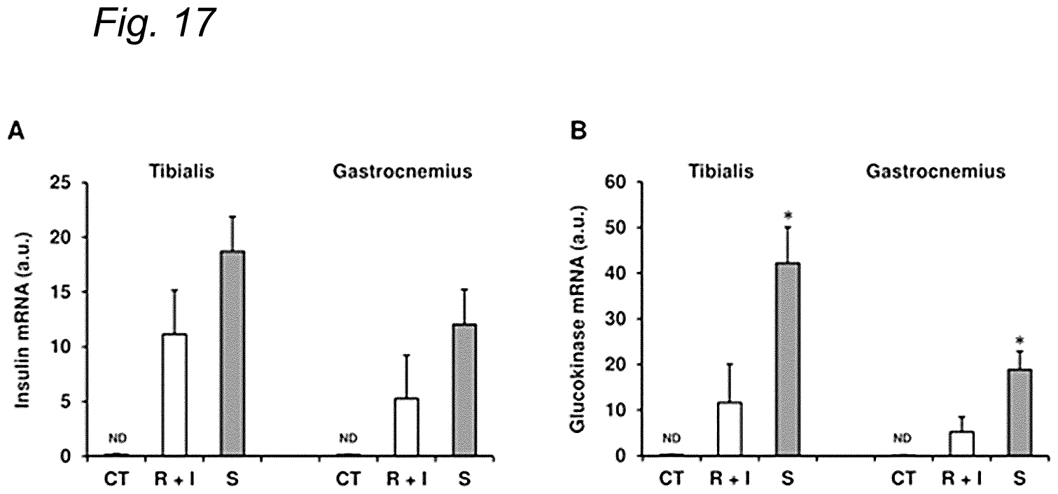

Three weeks after vector administration, animals were sacrificed and the expression of both transgenes (insulin and glucokinase) was analysed by real time quantitative PCR in the different experimental groups. We observed that the expression of both Insulin (FIG. 17A) and Glucokinase (FIG. 17B) was higher in the muscles obtained from the animals that received the double-gene vector (construct S), in comparison to the combination of the two single vectors (construct R+I).

### D.3. Increased Efficacy In Vivo

To demonstrate the efficacy of the newly designed S dual-gene construct (AAV1-miniCMV-hIns-SV40enhancer(rev)-RSV-hGck-bGH), the ability of the vector to enhance glucose disposal in vivo was assessed in the experimental groups previously described in section D.2. To this end, a glucose tolerance test was performed in which all groups of mice were injected intraperitoneally with 2 g glucose/kg body weight, and blood glucose levels were determined at different time points.

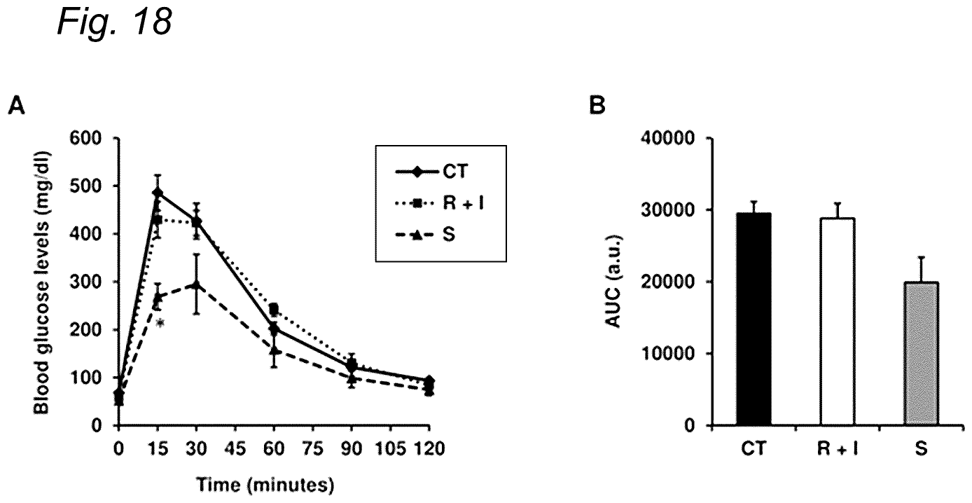

As observed in FIG. 18, animals injected with the S dual vector showed higher glucose tolerance than animals injected with the combination of the two single vectors (R+I).

In conclusion, we believe the new approach based on the use of the dual-gene AAV1-INS- -Gck vector allows for more -or at least the same- expression of therapeutic transgenes at considerably lower vector doses (half the vector genomes in dual-gene-treated mice), when compared to the combination of the two single vectors.

As the actions of insulin and glucokinase are synergic to create a glucose sensor in muscle, the use of dual-gene vectors allows the delivery of adequate amounts of both transgenes to the same cell. Therefore, the new approach based on the use of the dual-gene viral vector improves glucose metabolization to a higher extent when compared to the combination of the two single vectors. Moreover, it also allows for higher levels of expression of the transgenes using half the dose of viral genomes.

SEQUENCES

| SEQ ID NO: | Type of sequence |
|---|---|
| 1 | cDNA human insulin |
| 2 | cDNA human glucokinase |
| 3 | CMV promoter |
| 4 | Intronic sequence associated with |
| | CMV promoter |
| 5 | Mini CMV promoter |
| 6 | RSV promoter |
| 7 | bGH |
| 8 | Construct A |
| 9 | Construct D |
| 10 | Construct E |
| 11 | Construct F |
| 12 | Construct G |
| 13 | Construct J |
| 14 | Construct K |
| 15 | Construct L |
| 16 | Construct M |
| 17 | Construct B |
| 18 | Construct C |
| 19 | Construct H |
| 20 | Construct I |
| 21 | Construct N |
| 22 | SV40 polyadenylation signal |
| 23 | Intronic sequence associated with RSV |
| | promoter |
| 24 | Equivalent mini CMV promoter |
| 25 | Construct O |
| 26 | Construct P |
| 27 | Construct Q |
| 28 | Construct R |
| 29 | Construct S |
| 30 | SV40 polyadenylation signal and |
| | enhancer sequence |
| 31 | 5′ITR |
| 32 | 3′ITR |
| 33 | SV40 enhancer sequence |

CMV Promoter (Full):
Human cytomegalovirus (CMV) immediate early enhancer and promoter
(SEQ ID NO: 3)
TGTAGTTAATGATTAACCCGCCATGCTACTTATCTACAGATCTCAATAT
TGGCCATTAGCCATATTATTCATTGGTTATATAGCATAAATCAATATTG
GCTATTGGCCATTGCATACGTTGTATCTATATCATAATATGTACATTTA
TATTGGCTCATGTCCAATATGACCGCCATGTTGGCATTGATTATTGACT
AGTTATTAATAGTAATCAATTACGGGGTCATTAGTTCATAGCCCATATA
TGGAGTTCCGCGTTACATAACTTACGGTAAATGGCCCGCCTGGCTGAC
CGCCCAACGACCCCCGCCCATTGACGTCAATAATGACGTATGTTCCCA
TAGTAACGCCAATAGGGACTTTCCATTGACGTCAATGGGTGGAGTATT
TACGGTAAACTGCCCACTTGGCAGTACATCAAGTGTATCATATGCCAA
GTCCGCCCCCTATTGACGTCAATGACGGTAAATGGCCCGCCTGGCATT
ATGCCCAGTACATGACCTTACGGGACTTTCCTACTTGGCAGTACATCTA
CGTATTAGTCATCGCTATTACCATGGTGATGCGGTTTTGGCAGTACACC
AATGGGCGTGGATAGCGGTTTGACTCACGGGGATTTCCAAGTCTCCAC
CCCATTGACGTCAATGGGAGTTTGTTTTGGCACCAAAATCAACGGGAC
TTTCCAAAATGTCGTAACAACTGCGATCGCCCGCCCCGTTGACGCAAA
TGGGCGGTAGGCGTGTACGGTGGGAGGTCTATATAAGCAGAGCTCGTT
TAGTGAACCGTCAGATCACTAG
Intronic sequence associated with CMV promoter
(SEQ ID NO: 4)
TATTGCGGTAGTTTATCACAGTTAAATTGCTAACGCAGTCAGTGCTTCT
GACACAACAGTCTCGAACTTAAGCTGCAGTGACTCTCTTAAGGTAGCC
TTGCAGAAGTTGGTCGTGAGGCACTGGGCAGGTAAGTATCAAGGTTAC
AAGACAGGTTTAAGGAGACCAATAGAAACTGGGCTTGTCGAGACAGAG
AAGACTCTTGCGTTTCTGATAGGCACCTATTGGTCTTACTGACATCCAC
TTTGCCTTTCTCTCCACAGGTGTCCACTCCCAGTTCAATTACAGCTCTT
AAGGCTAGAGTACTTAATACGACTCACTATAGAATACGACTCACTATAG
GGAGAC
Human Insulin (A718) Cdna
(SEQ ID NO: 1)
CTTCTGCCATGGCCCTGTGGATGCGCCTCCTGCCCCTGCTGGCGCTGC
TGGCCCTCTGGGGACCTGACCCAGCCGCAGCCTTTGTGAACCAACACC
TGTGCGGCTCAGATCTGGTGGAAGCTCTCTACCTAGTGTGCGGGGAAC
GAGGCTTCTTCTACACACCCAGGACCAAGCGGGAGGCAGAGGACCTGC
AGGTGGGGCAGGTGGAGCTGGGCGGGGGCCCTGGTGCAGGCAGCCTG
CAGCCCTTGGCCCTGGAGGGGTCGCGACAGAAGCGTGGCATTGTGGA
ACAATGCTGTACCAGCATCTGCTCCCTCTACCAGCTGGAGAACTACTG
CAACTAGACGCAGCC
SV40 PolyA
(SEQ ID NO: 22)
GGTACCAGCGCTGTCGAGGCCGCTTCGAGCAGACATGATAAGATACAT
TGATGAGTTTGGACAAACCACAACTAGAATGCAGTGAAAAAAATGCTT
TATTTGTGAAATTTGTGATGCTATTGCTTTATTTGTAACCATTATAAGC
TGCAATAAACAAGTTAACAACAACAATTGCATTCATTTTATGTTTCAGG
TTCAGGGGGAGATGTGGGAGGTTTTTTAAAGCAAGTAAAACCTCTACA
AATGTGGTAAAATCGATTAGGATCTTCCTAGAGCATGGCTACCTAGAC
ATGGCTCGACAGATCAGCGCTCATGCTCTGGAAGATCTCG
RSV promoter
(SEQ ID NO: 6)
CATGTTTGACAGCTTATCATCGCAGATCCGTATGGTGCACTCTCAGTAC
AATCTGCTCTGATGCCGCATAGTTAAGCCAGTATCTGCTCCCTGCTTGT
GTGTTGGAGGTCGCTGAGTAGTGCGCGAGCAAAATTTAAGCTACAACA
AGGCAAGGCTTGACCGACAATTGCATGAAGAATCTGCTTAGGGTTAGG
CGTTTTGCGCTGCTTCGCGATGTACGGGCCAGATATTCGCGTATCTGA
GGGGACTAGGGTGTGTTTAGGCGAAAAGCGGGGCTTCGGTTGTACGC
GGTTAGGAGTCCCCTCAGGATATAGTAGTTTCGCTTTTGCATAGGGAG
GGGGAAATGTAGTCTTATGCAATACTCTTGTAGTCTTGCAACATGGTAA
CGATGAGTTAGCAACATGCCTTACAAGGAGAGAAAAAGCACCGTGCAT
GCCGATTGGTGGAAGTAAGGTGGTACGATCGTGCCTTATTAGGAAGGC
AACAGACGGGTCTGACATGGATTGGACGAACCACTAAATTCCGCATTG
CAGAGATATTGTATTTAAGTGCCTAGCTCGATACAATAAACGCCATTTG
ACCATTCACCACATTGGTGTGCACCTCCAAGCTGGGTACCAGCT
Intronic sequence associated with RSV promoter
(SEQ ID NO: 23)
GAGATCTGCTTCAGCTGGAGGCACTGGGCAGGTAAGTATCAAGGTTAC
AAGACAGGTTTAAGGAGACCAATAGAAACTGGGCTTGTCGAGACAGAG
AAGACTCTTGCGTTTCTGATAGGCACCTATTGGTCTTACTGACATCCAC
TTTGCCTTTCTCTCCACAGGTGCAGCTGCTGCAGCGG
Human GcK
(SEQ ID NO: 2)
TCGAGACCATGGCGATGGATGTCACAAGGAGCCAGGCCCAGACAGCCT
TGACTCTGGTAGAGCAGATCCTGGCAGAGTTCCAGCTGCAGGAGGAGG
ACCTGAAGAAGGTGATGAGACGGATGCAGAAGGAGATGGACCGCGGC
CTGAGGCTGGAGACCCATGAAGAGGCCAGTGTGAAGATGCTGCCCACC
TACGTGCGCTCCACCCCAGAAGGCTCAGAAGTCGGGGACTTCCTCTCC
CTGGACCTGGGTGGCACTAACTTCAGGGTGATGCTGGTGAAGGTGGGA
GAAGGTGAGGAGGGGCAGTGGAGCGTGAAGACCAAACACCAGATGTA
CTCCATCCCCGAGGACGCCATGACCGGCACTGCTGAGATGCTCTTCGA
CTACATCTCTGAGTGCATCTCCGACTTCCTGGACAAGCATCAGATGAA
ACACAAGAAGCTGCCCCTGGGCTTCACCTTCTCCTTTCCTGTGAGGCA
CGAAGACATCGATAAGGGCATCCTTCTCAACTGGACCAAGGGCTTCAA
GGCCTCAGGAGCAGAAGGGAACAATGTCGTGGGGCTTCTGCGAGACG
CTATCAAACGGAGAGGGGACTTTGAAATGGATGTGGTGGCAATGGTGA
ATGACACGGTGGCCACGATGATCTCCTGCTACTACGAAGACCATCAGT
GCGAGGTCGGCATGATCGTGGGCACGGGCTGCAATGCCTGCTACATG
GAGGAGATGCAGAATGTGGAGCTGGTGGAGGGGGACGAGGGCCGCAT
GTGCGTCAATACCGAGTGGGGCGCCTTCGGGGACTCCGGCGAGCTGG
ACGAGTTCCTGCTGGAGTATGACCGCCTGGTGGACGAGAGCTCTGCAA
ACCCCGGTCAGCAGCTGTATGAGAAGCTCATAGGTGGCAAGTACATGG
GCGAGCTGGTGCGGCTTGTGCTGCTCAGGCTCGTGGACGAAAACCTGC
TCTTCCACGGGGAGGCCTCCGAGCAGCTGCGCACACGCGGAGCCTTCG
AGACGCGCTTCGTGTCGCAGGTGGAGAGCGACACGGGCGACCGCAAG
CAGATCTACAACATCCTGAGCACGCTGGGGCTGCGACCCTCGACCACC
GACTGCGACATCGTGCGCCGCGCCTGCGAGAGCGTGTCTACGCGCGCT
GCGCACATGTGCTCGGCGGGGCTGGCGGGCGTCATCAACCGCATGCG
CGAGAGCCGCAGCGAGGACGTAATGCGCATCACTGTGGGCGTGGATG
GCTCCGTGTACAAGCTGCACCCCAGCTTCAAGGAGCGGTTCCATGCCA
GCGTGCGCAGGCTGACGCCCAGCTGCGAGATCACCTTCATCGAGTCGG
AGGAGGGCAGTGGCCGGGGCGCGGCCCTGGTCTCGGCGGTGGCCTGT
AAGAAGGCCTGTATGCTGGGCCAGTGA
bGH PolyA
(SEQ ID NO: 7)
CACGTGGAGCTCGCTGATCAGCCTCGACTGTGCCTTCTAGTTGCCAGC
CATCTGTTGTTTGCCCCTCCCCCGTGCCTTCCTTGACCCTGGAAGGTGC
CACTCCCACTGTCCTTTCCTAATAAAATGAGGAAATTGCATCGCATTGT
CTGAGTAGGTGTCATTCTATTCTGGGGGGTGGGGTGGGGCAGGACAG
CAAGGGGGAGGATTGGGAAGACAATAGCAGGCATGCTGGGGATGCGG
TGGGCTCTATGGCCACGTG
Mini-CMV: cmv intermediate early promoter
(SEQ ID NO: 5)
TATGCCAAGTACGCCCCCTATTGACGTCAATGACGGTAAATGGCCCGC
CTGGCATTATGCCCAGTACATGACCTTATGGGACTTTCCTACTTGGCAG
TACATCTACGTATTAGTCATCGCTATTACCATGGTGATGCGGTTTTGGC
AGTACATCAATGGGCGTGGATAGCGGTTTGACTCACGGGGATTTCCAA
GTCTCCACCCCATTGACGTCAATGGGAGTTTGTTTTGGCACCAAAATCA
ACGGGACTTTCCAAAATGTCGTAACAACTCCGCCCCATTGACGCAAAT
GGGCGGTAGGCGTGTACGGTGGGAGGTCTATATAAGCAGAGCTCTCTG
GCTAACTAGAGAACCCACTGCTTAACTGGCTTATCGAAATTAATACGAC
TCACTATAGGGAGACCCAAGCTT
A: RSV-rGck-CMV-hIns

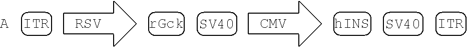

pGG2-RSV-rGck-CMV-hIns plasmid sequence
(SEQ ID NO: 8)

| | |
|---|---|
| 1 | CTAGACATGG CTCGACAGAT CTCAATATTG GCCATTAGCC ATATTATTCA |
| 51 | TTGGTTATAT AGCATAAATC AATATTGGCT ATTGGCCATT GCATACGTTG |
| 101 | TATCTATATC ATAATATGTA CATTTATATT GGCTCATGTC CAATATGACC |
| 151 | GCCATGTTGG CATTGATTAT TGACTAGTTA TTAATAGTAA TCAATTACGG |
| 201 | GGTCATTAGT TCATAGCCCA TATATGGAGT TCCGCGTTAC ATAACTTACG |
| 251 | GTAAATGGCC CGCCTGGCTG ACCGCCCAAC GACCCCCGCC CATTGACGTC |
| 301 | AATAATGACG TATGTTCCCA TAGTAACGCC AATAGGGACT TTCCATTGAC |
| 351 | GTCAATGGGT GGAGTATTTA CGGTAAACTG CCCACTTGGC AGTACATCAA |
| 401 | GTGTATCATA TGCCAAGTCC GCCCCCTATT GACGTCAATG ACGGTAAATG |
| 451 | GCCCGCCTGG CATTATGCCC AGTACATGAC CTTACGGGAC TTTCCTACTT |
| 501 | GGCAGTACAT CTACGTATTA GTCATCGCTA TTACCATGGT GATGCGGTTT |
| 551 | TGGCAGTACA CCAATGGGCG TGGATAGCGG TTTGACTCAC GGGGATTTCC |
| 601 | AAGTCTCCAC CCCATTGACG TCAATGGGAG TTTGTTTTGG CACCAAAATC |
| 651 | AACGGGACTT TCCAAAATGT CGTAACAACT GCGATCGCCC GCCCCGTTGA |
| 701 | CGCAAATGGG CGGTAGGCGT GTACGGTGGG AGGTCTATAT AAGCAGAGCT |
| 751 | CGTTTAGTGA ACCGTCAGAT CACTAGAAGC TTTATTGCGG TAGTTTATCA |
| 801 | CAGTTAAATT GCTAACGCAG TCAGTGCTTC TGACACAACA GTCTCGAACT |
| 851 | TAAGCTGCAG TGACTCTCTT AAGGTAGCCT TGCAGAAGTT GGTCGTGAGG |
| 901 | CACTGGGCAG GTAAGTATCA AGGTTACAAG ACAGGTTTAA GGAGACCAAT |
| 951 | AGAAACTGGG CTTGTCGAGA CAGAGAAGAC TCTTGCGTTT CTGATAGGCA |
| 1001 | CCTATTGGTC TTACTGACAT CCACTTTGCC TTTCTCTCCA CAGGTGTCCA |
| 1051 | CTCCCAGTTC AATTACAGCT CTTAAGGCTA GAGTACTTAA TACGACTCAC |
| 1101 | TATAGGCTAG CCTCGAGAAT TCTGCCATGG CCCTGTGGAT GCGCCTCCTG |
| 1151 | CCCCTGCTGG CGCTGCTGGC CCTCTGGGGA CCTGACCCAG CCGCAGCCTT |
| 1201 | TGTGAACCAA CACCTGTGCG GCTCAGATCT GGTGGAAGCT CTCTACCTAG |
| 1251 | TGTGCGGGGA ACGAGGCTTC TTCTACACAC CCAGGACCAA GCGGGAGGCA |
| 1301 | GAGGACCTGC AGGTGGGGCA GGTGGAGCTG GGCGGGGGCC CTGGTGCAGG |
| 1351 | CAGCCTGCAG CCCTTGGCCC TGGAGGGGTC GCGACAGAAG CGTGGCATTG |
| 1401 | TGGAACAATG CTGTACCAGC ATCTGCTCCC TCTACCAGCT GGAGAACTAC |
| 1451 | TGCAACTAGA CGCAGCTGCA AGCTTATCGA TACCGTCGAC CCGGGCGGCC |
| 1501 | GCTTCCCTTT AGTGAGGGTT AATGCTTCGA GCAGACATGA TAAGATACAT |
| 1551 | TGATGAGTTT GGACAAACCA CAACTAGAAT GCAGTGAAAA AAATGCTTTA |
| 1601 | TTTGTGAAAT TTGTGATGCT ATTGCTTTAT TTGTAACCAT TATAAGCTGC |
| 1651 | AATAAACAAG TTAACAACAA CAATTGCATT CATTTTATGT TTCAGGTTCA |
| 1701 | GGGGGAGATG TGGGAGGTTT TTTAAAGCAA GTAAAACCTC TACAAATGTG |
| 1751 | GTAAAATCCG ATAAGGGACT AGAGCATGGC TACGTAGATA AGTAGCATGG |
| 1801 | CGGGTTAATC ATTAACTACA AGGAACCCCT AGTGATGGAG TTGGCCACTC |
| 1851 | CCTCTCTGCG CGCTCGCTCG CTCACTGAGG CCGGGCGACC AAAGGTCGCC |
| 1901 | CGACGCCCGG GCTTTGCCCG GGCGGCCTCA GTGAGCGAGC GAGCGCGCCA |
| 1951 | GCTGGCGTAA TAGCGAAGAG GCCCGCACCG ATCGCCCTTC CCAACAGTTG |
| 2001 | CGCAGCCTGA ATGGCGAATG GAATTCCAGA CGATTGAGCG TCAAAATGTA |
| 2051 | GGTATTTCCA TGAGCGTTTT TCCGTTGCAA TGGCTGGCGG TAATATTGTT |
| 2101 | CTGGATATTA CCAGCAAGGC CGATAGTTTG AGTTCTTCTA CTCAGGCAAG |
| 2151 | TGATGTTATT ACTAATCAAA GAAGTATTGC GACAACGGTT AATTTGCGTG |
| 2201 | ATGGACAGAC TCTTTTACTC GGTGGCCTCA CTGATTATAA AAACACTTCT |
| 2251 | CAGGATTCTG GCGTACCGTT CCTGTCTAAA ATCCCTTTAA TCGGCCTCCT |
| 2301 | GTTTAGCTCC CGCTCTGATT CTAACGAGGA AAGCACGTTA TACGTGCTCG |
| 2351 | TCAAAGCAAC CATAGTACGC GCCCTGTAGC GGCGCATTAA GCGCGGCGGG |
| 2401 | TGTGGTGGTT ACGCGCAGCG TGACCGCTAC ACTTGCCAGC GCCCTAGCGC |
| 2451 | CCGCTCCTTT CGCTTTCTTC CCTTCCTTTC TCGCCACGTT CGCCGGCTTT |
| 2501 | CCCCGTCAAG CTCTAAATCG GGGGCTCCCT TTAGGGTTCC GATTTAGTGC |
| 2551 | TTTACGGCAC CTCGACCCCA AAAAACTTGA TTAGGGTGAT GGTTCACGTA |
| 2601 | GTGGGCCATC GCCCTGATAG ACGGTTTTTC GCCCTTTGAC GTTGGAGTCC |
| 2061 | ACGTTCTTTA ATAGTGGACT CTTGTTCCAA ACTGGAACAA CACTCAACCC |
| 2701 | TATCTCGGTC TATTCTTTTG ATTTATAAGG GATTTTGCCG ATTTCGGCCT |
| 2751 | ATTGGTTAAA AAATGAGCTG ATTTAACAAA AATTTAACGC GAATTTTAAC |
| 2801 | AAAATATTAA CGTCTACAAT TTAAATATTT GCTTATACAA TCTTCCTGTT |
| 2851 | TTTGGGGCTT TTCTGATTAT CAACCGGGGT ACATATGATT GACATGCTAG |
| 2901 | TTTTACGATT ACCGTTCATC GATTCTCTTG TTTGCTCCAG ACTCTCAGGC |
| 2951 | AATGACCTGA TAGCCTTTGT AGAGACCTCT CAAAAATAGC TACCCTCTCC |
| 3001 | GGCATGAATT TATCAGCTAG AACGGTTGAA TATCATATTG ATGGTGATTT |
| 3051 | GACTGTCTCC GGCCTTTCTC ACCCGTTTGA ATCTTTACCT ACACATTACT |
| 3101 | CAGGCATTGC ATTTAAAATA TATGAGGGTT CTAAAAATTT TTATCCTTGC |
| 3151 | GTTGAAATAA AGGCTTCTCC CGCAAAAGTA TTACAGGGTC ATAATGTTTT |
| 3201 | TGGTACAACC GATTTAGCTT TATGCTCTGA GGCTTTATTG CTTAATTTTG |
| 3251 | CTAATTCTTT GCCTTGCCTG TATGATTTAT TGGATGTTGG AATCGCCTGA |
| 3301 | TGCGGTATTT TCTCCTTACG CATCTGTGCG GTATTTCACA CCGCATATGG |
| 3351 | TGCACTCTCA GTACAATCTG CTCTGATGCC GCATAGTTAA GCCAGCCCCG |
| 3401 | ACACCCGCCA ACACCCGCTG ACGCGCCCTG ACGGGCTTGT CTGCTCCCGG |
| 3451 | CATCCGCTTA CAGACAAGCT GTGACCGTCT CCGGGAGCTG CATGTGTCAG |
| 3501 | AGGTTTTCAC CGTCATCACC GAAACGCGCG AGACGAAAGG GCCTCGTGAT |
| 3551 | ACGCCTATTT TTATAGGTTA ATGTCATGAT AATAATGGTT TCTTAGACGT |
| 3601 | CAGGTGGCAC TTTTCGGGGA AATGTGCGCG GAACCCCTAT TTGTTTATTT |
| 3651 | TTCTAAATAC ATTCAAATAT GTATCCGCTC ATGAGACAAT AACCCTGATA |
| 3701 | AATGCTTCAA TAATATTGAA AAAGGAAGAG TATGAGTATT CAACATTTCC |
| 3751 | GTGTCGCCCT TATTCCCTTT TTTGCGGCAT TTTGCCTTCC TGTTTTTGCT |
| 3801 | CACCCAGAAA CGCTGGTGAA AGTAAAAGAT GCTGAAGATC AGTTGGGTGC |
| 3851 | ACGAGTGGGT TACATCGAAC TGGATCTCAA CAGCGGTAAG ATCCTTGAGA |
| 3901 | GTTTTCGCCC CGAAGAACGT TTTCCAATGA TGAGCACTTT TAAAGTTCTG |
| 3951 | CTATGTGGCG CGGTATTATC CCGTATTGAC GCCGGGCAAG AGCAACTCGG |
| 4001 | TCGCCGCATA CACTATTCTC AGAATGACTT GGTTGAGTAC TCACCAGTCA |
| 4051 | CAGAAAAGCA TCTTACGGAT GGCATGACAG TAAGAGAATT ATGCAGTGCT |
| 4101 | GCCATAACCA TGAGTGATAA CACTGCGGCC AACTTACTTC TGACAACGAT |
| 4151 | CGGAGGACCG AAGGAGCTAA CCGCTTTTTT GCACAACATG GGGGATCATG |
| 4201 | TAACTCGCCT TGATCGTTGG GAACCGGAGC TGAATGAAGC CATACCAAAC |
| 4251 | GACGAGCGTG ACACCACGAT GCCTGTAGCA ATGGCAACAA CGTTGCGCAA |
| 4301 | ACTATTAACT GGCGAACTAC TTACTCTAGC TTCCCGGCAA CAATTAATAG |
| 4351 | ACTGGATGGA GGCGGATAAA GTTGCAGGAC CACTTCTGCG CTCGGCCCTT |
| 4401 | CCGGCTGGCT GGTTTATTGC TGATAAATCT GGAGCCGGTG AGCGTGGGTC |
| 4451 | TCGCGGTATC ATTGCAGCAC TGGGGCCAGA TGGTAAGCCC TCCCGTATCG |
| 4501 | TAGTTATCTA CACGACGGGG AGTCAGGCAA CTATGGATGA ACGAAATAGA |
| 4551 | CAGATCGCTG AGATAGGTGC CTCACTGATT AAGCATTGGT AACTGTCAGA |
| 4601 | CCAAGTTTAC TCATATATAC TTTAGATTGA TTTAAAACTT CATTTTTAAT |
| 4651 | TTAAAAGGAT CTAGGTGAAG ATCCTTTTTG ATAATCTCAT GACCAAAATC |
| 4701 | CCTTAACGTG AGTTTTCGTT CCACTGAGCG TCAGACCCCG TAGAAAAGAT |
| 4751 | CAAAGGATCT TCTTGAGATC CTTTTTTTCT GCGCGTAATC TGCTGCTTGC |
| 4801 | AAACAAAAAA ACCACCGCTA CCAGCGGTGG TTTGTTTGCC GGATCAAGAG |
| 4851 | CTACCAACTC TTTTTCCGAA GGTAACTGGC TTCAGCAGAG CGCAGATACC |
| 4901 | AAATACTGTC CTTCTAGTGT AGCCGTAGTT AGGCCACCAC TTCAAGAACT |
| 4951 | CTGTAGCACC GCCTACATAC CTCGCTCTGC TAATCCTGTT ACCAGTGGCT |
| 5001 | GCTGCCAGTG GCGATAAGTC GTGTCTTACC GGGTTGGACT CAAGACGATA |
| 5051 | GTTACCGGAT AAGGCGCAGC GGTCGGGCTG AACGGGGGGT TCGTGCACAC |
| 5101 | AGCCCAGCTT GGAGCGAACG ACCTACACCG AACTGAGATA CCTACAGCGT |
| 5151 | GAGCTATGAG AAAGCGCCAC GCTTCCCGAA GGGAGAAAGG CGGACAGGTA |
| 5201 | TCCGGTAAGC GGCAGGGTCG GAACAGGAGA GCGCACGAGG GAGCTTCCAG |
| 5251 | GGGGAAACGC CTGGTATCTT TATAGTCCTG TCGGGTTTCG CCACCTCTGA |
| 5301 | CTTGAGCGTC GATTTTTGTG ATGCTCGTCA GGGGGGCGGA GCCTATGGAA |
| 5351 | AAACGCCAGC AACGCGGCCT TTTTACGGTT CCTGGCCTTT TGCTGGCCTT |
| 5401 | TTGCTCACAT GTTCTTTCCT GCGTTATCCC CTGATTCTGT GGATAACCGT |
| 5451 | ATTACCGCCT TTGAGTGAGC TGATACCGCT CGCCGCAGCC GAACGACCGA |
| 5501 | GCGCAGCGAG TCAGTGAGCG AGGAAGCGGA AGAGCGCCCA ATACGCAAAC |
| 5551 | CGCCTCTCCC CGCGCGTTGG CCGATTCATT AATGCAGCAG CTGCGCGCTC |
| 5601 | GCTCGCTCAC TGAGGCCGCC CGGGCAAAGC CCGGGCGTCG GGCGACCTTT |
| 5651 | GGTCGCCCGG CCTCAGTGAG CGAGCGAGCG CGCAGAGAGG GAGTGGCCAA |
| 5701 | CTCCATCACT AGGGGTTCCT TGTAGTTAAT GATTAACCCG CCATGCTACT |
| 5751 | TATCTACGTA GCCATGCTCT AGGTAGCCAT GCTCTGGAAG ATCTCGACGC |
| 5801 | GTCATGTTTG ACAGCTTATC ATCGCAGATC CGTATGGTGC ACTCTCAGTA |
| 5851 | CAATCTGCTC TGATGCCGCA TAGTTAAGCC AGTATCTGCT CCCTGCTTGT |
| 5901 | GTGTTGGAGG TCGCTGAGTA GTGCGCGAGC AAAATTTAAG CTACAACAAG |
| 5951 | GCAAGGCTTG ACCGACAATT GCATGAAGAA TCTGCTTAGG GTTAGGCGTT |
| 6001 | TTGCGCTGCT TCGCGATGTA CGGGCCAGAT ATTCGCGTAT CTGAGGGGAC |
| 6051 | TAGGGTGTGT TTAGGCGAAA AGCGGGGCTT CGGTTGTACG CGGTTAGGAG |
| 6101 | TCCCCTCAGG ATATAGTAGT TTCGCTTTTG CATAGGGAGG GGGAAATGTA |
| 6151 | GTCTTATGCA ATACTCTTGT AGTCTTGCAA CATGGTAACG ATGAGTTAGC |
| 6201 | AACATGCCTT ACAAGGAGAG AAAAAGCACC GTGCATGCCG ATTGGTGGAA |
| 6251 | GTAAGGTGGT ACGATCGTGC CTTATTAGGA AGGCAACAGA CGGGTCTGAC |
| 6301 | ATGGATTGGA CGAACCACTA AATTCCGCAT TGCAGAGATA TTGTATTTAA |
| 6351 | GTGCCTAGCT CGATACAATA AACGCCATTT GACCATTCAC CACATTGGTG |
| 6401 | TGCACCTCCA AGCTGGGTAC CAGCTGCTAG CAAGCTTGAG ATCTGCTTCA |
| 6451 | GCTGGAGGCA CTGGGCAGGT AAGTATCAAG GTTACAAGAC AGGTTTAAGG |
| 6501 | AGACCAATAG AAACTGGGCT TGTCGAGACA GAGAAGACTC TTGCGTTTCT |
| 6551 | GATAGGCACC TATTGGTCTT ACTGACATCC ACTTTGCCTT TCTCTCCACA |
| 6601 | GGTGCAGCTG CTGCAGCGGG AATTCAACAG GTGGCCTCAG GAGTCAGGAA |
| 6651 | CATCTCTACT TCCCCAACGA CCCCTGGGTT GTCCTCTCAG AGATGGCTAT |
| 6701 | GGATACTACA AGGTGTGGAG CCCAGTTGTT GACTCTGGTC GAGCAGATCC |
| 6751 | TGGCAGAGTT CCAGCTGCAG GAGGAAGACC TGAAGAAGGT GATGAGCCGG |
| 6801 | ATGCAGAAGG AGATGGACCG TGGCCTGAGG CTGGAGACCC ACGAGGAGGC |
| 6851 | CAGTGTAAAG ATGTTACCCA CCTACGTGCG TTCCACCCCA GAAGGCTCAG |
| 6901 | AAGTCGGAGA CTTTCTCTCC TTAGACCTGG GAGGAACCAA CTTCAGAGTG |
| 6951 | ATGCTGGTCA AAGTGGGAGA GGGGGAGGCA GGGCAGTGGA GCGTGAAGAC |
| 7001 | AAAACACCAG ATGTACTCCA TCCCCGAGGA CGCCATGACG GGCACTGCCG |
| 7051 | AGATGCTCTT TGACTACATC TCTGAATGCA TCTCTGACTT CCTTGACAAG |
| 7101 | CATCAGATGA AGCACAAGAA ACTGCCCCTG GGCTTCACCT TCTCCTTCCC |
| 7151 | TGTGAGGCAC GAAGACCTAG ACAAGGGCAT CCTCCTCAAT TGGACCAAGG |
| 7201 | GCTTCAAGGC CTCTGGAGCA GAAGGGAACA ACATCGTAGG ACTTCTCCGA |
| 7251 | GATGCTATCA AGAGGAGAGG GGACTTTGAG ATGGATGTGG TGGCAATGGT |
| 7301 | GAACGACACA GTGGCCACAA TGATCTCCTG CTACTATGAA GACCGCCAAT |
| 7351 | GTGAGGTCGG CATGATTGTG GGCACTGGCT GCAATGCCTG CTACATGGAG |
| 7401 | GAAATGCAGA ATGTGGAGCT GGTGGAAGGG GATGAGGGAC GCATGTGCGT |
| 7451 | CAACACGGAG TGGGGCGCCT TCGGGGACTC GGGCGAGCTG GATGAGTTCC |
| 7501 | TACTGGAGTA TGACCGGATG GTGGATGAAA GCTCAGCGAA CCCCGGTCAG |
| 7551 | CAGCTGTACG AGAAGATCAT CGGTGGGAAG TATATGGGCG AGCTGGTACG |
| 7601 | ACTTGTGCTG CTTAAGCTGG TGGACGAGAA CCTTCTGTTC CACGGAGAGG |
| 7651 | CCTCGGAGCA GCTGCGCACG CGTGGTGCTT TTGAGACCCG TTTCGTGTCA |
| 7701 | CAAGTGGAGA GCGACTCCGG GGACCGAAAG CAGATCCACA ACATCCTAAG |
| 7751 | CACTCTGGGG CTTCGACCCT CTGTCACCGA CTGCGACATT GTGCGCCGTG |
| 7801 | CCTGTGAAAG CGTGTCCACT CGCGCCGCCC ATATGTGCTC CGCAGGACTA |
| 7851 | GCTGGGGTCA TAAATCGCAT GCGCGAAAGC CGCAGTGAGG ACGTGATGCG |
| 7901 | CATCACTGTG GGCGTGGATG GCTCCGTGTA CAAGCTGCAC CCGAGCTTCA |
| 7951 | AGGAGCGGTT TCACGCCAGT GTGCGCAGGC TGACACCCAA CTGCGAAATC |
| 8001 | ACCTTCATCG AATCAGAGGA GGGCAGCGGC AGGGGAGCCG CACTGGTCTC |
| 8051 | TGCGGTGGCC TGCAAGAAGG CTTGCATGCT GGCCCAGTGA AATCCAGGTC |
| 8101 | ATATGGACCG GGACCTGGGT TCCACGGGGA CTCCACACAC CACAAATGCT |
| 8151 | CCCAGCCCAC CGGGGCAGGA GACCTATTCT GCTGCTACCC CTGGAAAATG |
| 8201 | GGGAGAGGCC CCTGCAAGCC GAGTCGGCCA GTGGGACAGC CCTAGGCTGG |
| 8251 | ATCGGCCGCT TCGAGCAGAC ATGATAAGAT ACATTGATGA GTTTGGACAA |
| 8301 | ACCACAACTA GAATGCAGTG AAAAAAATGC TTTATTTGTG AAATTTGTGA |
| 8351 | TGCTATTGCT TTATTTGTAA CCATTATAAG CTGCAATAAA CAAGTTAACA |
| 8401 | ACAACAATTG CATTCATTTT ATGTTTCAGG TTCAGGGGGA GATGTGGGAG |
| 8451 | GTTTTTTAAA GCAAGTAAAA CCTCTACAAA TGTGGTAAAA TCGATTAGGA |
| 8501 | TCTTCCTAGA GCATGGCTAC |

ITR 5′: 5585-5720 bp
CMV promoter: 22-1043 bp
hIns: 1120-1466 bp
SV40 polyA: 1532-1754
ITR 3′: 1821-1971 bp
B: CMV-hIns

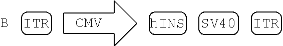

pGG2-CMV-hIns plasmid sequence
(SEQ ID NO: 17)

| | |
|---|---|
| 1 | CAGCAGCTGC GCGCTCGCTC GCTCACTGAG GCCGCCCGGG CAAAGCCCGG |
| 51 | GCGTCGGGCG ACCTTTGGTC GCCCGGCCTC AGTGAGCGAG CGAGCGCGCA |
| 101 | GAGAGGGAGT GGCCAACTCC ATCACTAGGG GTTCCTTGTA GTTAATGATT |
| 151 | AACCCGCCAT GCTACTTATC TACGTAGCCA TGCTCTAGAC ATGGCTCGAC |
| 201 | AGATCTCAAT ATTGGCCATT AGCCATATTA TTCATTGGTT ATATAGCATA |
| 251 | AATCAATATT GGCTATTGGC CATTGCATAC GTTGTATCTA TATCATAATA |
| 301 | TGTACATTTA TATTGGCTCA TGTCCAATAT GACCGCCATG TTGGCATTGA |
| 351 | TTATTGACTA GTTATTAATA GTAATCAATT ACGGGGTCAT TAGTTCATAG |
| 401 | CCCATATATG GAGTTCCGCG TTACATAACT TACGGTAAAT GGCCCGCCTG |
| 451 | GCTGACCGCC CAACGACCCC CGCCCATTGA CGTCAATAAT GACGTATGTT |
| 501 | CCCATAGTAA CGCCAATAGG GACTTTCCAT TGACGTCAAT GGGTGGAGTA |
| 551 | TTTACGGTAA ACTGCCCACT TGGCAGTACA TCAAGTGTAT CATATGCCAA |
| 601 | GTCCGCCCCC TATTGACGTC AATGACGGTA AATGGCCCGC CTGGCATTAT |
| 651 | GCCCAGTACA TGACCTTACG GGACTTTCCT ACTTGGCAGT ACATCTACGT |
| 701 | ATTAGTCATC GCTATTACCA TGGTGATGCG GTTTTGGCAG TACACCAATG |
| 751 | GGCGTGGATA GCGGTTTGAC TCACGGGGAT TTCCAAGTCT CCACCCCATT |
| 801 | GACGTCAATG GGAGTTTGTT TTGGCACCAA AATCAACGGG ACTTTCCAAA |
| 851 | ATGTCGTAAC AACTGCGATC GCCCGCCCCG TTGACGCAAA TGGGCGGTAG |
| 901 | GCGTGTACGG TGGGAGGTCT ATATAAGCAG AGCTCGTTTA GTGAACCGTC |
| 951 | AGATCACTAG AAGCTTTATT GCGGTAGTTT ATCACAGTTA AATTGCTAAC |
| 1001 | GCAGTCAGTG CTTCTGACAC AACAGTCTCG AACTTAAGCT GCAGTGACTC |
| 1051 | TCTTAAGGTA GCCTTGCAGA AGTTGGTCGT GAGGCACTGG GCAGGTAAGT |
| 1101 | ATCAAGGTTA CAAGACAGGT TTAAGGAGAC CAATAGAAAC TGGGCTTGTC |
| 1151 | GAGACAGAGA AGACTCTTGC GTTTCTGATA GGCACCTATT GGTCTTACTG |
| 1201 | ACATCCACTT TGCCTTTCTC TCCACAGGTG TCCACTCCCA GTTCAATTAC |
| 1251 | AGCTCTTAAG GCTAGAGTAC TTAATACGAC TCACTATAGG CTAGCCTCGA |
| 1301 | GAATTCTGCC ATGGCCCTGT GGATGCGCCT CCTGCCCCTG CTGGCGCTGC |
| 1351 | TGGCCCTCTG GGGACCTGAC CCAGCCGCAG CCTTTGTGAA CCAACACCTG |
| 1401 | TGCGGCTCAG ATCTGGTGGA AGCTCTCTAC CTAGTGTGCG GGGAACGAGG |
| 1451 | CTTCTTCTAC ACACCCAGGA CCAAGCGGGA GGCAGAGGAC CTGCAGGTGG |
| 1501 | GGCAGGTGGA GCTGGGCGGG GGCCCTGGTG CAGGCAGCCT GCAGCCCTTG |
| 1551 | GCCCTGGAGG GGTCGCGACA GAAGCGTGGC ATTGTGGAAC AATGCTGTAC |
| 1601 | CAGCATCTGC TCCCTCTACC AGCTGGAGAA CTACTGCAAC TAGACGCAGC |
| 1651 | TGCAAGCTTA TCGATACCGT CGACCTCGAG GAATTCACGC GTGGTACCTC |
| 1701 | TAGAGTCGAC CCGGGCGGCC GCTTCCCTTT AGTGAGGGTT AATGCTTCGA |
| 1751 | GCAGACATGA TAAGATACAT TGATGAGTTT GGACAAACCA CAACTAGAAT |
| 1801 | GCAGTGAAAA AAATGCTTTA TTTGTGAAAT TTGTGATGCT ATTGCTTTAT |
| 1851 | TTGTAACCAT TATAAGCTGC AATAAACAAG TTAACAACAA CAATTGCATT |
| 1901 | CATTTTATGT TTCAGGTTCA GGGGGAGATG TGGGAGGTTT TTTAAAGCAA |
| 1951 | GTAAAACCTC TACAAATGTG GTAAAATCCG ATAAGGGACT AGAGCATGGC |
| 2001 | TACGTAGATA AGTAGCATGG CGGGTTAATC ATTAACTACA AGGAACCCCT |
| 2051 | AGTGATGGAG TTGGCCACTC CCTCTCTGCG CGCTCGCTCG CTCACTGAGG |
| 2101 | CCGGGCGACC AAAGGTCGCC CGACGCCCGG GCTTTGCCCG GGCGGCCTCA |
| 2151 | GTGAGCGAGC GAGCGCGCCA GCTGGCGTAA TAGCGAAGAG GCCCGCACCG |
| 2201 | ATCGCCCTTC CCAACAGTTG CGCAGCCTGA ATGGCGAATG GAATTCCAGA |
| 2251 | CGATTGAGCG TCAAAATGTA GGTATTTCCA TGAGCGTTTT TCCGTTGCAA |
| 2301 | TGGCTGGCGG TAATATTGTT CTGGATATTA CCAGCAAGGC CGATAGTTTG |
| 2351 | AGTTCTTCTA CTCAGGCAAG TGATGTTATT ACTAATCAAA GAAGTATTGC |
| 2401 | GACAACGGTT AATTTGCGTG ATGGACAGAC TCTTTTACTC GGTGGCCTCA |
| 2451 | CTGATTATAA AAACACTTCT CAGGATTCTG GCGTACCGTT CCTGTCTAAA |
| 2501 | ATCCCTTTAA TCGGCCTCCT GTTTAGCTCC CGCTCTGATT CTAACGAGGA |
| 2551 | AAGCACGTTA TACGTGCTCG TCAAAGCAAC CATAGTACGC GCCCTGTAGC |
| 2601 | GGCGCATTAA GCGCGGCGGG TGTGGTGGTT ACGCGCAGCG TGACCGCTAC |
| 2651 | ACTTGCCAGC GCCCTAGCGC CCGCTCCTTT CGCTTTCTTC CCTTCCTTTC |
| 2701 | TCGCCACGTT CGCCGGCTTT CCCCGTCAAG CTCTAAATCG GGGGCTCCCT |
| 2751 | TTAGGGTTCC GATTTAGTGC TTTACGGCAC CTCGACCCCA AAAAACTTGA |
| 2801 | TTAGGGTGAT GGTTCACGTA GTGGGCCATC GCCCTGATAG ACGGTTTTTC |
| 2851 | GCCCTTTGAC GTTGGAGTCC ACGTTCTTTA ATAGTGGACT CTTGTTCCAA |
| 2901 | ACTGGAACAA CACTCAACCC TATCTCGGTC TATTCTTTTG ATTTATAAGG |
| 2951 | GATTTTGCCG ATTTCGGCCT ATTGGTTAAA AAATGAGCTG ATTTAACAAA |
| 3001 | AATTTAACGC GAATTTTAAC AAAATATTAA CGTCTACAAT TTAAATATTT |
| 3051 | GCTTATACAA TCTTCCTGTT TTTGGGGCTT TTCTGATTAT CAACCGGGGT |
| 3101 | ACATATGATT GACATGCTAG TTTTACGATT ACCGTTCATC GATTCTCTTG |
| 3151 | TTTGCTCCAG ACTCTCAGGC AATGACCTGA TAGCCTTTGT AGAGACCTCT |
| 3201 | CAAAAATAGC TACCCTCTCC GGCATGAATT TATCAGCTAG AACGGTTGAA |
| 3251 | TATCATATTG ATGGTGATTT GACTGTCTCC GGCCTTTCTC ACCCGTTTGA |
| 3301 | ATCTTTACCT ACACATTACT CAGGCATTGC ATTTAAAATA TATGAGGGTT |
| 3351 | CTAAAAATTT TTATCCTTGC GTTGAAATAA AGGCTTCTCC CGCAAAAGTA |
| 3401 | TTACAGGGTC ATAATGTTTT TGGTACAACC GATTTAGCTT TATGCTCTGA |
| 3451 | GGCTTTATTG CTTAATTTTG CTAATTCTTT GCCTTGCCTG TATGATTTAT |
| 3501 | TGGATGTTGG AATCGCCTGA TGCGGTATTT TCTCCTTACG CATCTGTGCG |
| 3551 | GTATTTCACA CCGCATATGG TGCACTCTCA GTACAATCTG CTCTGATGCC |
| 5601 | GCATAGTTAA GCCAGCCCCG ACACCCGCCA ACACCCGCTG ACGCGCCCTG |
| 3651 | ACGGGCTTGT CTGCTCCCGG CATCCGCTTA CAGACAAGCT GTGACCGTCT |
| 3701 | CCGGGAGCTG CATGTGTCAG AGGTTTTCAC CGTCATCACC GAAACGCGCG |
| 3751 | AGACGAAAGG GCCTCGTGAT ACGCCTATTT TTATAGGTTA ATGTCATGAT |
| 3801 | AATAATGGTT TCTTAGACGT CAGGTGGCAC TTTTCGGGGA AATGTGCGCG |
| 3851 | GAACCCCTAT TTGTTTATTT TTCTAAATAC ATTCAAATAT GTATCCGCTC |
| 3901 | ATGAGACAAT AACCCTGATA AATGCTTCAA TAATATTGAA AAAGGAAGAG |
| 3951 | TATGAGTATT CAACATTTCC GTGTCGCCCT TATTCCCTTT TTTGCGGCAT |
| 4001 | TTTGCCTTCC TGTTTTTGCT CACCCAGAAA CGCTGGTGAA AGTAAAAGAT |
| 4051 | GCTGAAGATC AGTTGGGTGC ACGAGTGGGT TACATCGAAC TGGATCTCAA |
| 4101 | CAGCGGTAAG ATCCTTGAGA GTTTTCGCCC CGAAGAACGT TTTCCAATGA |
| 4151 | TGAGCACTTT TAAAGTTCTG CTATGTGGCG CGGTATTATC CCGTATTGAC |
| 4201 | GCCGGGCAAG AGCAACTCGG TCGCCGCATA CACTATTCTC AGAATGACTT |
| 4251 | GGTTGAGTAC TCACCAGTCA CAGAAAAGCA TCTTACGGAT GGCATGACAG |
| 4301 | TAAGAGAATT ATGCAGTGCT GCCATAACCA TGAGTGATAA CACTGCGGCC |
| 4351 | AACTTACTTC TGACAACGAT CGGAGGACCG AAGGAGCTAA CCGCTTTTTT |
| 4401 | GCACAACATG GGGGATCATG TAACTCGCCT TGATCGTTGG GAACCGGAGC |
| 4451 | TGAATGAAGC CATACCAAAC GACGAGCGTG ACACCACGAT GCCTGTAGCA |
| 4501 | ATGGCAACAA CGTTGCGCAA ACTATTAACT GGCGAACTAC TTACTCTAGC |
| 4551 | TTCCCGGCAA CAATTAATAG ACTGGATGGA GGCGGATAAA GTTGCAGGAC |
| 4601 | CACTTCTGCG CTCGGCCCTT CCGGCTGGCT GGTTTATTGC TGATAAATCT |
| 4651 | GGAGCCGGTG AGCGTGGGTC TCGCGGTATC ATTGCAGCAC TGGGGCCAGA |
| 4701 | TGGTAAGCCC TCCCGTATCG TAGTTATCTA CACGACGGGG AGTCAGGCAA |
| 4751 | CTATGGATGA ACGAAATAGA CAGATCGCTG AGATAGGTGC CTCACTGATT |
| 4801 | AAGCATTGGT AACTGTCAGA CCAAGTTTAC TCATATATAC TTTAGATTGA |
| 4851 | TTTAAAACTT CATTTTTAAT TTAAAAGGAT CTAGGTGAAG ATCCTTTTTG |
| 4901 | ATAATCTCAT GACCAAAATC CCTTAACGTG AGTTTTCGTT CCACTGAGCG |
| 4951 | TCAGACCCCG TAGAAAAGAT CAAAGGATCT TCTTGAGATC CTTTTTTTCT |
| 5001 | GCGCGTAATC TGCTGCTTGC AAACAAAAAA ACCACCGCTA CCAGCGGTGG |
| 5051 | TTTGTTTGCC GGATCAAGAG CTACCAACTC TTTTTCCGAA GGTAACTGGC |
| 5101 | TTCAGCAGAG CGCAGATACC AAATACTGTC CTTCTAGTGT AGCCGTAGTT |
| 5151 | AGGCCACCAC TTCAAGAACT CTGTAGCACC GCCTACATAC CTCGCTCTGC |
| 5201 | TAATCCTGTT ACCAGTGGCT GCTGCCAGTG GCGATAAGTC GTGTCTTACC |
| 5251 | GGGTTGGACT CAAGACGATA GTTACCGGAT AAGGCGCAGC GGTCGGGCTG |
| 5301 | AACGGGGGGT TCGTGCACAC AGCCCAGCTT GGAGCGAACG ACCTACACCG |
| 5351 | AACTGAGATA CCTACAGCGT GAGCTATGAG AAAGCGCCAC GCTTCCCGAA |
| 5401 | GGGAGAAAGG CGGACAGGTA TCCGGTAAGC GGCAGGGTCG GAACAGGAGA |
| 5451 | GCGCACGAGG GAGCTTCCAG GGGGAAACGC CTGGTATCTT TATAGTCCTG |
| 5501 | TCGGGTTTCG CCACCTCTGA CTTGAGCGTC GATTTTTGTG ATGCTCGTCA |
| 5551 | GGGGGGCGGA GCCTATGGAA AAACGCCAGC AACGCGGCCT TTTTACGGTT |
| 5601 | CCTGGCCTTT TGCTGGCCTT TTGCTCACAT GTTCTTTCCT GCGTTATCCC |
| 5651 | CTGATTCTGT GGATAACCGT ATTACCGCCT TTGAGTGAGC TGATACCGCT |
| 5701 | CGCCGCAGCC GAACGACCGA GCGCAGCGAG TCAGTGAGCG AGGAAGCGGA |
| 5751 | AGAGCGCCCA ATACGCAAAC CGCCTCTCCC CGCGCGTTGG CCGATTCATT |
| 5801 | AATG |

ITR 5′: 1-136 bp
CMV promoter: 206-1227 bp
hIns: 1304-1650
SV40 polyA: 1752-1974 bp
ITR 3′: 2041-2191 bp
C: RSV-rGck

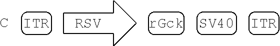

pGG2-RSV-rGck plasmid sequence
(SEQ ID NO: 18)

| | |
|---|---|
| 1 | GTAGATAAGT AGCATGGCGG GTTAATCATT AACTACAAGG AACCCCTAGT |
| 51 | GATGGAGTTG GCCACTCCCT CTCTGCGCGC TCGCTCGCTC ACTGAGGCCG |
| 101 | GGCGACCAAA GGTCGCCCGA CGCCCGGGCT TTGCCCGGGC GGCCTCAGTG |
| 151 | AGCGAGCGAG CGCGCCAGCT GGCGTAATAG CGAAGAGGCC CGCACCGATC |
| 201 | GCCCTTCCCA ACAGTTGCGC AGCCTGAATG GCGAATGGAA TTCCAGACGA |
| 251 | TTGAGCGTCA AAATGTAGGT ATTTCCATGA GCGTTTTTCC GTTGCAATGG |
| 301 | CTGGCGGTAA TATTGTTCTG GATATTACCA GCAAGGCCGA TAGTTTGAGT |
| 351 | TCTTCTACTC AGGCAAGTGA TGTTATTACT AATCAAAGAA GTATTGCGAC |
| 401 | AACGGTTAAT TTGCGTGATG GACAGACTCT TTTACTCGGT GGCCTCACTG |
| 451 | ATTATAAAAA CACTTCTCAG GATTCTGGCG TACCGTTCCT GTCTAAAATC |
| 501 | CCTTTAATCG GCCTCCTGTT TAGCTCCCGC TCTGATTCTA ACGAGGAAAG |
| 551 | CACGTTATAC GTGCTCGTCA AAGCAACCAT AGTACGCGCC CTGTAGCGGC |
| 601 | GCATTAAGCG CGGCGGGTGT GGTGGTTACG CGCAGCGTGA CCGCTACACT |
| 651 | TGCCAGCGCC CTAGCGCCCG CTCCTTTCGC TTTCTTCCCT TCCTTTCTCG |
| 701 | CCACGTTCGC CGGCTTTCCC CGTCAAGCTC TAAATCGGGG GCTCCCTTTA |
| 751 | GGGTTCCGAT TTAGTGCTTT ACGGCACCTC GACCCCAAAA AACTTGATTA |
| 801 | GGGTGATGGT TCACGTAGTG GGCCATCGCC CTGATAGACG GTTTTTCGCC |
| 851 | CTTTGACGTT GGAGTCCACG TTCTTTAATA GTGGACTCTT GTTCCAAACT |
| 901 | GGAACAACAC TCAACCCTAT CTCGGTCTAT TCTTTTGATT TATAAGGGAT |
| 951 | TTTGCCGATT TCGGCCTATT GGTTAAAAAA TGAGCTGATT TAACAAAAAT |
| 1001 | TTAACGCGAA TTTTAACAAA ATATTAACGT CTACAATTTA AATATTTGCT |
| 1051 | TATACAATCT TCCTGTTTTT GGGGCTTTTC TGATTATCAA CCGGGGTACA |
| 1101 | TATGATTGAC ATGCTAGTTT TACGATTACC GTTCATCGAT TCTCTTGTTT |
| 1151 | GCTCCAGACT CTCAGGCAAT GACCTGATAG CCTTTGTAGA GACCTCTCAA |
| 1201 | AAATAGCTAC CCTCTCCGGC ATGAATTTAT CAGCTAGAAC GGTTGAATAT |
| 1251 | CATATTGATG GTGATTTGAC TGTCTCCGGC CTTTCTCACC CGTTTGAATC |
| 1301 | TTTACCTACA CATTACTCAG GCATTGCATT TAAAATATAT GAGGGTTCTA |
| 1351 | AAAATTTTTA TCCTTGCGTT GAAATAAAGG CTTCTCCCGC AAAAGTATTA |
| 1401 | CAGGGTCATA ATGTTTTTGG TACAACCGAT TTAGCTTTAT GCTCTGAGGC |
| 1451 | TTTATTGCTT AATTTTGCTA ATTCTTTGCC TTGCCTGTAT GATTTATTGG |
| 1501 | ATGTTGGAAT CGCCTGATGC GGTATTTTCT CCTTACGCAT CTGTGCGGTA |
| 1551 | TTTCACACCG CATATGGTGC ACTCTCAGTA CAATCTGCTC TGATGCCGCA |
| 1601 | TAGTTAAGCC AGCCCCGACA CCCGCCAACA CCCGCTGACG CGCCCTGACG |
| 1651 | GGCTTGTCTG CTCCCGGCAT CCGCTTACAG ACAAGCTGTG ACCGTCTCCG |
| 1701 | GGAGCTGCAT GTGTCAGAGG TTTTCACCGT CATCACCGAA ACGCGCGAGA |
| 1751 | CGAAAGGGCC TCGTGATACG CCTATTTTTA TAGGTTAATG TCATGATAAT |
| 1801 | AATGGTTTCT TAGACGTCAG GTGGCACTTT TCGGGGAAAT GTGCGCGGAA |
| 1851 | CCCCTATTTG TTTATTTTTC TAAATACATT CAAATATGTA TCCGCTCATG |
| 1901 | AGACAATAAC CCTGATAAAT GCTTCAATAA TATTGAAAAA GGAAGAGTAT |
| 1951 | GAGTATTCAA CATTTCCGTG TCGCCCTTAT TCCCTTTTTT GCGGCATTTT |
| 2001 | GCCTTCCTGT TTTTGCTCAC CCAGAAACGC TGGTGAAAGT AAAAGATGCT |
| 2051 | GAAGATCAGT TGGGTGCACG AGTGGGTTAC ATCGAACTGG ATCTCAACAG |
| 2101 | CGGTAAGATC CTTGAGAGTT TTCGCCCCGA AGAACGTTTT CCAATGATGA |
| 2151 | GCACTTTTAA AGTTCTGCTA TGTGGCGCGG TATTATCCCG TATTGACGCC |
| 2201 | GGGCAAGAGC AACTCGGTCG CCGCATACAC TATTCTCAGA ATGACTTGGT |
| 2251 | TGAGTACTCA CCAGTCACAG AAAAGCATCT TACGGATGGC ATGACAGTAA |
| 2301 | GAGAATTATG CAGTGCTGCC ATAACCATGA GTGATAACAC TGCGGCCAAC |
| 2351 | TTACTTCTGA CAACGATCGG AGGACCGAAG GAGCTAACCG CTTTTTTGCA |
| 2401 | CAACATGGGG GATCATGTAA CTCGCCTTGA TCGTTGGGAA CCGGAGCTGA |
| 2451 | ATGAAGCCAT ACCAAACGAC GAGCGTGACA CCACGATGCC TGTAGCAATG |
| 2501 | GCAACAACGT TGCGCAAACT ATTAACTGGC GAACTACTTA CTCTAGCTTC |
| 2551 | CCGGCAACAA TTAATAGACT GGATGGAGGC GGATAAAGTT GCAGGACCAC |
| 2601 | TTCTGCGCTC GGCCCTTCCG GCTGGCTGGT TTATTGCTGA TAAATCTGGA |
| 2651 | GCCGGTGAGC GTGGGTCTCG CGGTATCATT GCAGCACTGG GGCCAGATGG |
| 2701 | TAAGCCCTCC CGTATCGTAG TTATCTACAC GACGGGGAGT CAGGCAACTA |
| 2751 | TGGATGAACG AAATAGACAG ATCGCTGAGA TAGGTGCCTC ACTGATTAAG |
| 2801 | CATTGGTAAC TGTCAGACCA AGTTTACTCA TATATACTTT AGATTGATTT |
| 2851 | AAAACTTCAT TTTTAATTTA AAAGGATCTA GGTGAAGATC CTTTTTGATA |
| 2901 | ATCTCATGAC CAAAATCCCT TAACGTGAGT TTTCGTTCCA CTGAGCGTCA |
| 2951 | GACCCCGTAG AAAAGATCAA AGGATCTTCT TGAGATCCTT TTTTTCTGCG |
| 3001 | CGTAATCTGC TGCTTGCAAA CAAAAAAACC ACCGCTACCA GCGGTGGTTT |
| 3051 | GTTTGCCGGA TCAAGAGCTA CCAACTCTTT TTCCGAAGGT AACTGGCTTC |
| 3101 | AGCAGAGCGC AGATACCAAA TACTGTCCTT CTAGTGTAGC CGTAGTTAGG |
| 3151 | CCACCACTTC AAGAACTCTG TAGCACCGCC TACATACCTC GCTCTGCTAA |
| 3201 | TCCTGTTACC AGTGGCTGCT GCCAGTGGCG ATAAGTCGTG TCTTACCGGG |
| 3251 | TTGGACTCAA GACGATAGTT ACCGGATAAG GCGCAGCGGT CGGGCTGAAC |
| 3301 | GGGGGGTTCG TGCACACAGC CCAGCTTGGA GCGAACGACC TACACCGAAC |
| 3351 | TGAGATACCT ACAGCGTGAG CTATGAGAAA GCGCCACGCT TCCCGAAGGG |
| 3401 | AGAAAGGCGG ACAGGTATCC GGTAAGCGGC AGGGTCGGAA CAGGAGAGCG |
| 3451 | CACGAGGGAG CTTCCAGGGG GAAACGCCTG GTATCTTTAT AGTCCTGTCG |
| 3501 | GGTTTCGCCA CCTCTGACTT GAGCGTCGAT TTTTGTGATG CTCGTCAGGG |
| 3551 | GGGCGGAGCC TATGGAAAAA CGCCAGCAAC GCGGCCTTTT TACGGTTCCT |
| 3601 | GGCCTTTTGC TGGCCTTTTG CTCACATGTT CTTTCCTGCG TTATCCCCTG |
| 3651 | ATTCTGTGGA TAACCGTATT ACCGCCTTTG AGTGAGCTGA TACCGCTCGC |
| 5701 | CGCAGCCGAA CGACCGAGCG CAGCGAGTCA GTGAGCGAGG AAGCGGAAGA |
| 5751 | GCGCCCAATA CGCAAACCGC CTCTCCCCGC GCGTTGGCCG ATTCATTAAT |
| 3801 | GCAGCAGCTG CGCGCTCGCT CGCTCACTGA GGCCGCCCGG GCAAAGCCCG |
| 3851 | GGCGTCGGGC GACCTTTGGT CGCCCGGCCT CAGTGAGCGA GCGAGCGCGC |
| 3901 | AGAGAGGGAG TGGCCAACTC CATCACTAGG GGTTCCTTGT AGTTAATGAT |
| 3951 | TAACCCGCCA TGCTACTTAT CTACGTAGCC ATGCTCTGGA AGATCTCGAC |
| 4001 | GCGTCATGTT TGACAGCTTA TCATCGCAGA TCCGTATGGT GCACTCTCAG |
| 4051 | TACAATCTGC TCTGATGCCG CATAGTTAAG CCAGTATCTG CTCCCTGCTT |
| 4101 | GTGTGTTGGA GGTCGCTGAG TAGTGCGCGA GCAAAATTTA AGCTACAACA |
| 4151 | AGGCAAGGCT TGACCGACAA TTGCATGAAG AATCTGCTTA GGGTTAGGCG |
| 4201 | TTTTGCGCTG CTTCGCGATG TACGGGCCAG ATATTCGCGT ATCTGAGGGG |
| 4251 | ACTAGGGTGT GTTTAGGCGA AAAGCGGGGC TTCGGTTGTA CGCGGTTAGG |
| 4301 | AGTCCCCTCA GGATATAGTA GTTTCGCTTT TGCATAGGGA GGGGGAAATG |
| 4351 | TAGTCTTATG CAATACTCTT GTAGTCTTGC AACATGGTAA CGATGAGTTA |
| 4401 | GCAACATGCC TTACAAGGAG AGAAAAAGCA CCGTGCATGC CGATTGGTGG |
| 4451 | AAGTAAGGTG GTACGATCGT GCCTTATTAG GAAGGCAACA GACGGGTCTG |
| 4501 | ACATGGATTG GACGAACCAC TAAATTCCGC ATTGCAGAGA TATTGTATTT |
| 4551 | AAGTGCCTAG CTCGATACAA TAAACGCCAT TTGACCATTC ACCACATTGG |
| 4601 | TGTGCACCTC CAAGCTGGGT ACCAGCTGCT AGCAAGCTTG AGATCTGCTT |
| 4651 | CAGCTGGAGG CACTGGGCAG GTAAGTATCA AGGTTACAAG ACAGGTTTAA |
| 4701 | GGAGACCAAT AGAAACTGGG CTTGTCGAGA CAGAGAAGAC TCTTGCGTTT |
| 4751 | CTGATAGGCA CCTATTGGTC TTACTGACAT CCACTTTGCC TTTCTCTCCA |
| 4801 | CAGGTGCAGC TGCTGCAGCG GGAATTCAAC AGGTGGCCTC AGGAGTCAGG |
| 4851 | AACATCTCTA CTTCCCCAAC GACCCCTGGG TTGTCCTCTC AGAGATGGCT |
| 4901 | ATGGATACTA CAAGGTGTGG AGCCCAGTTG TTGACTCTGG TCGAGCAGAT |
| 4951 | CCTGGCAGAG TTCCAGCTGC AGGAGGAAGA CCTGAAGAAG GTGATGAGCC |
| 5001 | GGATGCAGAA GGAGATGGAC CGTGGCCTGA GGCTGGAGAC CCACGAGGAG |
| 5051 | GCCAGTGTAA AGATGTTACC CACCTACGTG CGTTCCACCC CAGAAGGCTC |
| 5101 | AGAAGTCGGA GACTTTCTCT CCTTAGACCT GGGAGGAACC AACTTCAGAG |
| 5151 | TGATGCTGGT CAAAGTGGGA GAGGGGGAGG CAGGGCAGTG GAGCGTGAAG |
| 5201 | ACAAAACACC AGATGTACTC CATCCCCGAG GACGCCATGA CGGGCACTGC |
| 5251 | CGAGATGCTC TTTGACTACA TCTCTGAATG CATCTCTGAC TTCCTTGACA |
| 5301 | AGCATCAGAT GAAGCACAAG AAACTGCCCC TGGGCTTCAC CTTCTCCTTC |
| 5351 | CCTGTGAGGC ACGAAGACCT AGACAAGGGC ATCCTCCTCA ATTGGACCAA |
| 5401 | GGGCTTCAAG GCCTCTGGAG CAGAAGGGAA CAACATCGTA GGACTTCTCC |
| 5451 | GAGATGCTAT CAAGAGGAGA GGGGACTTTG AGATGGATGT GGTGGCAATG |
| 5501 | GTGAACGACA CAGTGGCCAC AATGATCTCC TGCTACTATG AAGACCGCCA |
| 5551 | ATGTGAGGTC GGCATGATTG TGGGCACTGG CTGCAATGCC TGCTACATGG |
| 5601 | AGGAAATGCA GAATGTGGAG CTGGTGGAAG GGGATGAGGG ACGCATGTGC |
| 5651 | GTCAACACGG AGTGGGGCGC CTTCGGGGAC TCGGGCGAGC TGGATGAGTT |
| 5701 | CCTACTGGAG TATGACCGGA TGGTGGATGA AAGCTCAGCG AACCCCGGTC |
| 5751 | AGCAGCTGTA CGAGAAGATC ATCGGTGGGA AGTATATGGG CGAGCTGGTA |
| 5801 | CGACTTGTGC TGCTTAAGCT GGTGGACGAG AACCTTCTGT TCCACGGAGA |
| 5851 | GGCCTCGGAG CAGCTGCGCA CGCGTGGTGC TTTTGAGACC CGTTTCGTGT |
| 5901 | CACAAGTGGA GAGCGACTCC GGGGACCGAA AGCAGATCCA CAACATCCTA |
| 5951 | AGCACTCTGG GGCTTCGACC CTCTGTCACC GACTGCGACA TTGTGCGCCG |
| 6001 | TGCCTGTGAA AGCGTGTCCA CTCGCGCCGC CCATATGTGC TCCGCAGGAC |
| 6051 | TAGCTGGGGT CATAAATCGC ATGCGCGAAA GCCGCAGTGA GGACGTGATG |
| 6101 | CGCATCACTG TGGGCGTGGA TGGCTCCGTG TACAAGCTGC ACCCGAGCTT |
| 6151 | CAAGGAGCGG TTTCACGCCA GTGTGCGCAG GCTGACACCC AACTGCGAAA |
| 6201 | TCACCTTCAT CGAATCAGAG GAGGGCAGCG GCAGGGGAGC CGCACTGGTC |
| 6251 | TCTGCGGTGG CCTGCAAGAA GGCTTGCATG CTGGCCCAGT GAAATCCAGG |
| 6301 | TCATATGGAC CGGGACCTGG GTTCCACGGG GACTCCACAC ACCACAAATG |
| 6351 | CTCCCAGCCC ACCGGGGCAG GAGACCTATT CTGCTGCTAC CCCTGGAAAA |
| 6401 | TGGGGAGAGG CCCCTGCAAG CCGAGTCGGC CAGTGGGACA GCCCTAGGCT |
| 6451 | GGATCGGCCG CTTCGAGCAG ACATGATAAG ATACATTGAT GAGTTTGGAC |
| 6501 | AAACCACAAC TAGAATGCAG TGAAAAAAAT GCTTTATTTG TGAAATTTGT |
| 6551 | GATGCTATTG CTTTATTTGT AACCATTATA AGCTGCAATA AACAAGTTAA |
| 6601 | CAACAACAAT TGCATTCATT TTATGTTTCA GGTTCAGGGG GAGATGTGGG |
| 6651 | AGGTTTTTTA AAGCAAGTAA AACCTCTACA AATGTGGTAA AATCGATTAG |
| 6701 | GATCTTCCTA GAGCATGGCT AC |

ITR 5′: 3802-3937 bp
RSV promoter: 4088-4803 bp
rGck: 4915-6292 bp
SV40 polyA: 6456-6694 bp
ITR 3′: 38-188 bp
D: CMV-hIns-RSV-hGck

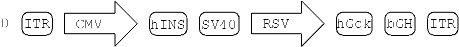

pAAV-CMV-hIns-RSV-hGck plasmid sequence
(SEQ ID NO: 9)

| | |
|---|---|
| 1 | CCTGCAGGCA GCTGCGCGCT CGCTCGCTCA CTGAGGCCGC CCGGGCAAAG |
| 51 | CCCGGGCGTC GGGCGACCTT TGGTCGCCCG GCCTCAGTGA GCGAGCGAGC |
| 101 | GCGCAGAGAG GGAGTGGCCA ACTCCATCAC TAGGGGTTCC TGCGGCCGCG |
| 151 | ATATCTGTAG TTAATGATTA ACCCGCCATG CTACTTATCT ACAGATCTCA |
| 201 | ATATTGGCCA TTAGCCATAT TATTCATTGG TTATATAGCA TAAATCAATA |
| 251 | TTGGCTATTG GCCATTGCAT ACGTTGTATC TATATCATAA TATGTACATT |
| 301 | TATATTGGCT CATGTCCAAT ATGACCGCCA TGTTGGCATT GATTATTGAC |
| 351 | TAGTTATTAA TAGTAATCAA TTACGGGGTC ATTAGTTCAT AGCCCATATA |
| 401 | TGGAGTTCCG CGTTACATAA CTTACGGTAA ATGGCCCGCC TGGCTGACCG |
| 451 | CCCAACGACC CCCGCCCATT GACGTCAATA ATGACGTATG TTCCCATAGT |
| 501 | AACGCCAATA GGGACTTTCC ATTGACGTCA ATGGGTGGAG TATTTACGGT |
| 551 | AAACTGCCCA CTTGGCAGTA CATCAAGTGT ATCATATGCC AAGTCCGCCC |
| 601 | CCTATTGACG TCAATGACGG TAAATGGCCC GCCTGGCATT ATGCCCAGTA |
| 651 | CATGACCTTA CGGGACTTTC CTACTTGGCA GTACATCTAC GTATTAGTCA |
| 701 | TCGCTATTAC CATGGTGATG CGGTTTTGGC AGTACACCAA TGGGCGTGGA |
| 751 | TAGCGGTTTG ACTCACGGGG ATTTCCAAGT CTCCACCCCA TTGACGTCAA |
| 801 | TGGGAGTTTG TTTTGGCACC AAAATCAACG GGACTTTCCA AAATGTCGTA |
| 851 | ACAACTGCGA TCGCCCGCCC CGTTGACGCA AATGGGCGGT AGGCGTGTAC |
| 901 | GGTGGGAGGT CTATATAAGC AGAGCTCGTT TAGTGAACCG TCAGATCACT |
| 951 | AGGCTAGCTA TTGCGGTAGT TTATCACAGT TAAATTGCTA ACGCAGTCAG |
| 1001 | TGCTTCTGAC ACAACAGTCT CGAACTTAAG CTGCAGTGAC TCTCTTAAGG |
| 1051 | TAGCCTTGCA GAAGTTGGTC GTGAGGCACT GGGCAGGTAA GTATCAAGGT |
| 1101 | TACAAGACAG GTTTAAGGAG ACCAATAGAA ACTGGGCTTG TCGAGACAGA |
| 1151 | GAAGACTCTT GCGTTTCTGA TAGGCACCTA TTGGTCTTAC TGACATCCAC |
| 1201 | TTTGCCTTTC TCTCCACAGG TGTCCACTCC CAGTTCAATT ACAGCTCTTA |
| 1251 | AGGCTAGAGT ACTTAATACG ACTCACTATA GAATACGACT CACTATAGGG |
| 1301 | AGACGCTAGC GTCGACCTTC TGCCATGGCC CTGTGGATGC GCCTCCTGCC |
| 1351 | CCTGCTGGCG CTGCTGGCCC TCTGGGGACC TGACCCAGCC GCAGCCTTTG |
| 1401 | TGAACCAACA CCTGTGCGGC TCAGATCTGG TGGAAGCTCT CTACCTAGTG |
| 1451 | TGCGGGGAAC GAGGCTTCTT CTACACACCC AGGACCAAGC GGGAGGCAGA |
| 1501 | GGACCTGCAG GTGGGGCAGG TGGAGCTGGG CGGGGGCCCT GGTGCAGGCA |
| 1551 | GCCTGCAGCC CTTGGCCCTG GAGGGGTCGC GACAGAAGCG TGGCATTGTG |
| 1601 | GAACAATGCT GTACCAGCAT CTGCTCCCTC TACCAGCTGG AGAACTACTG |
| 1651 | CAACTAGACG CAGCCGTCGA CGGTACCAGC GCTGTCGAGG CCGCTTCGAG |
| 1701 | CAGACATGAT AAGATACATT GATGAGTTTG GACAAACCAC AACTAGAATG |
| 1751 | CAGTGAAAAA AATGCTTTAT TTGTGAAATT TGTGATGCTA TTGCTTTATT |
| 1801 | TGTAACCATT ATAAGCTGCA ATAAACAAGT TAACAACAAC AATTGCATTC |
| 1851 | ATTTTATGTT TCAGGTTCAG GGGGAGATGT GGGAGGTTTT TTAAAGCAAG |
| 1901 | TAAAACCTCT ACAAATGTGG TAAAATCGAT TAGGATCTTC CTAGAGCATG |
| 1951 | GCTACCTAGA CATGGCTCGA CAGATCAGCG CTCATGCTCT GGAAGATCTC |
| 2001 | GATTTATCCA TGTTTGACAG CTTATCATCG CAGATCCGTA TGGTGCACTC |
| 2051 | TCAGTACAAT CTGCTCTGAT GCCGCATAGT TAAGCCAGTA TCTGCTCCCT |
| 2101 | GCTTGTGTGT TGGAGGTCGC TGAGTAGTGC GCGAGCAAAA TTTAAGCTAC |
| 2151 | AACAAGGCAA GGCTTGACCG ACAATTGCAT GAAGAATCTG CTTAGGGTTA |
| 2201 | GGCGTTTTGC GCTGCTTCGC GATGTACGGG CCAGATATTC GCGTATCTGA |
| 2251 | GGGGACTAGG GTGTGTTTAG GCGAAAAGCG GGGCTTCGGT TGTACGCGGT |
| 2301 | TAGGAGTCCC CTCAGGATAT AGTAGTTTCG CTTTTGCATA GGGAGGGGGA |
| 2351 | AATGTAGTCT TATGCAATAC TCTTGTAGTC TTGCAACATG GTAACGATGA |
| 2401 | GTTAGCAACA TGCCTTACAA GGAGAGAAAA AGCACCGTGC ATGCCGATTG |
| 2451 | GTGGAAGTAA GGTGGTACGA TCGTGCCTTA TTAGGAAGGC AACAGACGGG |
| 2501 | TCTGACATGG ATTGGACGAA CCACTAAATT CCGCATTGCA GAGATATTGT |
| 2551 | ATTTAAGTGC CTAGCTCGAT ACAATAAACG CCATTTGACC ATTCACCACA |
| 2601 | TTGGTGTGCA CCTCCAAGCT GGGTACCAGC TTCTAGAGAG ATCTGCTTCA |
| 2651 | GCTGGAGGCA CTGGGCAGGT AAGTATCAAG GTTACAAGAC AGGTTTAAGG |
| 2701 | AGACCAATAG AAACTGGGCT TGTCGAGACA GAGAAGACTC TTGCGTTTCT |
| 2751 | GATAGGCACC TATTGGTCTT ACTGACATCC ACTTTGCCTT TCTCTCCACA |
| 2801 | GGTGCAGCTG CTGCAGCGGT CTAGAACTCG AGTCGAGACC ATGGCGATGG |
| 2851 | ATGTCACAAG GAGCCAGGCC CAGACAGCCT TGACTCTGGT AGAGCAGATC |
| 2901 | CTGGCAGAGT TCCAGCTGCA GGAGGAGGAC CTGAAGAAGG TGATGAGACG |
| 2951 | GATGCAGAAG GAGATGGACC GCGGCCTGAG GCTGGAGACC CATGAAGAGG |
| 3001 | CCAGTGTGAA GATGCTGCCC ACCTACGTGC GCTCCACCCC AGAAGGCTCA |
| 3051 | GAAGTCGGGG ACTTCCTCTC CCTGGACCTG GGTGGCACTA ACTTCAGGGT |
| 3101 | GATGCTGGTG AAGGTGGGAG AAGGTGAGGA GGGGCAGTGG AGCGTGAAGA |
| 3151 | CCAAACACCA GATGTACTCC ATCCCCGAGG ACGCCATGAC CGGCACTGCT |
| 3201 | GAGATGCTCT TCGACTACAT CTCTGAGTGC ATCTCCGACT TCCTGGACAA |
| 3251 | GCATCAGATG AAACACAAGA AGCTGCCCCT GGGCTTCACC TTCTCCTTTC |
| 3301 | CTGTGAGGCA CGAAGACATC GATAAGGGCA TCCTTCTCAA CTGGACCAAG |
| 3351 | GGCTTCAAGG CCTCAGGAGC AGAAGGGAAC AATGTCGTGG GGCTTCTGCG |
| 3401 | AGACGCTATC AAACGGAGAG GGGACTTTGA AATGGATGTG GTGGCAATGG |
| 3451 | TGAATGACAC GGTGGCCACG ATGATCTCCT GCTACTACGA AGACCATCAG |
| 3501 | TGCGAGGTCG GCATGATCGT GGGCACGGGC TGCAATGCCT GCTACATGGA |
| 3551 | GGAGATGCAG AATGTGGAGC TGGTGGAGGG GGACGAGGGC CGCATGTGCG |
| 5601 | TCAATACCGA GTGGGGCGCC TTCGGGGACT CCGGCGAGCT GGACGAGTTC |
| 3651 | CTGCTGGAGT ATGACCGCCT GGTGGACGAG AGCTCTGCAA ACCCCGGTCA |
| 3701 | GCAGCTGTAT GAGAAGCTCA TAGGTGGCAA GTACATGGGC GAGCTGGTGC |
| 3751 | GGCTTGTGCT GCTCAGGCTC GTGGACGAAA ACCTGCTCTT CCACGGGGAG |
| 3801 | GCCTCCGAGC AGCTGCGCAC ACGCGGAGCC TTCGAGACGC GCTTCGTGTC |
| 3851 | GCAGGTGGAG AGCGACACGG GCGACCGCAA GCAGATCTAC AACATCCTGA |
| 3901 | GCACGCTGGG GCTGCGACCC TCGACCACCG ACTGCGACAT CGTGCGCCGC |
| 3951 | GCCTGCGAGA GCGTGTCTAC GCGCGCTGCG CACATGTGCT CGGCGGGGCT |
| 4001 | GGCGGGCGTC ATCAACCGCA TGCGCGAGAG CCGCAGCGAG GACGTAATGC |
| 4051 | GCATCACTGT GGGCGTGGAT GGCTCCGTGT ACAAGCTGCA CCCCAGCTTC |
| 4101 | AAGGAGCGGT TCCATGCCAG CGTGCGCAGG CTGACGCCCA GCTGCGAGAT |
| 4151 | CACCTTCATC GAGTCGGAGG AGGGCAGTGG CCGGGGCGCG GCCCTGGTCT |
| 4201 | CGGCGGTGGC CTGTAAGAAG GCCTGTATGC TGGGCCAGTG ACTCGAGCAC |
| 4251 | GTGGAGCTCG CTGATCAGCC TCGACTGTGC CTTCTAGTTG CCAGCCATCT |
| 4301 | GTTGTTTGCC CCTCCCCCGT GCCTTCCTTG ACCCTGGAAG GTGCCACTCC |
| 4351 | CACTGTCCTT TCCTAATAAA ATGAGGAAAT TGCATCGCAT TGTCTGAGTA |
| 4401 | GGTGTCATTC TATTCTGGGG GGTGGGGTGG GGCAGGACAG CAAGGGGGAG |
| 4451 | GATTGGGAAG ACAATAGCAG GCATGCTGGG GATGCGGTGG GCTCTATGGC |
| 4501 | CACGTGATTT AAATGCGGCC GCAGGAACCC CTAGTGATGG AGTTGGCCAC |
| 4551 | TCCCTCTCTG CGCGCTCGCT CGCTCACTGA GGCCGGGCGA CCAAAGGTCG |
| 4601 | CCCGACGCCC GGGCTTTGCC CGGGCGGCCT CAGTGAGCGA GCGAGCGCGC |
| 4651 | AGCTGCCTGC AGGGGCGCCT GATGCGGTAT TTTCTCCTTA CGCATCTGTG |
| 4701 | CGGTATTTCA CACCGCATAC GTCAAAGCAA CCATAGTACG CGCCCTGTAG |
| 4751 | CGGCGCATTA AGCGCGGCGG GTGTGGTGGT TACGCGCAGC GTGACCGCTA |
| 4801 | CACTTGCCAG CGCCCTAGCG CCCGCTCCTT TCGCTTTCTT CCCTTCCTTT |
| 4851 | CTCGCCACGT TCGCCGGCTT TCCCCGTCAA GCTCTAAATC GGGGGCTCCC |
| 4901 | TTTAGGGTTC CGATTTAGTG CTTTACGGCA CCTCGACCCC AAAAAACTTG |
| 4951 | ATTTGGGTGA TGGTTCACGT AGTGGGCCAT CGCCCTGATA GACGGTTTTT |
| 5001 | CGCCCTTTGA CGTTGGAGTC CACGTTCTTT AATAGTGGAC TCTTGTTCCA |
| 5051 | AACTGGAACA ACACTCAACC CTATCTCGGG CTATTCTTTT GATTTATAAG |
| 5101 | GGATTTTGCC GATTTCGGCC TATTGGTTAA AAAATGAGCT GATTTAACAA |
| 5151 | AAATTTAACG CGAATTTTAA CAAAATATTA ACGTTTACAA TTTTATGGTG |
| 5201 | CACTCTCAGT ACAATCTGCT CTGATGCCGC ATAGTTAAGC CAGCCCCGAC |
| 5251 | ACCCGCCAAC ACCCGCTGAC GCGCCCTGAC GGGCTTGTCT GCTCCCGGCA |
| 5301 | TCCGCTTACA GACAAGCTGT GACCGTCTCC GGGAGCTGCA TGTGTCAGAG |
| 5351 | GTTTTCACCG TCATCACCGA AACGCGCGAG ACGAAAGGGC CTCGTGATAC |
| 5401 | GCCTATTTTT ATAGGTTAAT GTCATGATAA TAATGGTTTC TTAGACGTCA |
| 5451 | GGTGGCACTT TTCGGGGAAA TGTGCGCGGA ACCCCTATTT GTTTATTTTT |
| 5501 | CTAAATACAT TCAAATATGT ATCCGCTCAT GAGACAATAA CCCTGATAAA |
| 5551 | TGCTTCAATA ATATTGAAAA AGGAAGAGTA TGAGTATTCA ACATTTCCGT |
| 5601 | GTCGCCCTTA TTCCCTTTTT TGCGGCATTT TGCCTTCCTG TTTTTGCTCA |
| 5651 | CCCAGAAACG CTGGTGAAAG TAAAAGATGC TGAAGATCAG TTGGGTGCAC |
| 5701 | GAGTGGGTTA CATCGAACTG GATCTCAACA GCGGTAAGAT CCTTGAGAGT |
| 5751 | TTTCGCCCCG AAGAACGTTT TCCAATGATG AGCACTTTTA AAGTTCTGCT |
| 5801 | ATGTGGCGCG GTATTATCCC GTATTGACGC CGGGCAAGAG CAACTCGGTC |
| 5851 | GCCGCATACA CTATTCTCAG AATGACTTGG TTGAGTACTC ACCAGTCACA |
| 5901 | GAAAAGCATC TTACGGATGG CATGACAGTA AGAGAATTAT GCAGTGCTGC |
| 5951 | CATAACCATG AGTGATAACA CTGCGGCCAA CTTACTTCTG ACAACGATCG |
| 6001 | GAGGACCGAA GGAGCTAACC GCTTTTTTGC ACAACATGGG GGATCATGTA |
| 6051 | ACTCGCCTTG ATCGTTGGGA ACCGGAGCTG AATGAAGCCA TACCAAACGA |
| 6101 | CGAGCGTGAC ACCACGATGC CTGTAGCAAT GGCAACAACG TTGCGCAAAC |
| 6151 | TATTAACTGG CGAACTACTT ACTCTAGCTT CCCGGCAACA ATTAATAGAC |
| 6201 | TGGATGGAGG CGGATAAAGT TGCAGGACCA CTTCTGCGCT CGGCCCTTCC |
| 6251 | GGCTGGCTGG TTTATTGCTG ATAAATCTGG AGCCGGTGAG CGTGGGTCTC |
| 6301 | GCGGTATCAT TGCAGCACTG GGGCCAGATG GTAAGCCCTC CCGTATCGTA |
| 6351 | GTTATCTACA CGACGGGGAG TCAGGCAACT ATGGATGAAC GAAATAGACA |
| 6401 | GATCGCTGAG ATAGGTGCCT CACTGATTAA GCATTGGTAA CTGTCAGACC |
| 6451 | AAGTTTACTC ATATATACTT TAGATTGATT TAAAACTTCA TTTTTAATTT |
| 6501 | AAAAGGATCT AGGTGAAGAT CCTTTTTGAT AATCTCATGA CCAAAATCCC |
| 6551 | TTAACGTGAG TTTTCGTTCC ACTGAGCGTC AGACCCCGTA GAAAAGATCA |
| 6601 | AAGGATCTTC TTGAGATCCT TTTTTTCTGC GCGTAATCTG CTGCTTGCAA |
| 6651 | ACAAAAAAAC CACCGCTACC AGCGGTGGTT TGTTTGCCGG ATCAAGAGCT |
| 6701 | ACCAACTCTT TTTCCGAAGG TAACTGGCTT CAGCAGAGCG CAGATACCAA |
| 6751 | ATACTGTCCT TCTAGTGTAG CCGTAGTTAG GCCACCACTT CAAGAACTCT |
| 6801 | GTAGCACCGC CTACATACCT CGCTCTGCTA ATCCTGTTAC CAGTGGCTGC |
| 6851 | TGCCAGTGGC GATAAGTCGT GTCTTACCGG GTTGGACTCA AGACGATAGT |
| 6901 | TACCGGATAA GGCGCAGCGG TCGGGCTGAA CGGGGGGTTC GTGCACACAG |
| 6951 | CCCAGCTTGG AGCGAACGAC CTACACCGAA CTGAGATACC TACAGCGTGA |
| 7001 | GCTATGAGAA AGCGCCACGC TTCCCGAAGG GAGAAAGGCG GACAGGTATC |
| 7051 | CGGTAAGCGG CAGGGTCGGA ACAGGAGAGC GCACGAGGGA GCTTCCAGGG |
| 7101 | GGAAACGCCT GGTATCTTTA TAGTCCTGTC GGGTTTCGCC ACCTCTGACT |
| 7151 | TGAGCGTCGA TTTTTGTGAT GCTCGTCAGG GGGGCGGAGC CTATGGAAAA |
| 7201 | ACGCCAGCAA CGCGGCCTTT TTACGGTTCC TGGCCTTTTG CTGGCCTTTT |
| 7251 | GCTCACATGT |

ITR 5′: 1-141 bp
CMV promoter: 193-1310 bp
hIns: 1318-1664 bp
SV40 polyA: 1678-1976 bp
RSV promoter: 2092-2801 bp
hGck: 2826-4240 bp
bGH polyA: 4248-4506 bp
ITR 3′: 4523-4663 bp
E: RSV-hGck-CMV-hIns

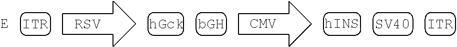

pAAV-RSV-hGck-CMV-hIns plasmid sequence
(SEQ ID NO: 10)

| | |
|---|---|
| 1 | CCTGCAGGCA GCTGCGCGCT CGCTCGCTCA CTGAGGCCGC CCGGGCAAAG |
| 51 | CCCGGGCGTC GGGCGACCTT TGGTCGCCCG GCCTCAGTGA GCGAGCGAGC |
| 101 | GCGCAGAGAG GGAGTGGCCA ACTCCATCAC TAGGGGTTCC TGCGGCCGCG |
| 151 | ATATCCATGT TTGACAGCTT ATCATCGCAG ATCCGTATGG TGCACTCTCA |
| 201 | GTACAATCTG CTCTGATGCC GCATAGTTAA GCCAGTATCT GCTCCCTGCT |
| 251 | TGTGTGTTGG AGGTCGCTGA GTAGTGCGCG AGCAAAATTT AAGCTACAAC |
| 301 | AAGGCAAGGC TTGACCGACA ATTGCATGAA GAATCTGCTT AGGGTTAGGC |
| 351 | GTTTTGCGCT GCTTCGCGAT GTACGGGCCA GATATTCGCG TATCTGAGGG |
| 401 | GACTAGGGTG TGTTTAGGCG AAAAGCGGGG CTTCGGTTGT ACGCGGTTAG |
| 451 | GAGTCCCCTC AGGATATAGT AGTTTCGCTT TTGCATAGGG AGGGGGAAAT |
| 501 | GTAGTCTTAT GCAATACTCT TGTAGTCTTG CAACATGGTA ACGATGAGTT |
| 551 | AGCAACATGC CTTACAAGGA GAGAAAAAGC ACCGTGCATG CCGATTGGTG |
| 601 | GAAGTAAGGT GGTACGATCG TGCCTTATTA GGAAGGCAAC AGACGGGTCT |
| 651 | GACATGGATT GGACGAACCA CTAAATTCCG CATTGCAGAG ATATTGTATT |
| 701 | TAAGTGCCTA GCTCGATACA ATAAACGCCA TTTGACCATT CACCACATTG |
| 751 | GTGTGCACCT CCAAGCTGGG TACCAGCTTC TAGAGAGATC TGCTTCAGCT |
| 801 | GGAGGCACTG GGCAGGTAAG TATCAAGGTT ACAAGACAGG TTTAAGGAGA |
| 851 | CCAATAGAAA CTGGGCTTGT CGAGACAGAG AAGACTCTTG CGTTTCTGAT |
| 901 | AGGCACCTAT TGGTCTTACT GACATCCACT TTGCCTTTCT CTCCACAGGT |
| 951 | GCAGCTGCTG CAGCGGTCTA GAACTCGAGT CGAGACCATG GCGATGGATG |
| 1001 | TCACAAGGAG CCAGGCCCAG ACAGCCTTGA CTCTGGTAGA GCAGATCCTG |
| 1051 | GCAGAGTTCC AGCTGCAGGA GGAGGACCTG AAGAAGGTGA TGAGACGGAT |
| 1101 | GCAGAAGGAG ATGGACCGCG GCCTGAGGCT GGAGACCCAT GAAGAGGCCA |
| 1151 | GTGTGAAGAT GCTGCCCACC TACGTGCGCT CCACCCCAGA AGGCTCAGAA |
| 1201 | GTCGGGGACT TCCTCTCCCT GGACCTGGGT GGCACTAACT TCAGGGTGAT |
| 1251 | GCTGGTGAAG GTGGGAGAAG GTGAGGAGGG GCAGTGGAGC GTGAAGACCA |
| 1301 | AACACCAGAT GTACTCCATC CCCGAGGACG CCATGACCGG CACTGCTGAG |
| 1351 | ATGCTCTTCG ACTACATCTC TGAGTGCATC TCCGACTTCC TGGACAAGCA |
| 1401 | TCAGATGAAA CACAAGAAGC TGCCCCTGGG CTTCACCTTC TCCTTTCCTG |
| 1451 | TGAGGCACGA AGACATCGAT AAGGGCATCC TTCTCAACTG GACCAAGGGC |
| 1501 | TTCAAGGCCT CAGGAGCAGA AGGGAACAAT GTCGTGGGGC TTCTGCGAGA |
| 1551 | CGCTATCAAA CGGAGAGGGG ACTTTGAAAT GGATGTGGTG GCAATGGTGA |
| 1601 | ATGACACGGT GGCCACGATG ATCTCCTGCT ACTACGAAGA CCATCAGTGC |
| 1651 | GAGGTCGGCA TGATCGTGGG CACGGGCTGC AATGCCTGCT ACATGGAGGA |
| 1701 | GATGCAGAAT GTGGAGCTGG TGGAGGGGGA CGAGGGCCGC ATGTGCGTCA |
| 1751 | ATACCGAGTG GGGCGCCTTC GGGGACTCCG GCGAGCTGGA CGAGTTCCTG |
| 1801 | CTGGAGTATG ACCGCCTGGT GGACGAGAGC TCTGCAAACC CCGGTCAGCA |
| 1851 | GCTGTATGAG AAGCTCATAG GTGGCAAGTA CATGGGCGAG CTGGTGCGGC |
| 1901 | TTGTGCTGCT CAGGCTCGTG GACGAAAACC TGCTCTTCCA CGGGGAGGCC |
| 1951 | TCCGAGCAGC TGCGCACACG CGGAGCCTTC GAGACGCGCT TCGTGTCGCA |
| 2001 | GGTGGAGAGC GACACGGGCG ACCGCAAGCA GATCTACAAC ATCCTGAGCA |
| 2051 | CGCTGGGGCT GCGACCCTCG ACCACCGACT GCGACATCGT GCGCCGCGCC |
| 2101 | TGCGAGAGCG TGTCTACGCG CGCTGCGCAC ATGTGCTCGG CGGGGCTGGC |
| 2151 | GGGCGTCATC AACCGCATGC GCGAGAGCCG CAGCGAGGAC GTAATGCGCA |
| 2201 | TCACTGTGGG CGTGGATGGC TCCGTGTACA AGCTGCACCC CAGCTTCAAG |
| 2251 | GAGCGGTTCC ATGCCAGCGT GCGCAGGCTG ACGCCCAGCT GCGAGATCAC |
| 2301 | CTTCATCGAG TCGGAGGAGG GCAGTGGCCG GGGCGCGGCC CTGGTCTCGG |
| 2351 | CGGTGGCCTG TAAGAAGGCC TGTATGCTGG GCCAGTGACT CGAGCACGTG |
| 2401 | GAGCTCGCTG ATCAGCCTCG ACTGTGCCTT CTAGTTGCCA GCCATCTGTT |
| 2451 | GTTTGCCCCT CCCCCGTGCC TTCCTTGACC CTGGAAGGTG CCACTCCCAC |
| 2501 | TGTCCTTTCC TAATAAAATG AGGAAATTGC ATCGCATTGT CTGAGTAGGT |
| 2551 | GTCATTCTAT TCTGGGGGGT GGGGTGGGGC AGGACAGCAA GGGGGAGGAT |
| 2601 | TGGGAAGACA ATAGCAGGCA TGCTGGGGAT GCGGTGGGCT CTATGGCCAC |
| 2651 | GTGATTTATC TGTAGTTAAT GATTAACCCG CCATGCTACT TATCTACAGA |
| 2701 | TCTCAATATT GGCCATTAGC CATATTATTC ATTGGTTATA TAGCATAAAT |
| 2751 | CAATATTGGC TATTGGCCAT TGCATACGTT GTATCTATAT CATAATATGT |
| 2801 | ACATTTATAT TGGCTCATGT CCAATATGAC CGCCATGTTG GCATTGATTA |
| 2851 | TTGACTAGTT ATTAATAGTA ATCAATTACG GGGTCATTAG TTCATAGCCC |
| 2901 | ATATATGGAG TTCCGCGTTA CATAACTTAC GGTAAATGGC CCGCCTGGCT |
| 2951 | GACCGCCCAA CGACCCCCGC CCATTGACGT CAATAATGAC GTATGTTCCC |
| 3001 | ATAGTAACGC CAATAGGGAC TTTCCATTGA CGTCAATGGG TGGAGTATTT |
| 3051 | ACGGTAAACT GCCCACTTGG CAGTACATCA AGTGTATCAT ATGCCAAGTC |
| 3101 | CGCCCCCTAT TGACGTCAAT GACGGTAAAT GGCCCGCCTG GCATTATGCC |
| 3151 | CAGTACATGA CCTTACGGGA CTTTCCTACT TGGCAGTACA TCTACGTATT |
| 3201 | AGTCATCGCT ATTACCATGG TGATGCGGTT TTGGCAGTAC ACCAATGGGC |
| 3251 | GTGGATAGCG GTTTGACTCA CGGGGATTTC CAAGTCTCCA CCCCATTGAC |
| 3301 | GTCAATGGGA GTTTGTTTTG GCACCAAAAT CAACGGGACT TTCCAAAATG |
| 3351 | TCGTAACAAC TGCGATCGCC CGCCCCGTTG ACGCAAATGG GCGGTAGGCG |
| 3401 | TGTACGGTGG GAGGTCTATA TAAGCAGAGC TCGTTTAGTG AACCGTCAGA |
| 3451 | TCACTAGGCT AGCTATTGCG GTAGTTTATC ACAGTTAAAT TGCTAACGCA |
| 3501 | GTCAGTGCTT CTGACACAAC AGTCTCGAAC TTAAGCTGCA GTGACTCTCT |
| 3551 | TAAGGTAGCC TTGCAGAAGT TGGTCGTGAG GCACTGGGCA GGTAAGTATC |
| 3601 | AAGGTTACAA GACAGGTTTA AGGAGACCAA TAGAAACTGG GCTTGTCGAG |
| 3651 | ACAGAGAAGA CTCTTGCGTT TCTGATAGGC ACCTATTGGT CTTACTGACA |
| 3701 | TCCACTTTGC CTTTCTCTCC ACAGGTGTCC ACTCCCAGTT CAATTACAGC |
| 3751 | TCTTAAGGCT AGAGTACTTA ATACGACTCA CTATAGAATA CGACTCACTA |
| 3801 | TAGGGAGACG CTAGCGTCGA CCTTCTGCCA TGGCCCTGTG GATGCGCCTC |
| 3851 | CTGCCCCTGC TGGCGCTGCT GGCCCTCTGG GGACCTGACC CAGCCGCAGC |
| 3901 | CTTTGTGAAC CAACACCTGT GCGGCTCAGA TCTGGTGGAA GCTCTCTACC |
| 3951 | TAGTGTGCGG GGAACGAGGC TTCTTCTACA CACCCAGGAC CAAGCGGGAG |
| 4001 | GCAGAGGACC TGCAGGTGGG GCAGGTGGAG CTGGGCGGGG GCCCTGGTGC |
| 4051 | AGGCAGCCTG CAGCCCTTGG CCCTGGAGGG GTCGCGACAG AAGCGTGGCA |
| 4101 | TTGTGGAACA ATGCTGTACC AGCATCTGCT CCCTCTACCA GCTGGAGAAC |
| 4151 | TACTGCAACT AGACGCAGCC GTCGACGGTA CCAGCGCTGT CGAGGCCGCT |
| 4201 | TCGAGCAGAC ATGATAAGAT ACATTGATGA GTTTGGACAA ACCACAACTA |
| 4251 | GAATGCAGTG AAAAAAATGC TTTATTTGTG AAATTTGTGA TGCTATTGCT |
| 4301 | TTATTTGTAA CCATTATAAG CTGCAATAAA CAAGTTAACA ACAACAATTG |
| 4351 | CATTCATTTT ATGTTTCAGG TTCAGGGGGA GATGTGGGAG GTTTTTTAAA |
| 4401 | GCAAGTAAAA CCTCTACAAA TGTGGTAAAA TCGATTAGGA TCTTCCTAGA |
| 4451 | GCATGGCTAC CTAGACATGG CTCGACAGAT CAGCGCTCAT GCTCTGGAAG |
| 4501 | ATCTCGATTT AAATGCGGCC GCAGGAACCC CTAGTGATGG AGTTGGCCAC |
| 4551 | TCCCTCTCTG CGCGCTCGCT CGCTCACTGA GGCCGGGCGA CCAAAGGTCG |
| 4601 | CCCGACGCCC GGGCTTTGCC CGGGCGGCCT CAGTGAGCGA GCGAGCGCGC |
| 4651 | AGCTGCCTGC AGGGGCGCCT GATGCGGTAT TTTCTCCTTA CGCATCTGTG |
| 4701 | CGGTATTTCA CACCGCATAC GTCAAAGCAA CCATAGTACG CGCCCTGTAG |
| 4751 | CGGCGCATTA AGCGCGGCGG GTGTGGTGGT TACGCGCAGC GTGACCGCTA |
| 4801 | CACTTGCCAG CGCCCTAGCG CCCGCTCCTT TCGCTTTCTT CCCTTCCTTT |
| 4851 | CTCGCCACGT TCGCCGGCTT TCCCCGTCAA GCTCTAAATC GGGGGCTCCC |
| 4901 | TTTAGGGTTC CGATTTAGTG CTTTACGGCA CCTCGACCCC AAAAAACTTG |
| 4951 | ATTTGGGTGA TGGTTCACGT AGTGGGCCAT CGCCCTGATA GACGGTTTTT |
| 5001 | CGCCCTTTGA CGTTGGAGTC CACGTTCTTT AATAGTGGAC TCTTGTTCCA |
| 5051 | AACTGGAACA ACACTCAACC CTATCTCGGG CTATTCTTTT GATTTATAAG |
| 5101 | GGATTTTGCC GATTTCGGCC TATTGGTTAA AAAATGAGCT GATTTAACAA |
| 5151 | AAATTTAACG CGAATTTTAA CAAAATATTA ACGTTTACAA TTTTATGGTG |
| 5201 | CACTCTCAGT ACAATCTGCT CTGATGCCGC ATAGTTAAGC CAGCCCCGAC |
| 5251 | ACCCGCCAAC ACCCGCTGAC GCGCCCTGAC GGGCTTGTCT GCTCCCGGCA |
| 5301 | TCCGCTTACA GACAAGCTGT GACCGTCTCC GGGAGCTGCA TGTGTCAGAG |
| 5351 | GTTTTCACCG TCATCACCGA AACGCGCGAG ACGAAAGGGC CTCGTGATAC |
| 5401 | GCCTATTTTT ATAGGTTAAT GTCATGATAA TAATGGTTTC TTAGACGTCA |
| 5451 | GGTGGCACTT TTCGGGGAAA TGTGCGCGGA ACCCCTATTT GTTTATTTTT |
| 5501 | CTAAATACAT TCAAATATGT ATCCGCTCAT GAGACAATAA CCCTGATAAA |
| 5551 | TGCTTCAATA ATATTGAAAA AGGAAGAGTA TGAGTATTCA ACATTTCCGT |
| 5601 | GTCGCCCTTA TTCCCTTTTT TGCGGCATTT TGCCTTCCTG TTTTTGCTCA |
| 5651 | CCCAGAAACG CTGGTGAAAG TAAAAGATGC TGAAGATCAG TTGGGTGCAC |
| 5701 | GAGTGGGTTA CATCGAACTG GATCTCAACA GCGGTAAGAT CCTTGAGAGT |
| 5751 | TTTCGCCCCG AAGAACGTTT TCCAATGATG AGCACTTTTA AAGTTCTGCT |
| 5801 | ATGTGGCGCG GTATTATCCC GTATTGACGC CGGGCAAGAG CAACTCGGTC |
| 5851 | GCCGCATACA CTATTCTCAG AATGACTTGG TTGAGTACTC ACCAGTCACA |
| 5901 | GAAAAGCATC TTACGGATGG CATGACAGTA AGAGAATTAT GCAGTGCTGC |
| 5951 | CATAACCATG AGTGATAACA CTGCGGCCAA CTTACTTCTG ACAACGATCG |
| 6001 | GAGGACCGAA GGAGCTAACC GCTTTTTTGC ACAACATGGG GGATCATGTA |
| 6051 | ACTCGCCTTG ATCGTTGGGA ACCGGAGCTG AATGAAGCCA TACCAAACGA |
| 6101 | CGAGCGTGAC ACCACGATGC CTGTAGCAAT GGCAACAACG TTGCGCAAAC |
| 6151 | TATTAACTGG CGAACTACTT ACTCTAGCTT CCCGGCAACA ATTAATAGAC |
| 6201 | TGGATGGAGG CGGATAAAGT TGCAGGACCA CTTCTGCGCT CGGCCCTTCC |
| 6251 | GGCTGGCTGG TTTATTGCTG ATAAATCTGG AGCCGGTGAG CGTGGGTCTC |
| 6301 | GCGGTATCAT TGCAGCACTG GGGCCAGATG GTAAGCCCTC CCGTATCGTA |
| 6351 | GTTATCTACA CGACGGGGAG TCAGGCAACT ATGGATGAAC GAAATAGACA |
| 6401 | GATCGCTGAG ATAGGTGCCT CACTGATTAA GCATTGGTAA CTGTCAGACC |
| 6451 | AAGTTTACTC ATATATACTT TAGATTGATT TAAAACTTCA TTTTTAATTT |
| 6501 | AAAAGGATCT AGGTGAAGAT CCTTTTTGAT AATCTCATGA CCAAAATCCC |
| 6551 | TTAACGTGAG TTTTCGTTCC ACTGAGCGTC AGACCCCGTA GAAAAGATCA |
| 6601 | AAGGATCTTC TTGAGATCCT TTTTTTCTGC GCGTAATCTG CTGCTTGCAA |
| 6651 | ACAAAAAAAC CACCGCTACC AGCGGTGGTT TGTTTGCCGG ATCAAGAGCT |
| 6701 | ACCAACTCTT TTTCCGAAGG TAACTGGCTT CAGCAGAGCG CAGATACCAA |
| 6751 | ATACTGTCCT TCTAGTGTAG CCGTAGTTAG GCCACCACTT CAAGAACTCT |
| 6801 | GTAGCACCGC CTACATACCT CGCTCTGCTA ATCCTGTTAC CAGTGGCTGC |
| 6851 | TGCCAGTGGC GATAAGTCGT GTCTTACCGG GTTGGACTCA AGACGATAGT |
| 6901 | TACCGGATAA GGCGCAGCGG TCGGGCTGAA CGGGGGGTTC GTGCACACAG |
| 6951 | CCCAGCTTGG AGCGAACGAC CTACACCGAA CTGAGATACC TACAGCGTGA |
| 7001 | GCTATGAGAA AGCGCCACGC TTCCCGAAGG GAGAAAGGCG GACAGGTATC |
| 7051 | CGGTAAGCGG CAGGGTCGGA ACAGGAGAGC GCACGAGGGA GCTTCCAGGG |
| 7101 | GGAAACGCCT GGTATCTTTA TAGTCCTGTC GGGTTTCGCC ACCTCTGACT |
| 7151 | TGAGCGTCGA TTTTTGTGAT GCTCGTCAGG GGGGCGGAGC CTATGGAAAA |
| 7201 | ACGCCAGCAA CGCGGCCTTT TTACGGTTCC TGGCCTTTTG CTGGCCTTTT |
| 7251 | GCTCACATGT |

ITR 5′: 1-141 bp
RSV promoter: 239-948 bp
hGck: 973-2387 bp
bGH polyA: 2395-2653 bp
CMV promoter: 2698-3815 bp
hIns: 3823-4169 bp
SV40 polyA: 4183-4481 bp
ITR 3′: 4523-4663 bp
F: CMV-hIns(rev)-RSV-hGck

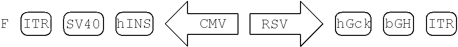

pAAV-CMV-hIns(rev)-RSV-hGck plasmid sequence
(SEQ ID NO: 11)

| | |
|---|---|
| 1 | CCTGCAGGCA GCTGCGCGCT CGCTCGCTCA CTGAGGCCGC CCGGGCAAAG |
| 51 | CCCGGGCGTC GGGCGACCTT TGGTCGCCCG GCCTCAGTGA GCGAGCGAGC |
| 101 | GCGCAGAGAG GGAGTGGCCA ACTCCATCAC TAGGGGTTCC TGCGGCCGCG |
| 151 | ATAAATCGAG ATCTTCCAGA GCATGAGCGC TGATCTGTCG AGCCATGTCT |
| 201 | AGGTAGCCAT GCTCTAGGAA GATCCTAATC GATTTTACCA CATTTGTAGA |
| 251 | GGTTTTACTT GCTTTAAAAA ACCTCCCACA TCTCCCCCTG AACCTGAAAC |
| 301 | ATAAAATGAA TGCAATTGTT GTTGTTAACT TGTTTATTGC AGCTTATAAT |
| 351 | GGTTACAAAT AAAGCAATAG CATCACAAAT TTCACAAATA AAGCATTTTT |
| 401 | TTCACTGCAT TCTAGTTGTG GTTTGTCCAA ACTCATCAAT GTATCTTATC |
| 451 | ATGTCTGCTC GAAGCGGCCT CGACAGCGCT GGTACCGTCG ACGGCTGCGT |
| 501 | CTAGTTGCAG TAGTTCTCCA GCTGGTAGAG GGAGCAGATG CTGGTACAGC |
| 551 | ATTGTTCCAC AATGCCACGC TTCTGTCGCG ACCCCTCCAG GGCCAAGGGC |
| 601 | TGCAGGCTGC CTGCACCAGG GCCCCCGCCC AGCTCCACCT GCCCCACCTG |
| 651 | CAGGTCCTCT GCCTCCCGCT TGGTCCTGGG TGTGTAGAAG AAGCCTCGTT |
| 701 | CCCCGCACAC TAGGTAGAGA GCTTCCACCA GATCTGAGCC GCACAGGTGT |
| 751 | TGGTTCACAA AGGCTGCGGC TGGGTCAGGT CCCCAGAGGG CCAGCAGCGC |
| 801 | CAGCAGGGGC AGGAGGCGCA TCCACAGGGC CATGGCAGAA GGTCGACGCT |
| 851 | AGCGTCTCCC TATAGTGAGT CGTATTCTAT AGTGAGTCGT ATTAAGTACT |
| 901 | CTAGCCTTAA GAGCTGTAAT TGAACTGGGA GTGGACACCT GTGGAGAGAA |
| 951 | AGGCAAAGTG GATGTCAGTA AGACCAATAG GTGCCTATCA GAAACGCAAG |
| 1001 | AGTCTTCTCT GTCTCGACAA GCCCAGTTTC TATTGGTCTC CTTAAACCTG |
| 1051 | TCTTGTAACC TTGATACTTA CCTGCCCAGT GCCTCACGAC CAACTTCTGC |
| 1101 | AAGGCTACCT TAAGAGAGTC ACTGCAGCTT AAGTTCGAGA CTGTTGTGTC |
| 1151 | AGAAGCACTG ACTGCGTTAG CAATTTAACT GTGATAAACT ACCGCAATAG |
| 1201 | CTAGCCTAGT GATCTGACGG TTCACTAAAC GAGCTCTGCT TATATAGACC |
| 1251 | TCCCACCGTA CACGCCTACC GCCCATTTGC GTCAACGGGG CGGGCGATCG |
| 1301 | CAGTTGTTAC GACATTTTGG AAAGTCCCGT TGATTTTGGT GCCAAAACAA |
| 1351 | ACTCCCATTG ACGTCAATGG GGTGGAGACT TGGAAATCCC CGTGAGTCAA |
| 1401 | ACCGCTATCC ACGCCCATTG GTGTACTGCC AAAACCGCAT CACCATGGTA |
| 1451 | ATAGCGATGA CTAATACGTA GATGTACTGC CAAGTAGGAA AGTCCCGTAA |
| 1501 | GGTCATGTAC TGGGCATAAT GCCAGGCGGG CCATTTACCG TCATTGACGT |
| 1551 | CAATAGGGGG CGGACTTGGC ATATGATACA CTTGATGTAC TGCCAAGTGG |
| 1601 | GCAGTTTACC GTAAATACTC CACCCATTGA CGTCAATGGA AAGTCCCTAT |
| 1651 | TGGCGTTACT ATGGGAACAT ACGTCATTAT TGACGTCAAT GGGCGGGGGT |
| 1701 | CGTTGGGCGG TCAGCCAGGC GGGCCATTTA CCGTAAGTTA TGTAACGCGG |
| 1751 | AACTCCATAT ATGGGCTATG AACTAATGAC CCCGTAATTG ATTACTATTA |
| 1801 | ATAACTAGTC AATAATCAAT GCCAACATGG CGGTCATATT GGACATGAGC |
| 1851 | CAATATAAAT GTACATATTA TGATATAGAT ACAACGTATG CAATGGCCAA |
| 1901 | TAGCCAATAT TGATTTATGC TATATAACCA ATGAATAATA TGGCTAATGG |
| 1951 | CCAATATTGA GATCTGTAGA TAAGTAGCAT GGCGGGTTAA TCATTAACTA |
| 2001 | CAGATATCCA TGTTTGACAG CTTATCATCG CAGATCCGTA TGGTGCACTC |
| 2051 | TCAGTACAAT CTGCTCTGAT GCCGCATAGT TAAGCCAGTA TCTGCTCCCT |
| 2101 | GCTTGTGTGT TGGAGGTCGC TGAGTAGTGC GCGAGCAAAA TTTAAGCTAC |
| 2151 | AACAAGGCAA GGCTTGACCG ACAATTGCAT GAAGAATCTG CTTAGGGTTA |
| 2201 | GGCGTTTTGC GCTGCTTCGC GATGTACGGG CCAGATATTC GCGTATCTGA |
| 2251 | GGGGACTAGG GTGTGTTTAG GCGAAAAGCG GGGCTTCGGT TGTACGCGGT |
| 2301 | TAGGAGTCCC CTCAGGATAT AGTAGTTTCG CTTTTGCATA GGGAGGGGGA |
| 2351 | AATGTAGTCT TATGCAATAC TCTTGTAGTC TTGCAACATG GTAACGATGA |
| 2401 | GTTAGCAACA TGCCTTACAA GGAGAGAAAA AGCACCGTGC ATGCCGATTG |
| 2451 | GTGGAAGTAA GGTGGTACGA TCGTGCCTTA TTAGGAAGGC AACAGACGGG |
| 2501 | TCTGACATGG ATTGGACGAA CCACTAAATT CCGCATTGCA GAGATATTGT |
| 2551 | ATTTAAGTGC CTAGCTCGAT ACAATAAACG CCATTTGACC ATTCACCACA |
| 2601 | TTGGTGTGCA CCTCCAAGCT GGGTACCAGC TTCTAGAGAG ATCTGCTTCA |
| 2651 | GCTGGAGGCA CTGGGCAGGT AAGTATCAAG GTTACAAGAC AGGTTTAAGG |
| 2701 | AGACCAATAG AAACTGGGCT TGTCGAGACA GAGAAGACTC TTGCGTTTCT |
| 2751 | GATAGGCACC TATTGGTCTT ACTGACATCC ACTTTGCCTT TCTCTCCACA |
| 2801 | GGTGCAGCTG CTGCAGCGGT CTAGAACTCG AGTCGAGACC ATGGCGATGG |
| 2851 | ATGTCACAAG GAGCCAGGCC CAGACAGCCT TGACTCTGGT AGAGCAGATC |
| 2901 | CTGGCAGAGT TCCAGCTGCA GGAGGAGGAC CTGAAGAAGG TGATGAGACG |
| 2951 | GATGCAGAAG GAGATGGACC GCGGCCTGAG GCTGGAGACC CATGAAGAGG |
| 3001 | CCAGTGTGAA GATGCTGCCC ACCTACGTGC GCTCCACCCC AGAAGGCTCA |
| 3051 | GAAGTCGGGG ACTTCCTCTC CCTGGACCTG GGTGGCACTA ACTTCAGGGT |
| 3101 | GATGCTGGTG AAGGTGGGAG AAGGTGAGGA GGGGCAGTGG AGCGTGAAGA |
| 3151 | CCAAACACCA GATGTACTCC ATCCCCGAGG ACGCCATGAC CGGCACTGCT |
| 3201 | GAGATGCTCT TCGACTACAT CTCTGAGTGC ATCTCCGACT TCCTGGACAA |
| 3251 | GCATCAGATG AAACACAAGA AGCTGCCCCT GGGCTTCACC TTCTCCTTTC |
| 3301 | CTGTGAGGCA CGAAGACATC GATAAGGGCA TCCTTCTCAA CTGGACCAAG |
| 3351 | GGCTTCAAGG CCTCAGGAGC AGAAGGGAAC AATGTCGTGG GGCTTCTGCG |
| 3401 | AGACGCTATC AAACGGAGAG GGGACTTTGA AATGGATGTG GTGGCAATGG |
| 3451 | TGAATGACAC GGTGGCCACG ATGATCTCCT GCTACTACGA AGACCATCAG |
| 3501 | TGCGAGGTCG GCATGATCGT GGGCACGGGC TGCAATGCCT GCTACATGGA |
| 3551 | GGAGATGCAG AATGTGGAGC TGGTGGAGGG GGACGAGGGC CGCATGTGCG |
| 3601 | TCAATACCGA GTGGGGCGCC TTCGGGGACT CCGGCGAGCT GGACGAGTTC |
| 3651 | CTGCTGGAGT ATGACCGCCT GGTGGACGAG AGCTCTGCAA ACCCCGGTCA |
| 3701 | GCAGCTGTAT GAGAAGCTCA TAGGTGGCAA GTACATGGGC GAGCTGGTGC |
| 3751 | GGCTTGTGCT GCTCAGGCTC GTGGACGAAA ACCTGCTCTT CCACGGGGAG |
| 3801 | GCCTCCGAGC AGCTGCGCAC ACGCGGAGCC TTCGAGACGC GCTTCGTGTC |
| 3851 | GCAGGTGGAG AGCGACACGG GCGACCGCAA GCAGATCTAC AACATCCTGA |
| 3901 | GCACGCTGGG GCTGCGACCC TCGACCACCG ACTGCGACAT CGTGCGCCGC |
| 3951 | GCCTGCGAGA GCGTGTCTAC GCGCGCTGCG CACATGTGCT CGGCGGGGCT |
| 4001 | GGCGGGCGTC ATCAACCGCA TGCGCGAGAG CCGCAGCGAG GACGTAATGC |
| 4051 | GCATCACTGT GGGCGTGGAT GGCTCCGTGT ACAAGCTGCA CCCCAGCTTC |
| 4101 | AAGGAGCGGT TCCATGCCAG CGTGCGCAGG CTGACGCCCA GCTGCGAGAT |
| 4151 | CACCTTCATC GAGTCGGAGG AGGGCAGTGG CCGGGGCGCG GCCCTGGTCT |
| 4201 | CGGCGGTGGC CTGTAAGAAG GCCTGTATGC TGGGCCAGTG ACTCGAGCAC |
| 4251 | GTGGAGCTCG CTGATCAGCC TCGACTGTGC CTTCTAGTTG CCAGCCATCT |
| 4301 | GTTGTTTGCC CCTCCCCCGT GCCTTCCTTG ACCCTGGAAG GTGCCACTCC |
| 4351 | CACTGTCCTT TCCTAATAAA ATGAGGAAAT TGCATCGCAT TGTCTGAGTA |
| 4401 | GGTGTCATTC TATTCTGGGG GGTGGGGTGG GGCAGGACAG CAAGGGGGAG |
| 4451 | GATTGGGAAG ACAATAGCAG GCATGCTGGG GATGCGGTGG GCTCTATGGC |
| 4501 | CACGTGATTT AAATGCGGCC GCAGGAACCC CTAGTGATGG AGTTGGCCAC |
| 4551 | TCCCTCTCTG CGCGCTCGCT CGCTCACTGA GGCCGGGCGA CCAAAGGTCG |
| 4601 | CCCGACGCCC GGGCTTTGCC CGGGCGGCCT CAGTGAGCGA GCGAGCGCGC |
| 4651 | AGCTGCCTGC AGGGGCGCCT GATGCGGTAT TTTCTCCTTA CGCATCTGTG |
| 4701 | CGGTATTTCA CACCGCATAC GTCAAAGCAA CCATAGTACG CGCCCTGTAG |
| 4751 | CGGCGCATTA AGCGCGGCGG GTGTGGTGGT TACGCGCAGC GTGACCGCTA |
| 4801 | CACTTGCCAG CGCCCTAGCG CCCGCTCCTT TCGCTTTCTT CCCTTCCTTT |
| 4851 | CTCGCCACGT TCGCCGGCTT TCCCCGTCAA GCTCTAAATC GGGGGCTCCC |
| 4901 | TTTAGGGTTC CGATTTAGTG CTTTACGGCA CCTCGACCCC AAAAAACTTG |
| 4951 | ATTTGGGTGA TGGTTCACGT AGTGGGCCAT CGCCCTGATA GACGGTTTTT |
| 5001 | CGCCCTTTGA CGTTGGAGTC CACGTTCTTT AATAGTGGAC TCTTGTTCCA |
| 5051 | AACTGGAACA ACACTCAACC CTATCTCGGG CTATTCTTTT GATTTATAAG |
| 5101 | GGATTTTGCC GATTTCGGCC TATTGGTTAA AAAATGAGCT GATTTAACAA |
| 5151 | AAATTTAACG CGAATTTTAA CAAAATATTA ACGTTTACAA TTTTATGGTG |
| 5201 | CACTCTCAGT ACAATCTGCT CTGATGCCGC ATAGTTAAGC CAGCCCCGAC |
| 5251 | ACCCGCCAAC ACCCGCTGAC GCGCCCTGAC GGGCTTGTCT GCTCCCGGCA |
| 5301 | TCCGCTTACA GACAAGCTGT GACCGTCTCC GGGAGCTGCA TGTGTCAGAG |
| 5351 | GTTTTCACCG TCATCACCGA AACGCGCGAG ACGAAAGGGC CTCGTGATAC |
| 5401 | GCCTATTTTT ATAGGTTAAT GTCATGATAA TAATGGTTTC TTAGACGTCA |
| 5451 | GGTGGCACTT TTCGGGGAAA TGTGCGCGGA ACCCCTATTT GTTTATTTTT |
| 5501 | CTAAATACAT TCAAATATGT ATCCGCTCAT GAGACAATAA CCCTGATAAA |
| 5551 | TGCTTCAATA ATATTGAAAA AGGAAGAGTA TGAGTATTCA ACATTTCCGT |
| 5601 | GTCGCCCTTA TTCCCTTTTT TGCGGCATTT TGCCTTCCTG TTTTTGCTCA |
| 5651 | CCCAGAAACG CTGGTGAAAG TAAAAGATGC TGAAGATCAG TTGGGTGCAC |
| 5701 | GAGTGGGTTA CATCGAACTG GATCTCAACA GCGGTAAGAT CCTTGAGAGT |
| 5751 | TTTCGCCCCG AAGAACGTTT TCCAATGATG AGCACTTTTA AAGTTCTGCT |
| 5801 | ATGTGGCGCG GTATTATCCC GTATTGACGC CGGGCAAGAG CAACTCGGTC |
| 5851 | GCCGCATACA CTATTCTCAG AATGACTTGG TTGAGTACTC ACCAGTCACA |
| 5901 | GAAAAGCATC TTACGGATGG CATGACAGTA AGAGAATTAT GCAGTGCTGC |
| 5951 | CATAACCATG AGTGATAACA CTGCGGCCAA CTTACTTCTG ACAACGATCG |
| 6001 | GAGGACCGAA GGAGCTAACC GCTTTTTTGC ACAACATGGG GGATCATGTA |
| 6051 | ACTCGCCTTG ATCGTTGGGA ACCGGAGCTG AATGAAGCCA TACCAAACGA |
| 6101 | CGAGCGTGAC ACCACGATGC CTGTAGCAAT GGCAACAACG TTGCGCAAAC |
| 6151 | TATTAACTGG CGAACTACTT ACTCTAGCTT CCCGGCAACA ATTAATAGAC |
| 6201 | TGGATGGAGG CGGATAAAGT TGCAGGACCA CTTCTGCGCT CGGCCCTTCC |
| 6251 | GGCTGGCTGG TTTATTGCTG ATAAATCTGG AGCCGGTGAG CGTGGGTCTC |
| 6301 | GCGGTATCAT TGCAGCACTG GGGCCAGATG GTAAGCCCTC CCGTATCGTA |
| 6351 | GTTATCTACA CGACGGGGAG TCAGGCAACT ATGGATGAAC GAAATAGACA |
| 6401 | GATCGCTGAG ATAGGTGCCT CACTGATTAA GCATTGGTAA CTGTCAGACC |
| 6451 | AAGTTTACTC ATATATACTT TAGATTGATT TAAAACTTCA TTTTTAATTT |
| 6501 | AAAAGGATCT AGGTGAAGAT CCTTTTTGAT AATCTCATGA CCAAAATCCC |
| 6551 | TTAACGTGAG TTTTCGTTCC ACTGAGCGTC AGACCCCGTA GAAAAGATCA |
| 6601 | AAGGATCTTC TTGAGATCCT TTTTTTCTGC GCGTAATCTG CTGCTTGCAA |
| 6651 | ACAAAAAAAC CACCGCTACC AGCGGTGGTT TGTTTGCCGG ATCAAGAGCT |
| 6701 | ACCAACTCTT TTTCCGAAGG TAACTGGCTT CAGCAGAGCG CAGATACCAA |
| 6751 | ATACTGTCCT TCTAGTGTAG CCGTAGTTAG GCCACCACTT CAAGAACTCT |
| 6801 | GTAGCACCGC CTACATACCT CGCTCTGCTA ATCCTGTTAC CAGTGGCTGC |
| 6851 | TGCCAGTGGC GATAAGTCGT GTCTTACCGG GTTGGACTCA AGACGATAGT |
| 6901 | TACCGGATAA GGCGCAGCGG TCGGGCTGAA CGGGGGGTTC GTGCACACAG |
| 6951 | CCCAGCTTGG AGCGAACGAC CTACACCGAA CTGAGATACC TACAGCGTGA |
| 7001 | GCTATGAGAA AGCGCCACGC TTCCCGAAGG GAGAAAGGCG GACAGGTATC |
| 7051 | CGGTAAGCGG CAGGGTCGGA ACAGGAGAGC GCACGAGGGA GCTTCCAGGG |
| 7101 | GGAAACGCCT GGTATCTTTA TAGTCCTGTC GGGTTTCGCC ACCTCTGACT |
| 7151 | TGAGCGTCGA TTTTTGTGAT GCTCGTCAGG GGGGCGGAGC CTATGGAAAA |
| 7201 | ACGCCAGCAA CGCGGCCTTT TTACGGTTCC TGGCCTTTTG CTGGCCTTTT |
| 7251 | GCTCACATGT |

ITR 5′: 1-141 bp
SV40 polyA: 182-480 bp
hIns: 494-840 bp
CMV promoter: 4248-1965 bp
RSV promoter: 2092-2801 bp
hGck: 2826-4240 bp
bGH polyA: 4248-4506 bp
ITR 3′: 4523-4663 bp
G: RSV-hGck-CMV-hIns(rev)

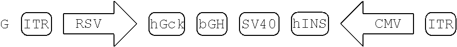

pAAV-RSV-hGck-CMV-hIns(rev) plasmid sequence
(SEQ ID NO: 12)

| | |
|---|---|
| 1 | CCTGCAGGCA GCTGCGCGCT CGCTCGCTCA CTGAGGCCGC CCGGGCAAAG |
| 51 | CCCGGGCGTC GGGCGACCTT TGGTCGCCCG GCCTCAGTGA GCGAGCGAGC |
| 101 | GCGCAGAGAG GGAGTGGCCA ACTCCATCAC TAGGGGTTCC TGCGGCCGCG |
| 151 | ATATCCATGT TTGACAGCTT ATCATCGCAG ATCCGTATGG TGCACTCTCA |
| 201 | GTACAATCTG CTCTGATGCC GCATAGTTAA GCCAGTATCT GCTCCCTGCT |
| 251 | TGTGTGTTGG AGGTCGCTGA GTAGTGCGCG AGCAAAATTT AAGCTACAAC |
| 301 | AAGGCAAGGC TTGACCGACA ATTGCATGAA GAATCTGCTT AGGGTTAGGC |
| 351 | GTTTTGCGCT GCTTCGCGAT GTACGGGCCA GATATTCGCG TATCTGAGGG |
| 401 | GACTAGGGTG TGTTTAGGCG AAAAGCGGGG CTTCGGTTGT ACGCGGTTAG |
| 451 | GAGTCCCCTC AGGATATAGT AGTTTCGCTT TTGCATAGGG AGGGGGAAAT |
| 501 | GTAGTCTTAT GCAATACTCT TGTAGTCTTG CAACATGGTA ACGATGAGTT |
| 551 | AGCAACATGC CTTACAAGGA GAGAAAAAGC ACCGTGCATG CCGATTGGTG |
| 601 | GAAGTAAGGT GGTACGATCG TGCCTTATTA GGAAGGCAAC AGACGGGTCT |
| 651 | GACATGGATT GGACGAACCA CTAAATTCCG CATTGCAGAG ATATTGTATT |
| 701 | TAAGTGCCTA GCTCGATACA ATAAACGCCA TTTGACCATT CACCACATTG |
| 751 | GTGTGCACCT CCAAGCTGGG TACCAGCTTC TAGAGAGATC TGCTTCAGCT |
| 801 | GGAGGCACTG GGCAGGTAAG TATCAAGGTT ACAAGACAGG TTTAAGGAGA |
| 851 | CCAATAGAAA CTGGGCTTGT CGAGACAGAG AAGACTCTTG CGTTTCTGAT |
| 901 | AGGCACCTAT TGGTCTTACT GACATCCACT TTGCCTTTCT CTCCACAGGT |
| 951 | GCAGCTGCTG CAGCGGTCTA GAACTCGAGT CGAGACCATG GCGATGGATG |
| 1001 | TCACAAGGAG CCAGGCCCAG ACAGCCTTGA CTCTGGTAGA GCAGATCCTG |
| 1051 | GCAGAGTTCC AGCTGCAGGA GGAGGACCTG AAGAAGGTGA TGAGACGGAT |
| 1101 | GCAGAAGGAG ATGGACCGCG GCCTGAGGCT GGAGACCCAT GAAGAGGCCA |
| 1151 | GTGTGAAGAT GCTGCCCACC TACGTGCGCT CCACCCCAGA AGGCTCAGAA |
| 1201 | GTCGGGGACT TCCTCTCCCT GGACCTGGGT GGCACTAACT TCAGGGTGAT |
| 1251 | GCTGGTGAAG GTGGGAGAAG GTGAGGAGGG GCAGTGGAGC GTGAAGACCA |
| 1301 | AACACCAGAT GTACTCCATC CCCGAGGACG CCATGACCGG CACTGCTGAG |
| 1351 | ATGCTCTTCG ACTACATCTC TGAGTGCATC TCCGACTTCC TGGACAAGCA |
| 1401 | TCAGATGAAA CACAAGAAGC TGCCCCTGGG CTTCACCTTC TCCTTTCCTG |
| 1451 | TGAGGCACGA AGACATCGAT AAGGGCATCC TTCTCAACTG GACCAAGGGC |
| 1501 | TTCAAGGCCT CAGGAGCAGA AGGGAACAAT GTCGTGGGGC TTCTGCGAGA |
| 1551 | CGCTATCAAA CGGAGAGGGG ACTTTGAAAT GGATGTGGTG GCAATGGTGA |
| 1601 | ATGACACGGT GGCCACGATG ATCTCCTGCT ACTACGAAGA CCATCAGTGC |
| 1651 | GAGGTCGGCA TGATCGTGGG CACGGGCTGC AATGCCTGCT ACATGGAGGA |
| 1701 | GATGCAGAAT GTGGAGCTGG TGGAGGGGGA CGAGGGCCGC ATGTGCGTCA |
| 1751 | ATACCGAGTG GGGCGCCTTC GGGGACTCCG GCGAGCTGGA CGAGTTCCTG |
| 1801 | CTGGAGTATG ACCGCCTGGT GGACGAGAGC TCTGCAAACC CCGGTCAGCA |
| 1851 | GCTGTATGAG AAGCTCATAG GTGGCAAGTA CATGGGCGAG CTGGTGCGGC |
| 1901 | TTGTGCTGCT CAGGCTCGTG GACGAAAACC TGCTCTTCCA CGGGGAGGCC |
| 1951 | TCCGAGCAGC TGCGCACACG CGGAGCCTTC GAGACGCGCT TCGTGTCGCA |
| 2001 | GGTGGAGAGC GACACGGGCG ACCGCAAGCA GATCTACAAC ATCCTGAGCA |
| 2051 | CGCTGGGGCT GCGACCCTCG ACCACCGACT GCGACATCGT GCGCCGCGCC |
| 2101 | TGCGAGAGCG TGTCTACGCG CGCTGCGCAC ATGTGCTCGG CGGGGCTGGC |
| 2151 | GGGCGTCATC AACCGCATGC GCGAGAGCCG CAGCGAGGAC GTAATGCGCA |
| 2201 | TCACTGTGGG CGTGGATGGC TCCGTGTACA AGCTGCACCC CAGCTTCAAG |
| 2251 | GAGCGGTTCC ATGCCAGCGT GCGCAGGCTG ACGCCCAGCT GCGAGATCAC |
| 2301 | CTTCATCGAG TCGGAGGAGG GCAGTGGCCG GGGCGCGGCC CTGGTCTCGG |
| 2351 | CGGTGGCCTG TAAGAAGGCC TGTATGCTGG GCCAGTGACT CGAGCACGTG |
| 2401 | GAGCTCGCTG ATCAGCCTCG ACTGTGCCTT CTAGTTGCCA GCCATCTGTT |
| 2451 | GTTTGCCCCT CCCCCGTGCC TTCCTTGACC CTGGAAGGTG CCACTCCCAC |
| 2501 | TGTCCTTTCC TAATAAAATG AGGAAATTGC ATCGCATTGT CTGAGTAGGT |
| 2551 | GTCATTCTAT TCTGGGGGGT GGGGTGGGGC AGGACAGCAA GGGGGAGGAT |
| 2601 | TGGGAAGACA ATAGCAGGCA TGCTGGGGAT GCGGTGGGCT CTATGGCCAC |
| 2651 | GTGATTTAAA TCGAGATCTT CCAGAGCATG AGCGCTGATC TGTCGAGCCA |
| 2701 | TGTCTAGGTA GCCATGCTCT AGGAAGATCC TAATCGATTT TACCACATTT |
| 2751 | GTAGAGGTTT TACTTGCTTT AAAAAACCTC CCACATCTCC CCCTGAACCT |
| 2801 | GAAACATAAA ATGAATGCAA TTGTTGTTGT TAACTTGTTT ATTGCAGCTT |
| 2851 | ATAATGGTTA CAAATAAAGC AATAGCATCA CAAATTTCAC AAATAAAGCA |
| 2901 | TTTTTTTCAC TGCATTCTAG TTGTGGTTTG TCCAAACTCA TCAATGTATC |
| 2951 | TTATCATGTC TGCTCGAAGC GGCCTCGACA GCGCTGGTAC CGTCGACGGC |
| 3001 | TGCGTCTAGT TGCAGTAGTT CTCCAGCTGG TAGAGGGAGC AGATGCTGGT |
| 3051 | ACAGCATTGT TCCACAATGC CACGCTTCTG TCGCGACCCC TCCAGGGCCA |
| 3101 | AGGGCTGCAG GCTGCCTGCA CCAGGGCCCC CGCCCAGCTC CACCTGCCCC |
| 3151 | ACCTGCAGGT CCTCTGCCTC CCGCTTGGTC CTGGGTGTGT AGAAGAAGCC |
| 3201 | TCGTTCCCCG CACACTAGGT AGAGAGCTTC CACCAGATCT GAGCCGCACA |
| 3251 | GGTGTTGGTT CACAAAGGCT GCGGCTGGGT CAGGTCCCCA GAGGGCCAGC |
| 3301 | AGCGCCAGCA GGGGCAGGAG GCGCATCCAC AGGGCCATGG CAGAAGGTCG |
| 3351 | ACGCTAGCGT CTCCCTATAG TGAGTCGTAT TCTATAGTGA GTCGTATTAA |
| 3401 | GTACTCTAGC CTTAAGAGCT GTAATTGAAC TGGGAGTGGA CACCTGTGGA |
| 3451 | GAGAAAGGCA AAGTGGATGT CAGTAAGACC AATAGGTGCC TATCAGAAAC |
| 3501 | GCAAGAGTCT TCTCTGTCTC GACAAGCCCA GTTTCTATTG GTCTCCTTAA |
| 3551 | ACCTGTCTTG TAACCTTGAT ACTTACCTGC CCAGTGCCTC ACGACCAACT |
| 3601 | TCTGCAAGGC TACCTTAAGA GAGTCACTGC AGCTTAAGTT CGAGACTGTT |
| 3651 | GTGTCAGAAG CACTGACTGC GTTAGCAATT TAACTGTGAT AAACTACCGC |
| 3701 | AATAGCTAGC CTAGTGATCT GACGGTTCAC TAAACGAGCT CTGCTTATAT |
| 3751 | AGACCTCCCA CCGTACACGC CTACCGCCCA TTTGCGTCAA CGGGGCGGGC |
| 3801 | GATCGCAGTT GTTACGACAT TTTGGAAAGT CCCGTTGATT TTGGTGCCAA |
| 3851 | AACAAACTCC CATTGACGTC AATGGGGTGG AGACTTGGAA ATCCCCGTGA |
| 3901 | GTCAAACCGC TATCCACGCC CATTGGTGTA CTGCCAAAAC CGCATCACCA |
| 3951 | TGGTAATAGC GATGACTAAT ACGTAGATGT ACTGCCAAGT AGGAAAGTCC |
| 4001 | CGTAAGGTCA TGTACTGGGC ATAATGCCAG GCGGGCCATT TACCGTCATT |
| 4051 | GACGTCAATA GGGGGCGGAC TTGGCATATG ATACACTTGA TGTACTGCCA |
| 4101 | AGTGGGCAGT TTACCGTAAA TACTCCACCC ATTGACGTCA ATGGAAAGTC |
| 4151 | CCTATTGGCG TTACTATGGG AACATACGTC ATTATTGACG TCAATGGGCG |
| 4201 | GGGGTCGTTG GGCGGTCAGC CAGGCGGGCC ATTTACCGTA AGTTATGTAA |
| 4251 | CGCGGAACTC CATATATGGG CTATGAACTA ATGACCCCGT AATTGATTAC |
| 4301 | TATTAATAAC TAGTCAATAA TCAATGCCAA CATGGCGGTC ATATTGGACA |
| 4351 | TGAGCCAATA TAAATGTACA TATTATGATA TAGATACAAC GTATGCAATG |
| 4401 | GCCAATAGCC AATATTGATT TATGCTATAT AACCAATGAA TAATATGGCT |
| 4451 | AATGGCCAAT ATTGAGATCT GTAGATAAGT AGCATGGCGG GTTAATCATT |
| 4501 | AACTACAGAT AAATGCGGCC GCAGGAACCC CTAGTGATGG AGTTGGCCAC |
| 4551 | TCCCTCTCTG CGCGCTCGCT CGCTCACTGA GGCCGGGCGA CCAAAGGTCG |
| 4601 | CCCGACGCCC GGGCTTTGCC CGGGCGGCCT CAGTGAGCGA GCGAGCGCGC |
| 4651 | AGCTGCCTGC AGGGGCGCCT GATGCGGTAT TTTCTCCTTA CGCATCTGTG |
| 4701 | CGGTATTTCA CACCGCATAC GTCAAAGCAA CCATAGTACG CGCCCTGTAG |
| 4751 | CGGCGCATTA AGCGCGGCGG GTGTGGTGGT TACGCGCAGC GTGACCGCTA |
| 4801 | CACTTGCCAG CGCCCTAGCG CCCGCTCCTT TCGCTTTCTT CCCTTCCTTT |
| 4851 | CTCGCCACGT TCGCCGGCTT TCCCCGTCAA GCTCTAAATC GGGGGCTCCC |
| 4901 | TTTAGGGTTC CGATTTAGTG CTTTACGGCA CCTCGACCCC AAAAAACTTG |
| 4951 | ATTTGGGTGA TGGTTCACGT AGTGGGCCAT CGCCCTGATA GACGGTTTTT |
| 5001 | CGCCCTTTGA CGTTGGAGTC CACGTTCTTT AATAGTGGAC TCTTGTTCCA |
| 5051 | AACTGGAACA ACACTCAACC CTATCTCGGG CTATTCTTTT GATTTATAAG |
| 5101 | GGATTTTGCC GATTTCGGCC TATTGGTTAA AAAATGAGCT GATTTAACAA |
| 5151 | AAATTTAACG CGAATTTTAA CAAAATATTA ACGTTTACAA TTTTATGGTG |
| 5201 | CACTCTCAGT ACAATCTGCT CTGATGCCGC ATAGTTAAGC CAGCCCCGAC |
| 5251 | ACCCGCCAAC ACCCGCTGAC GCGCCCTGAC GGGCTTGTCT GCTCCCGGCA |
| 5301 | TCCGCTTACA GACAAGCTGT GACCGTCTCC GGGAGCTGCA TGTGTCAGAG |
| 5351 | GTTTTCACCG TCATCACCGA AACGCGCGAG ACGAAAGGGC CTCGTGATAC |
| 5401 | GCCTATTTTT ATAGGTTAAT GTCATGATAA TAATGGTTTC TTAGACGTCA |
| 5451 | GGTGGCACTT TTCGGGGAAA TGTGCGCGGA ACCCCTATTT GTTTATTTTT |
| 5501 | CTAAATACAT TCAAATATGT ATCCGCTCAT GAGACAATAA CCCTGATAAA |
| 5551 | TGCTTCAATA ATATTGAAAA AGGAAGAGTA TGAGTATTCA ACATTTCCGT |
| 5601 | GTCGCCCTTA TTCCCTTTTT TGCGGCATTT TGCCTTCCTG TTTTTGCTCA |
| 5651 | CCCAGAAACG CTGGTGAAAG TAAAAGATGC TGAAGATCAG TTGGGTGCAC |
| 5701 | GAGTGGGTTA CATCGAACTG GATCTCAACA GCGGTAAGAT CCTTGAGAGT |
| 5751 | TTTCGCCCCG AAGAACGTTT TCCAATGATG AGCACTTTTA AAGTTCTGCT |
| 5801 | ATGTGGCGCG GTATTATCCC GTATTGACGC CGGGCAAGAG CAACTCGGTC |
| 5851 | GCCGCATACA CTATTCTCAG AATGACTTGG TTGAGTACTC ACCAGTCACA |
| 5901 | GAAAAGCATC TTACGGATGG CATGACAGTA AGAGAATTAT GCAGTGCTGC |
| 5951 | CATAACCATG AGTGATAACA CTGCGGCCAA CTTACTTCTG ACAACGATCG |
| 6001 | GAGGACCGAA GGAGCTAACC GCTTTTTTGC ACAACATGGG GGATCATGTA |
| 6051 | ACTCGCCTTG ATCGTTGGGA ACCGGAGCTG AATGAAGCCA TACCAAACGA |
| 6101 | CGAGCGTGAC ACCACGATGC CTGTAGCAAT GGCAACAACG TTGCGCAAAC |
| 6151 | TATTAACTGG CGAACTACTT ACTCTAGCTT CCCGGCAACA ATTAATAGAC |
| 6201 | TGGATGGAGG CGGATAAAGT TGCAGGACCA CTTCTGCGCT CGGCCCTTCC |
| 6251 | GGCTGGCTGG TTTATTGCTG ATAAATCTGG AGCCGGTGAG CGTGGGTCTC |
| 6301 | GCGGTATCAT TGCAGCACTG GGGCCAGATG GTAAGCCCTC CCGTATCGTA |
| 6351 | GTTATCTACA CGACGGGGAG TCAGGCAACT ATGGATGAAC GAAATAGACA |
| 6401 | GATCGCTGAG ATAGGTGCCT CACTGATTAA GCATTGGTAA CTGTCAGACC |
| 6451 | AAGTTTACTC ATATATACTT TAGATTGATT TAAAACTTCA TTTTTAATTT |
| 6501 | AAAAGGATCT AGGTGAAGAT CCTTTTTGAT AATCTCATGA CCAAAATCCC |
| 6551 | TTAACGTGAG TTTTCGTTCC ACTGAGCGTC AGACCCCGTA GAAAAGATCA |
| 6601 | AAGGATCTTC TTGAGATCCT TTTTTTCTGC GCGTAATCTG CTGCTTGCAA |
| 6651 | ACAAAAAAAC CACCGCTACC AGCGGTGGTT TGTTTGCCGG ATCAAGAGCT |
| 6701 | ACCAACTCTT TTTCCGAAGG TAACTGGCTT CAGCAGAGCG CAGATACCAA |
| 6751 | ATACTGTCCT TCTAGTGTAG CCGTAGTTAG GCCACCACTT CAAGAACTCT |
| 6801 | GTAGCACCGC CTACATACCT CGCTCTGCTA ATCCTGTTAC CAGTGGCTGC |
| 6851 | TGCCAGTGGC GATAAGTCGT GTCTTACCGG GTTGGACTCA AGACGATAGT |
| 6901 | TACCGGATAA GGCGCAGCGG TCGGGCTGAA CGGGGGGTTC GTGCACACAG |
| 6951 | CCCAGCTTGG AGCGAACGAC CTACACCGAA CTGAGATACC TACAGCGTGA |
| 7001 | GCTATGAGAA AGCGCCACGC TTCCCGAAGG GAGAAAGGCG GACAGGTATC |
| 7051 | CGGTAAGCGG CAGGGTCGGA ACAGGAGAGC GCACGAGGGA GCTTCCAGGG |
| 7101 | GGAAACGCCT GGTATCTTTA TAGTCCTGTC GGGTTTCGCC ACCTCTGACT |
| 7151 | TGAGCGTCGA TTTTTGTGAT GCTCGTCAGG GGGGCGGAGC CTATGGAAAA |
| 7201 | ACGCCAGCAA CGCGGCCTTT TTACGGTTCC TGGCCTTTTG CTGGCCTTTT |
| 7251 | GCTCACATGT |

ITR 5′: 1-141 bp
RSV promoter: 239-948 bp
hGck: 973-2387 bp
bGH polyA: 2395-2653 bp
SV40 polya: 2687-2985 bp
hIns: 2999-3345 bp
CMV promoter: 3353-4470 bp
ITR 3′: 4523-4663 bp
H: CMV-hIns

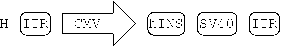

pAAV-CMV-hIns plasmid sequence
(SEQ ID NO: 19)

| | |
|---|---|
| 1 | CCTGCAGGCA GCTGCGCGCT CGCTCGCTCA CTGAGGCCGC CCGGGCAAAG |
| 51 | CCCGGGCGTC GGGCGACCTT TGGTCGCCCG GCCTCAGTGA GCGAGCGAGC |
| 101 | GCGCAGAGAG GGAGTGGCCA ACTCCATCAC TAGGGGTTCC TGCGGCCGCG |
| 151 | ATATCTGTAG TTAATGATTA ACCCGCCATG CTACTTATCT ACAGATCTCA |
| 201 | ATATTGGCCA TTAGCCATAT TATTCATTGG TTATATAGCA TAAATCAATA |
| 251 | TTGGCTATTG GCCATTGCAT ACGTTGTATC TATATCATAA TATGTACATT |
| 301 | TATATTGGCT CATGTCCAAT ATGACCGCCA TGTTGGCATT GATTATTGAC |
| 351 | TAGTTATTAA TAGTAATCAA TTACGGGGTC ATTAGTTCAT AGCCCATATA |
| 401 | TGGAGTTCCG CGTTACATAA CTTACGGTAA ATGGCCCGCC TGGCTGACCG |
| 431 | CCCAACGACC CCCGCCCATT GACGTCAATA ATGACGTATG TTCCCATAGT |
| 501 | AACGCCAATA GGGACTTTCC ATTGACGTCA ATGGGTGGAG TATTTACGGT |
| 551 | AAACTGCCCA CTTGGCAGTA CATCAAGTGT ATCATATGCC AAGTCCGCCC |
| 601 | CCTATTGACG TCAATGACGG TAAATGGCCC GCCTGGCATT ATGCCCAGTA |
| 651 | CATGACCTTA CGGGACTTTC CTACTTGGCA GTACATCTAC GTATTAGTCA |
| 701 | TCGCTATTAC CATGGTGATG CGGTTTTGGC AGTACACCAA TGGGCGTGGA |
| 751 | TAGCGGTTTG ACTCACGGGG ATTTCCAAGT CTCCACCCCA TTGACGTCAA |
| 801 | TGGGAGTTTG TTTTGGCACC AAAATCAACG GGACTTTCCA AAATGTCGTA |
| 851 | ACAACTGCGA TCGCCCGCCC CGTTGACGCA AATGGGCGGT AGGCGTGTAC |
| 901 | GGTGGGAGGT CTATATAAGC AGAGCTCGTT TAGTGAACCG TCAGATCACT |
| 951 | AGGCTAGCTA TTGCGGTAGT TTATCACAGT TAAATTGCTA ACGCAGTCAG |
| 1001 | TGCTTCTGAC ACAACAGTCT CGAACTTAAG CTGCAGTGAC TCTCTTAAGG |
| 1051 | TAGCCTTGCA GAAGTTGGTC GTGAGGCACT GGGCAGGTAA GTATCAAGGT |
| 1101 | TACAAGACAG GTTTAAGGAG ACCAATAGAA ACTGGGCTTG TCGAGACAGA |
| 1151 | GAAGACTCTT GCGTTTCTGA TAGGCACCTA TTGGTCTTAC TGACATCCAC |
| 1201 | TTTGCCTTTC TCTCCACAGG TGTCCACTCC CAGTTCAATT ACAGCTCTTA |
| 1251 | AGGCTAGAGT ACTTAATACG ACTCACTATA GAATACGACT CACTATAGGG |
| 1301 | AGACGCTAGC GTCGACCTTC TGCCATGGCC CTGTGGATGC GCCTCCTGCC |
| 1351 | CCTGCTGGCG CTGCTGGCCC TCTGGGGACC TGACCCAGCC GCAGCCTTTG |
| 1401 | TGAACCAACA CCTGTGCGGC TCAGATCTGG TGGAAGCTCT CTACCTAGTG |
| 1451 | TGCGGGGAAC GAGGCTTCTT CTACACACCC AGGACCAAGC GGGAGGCAGA |
| 1501 | GGACCTGCAG GTGGGGCAGG TGGAGCTGGG CGGGGGCCCT GGTGCAGGCA |
| 1551 | GCCTGCAGCC CTTGGCCCTG GAGGGGTCGC GACAGAAGCG TGGCATTGTG |
| 1601 | GAACAATGCT GTACCAGCAT CTGCTCCCTC TACCAGCTGG AGAACTACTG |
| 1651 | CAACTAGACG CAGCCGTCGA CGGTACCAGC GCTGTCGAGG CCGCTTCGAG |
| 1701 | CAGACATGAT AAGATACATT GATGAGTTTG GACAAACCAC AACTAGAATG |
| 1751 | CAGTGAAAAA AATGCTTTAT TTGTGAAATT TGTGATGCTA TTGCTTTATT |
| 1801 | TGTAACCATT ATAAGCTGCA ATAAACAAGT TAACAACAAC AATTGCATTC |
| 1851 | ATTTTATGTT TCAGGTTCAG GGGGAGATGT GGGAGGTTTT TTAAAGCAAG |
| 1901 | TAAAACCTCT ACAAATGTGG TAAAATCGAT TAGGATCTTC CTAGAGCATG |
| 1951 | GCTACCTAGA CATGGCTCGA CAGATCAGCG CTCATGCTCT GGAAGATCTC |
| 2001 | GATTTAAATG CGGCCGCAGG AACCCCTAGT GATGGAGTTG GCCACTCCCT |
| 2051 | CTCTGCGCGC TCGCTCGCTC ACTGAGGCCG GGCGACCAAA GGTCGCCCGA |
| 2101 | CGCCCGGGCT TTGCCCGGGC GGCCTCAGTG AGCGAGCGAG CGCGCAGCTG |
| 2151 | CCTGCAGGGG CGCCTGATGC GGTATTTTCT CCTTACGCAT CTGTGCGGTA |
| 2201 | TTTCACACCG CATACGTCAA AGCAACCATA GTACGCGCCC TGTAGCGGCG |
| 2251 | CATTAAGCGC GGCGGGTGTG GTGGTTACGC GCAGCGTGAC CGCTACACTT |
| 2301 | GCCAGCGCCC TAGCGCCCGC TCCTTTCGCT TTCTTCCCTT CCTTTCTCGC |
| 2351 | CACGTTCGCC GGCTTTCCCC GTCAAGCTCT AAATCGGGGG CTCCCTTTAG |
| 2401 | GGTTCCGATT TAGTGCTTTA CGGCACCTCG ACCCCAAAAA ACTTGATTTG |
| 2451 | GGTGATGGTT CACGTAGTGG GCCATCGCCC TGATAGACGG TTTTTCGCCC |
| 2501 | TTTGACGTTG GAGTCCACGT TCTTTAATAG TGGACTCTTG TTCCAAACTG |
| 2551 | GAACAACACT CAACCCTATC TCGGGCTATT CTTTTGATTT ATAAGGGATT |
| 2601 | TTGCCGATTT CGGCCTATTG GTTAAAAAAT GAGCTGATTT AACAAAAATT |
| 2651 | TAACGCGAAT TTTAACAAAA TATTAACGTT TACAATTTTA TGGTGCACTC |
| 2701 | TCAGTACAAT CTGCTCTGAT GCCGCATAGT TAAGCCAGCC CCGACACCCG |
| 2751 | CCAACACCCG CTGACGCGCC CTGACGGGCT TGTCTGCTCC CGGCATCCGC |
| 2801 | TTACAGACAA GCTGTGACCG TCTCCGGGAG CTGCATGTGT CAGAGGTTTT |
| 2851 | CACCGTCATC ACCGAAACGC GCGAGACGAA AGGGCCTCGT GATACGCCTA |
| 2901 | TTTTTATAGG TTAATGTCAT GATAATAATG GTTTCTTAGA CGTCAGGTGG |
| 2951 | CACTTTTCGG GGAAATGTGC GCGGAACCCC TATTTGTTTA TTTTTCTAAA |
| 3001 | TACATTCAAA TATGTATCCG CTCATGAGAC AATAACCCTG ATAAATGCTT |
| 3051 | CAATAATATT GAAAAAGGAA GAGTATGAGT ATTCAACATT TCCGTGTCGC |
| 3101 | CCTTATTCCC TTTTTTGCGG CATTTTGCCT TCCTGTTTTT GCTCACCCAG |
| 3151 | AAACGCTGGT GAAAGTAAAA GATGCTGAAG ATCAGTTGGG TGCACGAGTG |
| 3201 | GGTTACATCG AACTGGATCT CAACAGCGGT AAGATCCTTG AGAGTTTTCG |
| 3251 | CCCCGAAGAA CGTTTTCCAA TGATGAGCAC TTTTAAAGTT CTGCTATGTG |
| 3301 | GCGCGGTATT ATCCCGTATT GACGCCGGGC AAGAGCAACT CGGTCGCCGC |
| 3351 | ATACACTATT CTCAGAATGA CTTGGTTGAG TACTCACCAG TCACAGAAAA |
| 3401 | GCATCTTACG GATGGCATGA CAGTAAGAGA ATTATGCAGT GCTGCCATAA |
| 3451 | CCATGAGTGA TAACACTGCG GCCAACTTAC TTCTGACAAC GATCGGAGGA |
| 3501 | CCGAAGGAGC TAACCGCTTT TTTGCACAAC ATGGGGGATC ATGTAACTCG |
| 3551 | CCTTGATCGT TGGGAACCGG AGCTGAATGA AGCCATACCA AACGACGAGC |
| 3601 | GTGACACCAC GATGCCTGTA GCAATGGCAA CAACGTTGCG CAAACTATTA |
| 3651 | ACTGGCGAAC TACTTACTCT AGCTTCCCGG CAACAATTAA TAGACTGGAT |
| 3701 | GGAGGCGGAT AAAGTTGCAG GACCACTTCT GCGCTCGGCC CTTCCGGCTG |
| 3751 | GCTGGTTTAT TGCTGATAAA TCTGGAGCCG GTGAGCGTGG GTCTCGCGGT |
| 3801 | ATCATTGCAG CACTGGGGCC AGATGGTAAG CCCTCCCGTA TCGTAGTTAT |
| 3851 | CTACACGACG GGGAGTCAGG CAACTATGGA TGAACGAAAT AGACAGATCG |
| 3901 | CTGAGATAGG TGCCTCACTG ATTAAGCATT GGTAACTGTC AGACCAAGTT |
| 3951 | TACTCATATA TACTTTAGAT TGATTTAAAA CTTCATTTTT AATTTAAAAG |
| 4001 | GATCTAGGTG AAGATCCTTT TTGATAATCT CATGACCAAA ATCCCTTAAC |
| 4051 | GTGAGTTTTC GTTCCACTGA GCGTCAGACC CCGTAGAAAA GATCAAAGGA |
| 4101 | TCTTCTTGAG ATCCTTTTTT TCTGCGCGTA ATCTGCTGCT TGCAAACAAA |
| 4151 | AAAACCACCG CTACCAGCGG TGGTTTGTTT GCCGGATCAA GAGCTACCAA |
| 4201 | CTCTTTTTCC GAAGGTAACT GGCTTCAGCA GAGCGCAGAT ACCAAATACT |
| 4251 | GTCCTTCTAG TGTAGCCGTA GTTAGGCCAC CACTTCAAGA ACTCTGTAGC |
| 4301 | ACCGCCTACA TACCTCGCTC TGCTAATCCT GTTACCAGTG GCTGCTGCCA |
| 4351 | GTGGCGATAA GTCGTGTCTT ACCGGGTTGG ACTCAAGACG ATAGTTACCG |
| 4401 | GATAAGGCGC AGCGGTCGGG CTGAACGGGG GGTTCGTGCA CACAGCCCAG |
| 4451 | CTTGGAGCGA ACGACCTACA CCGAACTGAG ATACCTACAG CGTGAGCTAT |
| 4501 | GAGAAAGCGC CACGCTTCCC GAAGGGAGAA AGGCGGACAG GTATCCGGTA |
| 4551 | AGCGGCAGGG TCGGAACAGG AGAGCGCACG AGGGAGCTTC CAGGGGGAAA |
| 4601 | CGCCTGGTAT CTTTATAGTC CTGTCGGGTT TCGCCACCTC TGACTTGAGC |
| 4651 | GTCGATTTTT GTGATGCTCG TCAGGGGGGC GGAGCCTATG GAAAAACGCC |
| 4701 | AGCAACGCGG CCTTTTTACG GTTCCTGGCC TTTTGCTGGC CTTTTGCTCA |
| 4751 | CATGT |

ITR 5′: 1-141 bp
CMV promoter: 193-1310 bp
hIns: 1318-1664 bp
SV40 polyA: 1678-1976 bp
ITR 3′: 2018-2158 bp
I: RSV-hGck

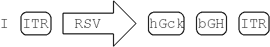

pAAV-RSV-hGck plasmid sequence
(SEQ ID NO: 20)

| | |
|---|---|
| 1 | CCTGCAGGCA GCTGCGCGCT CGCTCGCTCA CTGAGGCCGC CCGGGCAAAG |
| 51 | CCCGGGCGTC GGGCGACCTT TGGTCGCCCG GCCTCAGTGA GCGAGCGAGC |
| 101 | GCGCAGAGAG GGAGTGGCCA ACTCCATCAC TAGGGGTTCC TGCGGCCGCG |
| 151 | ATATCCATGT TTGACAGCTT ATCATCGCAG ATCCGTATGG TGCACTCTCA |
| 201 | GTACAATCTG CTCTGATGCC GCATAGTTAA GCCAGTATCT GCTCCCTGCT |
| 251 | TGTGTGTTGG AGGTCGCTGA GTAGTGCGCG AGCAAAATTT AAGCTACAAC |
| 301 | AAGGCAAGGC TTGACCGACA ATTGCATGAA GAATCTGCTT AGGGTTAGGC |
| 351 | GTTTTGCGCT GCTTCGCGAT GTACGGGCCA GATATTCGCG TATCTGAGGG |
| 401 | GACTAGGGTG TGTTTAGGCG AAAAGCGGGG CTTCGGTTGT ACGCGGTTAG |
| 451 | GAGTCCCCTC AGGATATAGT AGTTTCGCTT TTGCATAGGG AGGGGGAAAT |
| 501 | GTAGTCTTAT GCAATACTCT TGTAGTCTTG CAACATGGTA ACGATGAGTT |
| 551 | AGCAACATGC CTTACAAGGA GAGAAAAAGC ACCGTGCATG CCGATTGGTG |
| 601 | GAAGTAAGGT GGTACGATCG TGCCTTATTA GGAAGGCAAC AGACGGGTCT |
| 651 | GACATGGATT GGACGAACCA CTAAATTCCG CATTGCAGAG ATATTGTATT |
| 701 | TAAGTGCCTA GCTCGATACA ATAAACGCCA TTTGACCATT CACCACATTG |
| 751 | GTGTGCACCT CCAAGCTGGG TACCAGCTTC TAGAGAGATC TGCTTCAGCT |
| 801 | GGAGGCACTG GGCAGGTAAG TATCAAGGTT ACAAGACAGG TTTAAGGAGA |
| 851 | CCAATAGAAA CTGGGCTTGT CGAGACAGAG AAGACTCTTG CGTTTCTGAT |
| 901 | AGGCACCTAT TGGTCTTACT GACATCCACT TTGCCTTTCT CTCCACAGGT |
| 951 | GCAGCTGCTG CAGCGGTCTA GAACTCGAGT CGAGACCATG GCGATGGATG |
| 1001 | TCACAAGGAG CCAGGCCCAG ACAGCCTTGA CTCTGGTAGA GCAGATCCTG |
| 1051 | GCAGAGTTCC AGCTGCAGGA GGAGGACCTG AAGAAGGTGA TGAGACGGAT |
| 1101 | GCAGAAGGAG ATGGACCGCG GCCTGAGGCT GGAGACCCAT GAAGAGGCCA |
| 1151 | GTGTGAAGAT GCTGCCCACC TACGTGCGCT CCACCCCAGA AGGCTCAGAA |
| 1201 | GTCGGGGACT TCCTCTCCCT GGACCTGGGT GGCACTAACT TCAGGGTGAT |
| 1251 | GCTGGTGAAG GTGGGAGAAG GTGAGGAGGG GCAGTGGAGC GTGAAGACCA |
| 1301 | AACACCAGAT GTACTCCATC CCCGAGGACG CCATGACCGG CACTGCTGAG |
| 1351 | ATGCTCTTCG ACTACATCTC TGAGTGCATC TCCGACTTCC TGGACAAGCA |
| 1401 | TCAGATGAAA CACAAGAAGC TGCCCCTGGG CTTCACCTTC TCCTTTCCTG |
| 1451 | TGAGGCACGA AGACATCGAT AAGGGCATCC TTCTCAACTG GACCAAGGGC |
| 1501 | TTCAAGGCCT CAGGAGCAGA AGGGAACAAT GTCGTGGGGC TTCTGCGAGA |
| 1551 | CGCTATCAAA CGGAGAGGGG ACTTTGAAAT GGATGTGGTG GCAATGGTGA |
| 1601 | ATGACACGGT GGCCACGATG ATCTCCTGCT ACTACGAAGA CCATCAGTGC |
| 1651 | GAGGTCGGCA TGATCGTGGG CACGGGCTGC AATGCCTGCT ACATGGAGGA |
| 1701 | GATGCAGAAT GTGGAGCTGG TGGAGGGGGA CGAGGGCCGC ATGTGCGTCA |
| 1751 | ATACCGAGTG GGGCGCCTTC GGGGACTCCG GCGAGCTGGA CGAGTTCCTG |
| 1801 | CTGGAGTATG ACCGCCTGGT GGACGAGAGC TCTGCAAACC CCGGTCAGCA |
| 1851 | GCTGTATGAG AAGCTCATAG GTGGCAAGTA CATGGGCGAG CTGGTGCGGC |
| 1901 | TTGTGCTGCT CAGGCTCGTG GACGAAAACC TGCTCTTCCA CGGGGAGGCC |
| 1951 | TCCGAGCAGC TGCGCACACG CGGAGCCTTC GAGACGCGCT TCGTGTCGCA |
| 2001 | GGTGGAGAGC GACACGGGCG ACCGCAAGCA GATCTACAAC ATCCTGAGCA |
| 2051 | CGCTGGGGCT GCGACCCTCG ACCACCGACT GCGACATCGT GCGCCGCGCC |
| 2101 | TGCGAGAGCG TGTCTACGCG CGCTGCGCAC ATGTGCTCGG CGGGGCTGGC |
| 2151 | GGGCGTCATC AACCGCATGC GCGAGAGCCG CAGCGAGGAC GTAATGCGCA |
| 2201 | TCACTGTGGG CGTGGATGGC TCCGTGTACA AGCTGCACCC CAGCTTCAAG |
| 2251 | GAGCGGTTCC ATGCCAGCGT GCGCAGGCTG ACGCCCAGCT GCGAGATCAC |
| 2301 | CTTCATCGAG TCGGAGGAGG GCAGTGGCCG GGGCGCGGCC CTGGTCTCGG |
| 2351 | CGGTGGCCTG TAAGAAGGCC TGTATGCTGG GCCAGTGACT CGAGCACGTG |
| 2401 | GAGCTCGCTG ATCAGCCTCG ACTGTGCCTT CTAGTTGCCA GCCATCTGTT |
| 2451 | GTTTGCCCCT CCCCCGTGCC TTCCTTGACC CTGGAAGGTG CCACTCCCAC |
| 2501 | TGTCCTTTCC TAATAAAATG AGGAAATTGC ATCGCATTGT CTGAGTAGGT |
| 2551 | GTCATTCTAT TCTGGGGGGT GGGGTGGGGC AGGACAGCAA GGGGGAGGAT |
| 2601 | TGGGAAGACA ATAGCAGGCA TGCTGGGGAT GCGGTGGGCT CTATGGCCAC |
| 2651 | GTGATTTAAA TGCGGCCGCA GGAACCCCTA GTGATGGAGT TGGCCACTCC |
| 2701 | CTCTCTGCGC GCTCGCTCGC TCACTGAGGC CGGGCGACCA AAGGTCGCCC |
| 2751 | GACGCCCGGG CTTTGCCCGG GCGGCCTCAG TGAGCGAGCG AGCGCGCAGC |
| 2801 | TGCCTGCAGG GGCGCCTGAT GCGGTATTTT CTCCTTACGC ATCTGTGCGG |
| 2851 | TATTTCACAC CGCATACGTC AAAGCAACCA TAGTACGCGC CCTGTAGCGG |
| 2901 | CGCATTAAGC GCGGCGGGTG TGGTGGTTAC GCGCAGCGTG ACCGCTACAC |
| 2951 | TTGCCAGCGC CCTAGCGCCC GCTCCTTTCG CTTTCTTCCC TTCCTTTCTC |
| 3001 | GCCACGTTCG CCGGCTTTCC CCGTCAAGCT CTAAATCGGG GGCTCCCTTT |
| 3051 | AGGGTTCCGA TTTAGTGCTT TACGGCACCT CGACCCCAAA AAACTTGATT |
| 3101 | TGGGTGATGG TTCACGTAGT GGGCCATCGC CCTGATAGAC GGTTTTTCGC |
| 3151 | CCTTTGACGT TGGAGTCCAC GTTCTTTAAT AGTGGACTCT TGTTCCAAAC |
| 3201 | TGGAACAACA CTCAACCCTA TCTCGGGCTA TTCTTTTGAT TTATAAGGGA |
| 3251 | TTTTGCCGAT TTCGGCCTAT TGGTTAAAAA ATGAGCTGAT TTAACAAAAA |
| 3301 | TTTAACGCGA ATTTTAACAA AATATTAACG TTTACAATTT TATGGTGCAC |
| 3351 | TCTCAGTACA ATCTGCTCTG ATGCCGCATA GTTAAGCCAG CCCCGACACC |
| 3401 | CGCCAACACC CGCTGACGCG CCCTGACGGG CTTGTCTGCT CCCGGCATCC |
| 3451 | GCTTACAGAC AAGCTGTGAC CGTCTCCGGG AGCTGCATGT GTCAGAGGTT |
| 3501 | TTCACCGTCA TCACCGAAAC GCGCGAGACG AAAGGGCCTC GTGATACGCC |
| 3551 | TATTTTTATA GGTTAATGTC ATGATAATAA TGGTTTCTTA GACGTCAGGT |
| 3601 | GGCACTTTTC GGGGAAATGT GCGCGGAACC CCTATTTGTT TATTTTTCTA |
| 3651 | AATACATTCA AATATGTATC CGCTCATGAG ACAATAACCC TGATAAATGC |
| 3701 | TTCAATAATA TTGAAAAAGG AAGAGTATGA GTATTCAACA TTTCCGTGTC |
| 3751 | GCCCTTATTC CCTTTTTTGC GGCATTTTGC CTTCCTGTTT TTGCTCACCC |
| 3801 | AGAAACGCTG GTGAAAGTAA AAGATGCTGA AGATCAGTTG GGTGCACGAG |
| 3851 | TGGGTTACAT CGAACTGGAT CTCAACAGCG GTAAGATCCT TGAGAGTTTT |
| 3901 | CGCCCCGAAG AACGTTTTCC AATGATGAGC ACTTTTAAAG TTCTGCTATG |
| 3951 | TGGCGCGGTA TTATCCCGTA TTGACGCCGG GCAAGAGCAA CTCGGTCGCC |
| 4001 | GCATACACTA TTCTCAGAAT GACTTGGTTG AGTACTCACC AGTCACAGAA |
| 4051 | AAGCATCTTA CGGATGGCAT GACAGTAAGA GAATTATGCA GTGCTGCCAT |
| 4101 | AACCATGAGT GATAACACTG CGGCCAACTT ACTTCTGACA ACGATCGGAG |
| 4151 | GACCGAAGGA GCTAACCGCT TTTTTGCACA ACATGGGGGA TCATGTAACT |
| 4201 | CGCCTTGATC GTTGGGAACC GGAGCTGAAT GAAGCCATAC CAAACGACGA |
| 4251 | GCGTGACACC ACGATGCCTG TAGCAATGGC AACAACGTTG CGCAAACTAT |
| 4301 | TAACTGGCGA ACTACTTACT CTAGCTTCCC GGCAACAATT AATAGACTGG |
| 4351 | ATGGAGGCGG ATAAAGTTGC AGGACCACTT CTGCGCTCGG CCCTTCCGGC |
| 4401 | TGGCTGGTTT ATTGCTGATA AATCTGGAGC CGGTGAGCGT GGGTCTCGCG |
| 4451 | GTATCATTGC AGCACTGGGG CCAGATGGTA AGCCCTCCCG TATCGTAGTT |
| 4501 | ATCTACACGA CGGGGAGTCA GGCAACTATG GATGAACGAA ATAGACAGAT |
| 4551 | CGCTGAGATA GGTGCCTCAC TGATTAAGCA TTGGTAACTG TCAGACCAAG |
| 4601 | TTTACTCATA TATACTTTAG ATTGATTTAA AACTTCATTT TTAATTTAAA |
| 4651 | AGGATCTAGG TGAAGATCCT TTTTGATAAT CTCATGACCA AAATCCCTTA |
| 4701 | ACGTGAGTTT TCGTTCCACT GAGCGTCAGA CCCCGTAGAA AAGATCAAAG |
| 4751 | GATCTTCTTG AGATCCTTTT TTTCTGCGCG TAATCTGCTG CTTGCAAACA |
| 4801 | AAAAAACCAC CGCTACCAGC GGTGGTTTGT TTGCCGGATC AAGAGCTACC |
| 4851 | AACTCTTTTT CCGAAGGTAA CTGGCTTCAG CAGAGCGCAG ATACCAAATA |
| 4901 | CTGTCCTTCT AGTGTAGCCG TAGTTAGGCC ACCACTTCAA GAACTCTGTA |
| 4951 | GCACCGCCTA CATACCTCGC TCTGCTAATC CTGTTACCAG TGGCTGCTGC |
| 5001 | CAGTGGCGAT AAGTCGTGTC TTACCGGGTT GGACTCAAGA CGATAGTTAC |
| 5051 | CGGATAAGGC GCAGCGGTCG GGCTGAACGG GGGGTTCGTG CACACAGCCC |
| 5101 | AGCTTGGAGC GAACGACCTA CACCGAACTG AGATACCTAC AGCGTGAGCT |
| 5151 | ATGAGAAAGC GCCACGCTTC CCGAAGGGAG AAAGGCGGAC AGGTATCCGG |
| 5201 | TAAGCGGCAG GGTCGGAACA GGAGAGCGCA CGAGGGAGCT TCCAGGGGGA |
| 5251 | AACGCCTGGT ATCTTTATAG TCCTGTCGGG TTTCGCCACC TCTGACTTGA |
| 5301 | GCGTCGATTT TTGTGATGCT CGTCAGGGGG GCGGAGCCTA TGGAAAAACG |
| 5351 | CCAGCAACGC GGCCTTTTTA CGGTTCCTGG CCTTTTGCTG GCCTTTTGCT |
| 5401 | CACATGT |

ITR 5′: 1-141 bp
RSV promoter: 239-948 bp
hGck: 973-2387 bp
bGH polyA: 2395-2653 bp
ITR 3′: 2670-2810 bp
J: miniCMV-hIns-RSV-hGck

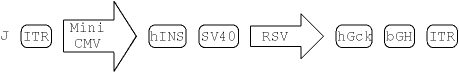

pAAV-miniCMV-hIns-RSV-hGck plasmid sequence
(SEQ ID NO: 13)

| | |
|---|---|
| 1 | CCTGCAGGCA GCTGCGCGCT CGCTCGCTCA CTGAGGCCGC CCGGGCAAAG |
| 51 | CCCGGGCGTC GGGCGACCTT TGGTCGCCCG GCCTCAGTGA GCGAGCGAGC |
| 101 | GCGCAGAGAG GGAGTGGCCA ACTCCATCAC TAGGGGTTCC TGCGGCCGCG |
| 151 | ATATCTATGC CAAGTACGCC CCCTATTGAC GTCAATGACG GTAAATGGCC |
| 201 | CGCCTGGCAT TATGCCCAGT ACATGACCTT ATGGGACTTT CCTACTTGGC |
| 251 | AGTACATCTA CGTATTAGTC ATCGCTATTA CCATGGTGAT GCGGTTTTGG |
| 301 | CAGTACATCA ATGGGCGTGG ATAGCGGTTT GACTCACGGG GATTTCCAAG |
| 351 | TCTCCACCCC ATTGACGTCA ATGGGAGTTT GTTTTGGCAC CAAAATCAAC |
| 401 | GGGACTTTCC AAAATGTCGT AACAACTCCG CCCCATTGAC GCAAATGGGC |
| 451 | GGTAGGCGTG TACGGTGGGA GGTCTATATA AGCAGAGCTC TCTGGCTAAC |
| 501 | TAGAGAACCC ACTGCTTAAC TGGCTTATCG AAATTAATAC GACTCACTAT |
| 551 | AGGGAGACCC AAGCTTGCTA GCGTCGACCT TCTGCCATGG CCCTGTGGAT |
| 601 | GCGCCTCCTG CCCCTGCTGG CGCTGCTGGC CCTCTGGGGA CCTGACCCAG |
| 651 | CCGCAGCCTT TGTGAACCAA CACCTGTGCG GCTCAGATCT GGTGGAAGCT |
| 701 | CTCTACCTAG TGTGCGGGGA ACGAGGCTTC TTCTACACAC CCAGGACCAA |
| 751 | GCGGGAGGCA GAGGACCTGC AGGTGGGGCA GGTGGAGCTG GGCGGGGGCC |
| 801 | CTGGTGCAGG CAGCCTGCAG CCCTTGGCCC TGGAGGGGTC GCGACAGAAG |
| 851 | CGTGGCATTG TGGAACAATG CTGTACCAGC ATCTGCTCCC TCTACCAGCT |
| 901 | GGAGAACTAC TGCAACTAGA CGCAGCCGTC GACGGTACCA GCGCTGTCGA |
| 951 | GGCCGCTTCG AGCAGACATG ATAAGATACA TTGATGAGTT TGGACAAACC |
| 1001 | ACAACTAGAA TGCAGTGAAA AAAATGCTTT ATTTGTGAAA TTTGTGATGC |
| 1051 | TATTGCTTTA TTTGTAACCA TTATAAGCTG CAATAAACAA GTTAACAACA |
| 1101 | ACAATTGCAT TCATTTTATG TTTCAGGTTC AGGGGGAGAT GTGGGAGGTT |
| 1151 | TTTTAAAGCA AGTAAAACCT CTACAAATGT GGTAAAATCG ATTAGGATCT |
| 1201 | TCCTAGAGCA TGGCTACCTA GACATGGCTC GACAGATCAG CGCTCATGCT |
| 1251 | CTGGAAGATC TCGATTTATC CATGTTTGAC AGCTTATCAT CGCAGATCCG |
| 1301 | TATGGTGCAC TCTCAGTACA ATCTGCTCTG ATGCCGCATA GTTAAGCCAG |
| 1351 | TATCTGCTCC CTGCTTGTGT GTTGGAGGTC GCTGAGTAGT GCGCGAGCAA |
| 1401 | AATTTAAGCT ACAACAAGGC AAGGCTTGAC CGACAATTGC ATGAAGAATC |
| 1451 | TGCTTAGGGT TAGGCGTTTT GCGCTGCTTC GCGATGTACG GGCCAGATAT |
| 1501 | TCGCGTATCT GAGGGGACTA GGGTGTGTTT AGGCGAAAAG CGGGGCTTCG |
| 1551 | GTTGTACGCG GTTAGGAGTC CCCTCAGGAT ATAGTAGTTT CGCTTTTGCA |
| 1601 | TAGGGAGGGG GAAATGTAGT CTTATGCAAT ACTCTTGTAG TCTTGCAACA |
| 1651 | TGGTAACGAT GAGTTAGCAA CATGCCTTAC AAGGAGAGAA AAAGCACCGT |
| 1701 | GCATGCCGAT TGGTGGAAGT AAGGTGGTAC GATCGTGCCT TATTAGGAAG |
| 1751 | GCAACAGACG GGTCTGACAT GGATTGGACG AACCACTAAA TTCCGCATTG |
| 1801 | CAGAGATATT GTATTTAAGT GCCTAGCTCG ATACAATAAA CGCCATTTGA |
| 1851 | CCATTCACCA CATTGGTGTG CACCTCCAAG CTGGGTACCA GCTTCTAGAG |
| 1901 | AGATCTGCTT CAGCTGGAGG CACTGGGCAG GTAAGTATCA AGGTTACAAG |
| 1951 | ACAGGTTTAA GGAGACCAAT AGAAACTGGG CTTGTCGAGA CAGAGAAGAC |
| 2001 | TCTTGCGTTT CTGATAGGCA CCTATTGGTC TTACTGACAT CCACTTTGCC |
| 2051 | TTTCTCTCCA CAGGTGCAGC TGCTGCAGCG GTCTAGAACT CGAGTCGAGA |
| 2101 | CCATGGCGAT GGATGTCACA AGGAGCCAGG CCCAGACAGC CTTGACTCTG |
| 2151 | GTAGAGCAGA TCCTGGCAGA GTTCCAGCTG CAGGAGGAGG ACCTGAAGAA |
| 2201 | GGTGATGAGA CGGATGCAGA AGGAGATGGA CCGCGGCCTG AGGCTGGAGA |
| 2251 | CCCATGAAGA GGCCAGTGTG AAGATGCTGC CCACCTACGT GCGCTCCACC |
| 2301 | CCAGAAGGCT CAGAAGTCGG GGACTTCCTC TCCCTGGACC TGGGTGGCAC |
| 2351 | TAACTTCAGG GTGATGCTGG TGAAGGTGGG AGAAGGTGAG GAGGGGCAGT |
| 2401 | GGAGCGTGAA GACCAAACAC CAGATGTACT CCATCCCCGA GGACGCCATG |
| 2451 | ACCGGCACTG CTGAGATGCT CTTCGACTAC ATCTCTGAGT GCATCTCCGA |
| 2501 | CTTCCTGGAC AAGCATCAGA TGAAACACAA GAAGCTGCCC CTGGGCTTCA |
| 2551 | CCTTCTCCTT TCCTGTGAGG CACGAAGACA TCGATAAGGG CATCCTTCTC |
| 2601 | AACTGGACCA AGGGCTTCAA GGCCTCAGGA GCAGAAGGGA ACAATGTCGT |
| 2651 | GGGGCTTCTG CGAGACGCTA TCAAACGGAG AGGGGACTTT GAAATGGATG |
| 2701 | TGGTGGCAAT GGTGAATGAC ACGGTGGCCA CGATGATCTC CTGCTACTAC |
| 2751 | GAAGACCATC AGTGCGAGGT CGGCATGATC GTGGGCACGG GCTGCAATGC |
| 2801 | CTGCTACATG GAGGAGATGC AGAATGTGGA GCTGGTGGAG GGGGACGAGG |
| 2851 | GCCGCATGTG CGTCAATACC GAGTGGGGCG CCTTCGGGGA CTCCGGCGAG |
| 2901 | CTGGACGAGT TCCTGCTGGA GTATGACCGC CTGGTGGACG AGAGCTCTGC |
| 2951 | AAACCCCGGT CAGCAGCTGT ATGAGAAGCT CATAGGTGGC AAGTACATGG |
| 3001 | GCGAGCTGGT GCGGCTTGTG CTGCTCAGGC TCGTGGACGA AAACCTGCTC |
| 3051 | TTCCACGGGG AGGCCTCCGA GCAGCTGCGC ACACGCGGAG CCTTCGAGAC |
| 3101 | GCGCTTCGTG TCGCAGGTGG AGAGCGACAC GGGCGACCGC AAGCAGATCT |
| 3151 | ACAACATCCT GAGCACGCTG GGGCTGCGAC CCTCGACCAC CGACTGCGAC |
| 3201 | ATCGTGCGCC GCGCCTGCGA GAGCGTGTCT ACGCGCGCTG CGCACATGTG |
| 3251 | CTCGGCGGGG CTGGCGGGCG TCATCAACCG CATGCGCGAG AGCCGCAGCG |
| 3301 | AGGACGTAAT GCGCATCACT GTGGGCGTGG ATGGCTCCGT GTACAAGCTG |
| 3351 | CACCCCAGCT TCAAGGAGCG GTTCCATGCC AGCGTGCGCA GGCTGACGCC |
| 3401 | CAGCTGCGAG ATCACCTTCA TCGAGTCGGA GGAGGGCAGT GGCCGGGGCG |
| 3451 | CGGCCCTGGT CTCGGCGGTG GCCTGTAAGA AGGCCTGTAT GCTGGGCCAG |
| 3501 | TGACTCGAGC ACGTGGAGCT CGCTGATCAG CCTCGACTGT GCCTTCTAGT |
| 3551 | TGCCAGCCAT CTGTTGTTTG CCCCTCCCCC GTGCCTTCCT TGACCCTGGA |
| 3601 | AGGTGCCACT CCCACTGTCC TTTCCTAATA AAATGAGGAA ATTGCATCGC |
| 3651 | ATTGTCTGAG TAGGTGTCAT TCTATTCTGG GGGGTGGGGT GGGGCAGGAC |
| 3701 | AGCAAGGGGG AGGATTGGGA AGACAATAGC AGGCATGCTG GGGATGCGGT |
| 3751 | GGGCTCTATG GCCACGTGAT TTAAATGCGG CCGCAGGAAC CCCTAGTGAT |
| 3801 | GGAGTTGGCC ACTCCCTCTC TGCGCGCTCG CTCGCTCACT GAGGCCGGGC |
| 3851 | GACCAAAGGT CGCCCGACGC CCGGGCTTTG CCCGGGCGGC CTCAGTGAGC |
| 3901 | GAGCGAGCGC GCAGCTGCCT GCAGGGGCGC CTGATGCGGT ATTTTCTCCT |
| 3951 | TACGCATCTG TGCGGTATTT CACACCGCAT ACGTCAAAGC AACCATAGTA |
| 4001 | CGCGCCCTGT AGCGGCGCAT TAAGCGCGGC GGGTGTGGTG GTTACGCGCA |
| 4051 | GCGTGACCGC TACACTTGCC AGCGCCCTAG CGCCCGCTCC TTTCGCTTTC |
| 4101 | TTCCCTTCCT TTCTCGCCAC GTTCGCCGGC TTTCCCCGTC AAGCTCTAAA |
| 4151 | TCGGGGGCTC CCTTTAGGGT TCCGATTTAG TGCTTTACGG CACCTCGACC |
| 4201 | CCAAAAAACT TGATTTGGGT GATGGTTCAC GTAGTGGGCC ATCGCCCTGA |
| 4251 | TAGACGGTTT TTCGCCCTTT GACGTTGGAG TCCACGTTCT TTAATAGTGG |
| 4301 | ACTCTTGTTC CAAACTGGAA CAACACTCAA CCCTATCTCG GGCTATTCTT |
| 4351 | TTGATTTATA AGGGATTTTG CCGATTTCGG CCTATTGGTT AAAAAATGAG |
| 4401 | CTGATTTAAC AAAAATTTAA CGCGAATTTT AACAAAATAT TAACGTTTAC |
| 4451 | AATTTTATGG TGCACTCTCA GTACAATCTG CTCTGATGCC GCATAGTTAA |
| 4501 | GCCAGCCCCG ACACCCGCCA ACACCCGCTG ACGCGCCCTG ACGGGCTTGT |
| 4551 | CTGCTCCCGG CATCCGCTTA CAGACAAGCT GTGACCGTCT CCGGGAGCTG |
| 4601 | CATGTGTCAG AGGTTTTCAC CGTCATCACC GAAACGCGCG AGACGAAAGG |
| 4651 | GCCTCGTGAT ACGCCTATTT TTATAGGTTA ATGTCATGAT AATAATGGTT |
| 4701 | TCTTAGACGT CAGGTGGCAC TTTTCGGGGA AATGTGCGCG GAACCCCTAT |
| 4751 | TTGTTTATTT TTCTAAATAC ATTCAAATAT GTATCCGCTC ATGAGACAAT |
| 4801 | AACCCTGATA AATGCTTCAA TAATATTGAA AAAGGAAGAG TATGAGTATT |
| 4851 | CAACATTTCC GTGTCGCCCT TATTCCCTTT TTTGCGGCAT TTTGCCTTCC |
| 4901 | TGTTTTTGCT CACCCAGAAA CGCTGGTGAA AGTAAAAGAT GCTGAAGATC |
| 4951 | AGTTGGGTGC ACGAGTGGGT TACATCGAAC TGGATCTCAA CAGCGGTAAG |
| 5001 | ATCCTTGAGA GTTTTCGCCC CGAAGAACGT TTTCCAATGA TGAGCACTTT |
| 5051 | TAAAGTTCTG CTATGTGGCG CGGTATTATC CCGTATTGAC GCCGGGCAAG |
| 5101 | AGCAACTCGG TCGCCGCATA CACTATTCTC AGAATGACTT GGTTGAGTAC |
| 5151 | TCACCAGTCA CAGAAAAGCA TCTTACGGAT GGCATGACAG TAAGAGAATT |
| 5201 | ATGCAGTGCT GCCATAACCA TGAGTGATAA CACTGCGGCC AACTTACTTC |
| 5251 | TGACAACGAT CGGAGGACCG AAGGAGCTAA CCGCTTTTTT GCACAACATG |
| 5301 | GGGGATCATG TAACTCGCCT TGATCGTTGG GAACCGGAGC TGAATGAAGC |
| 5351 | CATACCAAAC GACGAGCGTG ACACCACGAT GCCTGTAGCA ATGGCAACAA |
| 5401 | CGTTGCGCAA ACTATTAACT GGCGAACTAC TTACTCTAGC TTCCCGGCAA |
| 5451 | CAATTAATAG ACTGGATGGA GGCGGATAAA GTTGCAGGAC CACTTCTGCG |
| 5501 | CTCGGCCCTT CCGGCTGGCT GGTTTATTGC TGATAAATCT GGAGCCGGTG |
| 5551 | AGCGTGGGTC TCGCGGTATC ATTGCAGCAC TGGGGCCAGA TGGTAAGCCC |
| 5601 | TCCCGTATCG TAGTTATCTA CACGACGGGG AGTCAGGCAA CTATGGATGA |
| 5651 | ACGAAATAGA CAGATCGCTG AGATAGGTGC CTCACTGATT AAGCATTGGT |
| 5701 | AACTGTCAGA CCAAGTTTAC TCATATATAC TTTAGATTGA TTTAAAACTT |
| 5751 | CATTTTTAAT TTAAAAGGAT CTAGGTGAAG ATCCTTTTTG ATAATCTCAT |
| 5801 | GACCAAAATC CCTTAACGTG AGTTTTCGTT CCACTGAGCG TCAGACCCCG |
| 5851 | TAGAAAAGAT CAAAGGATCT TCTTGAGATC CTTTTTTTCT GCGCGTAATC |
| 5901 | TGCTGCTTGC AAACAAAAAA ACCACCGCTA CCAGCGGTGG TTTGTTTGCC |
| 5951 | GGATCAAGAG CTACCAACTC TTTTTCCGAA GGTAACTGGC TTCAGCAGAG |
| 6001 | CGCAGATACC AAATACTGTC CTTCTAGTGT AGCCGTAGTT AGGCCACCAC |
| 6051 | TTCAAGAACT CTGTAGCACC GCCTACATAC CTCGCTCTGC TAATCCTGTT |
| 6101 | ACCAGTGGCT GCTGCCAGTG GCGATAAGTC GTGTCTTACC GGGTTGGACT |
| 6151 | CAAGACGATA GTTACCGGAT AAGGCGCAGC GGTCGGGCTG AACGGGGGGT |
| 6201 | TCGTGCACAC AGCCCAGCTT GGAGCGAACG ACCTACACCG AACTGAGATA |
| 6251 | CCTACAGCGT GAGCTATGAG AAAGCGCCAC GCTTCCCGAA GGGAGAAAGG |
| 6301 | CGGACAGGTA TCCGGTAAGC GGCAGGGTCG GAACAGGAGA GCGCACGAGG |
| 6351 | GAGCTTCCAG GGGGAAACGC CTGGTATCTT TATAGTCCTG TCGGGTTTCG |
| 6401 | CCACCTCTGA CTTGAGCGTC GATTTTTGTG ATGCTCGTCA GGGGGGCGGA |
| 6451 | GCCTATGGAA AAACGCCAGC AACGCGGCCT TTTTACGGTT CCTGGCCTTT |
| 6501 | TGCTGGCCTT TTGCTCACAT GT |

ITR 5′: 1-141 bp
miniCMV promoter: 156-566 bp
hIns: 580-926 bp
SV40 polyA: 940-1238 bp
RSV promoter: 1354-2063 bp
hGck: 2088-3502 bp
bGH polyA: 3510-3768 bp
ITR 3′: 3785-3925 bp
K: RSV-hGck-miniCMV-hIns

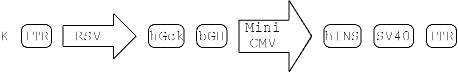

pAAV-RSV-hGck-miniCMV-hIns plasmid sequence
(SEQ ID NO: 14)

| | |
|---|---|
| 1 | CCTGCAGGCA GCTGCGCGCT CGCTCGCTCA CTGAGGCCGC CCGGGCAAAG |
| 51 | CCCGGGCGTC GGGCGACCTT TGGTCGCCCG GCCTCAGTGA GCGAGCGAGC |
| 101 | GCGCAGAGAG GGAGTGGCCA ACTCCATCAC TAGGGGTTCC TGCGGCCGCG |
| 151 | ATATCCATGT TTGACAGCTT ATCATCGCAG ATCCGTATGG TGCACTCTCA |
| 201 | GTACAATCTG CTCTGATGCC GCATAGTTAA GCCAGTATCT GCTCCCTGCT |
| 251 | TGTGTGTTGG AGGTCGCTGA GTAGTGCGCG AGCAAAATTT AAGCTACAAC |
| 301 | AAGGCAAGGC TTGACCGACA ATTGCATGAA GAATCTGCTT AGGGTTAGGC |
| 351 | GTTTTGCGCT GCTTCGCGAT GTACGGGCCA GATATTCGCG TATCTGAGGG |
| 401 | GACTAGGGTG TGTTTAGGCG AAAAGCGGGG CTTCGGTTGT ACGCGGTTAG |
| 451 | GAGTCCCCTC AGGATATAGT AGTTTCGCTT TTGCATAGGG AGGGGGAAAT |
| 501 | GTAGTCTTAT GCAATACTCT TGTAGTCTTG CAACATGGTA ACGATGAGTT |
| 551 | AGCAACATGC CTTACAAGGA GAGAAAAAGC ACCGTGCATG CCGATTGGTG |
| 601 | GAAGTAAGGT GGTACGATCG TGCCTTATTA GGAAGGCAAC AGACGGGTCT |
| 651 | GACATGGATT GGACGAACCA CTAAATTCCG CATTGCAGAG ATATTGTATT |
| 701 | TAAGTGCCTA GCTCGATACA ATAAACGCCA TTTGACCATT CACCACATTG |
| 751 | GTGTGCACCT CCAAGCTGGG TACCAGCTTC TAGAGAGATC TGCTTCAGCT |
| 801 | GGAGGCACTG GGCAGGTAAG TATCAAGGTT ACAAGACAGG TTTAAGGAGA |
| 851 | CCAATAGAAA CTGGGCTTGT CGAGACAGAG AAGACTCTTG CGTTTCTGAT |
| 901 | AGGCACCTAT TGGTCTTACT GACATCCACT TTGCCTTTCT CTCCACAGGT |
| 951 | GCAGCTGCTG CAGCGGTCTA GAACTCGAGT CGAGACCATG GCGATGGATG |
| 1001 | TCACAAGGAG CCAGGCCCAG ACAGCCTTGA CTCTGGTAGA GCAGATCCTG |
| 1051 | GCAGAGTTCC AGCTGCAGGA GGAGGACCTG AAGAAGGTGA TGAGACGGAT |
| 1101 | GCAGAAGGAG ATGGACCGCG GCCTGAGGCT GGAGACCCAT GAAGAGGCCA |
| 1151 | GTGTGAAGAT GCTGCCCACC TACGTGCGCT CCACCCCAGA AGGCTCAGAA |
| 1201 | GTCGGGGACT TCCTCTCCCT GGACCTGGGT GGCACTAACT TCAGGGTGAT |
| 1251 | GCTGGTGAAG GTGGGAGAAG GTGAGGAGGG GCAGTGGAGC GTGAAGACCA |
| 1301 | AACACCAGAT GTACTCCATC CCCGAGGACG CCATGACCGG CACTGCTGAG |
| 1351 | ATGCTCTTCG ACTACATCTC TGAGTGCATC TCCGACTTCC TGGACAAGCA |
| 1401 | TCAGATGAAA CACAAGAAGC TGCCCCTGGG CTTCACCTTC TCCTTTCCTG |
| 1451 | TGAGGCACGA AGACATCGAT AAGGGCATCC TTCTCAACTG GACCAAGGGC |
| 1501 | TTCAAGGCCT CAGGAGCAGA AGGGAACAAT GTCGTGGGGC TTCTGCGAGA |
| 1551 | CGCTATCAAA CGGAGAGGGG ACTTTGAAAT GGATGTGGTG GCAATGGTGA |
| 1601 | ATGACACGGT GGCCACGATG ATCTCCTGCT ACTACGAAGA CCATCAGTGC |
| 1651 | GAGGTCGGCA TGATCGTGGG CACGGGCTGC AATGCCTGCT ACATGGAGGA |
| 1701 | GATGCAGAAT GTGGAGCTGG TGGAGGGGGA CGAGGGCCGC ATGTGCGTCA |
| 1751 | ATACCGAGTG GGGCGCCTTC GGGGACTCCG GCGAGCTGGA CGAGTTCCTG |
| 1801 | CTGGAGTATG ACCGCCTGGT GGACGAGAGC TCTGCAAACC CCGGTCAGCA |
| 1851 | GCTGTATGAG AAGCTCATAG GTGGCAAGTA CATGGGCGAG CTGGTGCGGC |
| 1901 | TTGTGCTGCT CAGGCTCGTG GACGAAAACC TGCTCTTCCA CGGGGAGGCC |
| 1951 | TCCGAGCAGC TGCGCACACG CGGAGCCTTC GAGACGCGCT TCGTGTCGCA |
| 2001 | GGTGGAGAGC GACACGGGCG ACCGCAAGCA GATCTACAAC ATCCTGAGCA |
| 2051 | CGCTGGGGCT GCGACCCTCG ACCACCGACT GCGACATCGT GCGCCGCGCC |
| 2101 | TGCGAGAGCG TGTCTACGCG CGCTGCGCAC ATGTGCTCGG CGGGGCTGGC |
| 2151 | GGGCGTCATC AACCGCATGC GCGAGAGCCG CAGCGAGGAC GTAATGCGCA |
| 2201 | TCACTGTGGG CGTGGATGGC TCCGTGTACA AGCTGCACCC CAGCTTCAAG |
| 2251 | GAGCGGTTCC ATGCCAGCGT GCGCAGGCTG ACGCCCAGCT GCGAGATCAC |
| 2301 | CTTCATCGAG TCGGAGGAGG GCAGTGGCCG GGGCGCGGCC CTGGTCTCGG |
| 2351 | CGGTGGCCTG TAAGAAGGCC TGTATGCTGG GCCAGTGACT CGAGCACGTG |
| 2401 | GAGCTCGCTG ATCAGCCTCG ACTGTGCCTT CTAGTTGCCA GCCATCTGTT |
| 2451 | GTTTGCCCCT CCCCCGTGCC TTCCTTGACC CTGGAAGGTG CCACTCCCAC |
| 2501 | TGTCCTTTCC TAATAAAATG AGGAAATTGC ATCGCATTGT CTGAGTAGGT |
| 2551 | GTCATTCTAT TCTGGGGGGT GGGGTGGGGC AGGACAGCAA GGGGGAGGAT |
| 2601 | TGGGAAGACA ATAGCAGGCA TGCTGGGGAT GCGGTGGGCT CTATGGCCAC |
| 2651 | GTGATTTATC TATGCCAAGT ACGCCCCCTA TTGACGTCAA TGACGGTAAA |
| 2701 | TGGCCCGCCT GGCATTATGC CCAGTACATG ACCTTATGGG ACTTTCCTAC |
| 2751 | TTGGCAGTAC ATCTACGTAT TAGTCATCGC TATTACCATG GTGATGCGGT |
| 2801 | TTTGGCAGTA CATCAATGGG CGTGGATAGC GGTTTGACTC ACGGGGATTT |
| 2851 | CCAAGTCTCC ACCCCATTGA CGTCAATGGG AGTTTGTTTT GGCACCAAAA |
| 2901 | TCAACGGGAC TTTCCAAAAT GTCGTAACAA CTCCGCCCCA TTGACGCAAA |
| 2951 | TGGGCGGTAG GCGTGTACGG TGGGAGGTCT ATATAAGCAG AGCTCTCTGG |
| 3001 | CTAACTAGAG AACCCACTGC TTAACTGGCT TATCGAAATT AATACGACTC |
| 3051 | ACTATAGGGA GACCCAAGCT TGCTAGCGTC GACCTTCTGC CATGGCCCTG |
| 3101 | TGGATGCGCC TCCTGCCCCT GCTGGCGCTG CTGGCCCTCT GGGGACCTGA |
| 3151 | CCCAGCCGCA GCCTTTGTGA ACCAACACCT GTGCGGCTCA GATCTGGTGG |
| 3201 | AAGCTCTCTA CCTAGTGTGC GGGGAACGAG GCTTCTTCTA CACACCCAGG |
| 3251 | ACCAAGCGGG AGGCAGAGGA CCTGCAGGTG GGGCAGGTGG AGCTGGGCGG |
| 3301 | GGGCCCTGGT GCAGGCAGCC TGCAGCCCTT GGCCCTGGAG GGGTCGCGAC |
| 3351 | AGAAGCGTGG CATTGTGGAA CAATGCTGTA CCAGCATCTG CTCCCTCTAC |
| 3401 | CAGCTGGAGA ACTACTGCAA CTAGACGCAG CCGTCGACGG TACCAGCGCT |
| 3451 | GTCGAGGCCG CTTCGAGCAG ACATGATAAG ATACATTGAT GAGTTTGGAC |
| 3501 | AAACCACAAC TAGAATGCAG TGAAAAAAAT GCTTTATTTG TGAAATTTGT |
| 3551 | GATGCTATTG CTTTATTTGT AACCATTATA AGCTGCAATA AACAAGTTAA |
| 3601 | CAACAACAAT TGCATTCATT TTATGTTTCA GGTTCAGGGG GAGATGTGGG |
| 3651 | AGGTTTTTTA AAGCAAGTAA AACCTCTACA AATGTGGTAA AATCGATTAG |
| 3701 | GATCTTCCTA GAGCATGGCT ACCTAGACAT GGCTCGACAG ATCAGCGCTC |
| 3751 | ATGCTCTGGA AGATCTCGAT TTAAATGCGG CCGCAGGAAC CCCTAGTGAT |
| 3801 | GGAGTTGGCC ACTCCCTCTC TGCGCGCTCG CTCGCTCACT GAGGCCGGGC |
| 3851 | GACCAAAGGT CGCCCGACGC CCGGGCTTTG CCCGGGCGGC CTCAGTGAGC |
| 3901 | GAGCGAGCGC GCAGCTGCCT GCAGGGGCGC CTGATGCGGT ATTTTCTCCT |
| 3951 | TACGCATCTG TGCGGTATTT CACACCGCAT ACGTCAAAGC AACCATAGTA |
| 4001 | CGCGCCCTGT AGCGGCGCAT TAAGCGCGGC GGGTGTGGTG GTTACGCGCA |
| 4051 | GCGTGACCGC TACACTTGCC AGCGCCCTAG CGCCCGCTCC TTTCGCTTTC |
| 4101 | TTCCCTTCCT TTCTCGCCAC GTTCGCCGGC TTTCCCCGTC AAGCTCTAAA |
| 4151 | TCGGGGGCTC CCTTTAGGGT TCCGATTTAG TGCTTTACGG CACCTCGACC |
| 4201 | CCAAAAAACT TGATTTGGGT GATGGTTCAC GTAGTGGGCC ATCGCCCTGA |
| 4251 | TAGACGGTTT TTCGCCCTTT GACGTTGGAG TCCACGTTCT TTAATAGTGG |
| 4301 | ACTCTTGTTC CAAACTGGAA CAACACTCAA CCCTATCTCG GGCTATTCTT |
| 4351 | TTGATTTATA AGGGATTTTG CCGATTTCGG CCTATTGGTT AAAAAATGAG |
| 4401 | CTGATTTAAC AAAAATTTAA CGCGAATTTT AACAAAATAT TAACGTTTAC |
| 4451 | AATTTTATGG TGCACTCTCA GTACAATCTG CTCTGATGCC GCATAGTTAA |
| 4501 | GCCAGCCCCG ACACCCGCCA ACACCCGCTG ACGCGCCCTG ACGGGCTTGT |
| 4551 | CTGCTCCCGG CATCCGCTTA CAGACAAGCT GTGACCGTCT CCGGGAGCTG |
| 4601 | CATGTGTCAG AGGTTTTCAC CGTCATCACC GAAACGCGCG AGACGAAAGG |
| 4651 | GCCTCGTGAT ACGCCTATTT TTATAGGTTA ATGTCATGAT AATAATGGTT |
| 4701 | TCTTAGACGT CAGGTGGCAC TTTTCGGGGA AATGTGCGCG GAACCCCTAT |
| 4751 | TTGTTTATTT TTCTAAATAC ATTCAAATAT GTATCCGCTC ATGAGACAAT |
| 4801 | AACCCTGATA AATGCTTCAA TAATATTGAA AAAGGAAGAG TATGAGTATT |
| 4851 | CAACATTTCC GTGTCGCCCT TATTCCCTTT TTTGCGGCAT TTTGCCTTCC |
| 4901 | TGTTTTTGCT CACCCAGAAA CGCTGGTGAA AGTAAAAGAT GCTGAAGATC |
| 4951 | AGTTGGGTGC ACGAGTGGGT TACATCGAAC TGGATCTCAA CAGCGGTAAG |
| 5001 | ATCCTTGAGA GTTTTCGCCC CGAAGAACGT TTTCCAATGA TGAGCACTTT |
| 5051 | TAAAGTTCTG CTATGTGGCG CGGTATTATC CCGTATTGAC GCCGGGCAAG |
| 5101 | AGCAACTCGG TCGCCGCATA CACTATTCTC AGAATGACTT GGTTGAGTAC |
| 5151 | TCACCAGTCA CAGAAAAGCA TCTTACGGAT GGCATGACAG TAAGAGAATT |
| 5201 | ATGCAGTGCT GCCATAACCA TGAGTGATAA CACTGCGGCC AACTTACTTC |
| 5251 | TGACAACGAT CGGAGGACCG AAGGAGCTAA CCGCTTTTTT GCACAACATG |
| 5301 | GGGGATCATG TAACTCGCCT TGATCGTTGG GAACCGGAGC TGAATGAAGC |
| 5351 | CATACCAAAC GACGAGCGTG ACACCACGAT GCCTGTAGCA ATGGCAACAA |
| 5401 | CGTTGCGCAA ACTATTAACT GGCGAACTAC TTACTCTAGC TTCCCGGCAA |
| 5451 | CAATTAATAG ACTGGATGGA GGCGGATAAA GTTGCAGGAC CACTTCTGCG |
| 5501 | CTCGGCCCTT CCGGCTGGCT GGTTTATTGC TGATAAATCT GGAGCCGGTG |
| 5551 | AGCGTGGGTC TCGCGGTATC ATTGCAGCAC TGGGGCCAGA TGGTAAGCCC |
| 5601 | TCCCGTATCG TAGTTATCTA CACGACGGGG AGTCAGGCAA CTATGGATGA |
| 5651 | ACGAAATAGA CAGATCGCTG AGATAGGTGC CTCACTGATT AAGCATTGGT |
| 5701 | AACTGTCAGA CCAAGTTTAC TCATATATAC TTTAGATTGA TTTAAAACTT |
| 5751 | CATTTTTAAT TTAAAAGGAT CTAGGTGAAG ATCCTTTTTG ATAATCTCAT |
| 5801 | GACCAAAATC CCTTAACGTG AGTTTTCGTT CCACTGAGCG TCAGACCCCG |
| 5851 | TAGAAAAGAT CAAAGGATCT TCTTGAGATC CTTTTTTTCT GCGCGTAATC |
| 5901 | TGCTGCTTGC AAACAAAAAA ACCACCGCTA CCAGCGGTGG TTTGTTTGCC |
| 5951 | GGATCAAGAG CTACCAACTC TTTTTCCGAA GGTAACTGGC TTCAGCAGAG |
| 6001 | CGCAGATACC AAATACTGTC CTTCTAGTGT AGCCGTAGTT AGGCCACCAC |
| 6051 | TTCAAGAACT CTGTAGCACC GCCTACATAC CTCGCTCTGC TAATCCTGTT |
| 6101 | ACCAGTGGCT GCTGCCAGTG GCGATAAGTC GTGTCTTACC GGGTTGGACT |
| 6151 | CAAGACGATA GTTACCGGAT AAGGCGCAGC GGTCGGGCTG AACGGGGGGT |
| 6201 | TCGTGCACAC AGCCCAGCTT GGAGCGAACG ACCTACACCG AACTGAGATA |
| 6251 | CCTACAGCGT GAGCTATGAG AAAGCGCCAC GCTTCCCGAA GGGAGAAAGG |
| 6301 | CGGACAGGTA TCCGGTAAGC GGCAGGGTCG GAACAGGAGA GCGCACGAGG |
| 6351 | GAGCTTCCAG GGGGAAACGC CTGGTATCTT TATAGTCCTG TCGGGTTTCG |
| 6401 | CCACCTCTGA CTTGAGCGTC GATTTTTGTG ATGCTCGTCA GGGGGGCGGA |
| 6451 | GCCTATGGAA AAACGCCAGC AACGCGGCCT TTTTACGGTT CCTGGCCTTT |
| 6501 | TGCTGGCCTT TTGCTCACAT GT |

ITR 5′: 1-141 bp
RSV promoter: 239-948 bp
hGck: 973-2387 bp
bGH polyA: 2395-2653 bp
miniCMV promoter: 2661-3071 bp
hIns: 3085-3431 bp
SV40 polyA: 3445-3743 bp
ITR 3′: 3785-3925 bp
L: miniCMV-hIns(rev)-RSV-hGck

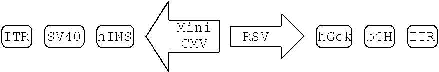

pAAV-miniCMV-hIns(rev)-RSV-hGck plasmid sequence
(SEQ ID NO: 15)

| | |
|---|---|
| 1 | CCTGCAGGCA GCTGCGCGCT CGCTCGCTCA CTGAGGCCGC CCGGGCAAAG |
| 51 | CCCGGGCGTC GGGCGACCTT TGGTCGCCCG GCCTCAGTGA GCGAGCGAGC |
| 101 | GCGCAGAGAG GGAGTGGCCA ACTCCATCAC TAGGGGTTCC TGCGGCCGCG |
| 151 | ATAAATCGAG ATCTTCCAGA GCATGAGCGC TGATCTGTCG AGCCATGTCT |
| 201 | AGGTAGCCAT GCTCTAGGAA GATCCTAATC GATTTTACCA CATTTGTAGA |
| 251 | GGTTTTACTT GCTTTAAAAA ACCTCCCACA TCTCCCCCTG AACCTGAAAC |
| 301 | ATAAAATGAA TGCAATTGTT GTTGTTAACT TGTTTATTGC AGCTTATAAT |
| 351 | GGTTACAAAT AAAGCAATAG CATCACAAAT TTCACAAATA AAGCATTTTT |
| 401 | TTCACTGCAT TCTAGTTGTG GTTTGTCCAA ACTCATCAAT GTATCTTATC |
| 451 | ATGTCTGCTC GAAGCGGCCT CGACAGCGCT GGTACCGTCG ACGGCTGCGT |
| 501 | CTAGTTGCAG TAGTTCTCCA GCTGGTAGAG GGAGCAGATG CTGGTACAGC |
| 551 | ATTGTTCCAC AATGCCACGC TTCTGTCGCG ACCCCTCCAG GGCCAAGGGC |
| 601 | TGCAGGCTGC CTGCACCAGG GCCCCCGCCC AGCTCCACCT GCCCCACCTG |
| 651 | CAGGTCCTCT GCCTCCCGCT TGGTCCTGGG TGTGTAGAAG AAGCCTCGTT |
| 701 | CCCCGCACAC TAGGTAGAGA GCTTCCACCA GATCTGAGCC GCACAGGTGT |
| 751 | TGGTTCACAA AGGCTGCGGC TGGGTCAGGT CCCCAGAGGG CCAGCAGCGC |
| 801 | CAGCAGGGGC AGGAGGCGCA TCCACAGGGC CATGGCAGAA GGTCGACGCT |
| 851 | AGCAAGCTTG GGTCTCCCTA TAGTGAGTCG TATTAATTTC GATAAGCCAG |
| 901 | TTAAGCAGTG GGTTCTCTAG TTAGCCAGAG AGCTCTGCTT ATATAGACCT |
| 951 | CCCACCGTAC ACGCCTACCG CCCATTTGCG TCAATGGGGC GGAGTTGTTA |
| 1001 | CGACATTTTG GAAAGTCCCG TTGATTTTGG TGCCAAAACA AACTCCCATT |
| 1051 | GACGTCAATG GGGTGGAGAC TTGGAAATCC CCGTGAGTCA AACCGCTATC |
| 1101 | CACGCCCATT GATGTACTGC CAAAACCGCA TCACCATGGT AATAGCGATG |
| 1151 | ACTAATACGT AGATGTACTG CCAAGTAGGA AAGTCCCATA AGGTCATGTA |
| 1201 | CTGGGCATAA TGCCAGGCGG GCCATTTACC GTCATTGACG TCAATAGGGG |
| 1251 | GCGTACTTGG CATAGATATC CATGTTTGAC AGCTTATCAT CGCAGATCCG |
| 1301 | TATGGTGCAC TCTCAGTACA ATCTGCTCTG ATGCCGCATA GTTAAGCCAG |
| 1351 | TATCTGCTCC CTGCTTGTGT GTTGGAGGTC GCTGAGTAGT GCGCGAGCAA |
| 1401 | AATTTAAGCT ACAACAAGGC AAGGCTTGAC CGACAATTGC ATGAAGAATC |
| 1451 | TGCTTAGGGT TAGGCGTTTT GCGCTGCTTC GCGATGTACG GGCCAGATAT |
| 1501 | TCGCGTATCT GAGGGGACTA GGGTGTGTTT AGGCGAAAAG CGGGGCTTCG |
| 1551 | GTTGTACGCG GTTAGGAGTC CCCTCAGGAT ATAGTAGTTT CGCTTTTGCA |
| 1601 | TAGGGAGGGG GAAATGTAGT CTTATGCAAT ACTCTTGTAG TCTTGCAACA |
| 1651 | TGGTAACGAT GAGTTAGCAA CATGCCTTAC AAGGAGAGAA AAAGCACCGT |
| 1701 | GCATGCCGAT TGGTGGAAGT AAGGTGGTAC GATCGTGCCT TATTAGGAAG |
| 1751 | GCAACAGACG GGTCTGACAT GGATTGGACG AACCACTAAA TTCCGCATTG |
| 1801 | CAGAGATATT GTATTTAAGT GCCTAGCTCG ATACAATAAA CGCCATTTGA |
| 1851 | CCATTCACCA CATTGGTGTG CACCTCCAAG CTGGGTACCA GCTTCTAGAG |
| 1901 | AGATCTGCTT CAGCTGGAGG CACTGGGCAG GTAAGTATCA AGGTTACAAG |
| 1951 | ACAGGTTTAA GGAGACCAAT AGAAACTGGG CTTGTCGAGA CAGAGAAGAC |
| 2001 | TCTTGCGTTT CTGATAGGCA CCTATTGGTC TTACTGACAT CCACTTTGCC |
| 2051 | TTTCTCTCCA CAGGTGCAGC TGCTGCAGCG GTCTAGAACT CGAGTCGAGA |
| 2101 | CCATGGCGAT GGATGTCACA AGGAGCCAGG CCCAGACAGC CTTGACTCTG |
| 2151 | GTAGAGCAGA TCCTGGCAGA GTTCCAGCTG CAGGAGGAGG ACCTGAAGAA |
| 2201 | GGTGATGAGA CGGATGCAGA AGGAGATGGA CCGCGGCCTG AGGCTGGAGA |
| 2251 | CCCATGAAGA GGCCAGTGTG AAGATGCTGC CCACCTACGT GCGCTCCACC |
| 2301 | CCAGAAGGCT CAGAAGTCGG GGACTTCCTC TCCCTGGACC TGGGTGGCAC |
| 2351 | TAACTTCAGG GTGATGCTGG TGAAGGTGGG AGAAGGTGAG GAGGGGCAGT |
| 2401 | GGAGCGTGAA GACCAAACAC CAGATGTACT CCATCCCCGA GGACGCCATG |
| 2451 | ACCGGCACTG CTGAGATGCT CTTCGACTAC ATCTCTGAGT GCATCTCCGA |
| 2501 | CTTCCTGGAC AAGCATCAGA TGAAACACAA GAAGCTGCCC CTGGGCTTCA |
| 2551 | CCTTCTCCTT TCCTGTGAGG CACGAAGACA TCGATAAGGG CATCCTTCTC |
| 2601 | AACTGGACCA AGGGCTTCAA GGCCTCAGGA GCAGAAGGGA ACAATGTCGT |
| 2651 | GGGGCTTCTG CGAGACGCTA TCAAACGGAG AGGGGACTTT GAAATGGATG |
| 2701 | TGGTGGCAAT GGTGAATGAC ACGGTGGCCA CGATGATCTC CTGCTACTAC |
| 2751 | GAAGACCATC AGTGCGAGGT CGGCATGATC GTGGGCACGG GCTGCAATGC |
| 2801 | CTGCTACATG GAGGAGATGC AGAATGTGGA GCTGGTGGAG GGGGACGAGG |
| 2851 | GCCGCATGTG CGTCAATACC GAGTGGGGCG CCTTCGGGGA CTCCGGCGAG |
| 2901 | CTGGACGAGT TCCTGCTGGA GTATGACCGC CTGGTGGACG AGAGCTCTGC |
| 2951 | AAACCCCGGT CAGCAGCTGT ATGAGAAGCT CATAGGTGGC AAGTACATGG |
| 3001 | GCGAGCTGGT GCGGCTTGTG CTGCTCAGGC TCGTGGACGA AAACCTGCTC |
| 3051 | TTCCACGGGG AGGCCTCCGA GCAGCTGCGC ACACGCGGAG CCTTCGAGAC |
| 3101 | GCGCTTCGTG TCGCAGGTGG AGAGCGACAC GGGCGACCGC AAGCAGATCT |
| 3151 | ACAACATCCT GAGCACGCTG GGGCTGCGAC CCTCGACCAC CGACTGCGAC |
| 3201 | ATCGTGCGCC GCGCCTGCGA GAGCGTGTCT ACGCGCGCTG CGCACATGTG |
| 3251 | CTCGGCGGGG CTGGCGGGCG TCATCAACCG CATGCGCGAG AGCCGCAGCG |
| 3301 | AGGACGTAAT GCGCATCACT GTGGGCGTGG ATGGCTCCGT GTACAAGCTG |
| 3351 | CACCCCAGCT TCAAGGAGCG GTTCCATGCC AGCGTGCGCA GGCTGACGCC |
| 3401 | CAGCTGCGAG ATCACCTTCA TCGAGTCGGA GGAGGGCAGT GGCCGGGGCG |
| 3451 | CGGCCCTGGT CTCGGCGGTG GCCTGTAAGA AGGCCTGTAT GCTGGGCCAG |
| 3501 | TGACTCGAGC ACGTGGAGCT CGCTGATCAG CCTCGACTGT GCCTTCTAGT |
| 3551 | TGCCAGCCAT CTGTTGTTTG CCCCTCCCCC GTGCCTTCCT TGACCCTGGA |
| 3601 | AGGTGCCACT CCCACTGTCC TTTCCTAATA AAATGAGGAA ATTGCATCGC |
| 3651 | ATTGTCTGAG TAGGTGTCAT TCTATTCTGG GGGGTGGGGT GGGGCAGGAC |
| 3701 | AGCAAGGGGG AGGATTGGGA AGACAATAGC AGGCATGCTG GGGATGCGGT |
| 3751 | GGGCTCTATG GCCACGTGAT TTAAATGCGG CCGCAGGAAC CCCTAGTGAT |
| 3801 | GGAGTTGGCC ACTCCCTCTC TGCGCGCTCG CTCGCTCACT GAGGCCGGGC |
| 3851 | GACCAAAGGT CGCCCGACGC CCGGGCTTTG CCCGGGCGGC CTCAGTGAGC |
| 3901 | GAGCGAGCGC GCAGCTGCCT GCAGGGGCGC CTGATGCGGT ATTTTCTCCT |
| 3951 | TACGCATCTG TGCGGTATTT CACACCGCAT ACGTCAAAGC AACCATAGTA |
| 4001 | CGCGCCCTGT AGCGGCGCAT TAAGCGCGGC GGGTGTGGTG GTTACGCGCA |
| 4051 | GCGTGACCGC TACACTTGCC AGCGCCCTAG CGCCCGCTCC TTTCGCTTTC |
| 4101 | TTCCCTTCCT TTCTCGCCAC GTTCGCCGGC TTTCCCCGTC AAGCTCTAAA |
| 4151 | TCGGGGGCTC CCTTTAGGGT TCCGATTTAG TGCTTTACGG CACCTCGACC |
| 4201 | CCAAAAAACT TGATTTGGGT GATGGTTCAC GTAGTGGGCC ATCGCCCTGA |
| 4251 | TAGACGGTTT TTCGCCCTTT GACGTTGGAG TCCACGTTCT TTAATAGTGG |
| 4301 | ACTCTTGTTC CAAACTGGAA CAACACTCAA CCCTATCTCG GGCTATTCTT |
| 4351 | TTGATTTATA AGGGATTTTG CCGATTTCGG CCTATTGGTT AAAAAATGAG |
| 4401 | CTGATTTAAC AAAAATTTAA CGCGAATTTT AACAAAATAT TAACGTTTAC |
| 4451 | AATTTTATGG TGCACTCTCA GTACAATCTG CTCTGATGCC GCATAGTTAA |
| 4501 | GCCAGCCCCG ACACCCGCCA ACACCCGCTG ACGCGCCCTG ACGGGCTTGT |
| 4551 | CTGCTCCCGG CATCCGCTTA CAGACAAGCT GTGACCGTCT CCGGGAGCTG |
| 4601 | CATGTGTCAG AGGTTTTCAC CGTCATCACC GAAACGCGCG AGACGAAAGG |
| 4651 | GCCTCGTGAT ACGCCTATTT TTATAGGTTA ATGTCATGAT AATAATGGTT |
| 4701 | TCTTAGACGT CAGGTGGCAC TTTTCGGGGA AATGTGCGCG GAACCCCTAT |
| 4751 | TTGTTTATTT TTCTAAATAC ATTCAAATAT GTATCCGCTC ATGAGACAAT |
| 4801 | AACCCTGATA AATGCTTCAA TAATATTGAA AAAGGAAGAG TATGAGTATT |
| 4851 | CAACATTTCC GTGTCGCCCT TATTCCCTTT TTTGCGGCAT TTTGCCTTCC |
| 4901 | TGTTTTTGCT CACCCAGAAA CGCTGGTGAA AGTAAAAGAT GCTGAAGATC |
| 4951 | AGTTGGGTGC ACGAGTGGGT TACATCGAAC TGGATCTCAA CAGCGGTAAG |
| 5001 | ATCCTTGAGA GTTTTCGCCC CGAAGAACGT TTTCCAATGA TGAGCACTTT |
| 5051 | TAAAGTTCTG CTATGTGGCG CGGTATTATC CCGTATTGAC GCCGGGCAAG |
| 5101 | AGCAACTCGG TCGCCGCATA CACTATTCTC AGAATGACTT GGTTGAGTAC |
| 5151 | TCACCAGTCA CAGAAAAGCA TCTTACGGAT GGCATGACAG TAAGAGAATT |
| 5201 | ATGCAGTGCT GCCATAACCA TGAGTGATAA CACTGCGGCC AACTTACTTC |
| 5251 | TGACAACGAT CGGAGGACCG AAGGAGCTAA CCGCTTTTTT GCACAACATG |
| 5301 | GGGGATCATG TAACTCGCCT TGATCGTTGG GAACCGGAGC TGAATGAAGC |
| 5351 | CATACCAAAC GACGAGCGTG ACACCACGAT GCCTGTAGCA ATGGCAACAA |
| 5401 | CGTTGCGCAA ACTATTAACT GGCGAACTAC TTACTCTAGC TTCCCGGCAA |
| 5451 | CAATTAATAG ACTGGATGGA GGCGGATAAA GTTGCAGGAC CACTTCTGCG |
| 5501 | CTCGGCCCTT CCGGCTGGCT GGTTTATTGC TGATAAATCT GGAGCCGGTG |
| 5551 | AGCGTGGGTC TCGCGGTATC ATTGCAGCAC TGGGGCCAGA TGGTAAGCCC |
| 5601 | TCCCGTATCG TAGTTATCTA CACGACGGGG AGTCAGGCAA CTATGGATGA |
| 5651 | ACGAAATAGA CAGATCGCTG AGATAGGTGC CTCACTGATT AAGCATTGGT |
| 5701 | AACTGTCAGA CCAAGTTTAC TCATATATAC TTTAGATTGA TTTAAAACTT |
| 5751 | CATTTTTAAT TTAAAAGGAT CTAGGTGAAG ATCCTTTTTG ATAATCTCAT |
| 5801 | GACCAAAATC CCTTAACGTG AGTTTTCGTT CCACTGAGCG TCAGACCCCG |
| 5851 | TAGAAAAGAT CAAAGGATCT TCTTGAGATC CTTTTTTTCT GCGCGTAATC |
| 5901 | TGCTGCTTGC AAACAAAAAA ACCACCGCTA CCAGCGGTGG TTTGTTTGCC |
| 5951 | GGATCAAGAG CTACCAACTC TTTTTCCGAA GGTAACTGGC TTCAGCAGAG |
| 6001 | CGCAGATACC AAATACTGTC CTTCTAGTGT AGCCGTAGTT AGGCCACCAC |
| 6051 | TTCAAGAACT CTGTAGCACC GCCTACATAC CTCGCTCTGC TAATCCTGTT |
| 6101 | ACCAGTGGCT GCTGCCAGTG GCGATAAGTC GTGTCTTACC GGGTTGGACT |
| 6151 | CAAGACGATA GTTACCGGAT AAGGCGCAGC GGTCGGGCTG AACGGGGGGT |
| 6201 | TCGTGCACAC AGCCCAGCTT GGAGCGAACG ACCTACACCG AACTGAGATA |
| 6251 | CCTACAGCGT GAGCTATGAG AAAGCGCCAC GCTTCCCGAA GGGAGAAAGG |
| 6301 | CGGACAGGTA TCCGGTAAGC GGCAGGGTCG GAACAGGAGA GCGCACGAGG |
| 6351 | GAGCTTCCAG GGGGAAACGC CTGGTATCTT TATAGTCCTG TCGGGTTTCG |
| 6401 | CCACCTCTGA CTTGAGCGTC GATTTTTGTG ATGCTCGTCA GGGGGGCGGA |
| 6451 | GCCTATGGAA AAACGCCAGC AACGCGGCCT TTTTACGGTT CCTGGCCTTT |
| 6501 | TGCTGGCCTT TTGCTCACAT GT |

ITR 5′: 1-141 bp
SV40 polyA: 182-480 bp
hIns: 494-840 bp
miniCMV promoter: 854-1264 bp
RSV promoter: 1354-2063 bp
hGck: 2088-3502 bp
bGH polyA: 3510-3768 bp
ITR 3′: 3785-3925 bp
M: RSV-hGck-miniCMV-hIns(rev)

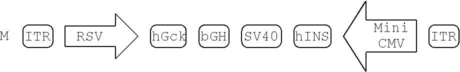

pAAV-RSV-hGck-miniCMV-hIns(rev) plasmid sequence
(SEQ ID NO: 16)

| | |
|---|---|
| 1 | CCTGCAGGCA GCTGCGCGCT CGCTCGCTCA CTGAGGCCGC CCGGGCAAAG |
| 51 | CCCGGGCGTC GGGCGACCTT TGGTCGCCCG GCCTCAGTGA GCGAGCGAGC |
| 101 | GCGCAGAGAG GGAGTGGCCA ACTCCATCAC TAGGGGTTCC TGCGGCCGCG |
| 151 | ATATCCATGT TTGACAGCTT ATCATCGCAG ATCCGTATGG TGCACTCTCA |
| 201 | GTACAATCTG CTCTGATGCC GCATAGTTAA GCCAGTATCT GCTCCCTGCT |
| 251 | TGTGTGTTGG AGGTCGCTGA GTAGTGCGCG AGCAAAATTT AAGCTACAAC |
| 301 | AAGGCAAGGC TTGACCGACA ATTGCATGAA GAATCTGCTT AGGGTTAGGC |
| 351 | GTTTTGCGCT GCTTCGCGAT GTACGGGCCA GATATTCGCG TATCTGAGGG |
| 401 | GACTAGGGTG TGTTTAGGCG AAAAGCGGGG CTTCGGTTGT ACGCGGTTAG |
| 451 | GAGTCCCCTC AGGATATAGT AGTTTCGCTT TTGCATAGGG AGGGGGAAAT |
| 501 | GTAGTCTTAT GCAATACTCT TGTAGTCTTG CAACATGGTA ACGATGAGTT |
| 551 | AGCAACATGC CTTACAAGGA GAGAAAAAGC ACCGTGCATG CCGATTGGTG |
| 601 | GAAGTAAGGT GGTACGATCG TGCCTTATTA GGAAGGCAAC AGACGGGTCT |
| 651 | GACATGGATT GGACGAACCA CTAAATTCCG CATTGCAGAG ATATTGTATT |
| 701 | TAAGTGCCTA GCTCGATACA ATAAACGCCA TTTGACCATT CACCACATTG |
| 751 | GTGTGCACCT CCAAGCTGGG TACCAGCTTC TAGAGAGATC TGCTTCAGCT |
| 801 | GGAGGCACTG GGCAGGTAAG TATCAAGGTT ACAAGACAGG TTTAAGGAGA |
| 851 | CCAATAGAAA CTGGGCTTGT CGAGACAGAG AAGACTCTTG CGTTTCTGAT |
| 901 | AGGCACCTAT TGGTCTTACT GACATCCACT TTGCCTTTCT CTCCACAGGT |
| 951 | GCAGCTGCTG CAGCGGTCTA GAACTCGAGT CGAGACCATG GCGATGGATG |
| 1001 | TCACAAGGAG CCAGGCCCAG ACAGCCTTGA CTCTGGTAGA GCAGATCCTG |
| 1051 | GCAGAGTTCC AGCTGCAGGA GGAGGACCTG AAGAAGGTGA TGAGACGGAT |
| 1101 | GCAGAAGGAG ATGGACCGCG GCCTGAGGCT GGAGACCCAT GAAGAGGCCA |
| 1151 | GTGTGAAGAT GCTGCCCACC TACGTGCGCT CCACCCCAGA AGGCTCAGAA |
| 1201 | GTCGGGGACT TCCTCTCCCT GGACCTGGGT GGCACTAACT TCAGGGTGAT |
| 1251 | GCTGGTGAAG GTGGGAGAAG GTGAGGAGGG GCAGTGGAGC GTGAAGACCA |
| 1301 | AACACCAGAT GTACTCCATC CCCGAGGACG CCATGACCGG CACTGCTGAG |
| 1351 | ATGCTCTTCG ACTACATCTC TGAGTGCATC TCCGACTTCC TGGACAAGCA |
| 1401 | TCAGATGAAA CACAAGAAGC TGCCCCTGGG CTTCACCTTC TCCTTTCCTG |
| 1451 | TGAGGCACGA AGACATCGAT AAGGGCATCC TTCTCAACTG GACCAAGGGC |
| 1501 | TTCAAGGCCT CAGGAGCAGA AGGGAACAAT GTCGTGGGGC TTCTGCGAGA |
| 1551 | CGCTATCAAA CGGAGAGGGG ACTTTGAAAT GGATGTGGTG GCAATGGTGA |
| 1601 | ATGACACGGT GGCCACGATG ATCTCCTGCT ACTACGAAGA CCATCAGTGC |
| 1651 | GAGGTCGGCA TGATCGTGGG CACGGGCTGC AATGCCTGCT ACATGGAGGA |
| 1701 | GATGCAGAAT GTGGAGCTGG TGGAGGGGGA CGAGGGCCGC ATGTGCGTCA |
| 1751 | ATACCGAGTG GGGCGCCTTC GGGGACTCCG GCGAGCTGGA CGAGTTCCTG |
| 1801 | CTGGAGTATG ACCGCCTGGT GGACGAGAGC TCTGCAAACC CCGGTCAGCA |
| 1851 | GCTGTATGAG AAGCTCATAG GTGGCAAGTA CATGGGCGAG CTGGTGCGGC |
| 1901 | TTGTGCTGCT CAGGCTCGTG GACGAAAACC TGCTCTTCCA CGGGGAGGCC |
| 1951 | TCCGAGCAGC TGCGCACACG CGGAGCCTTC GAGACGCGCT TCGTGTCGCA |
| 2001 | GGTGGAGAGC GACACGGGCG ACCGCAAGCA GATCTACAAC ATCCTGAGCA |
| 2051 | CGCTGGGGCT GCGACCCTCG ACCACCGACT GCGACATCGT GCGCCGCGCC |
| 2101 | TGCGAGAGCG TGTCTACGCG CGCTGCGCAC ATGTGCTCGG CGGGGCTGGC |
| 2151 | GGGCGTCATC AACCGCATGC GCGAGAGCCG CAGCGAGGAC GTAATGCGCA |
| 2201 | TCACTGTGGG CGTGGATGGC TCCGTGTACA AGCTGCACCC CAGCTTCAAG |
| 2251 | GAGCGGTTCC ATGCCAGCGT GCGCAGGCTG ACGCCCAGCT GCGAGATCAC |
| 2301 | CTTCATCGAG TCGGAGGAGG GCAGTGGCCG GGGCGCGGCC CTGGTCTCGG |
| 2351 | CGGTGGCCTG TAAGAAGGCC TGTATGCTGG GCCAGTGACT CGAGCACGTG |
| 2401 | GAGCTCGCTG ATCAGCCTCG ACTGTGCCTT CTAGTTGCCA GCCATCTGTT |
| 2451 | GTTTGCCCCT CCCCCGTGCC TTCCTTGACC CTGGAAGGTG CCACTCCCAC |
| 2501 | TGTCCTTTCC TAATAAAATG AGGAAATTGC ATCGCATTGT CTGAGTAGGT |
| 2551 | GTCATTCTAT TCTGGGGGGT GGGGTGGGGC AGGACAGCAA GGGGGAGGAT |
| 2601 | TGGGAAGACA ATAGCAGGCA TGCTGGGGAT GCGGTGGGCT CTATGGCCAC |
| 2651 | GTGATTTAAA TCGAGATCTT CCAGAGCATG AGCGCTGATC TGTCGAGCCA |
| 2701 | TGTCTAGGTA GCCATGCTCT AGGAAGATCC TAATCGATTT TACCACATTT |
| 2751 | GTAGAGGTTT TACTTGCTTT AAAAAACCTC CCACATCTCC CCCTGAACCT |
| 2801 | GAAACATAAA ATGAATGCAA TTGTTGTTGT TAACTTGTTT ATTGCAGCTT |
| 2851 | ATAATGGTTA CAAATAAAGC AATAGCATCA CAAATTTCAC AAATAAAGCA |
| 2901 | TTTTTTTCAC TGCATTCTAG TTGTGGTTTG TCCAAACTCA TCAATGTATC |
| 2951 | TTATCATGTC TGCTCGAAGC GGCCTCGACA GCGCTGGTAC CGTCGACGGC |
| 3001 | TGCGTCTAGT TGCAGTAGTT CTCCAGCTGG TAGAGGGAGC AGATGCTGGT |
| 3051 | ACAGCATTGT TCCACAATGC CACGCTTCTG TCGCGACCCC TCCAGGGCCA |
| 3101 | AGGGCTGCAG GCTGCCTGCA CCAGGGCCCC CGCCCAGCTC CACCTGCCCC |
| 3151 | ACCTGCAGGT CCTCTGCCTC CCGCTTGGTC CTGGGTGTGT AGAAGAAGCC |
| 3201 | TCGTTCCCCG CACACTAGGT AGAGAGCTTC CACCAGATCT GAGCCGCACA |
| 3251 | GGTGTTGGTT CACAAAGGCT GCGGCTGGGT CAGGTCCCCA GAGGGCCAGC |
| 3301 | AGCGCCAGCA GGGGCAGGAG GCGCATCCAC AGGGCCATGG CAGAAGGTCG |
| 3351 | ACGCTAGCAA GCTTGGGTCT CCCTATAGTG AGTCGTATTA ATTTCGATAA |
| 3401 | GCCAGTTAAG CAGTGGGTTC TCTAGTTAGC CAGAGAGCTC TGCTTATATA |
| 3451 | GACCTCCCAC CGTACACGCC TACCGCCCAT TTGCGTCAAT GGGGCGGAGT |
| 3501 | TGTTACGACA TTTTGGAAAG TCCCGTTGAT TTTGGTGCCA AAACAAACTC |
| 3551 | CCATTGACGT CAATGGGGTG GAGACTTGGA AATCCCCGTG AGTCAAACCG |
| 3601 | CTATCCACGC CCATTGATGT ACTGCCAAAA CCGCATCACC ATGGTAATAG |
| 3651 | CGATGACTAA TACGTAGATG TACTGCCAAG TAGGAAAGTC CCATAAGGTC |
| 3701 | ATGTACTGGG CATAATGCCA GGCGGGCCAT TTACCGTCAT TGACGTCAAT |
| 3751 | AGGGGGCGTA CTTGGCATAG ATAAATGCGG CCGCAGGAAC CCCTAGTGAT |
| 3801 | GGAGTTGGCC ACTCCCTCTC TGCGCGCTCG CTCGCTCACT GAGGCCGGGC |
| 3851 | GACCAAAGGT CGCCCGACGC CCGGGCTTTG CCCGGGCGGC CTCAGTGAGC |
| 3901 | GAGCGAGCGC GCAGCTGCCT GCAGGGGCGC CTGATGCGGT ATTTTCTCCT |
| 3951 | TACGCATCTG TGCGGTATTT CACACCGCAT ACGTCAAAGC AACCATAGTA |
| 4001 | CGCGCCCTGT AGCGGCGCAT TAAGCGCGGC GGGTGTGGTG GTTACGCGCA |
| 4051 | GCGTGACCGC TACACTTGCC AGCGCCCTAG CGCCCGCTCC TTTCGCTTTC |
| 4101 | TTCCCTTCCT TTCTCGCCAC GTTCGCCGGC TTTCCCCGTC AAGCTCTAAA |
| 4151 | TCGGGGGCTC CCTTTAGGGT TCCGATTTAG TGCTTTACGG CACCTCGACC |
| 4201 | CCAAAAAACT TGATTTGGGT GATGGTTCAC GTAGTGGGCC ATCGCCCTGA |
| 4251 | TAGACGGTTT TTCGCCCTTT GACGTTGGAG TCCACGTTCT TTAATAGTGG |
| 4301 | ACTCTTGTTC CAAACTGGAA CAACACTCAA CCCTATCTCG GGCTATTCTT |
| 4351 | TTGATTTATA AGGGATTTTG CCGATTTCGG CCTATTGGTT AAAAAATGAG |
| 4401 | CTGATTTAAC AAAAATTTAA CGCGAATTTT AACAAAATAT TAACGTTTAC |
| 4451 | AATTTTATGG TGCACTCTCA GTACAATCTG CTCTGATGCC GCATAGTTAA |
| 4501 | GCCAGCCCCG ACACCCGCCA ACACCCGCTG ACGCGCCCTG ACGGGCTTGT |
| 4551 | CTGCTCCCGG CATCCGCTTA CAGACAAGCT GTGACCGTCT CCGGGAGCTG |
| 4601 | CATGTGTCAG AGGTTTTCAC CGTCATCACC GAAACGCGCG AGACGAAAGG |
| 4651 | GCCTCGTGAT ACGCCTATTT TTATAGGTTA ATGTCATGAT AATAATGGTT |
| 4701 | TCTTAGACGT CAGGTGGCAC TTTTCGGGGA AATGTGCGCG GAACCCCTAT |
| 4751 | TTGTTTATTT TTCTAAATAC ATTCAAATAT GTATCCGCTC ATGAGACAAT |
| 4801 | AACCCTGATA AATGCTTCAA TAATATTGAA AAAGGAAGAG TATGAGTATT |
| 4851 | CAACATTTCC GTGTCGCCCT TATTCCCTTT TTTGCGGCAT TTTGCCTTCC |
| 4901 | TGTTTTTGCT CACCCAGAAA CGCTGGTGAA AGTAAAAGAT GCTGAAGATC |
| 4951 | AGTTGGGTGC ACGAGTGGGT TACATCGAAC TGGATCTCAA CAGCGGTAAG |
| 5001 | ATCCTTGAGA GTTTTCGCCC CGAAGAACGT TTTCCAATGA TGAGCACTTT |
| 5051 | TAAAGTTCTG CTATGTGGCG CGGTATTATC CCGTATTGAC GCCGGGCAAG |
| 5101 | AGCAACTCGG TCGCCGCATA CACTATTCTC AGAATGACTT GGTTGAGTAC |
| 5151 | TCACCAGTCA CAGAAAAGCA TCTTACGGAT GGCATGACAG TAAGAGAATT |
| 5201 | ATGCAGTGCT GCCATAACCA TGAGTGATAA CACTGCGGCC AACTTACTTC |
| 5251 | TGACAACGAT CGGAGGACCG AAGGAGCTAA CCGCTTTTTT GCACAACATG |
| 5301 | GGGGATCATG TAACTCGCCT TGATCGTTGG GAACCGGAGC TGAATGAAGC |
| 5351 | CATACCAAAC GACGAGCGTG ACACCACGAT GCCTGTAGCA ATGGCAACAA |
| 5401 | CGTTGCGCAA ACTATTAACT GGCGAACTAC TTACTCTAGC TTCCCGGCAA |
| 5451 | CAATTAATAG ACTGGATGGA GGCGGATAAA GTTGCAGGAC CACTTCTGCG |
| 5501 | CTCGGCCCTT CCGGCTGGCT GGTTTATTGC TGATAAATCT GGAGCCGGTG |
| 5551 | AGCGTGGGTC TCGCGGTATC ATTGCAGCAC TGGGGCCAGA TGGTAAGCCC |
| 5601 | TCCCGTATCG TAGTTATCTA CACGACGGGG AGTCAGGCAA CTATGGATGA |
| 5651 | ACGAAATAGA CAGATCGCTG AGATAGGTGC CTCACTGATT AAGCATTGGT |
| 5701 | AACTGTCAGA CCAAGTTTAC TCATATATAC TTTAGATTGA TTTAAAACTT |
| 5751 | CATTTTTAAT TTAAAAGGAT CTAGGTGAAG ATCCTTTTTG ATAATCTCAT |
| 5801 | GACCAAAATC CCTTAACGTG AGTTTTCGTT CCACTGAGCG TCAGACCCCG |
| 5851 | TAGAAAAGAT CAAAGGATCT TCTTGAGATC CTTTTTTTCT GCGCGTAATC |
| 5901 | TGCTGCTTGC AAACAAAAAA ACCACCGCTA CCAGCGGTGG TTTGTTTGCC |
| 5951 | GGATCAAGAG CTACCAACTC TTTTTCCGAA GGTAACTGGC TTCAGCAGAG |
| 6001 | CGCAGATACC AAATACTGTC CTTCTAGTGT AGCCGTAGTT AGGCCACCAC |
| 6051 | TTCAAGAACT CTGTAGCACC GCCTACATAC CTCGCTCTGC TAATCCTGTT |
| 6101 | ACCAGTGGCT GCTGCCAGTG GCGATAAGTC GTGTCTTACC GGGTTGGACT |
| 6151 | CAAGACGATA GTTACCGGAT AAGGCGCAGC GGTCGGGCTG AACGGGGGGT |
| 6201 | TCGTGCACAC AGCCCAGCTT GGAGCGAACG ACCTACACCG AACTGAGATA |
| 6251 | CCTACAGCGT GAGCTATGAG AAAGCGCCAC GCTTCCCGAA GGGAGAAAGG |
| 6301 | CGGACAGGTA TCCGGTAAGC GGCAGGGTCG GAACAGGAGA GCGCACGAGG |
| 6351 | GAGCTTCCAG GGGGAAACGC CTGGTATCTT TATAGTCCTG TCGGGTTTCG |
| 6401 | CCACCTCTGA CTTGAGCGTC GATTTTTGTG ATGCTCGTCA GGGGGGCGGA |
| 6451 | GCCTATGGAA AAACGCCAGC AACGCGGCCT TTTTACGGTT CCTGGCCTTT |
| 6501 | TGCTGGCCTT TTGCTCACAT GT |

ITR 5′: 1-141 bp
RSV promoter: 239-948 bp
hGck: 973-2387 bp
bGH polyA: 2395-2653 bp
SV40 polyA: 2687-2985 bp
hIns: 2999-3345 bp
miniCMV promoter: 3359-3769 ph
ITR 3′: 3785-3925 bp
N: miniCMV-hIns

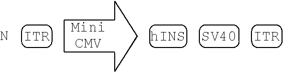

pAAV-miniCMV-hIns plasmid sequence
(SEQ ID NO: 21)

| | |
|---|---|
| 1 | CCTGCAGGCA GCTGCGCGCT CGCTCGCTCA CTGAGGCCGC CCGGGCAAAG |
| 51 | CCCGGGCGTC GGGCGACCTT TGGTCGCCCG GCCTCAGTGA GCGAGCGAGC |
| 101 | GCGCAGAGAG GGAGTGGCCA ACTCCATCAC TAGGGGTTCC TGCGGCCGCG |
| 151 | ATATCTATGC CAAGTACGCC CCCTATTGAC GTCAATGACG GTAAATGGCC |
| 201 | CGCCTGGCAT TATGCCCAGT ACATGACCTT ATGGGACTTT CCTACTTGGC |
| 251 | AGTACATCTA CGTATTAGTC ATCGCTATTA CCATGGTGAT GCGGTTTTGG |
| 301 | CAGTACATCA ATGGGCGTGG ATAGCGGTTT GACTCACGGG GATTTCCAAG |
| 351 | TCTCCACCCC ATTGACGTCA ATGGGAGTTT GTTTTGGCAC CAAAATCAAC |
| 401 | GGGACTTTCC AAAATGTCGT AACAACTCCG CCCCATTGAC GCAAATGGGC |
| 451 | GGTAGGCGTG TACGGTGGGA GGTCTATATA AGCAGAGCTC TCTGGCTAAC |
| 501 | TAGAGAACCC ACTGCTTAAC TGGCTTATCG AAATTAATAC GACTCACTAT |
| 551 | AGGGAGACCC AAGCTTGCTA GCGTCGACCT TCTGCCATGG CCCTGTGGAT |
| 601 | GCGCCTCCTG CCCCTGCTGG CGCTGCTGGC CCTCTGGGGA CCTGACCCAG |
| 651 | CCGCAGCCTT TGTGAACCAA CACCTGTGCG GCTCAGATCT GGTGGAAGCT |
| 701 | CTCTACCTAG TGTGCGGGGA ACGAGGCTTC TTCTACACAC CCAGGACCAA |
| 751 | GCGGGAGGCA GAGGACCTGC AGGTGGGGCA GGTGGAGCTG GGCGGGGGCC |
| 801 | CTGGTGCAGG CAGCCTGCAG CCCTTGGCCC TGGAGGGGTC GCGACAGAAG |
| 851 | CGTGGCATTG TGGAACAATG CTGTACCAGC ATCTGCTCCC TCTACCAGCT |
| 901 | GGAGAACTAC TGCAACTAGA CGCAGCCGTC GACGGTACCA GCGCTGTCGA |
| 851 | GGCCGCTTCG AGCAGACATG ATAAGATACA TTGATGAGTT TGGACAAACC |
| 1001 | ACAACTAGAA TGCAGTGAAA AAAATGCTTT ATTTGTGAAA TTTGTGATGC |
| 1051 | TATTGCTTTA TTTGTAACCA TTATAAGCTG CAATAAACAA GTTAACAACA |
| 1101 | ACAATTGCAT TCATTTTATG TTTCAGGTTC AGGGGGAGAT GTGGGAGGTT |
| 1151 | TTTTAAAGCA AGTAAAACCT CTACAAATGT GGTAAAATCG ATTAGGATCT |
| 1201 | TCCTAGAGCA TGGCTACCTA GACATGGCTC GACAGATCAG CGCTCATGCT |
| 1251 | CTGGAAGATC TCGATTTAAA TGCGGCCGCA GGAACCCCTA GTGATGGAGT |
| 1301 | TGGCCACTCC CTCTCTGCGC GCTCGCTCGC TCACTGAGGC CGGGCGACCA |
| 1351 | AAGGTCGCCC GACGCCCGGG CTTTGCCCGG GCGGCCTCAG TGAGCGAGCG |
| 1401 | AGCGCGCAGC TGCCTGCAGG GGCGCCTGAT GCGGTATTTT CTCCTTACGC |
| 1451 | ATCTGTGCGG TATTTCACAC CGCATACGTC AAAGCAACCA TAGTACGCGC |
| 1501 | CCTGTAGCGG CGCATTAAGC GCGGCGGGTG TGGTGGTTAC GCGCAGCGTG |
| 1551 | ACCGCTACAC TTGCCAGCGC CCTAGCGCCC GCTCCTTTCG CTTTCTTCCC |
| 1601 | TTCCTTTCTC GCCACGTTCG CCGGCTTTCC CCGTCAAGCT CTAAATCGGG |
| 1651 | GGCTCCCTTT AGGGTTCCGA TTTAGTGCTT TACGGCACCT CGACCCCAAA |
| 1701 | AAACTTGATT TGGGTGATGG TTCACGTAGT GGGCCATCGC CCTGATAGAC |
| 1751 | GGTTTTTCGC CCTTTGACGT TGGAGTCCAC GTTCTTTAAT AGTGGACTCT |
| 1801 | TGTTCCAAAC TGGAACAACA CTCAACCCTA TCTCGGGCTA TTCTTTTGAT |
| 1851 | TTATAAGGGA TTTTGCCGAT TTCGGCCTAT TGGTTAAAAA ATGAGCTGAT |
| 1901 | TTAACAAAAA TTTAACGCGA ATTTTAACAA AATATTAACG TTTACAATTT |
| 1951 | TATGGTGCAC TCTCAGTACA ATCTGCTCTG ATGCCGCATA GTTAAGCCAG |
| 2001 | CCCCGACACC CGCCAACACC CGCTGACGCG CCCTGACGGG CTTGTCTGCT |
| 2051 | CCCGGCATCC GCTTACAGAC AAGCTGTGAC CGTCTCCGGG AGCTGCATGT |
| 2101 | GTCAGAGGTT TTCACCGTCA TCACCGAAAC GCGCGAGACG AAAGGGCCTC |
| 2151 | GTGATACGCC TATTTTTATA GGTTAATGTC ATGATAATAA TGGTTTCTTA |
| 2201 | GACGTCAGGT GGCACTTTTC GGGGAAATGT GCGCGGAACC CCTATTTGTT |
| 2251 | TATTTTTCTA AATACATTCA AATATGTATC CGCTCATGAG ACAATAACCC |
| 2301 | TGATAAATGC TTCAATAATA TTGAAAAAGG AAGAGTATGA GTATTCAACA |
| 2351 | TTTCCGTGTC GCCCTTATTC CCTTTTTTGC GGCATTTTGC CTTCCTGTTT |
| 2401 | TTGCTCACCC AGAAACGCTG GTGAAAGTAA AAGATGCTGA AGATCAGTTG |
| 2451 | GGTGCACGAG TGGGTTACAT CGAACTGGAT CTCAACAGCG GTAAGATCCT |
| 2501 | TGAGAGTTTT CGCCCCGAAG AACGTTTTCC AATGATGAGC ACTTTTAAAG |
| 2551 | TTCTGCTATG TGGCGCGGTA TTATCCCGTA TTGACGCCGG GCAAGAGCAA |
| 2601 | CTCGGTCGCC GCATACACTA TTCTCAGAAT GACTTGGTTG AGTACTCACC |
| 2651 | AGTCACAGAA AAGCATCTTA CGGATGGCAT GACAGTAAGA GAATTATGCA |
| 2701 | GTGCTGCCAT AACCATGAGT GATAACACTG CGGCCAACTT ACTTCTGACA |
| 2751 | ACGATCGGAG GACCGAAGGA GCTAACCGCT TTTTTGCACA ACATGGGGGA |
| 2801 | TCATGTAACT CGCCTTGATC GTTGGGAACC GGAGCTGAAT GAAGCCATAC |
| 2851 | CAAACGACGA GCGTGACACC ACGATGCCTG TAGCAATGGC AACAACGTTG |
| 2901 | CGCAAACTAT TAACTGGCGA ACTACTTACT CTAGCTTCCC GGCAACAATT |
| 2951 | AATAGACTGG ATGGAGGCGG ATAAAGTTGC AGGACCACTT CTGCGCTCGG |
| 3001 | CCCTTCCGGC TGGCTGGTTT ATTGCTGATA AATCTGGAGC CGGTGAGCGT |
| 3051 | GGGTCTCGCG GTATCATTGC AGCACTGGGG CCAGATGGTA AGCCCTCCCG |
| 3101 | TATCGTAGTT ATCTACACGA CGGGGAGTCA GGCAACTATG GATGAACGAA |
| 3151 | ATAGACAGAT CGCTGAGATA GGTGCCTCAC TGATTAAGCA TTGGTAACTG |
| 3201 | TCAGACCAAG TTTACTCATA TATACTTTAG ATTGATTTAA AACTTCATTT |
| 3251 | TTAATTTAAA AGGATCTAGG TGAAGATCCT TTTTGATAAT CTCATGACCA |
| 3301 | AAATCCCTTA ACGTGAGTTT TCGTTCCACT GAGCGTCAGA CCCCGTAGAA |
| 3351 | AAGATCAAAG GATCTTCTTG AGATCCTTTT TTTCTGCGCG TAATCTGCTG |
| 3401 | CTTGCAAACA AAAAAACCAC CGCTACCAGC GGTGGTTTGT TTGCCGGATC |
| 3451 | AAGAGCTACC AACTCTTTTT CCGAAGGTAA CTGGCTTCAG CAGAGCGCAG |
| 3501 | ATACCAAATA CTGTCCTTCT AGTGTAGCCG TAGTTAGGCC ACCACTTCAA |
| 3551 | GAACTCTGTA GCACCGCCTA CATACCTCGC TCTGCTAATC CTGTTACCAG |
| 3601 | TGGCTGCTGC CAGTGGCGAT AAGTCGTGTC TTACCGGGTT GGACTCAAGA |
| 3651 | CGATAGTTAC CGGATAAGGC GCAGCGGTCG GGCTGAACGG GGGGTTCGTG |
| 3701 | CACACAGCCC AGCTTGGAGC GAACGACCTA CACCGAACTG AGATACCTAC |
| 3751 | AGCGTGAGCT ATGAGAAAGC GCCACGCTTC CCGAAGGGAG AAAGGCGGAC |
| 3801 | AGGTATCCGG TAAGCGGCAG GGTCGGAACA GGAGAGCGCA CGAGGGAGCT |
| 3851 | TCCAGGGGGA AACGCCTGGT ATCTTTATAG TCCTGTCGGG TTTCGCCACC |
| 3901 | TCTGACTTGA GCGTCGATTT TTGTGATGCT CGTCAGGGGG GCGGAGCCTA |
| 3951 | TGGAAAAACG CCAGCAACGC GGCCTTTTTA CGGTTCCTGG CCTTTTGCTG |
| 4001 | GCCTTTTGCT CACATGT |

ITR 5′: 1-141 bp
miniCMV promoter: 156-566 bp
hIns: 580-926 bp
SV40 polyA: 940-1238 bp
ITR 3′: 1280-1420 bp
Equivalent mini CMV promoter
SEQ ID NO: 24
TAGGCGTGTACGGTGGGAGGTCTATATAAGCAGAGCTCTCTGGCTAACTAGAGAACCCACTGCTTAAC
TGGCTTATCGAAATTAATACGACTCA
O: miniCMV-hIns-bGH

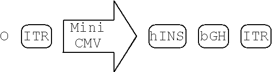

pAAV-miniCMV-hIns-bGH plasmid sequence
(SEQ ID NO: 25)

| | |
|---|---|
| 1 | GCTCATGCTC TGGAAGATCT CGATTTAAAT GCGGCCGCAG GAACCCCTAG |
| 51 | TGATGGAGTT GGCCACTCCC TCTCTGCGCG CTCGCTCGCT CACTGAGGCC |
| 101 | GGGCGACCAA AGGTCGCCCG ACGCCCGGGC TTTGCCCGGG CGGCCTCAGT |
| 151 | GAGCGAGCGA GCGCGCAGCT GCCTGCAGGG GCGCCTGATG CGGTATTTTC |
| 201 | TCCTTACGCA TCTGTGCGGT ATTTCACACC GCATACGTCA AAGCAACCAT |
| 251 | AGTACGCGCC CTGTAGCGGC GCATTAAGCG CGGCGGGTGT GGTGGTTACG |
| 301 | CGCAGCGTGA CCGCTACACT TGCCAGCGCC CTAGCGCCCG CTCCTTTCGC |
| 351 | TTTCTTCCCT TCCTTTCTCG CCACGTTCGC CGGCTTTCCC CGTCAAGCTC |
| 401 | TAAATCGGGG GCTCCCTTTA GGGTTCCGAT TTAGTGCTTT ACGGCACCTC |
| 451 | GACCCCAAAA AACTTGATTT GGGTGATGGT TCACGTAGTG GGCCATCGCC |
| 501 | CTGATAGACG GTTTTTCGCC CTTTGACGTT GGAGTCCACG TTCTTTAATA |
| 551 | GTGGACTCTT GTTCCAAACT GGAACAACAC TCAACCCTAT CTCGGGCTAT |
| 601 | TCTTTTGATT TATAAGGGAT TTTGCCGATT TCGGCCTATT GGTTAAAAAA |
| 651 | TGAGCTGATT TAACAAAAAT TTAACGCGAA TTTTAACAAA ATATTAACGT |
| 701 | TTACAATTTT ATGGTGCACT CTCAGTACAA TCTGCTCTGA TGCCGCATAG |
| 751 | TTAAGCCAGC CCCGACACCC GCCAACACCC GCTGACGCGC CCTGACGGGC |
| 801 | TTGTCTGCTC CCGGCATCCG CTTACAGACA AGCTGTGACC GTCTCCGGGA |
| 851 | GCTGCATGTG TCAGAGGTTT TCACCGTCAT CACCGAAACG CGCGAGACGA |
| 901 | AAGGGCCTCG TGATACGCCT ATTTTTATAG GTTAATGTCA TGATAATAAT |
| 951 | GGTTTCTTAG ACGTCAGGTG GCACTTTTCG GGGAAATGTG CGCGGAACCC |
| 1001 | CTATTTGTTT ATTTTTCTAA ATACATTCAA ATATGTATCC GCTCATGAGA |
| 1051 | CAATAACCCT GATAAATGCT TCAATAATAT TGAAAAAGGA AGAGTATGAG |
| 1101 | TATTCAACAT TTCCGTGTCG CCCTTATTCC CTTTTTTGCG GCATTTTGCC |
| 1151 | TTCCTGTTTT TGCTCACCCA GAAACGCTGG TGAAAGTAAA AGATGCTGAA |
| 1201 | GATCAGTTGG GTGCACGAGT GGGTTACATC GAACTGGATC TCAACAGCGG |
| 1251 | TAAGATCCTT GAGAGTTTTC GCCCCGAAGA ACGTTTTCCA ATGATGAGCA |
| 1301 | CTTTTAAAGT TCTGCTATGT GGCGCGGTAT TATCCCGTAT TGACGCCGGG |
| 1351 | CAAGAGCAAC TCGGTCGCCG CATACACTAT TCTCAGAATG ACTTGGTTGA |
| 1401 | GTACTCACCA GTCACAGAAA AGCATCTTAC GGATGGCATG ACAGTAAGAG |
| 1431 | AATTATGCAG TGCTGCCATA ACCATGAGTG ATAACACTGC GGCCAACTTA |
| 1501 | CTTCTGACAA CGATCGGAGG ACCGAAGGAG CTAACCGCTT TTTTGCACAA |
| 1551 | CATGGGGGAT CATGTAACTC GCCTTGATCG TTGGGAACCG GAGCTGAATG |
| 1601 | AAGCCATACC AAACGACGAG CGTGACACCA CGATGCCTGT AGCAATGGCA |
| 1651 | ACAACGTTGC GCAAACTATT AACTGGCGAA CTACTTACTC TAGCTTCCCG |
| 1701 | GCAACAATTA ATAGACTGGA TGGAGGCGGA TAAAGTTGCA GGACCACTTC |
| 1751 | TGCGCTCGGC CCTTCCGGCT GGCTGGTTTA TTGCTGATAA ATCTGGAGCC |
| 1801 | GGTGAGCGTG GGTCTCGCGG TATCATTGCA GCACTGGGGC CAGATGGTAA |
| 1851 | GCCCTCCCGT ATCGTAGTTA TCTACACGAC GGGGAGTCAG GCAACTATGG |
| 1901 | ATGAACGAAA TAGACAGATC GCTGAGATAG GTGCCTCACT GATTAAGCAT |
| 1951 | TGGTAACTGT CAGACCAAGT TTACTCATAT ATACTTTAGA TTGATTTAAA |
| 2001 | ACTTCATTTT TAATTTAAAA GGATCTAGGT GAAGATCCTT TTTGATAATC |
| 2051 | TCATGACCAA AATCCCTTAA CGTGAGTTTT CGTTCCACTG AGCGTCAGAC |
| 2101 | CCCGTAGAAA AGATCAAAGG ATCTTCTTGA GATCCTTTTT TTCTGCGCGT |
| 2151 | AATCTGCTGC TTGCAAACAA AAAAACCACC GCTACCAGCG GTGGTTTGTT |
| 2201 | TGCCGGATCA AGAGCTACCA ACTCTTTTTC CGAAGGTAAC TGGCTTCAGC |
| 2251 | AGAGCGCAGA TACCAAATAC TGTCCTTCTA GTGTAGCCGT AGTTAGGCCA |
| 2301 | CCACTTCAAG AACTCTGTAG CACCGCCTAC ATACCTCGCT CTGCTAATCC |
| 2351 | TGTTACCAGT GGCTGCTGCC AGTGGCGATA AGTCGTGTCT TACCGGGTTG |
| 2401 | GACTCAAGAC GATAGTTACC GGATAAGGCG CAGCGGTCGG GCTGAACGGG |
| 2451 | GGGTTCGTGC ACACAGCCCA GCTTGGAGCG AACGACCTAC ACCGAACTGA |
| 2501 | GATACCTACA GCGTGAGCTA TGAGAAAGCG CCACGCTTCC CGAAGGGAGA |
| 2551 | AAGGCGGACA GGTATCCGGT AAGCGGCAGG GTCGGAACAG GAGAGCGCAC |
| 2601 | GAGGGAGCTT CCAGGGGGAA ACGCCTGGTA TCTTTATAGT CCTGTCGGGT |
| 2651 | TTCGCCACCT CTGACTTGAG CGTCGATTTT TGTGATGCTC GTCAGGGGGG |
| 2701 | CGGAGCCTAT GGAAAAACGC CAGCAACGCG GCCTTTTTAC GGTTCCTGGC |
| 2751 | CTTTTGCTGG CCTTTTGCTC ACATGTCCTG CAGGCAGCTG CGCGCTCGCT |
| 2801 | CGCTCACTGA GGCCGCCCGG GCAAAGCCCG GGCGTCGGGC GACCTTTGGT |
| 2851 | CGCCCGGCCT CAGTGAGCGA GCGAGCGCGC AGAGAGGGAG TGGCCAACTC |
| 2901 | CATCACTAGG GGTTCCTGCG GCCGCGATAT CTATGCCAAG TACGCCCCCT |
| 2951 | ATTGACGTCA ATGACGGTAA ATGGCCCGCC TGGCATTATG CCCAGTACAT |
| 3001 | GACCTTATGG GACTTTCCTA CTTGGCAGTA CATCTACGTA TTAGTCATCG |
| 3051 | CTATTACCAT GGTGATGCGG TTTTGGCAGT ACATCAATGG GCGTGGATAG |
| 3101 | CGGTTTGACT CACGGGGATT TCCAAGTCTC CACCCCATTG ACGTCAATGG |
| 3151 | GAGTTTGTTT TGGCACCAAA ATCAACGGGA CTTTCCAAAA TGTCGTAACA |
| 3201 | ACTCCGCCCC ATTGACGCAA ATGGGCGGTA GGCGTGTACG GTGGGAGGTC |
| 3251 | TATATAAGCA GAGCTCTCTG GCTAACTAGA GAACCCACTG CTTAACTGGC |
| 3301 | TTATCGAAAT TAATACGACT CACTATAGGG AGACCCAAGC TTGCTAGCGT |
| 3351 | CGACCTTCTG CCATGGCCCT GTGGATGCGC CTCCTGCCCC TGCTGGCGCT |
| 3401 | GCTGGCCCTC TGGGGACCTG ACCCAGCCGC AGCCTTTGTG AACCAACACC |
| 3451 | TGTGCGGCTC AGATCTGGTG GAAGCTCTCT ACCTAGTGTG CGGGGAACGA |
| 3501 | GGCTTCTTCT ACACACCCAG GACCAAGCGG GAGGCAGAGG ACCTGCAGGT |
| 3551 | GGGGCAGGTG GAGCTGGGCG GGGGCCCTGG TGCAGGCAGC CTGCAGCCCT |
| 3601 | TGGCCCTGGA GGGGTCGCGA CAGAAGCGTG GCATTGTGGA ACAATGCTGT |
| 3651 | ACCAGCATCT GCTCCCTCTA CCAGCTGGAG AACTACTGCA ACTAGACGCA |
| 3701 | GCCGTCGACG GTACCAGCGT GGAGCTCGCT GATCAGCCTC GACTGTGCCT |
| 3751 | TCTAGTTGCC AGCCATCTGT TGTTTGCCCC TCCCCCGTGC CTTCCTTGAC |
| 3801 | CCTGGAAGGT GCCACTCCCA CTGTCCTTTC CTAATAAAAT GAGGAAATTG |
| 3851 | CATCGCATTG TCTGAGTAGG TGTCATTCTA TTCTGGGGGG TGGGGTGGGG |
| 3901 | CAGGACAGCA AGGGGGAGGA TTGGGAAGAC AATAGCAGGC ATGCTGGGGA |
| 3951 | TGCGGTGGGC TCTATGGCCA C |

ITR 5′: 2777-2917 bp
miniCMV promoter: 2932-3342 bp
hIns: 3356-3702 bp
bGH polyA: 3719-3971 bp
ITR 3′: 39-179 bp
P: RSV-hGck-SV40

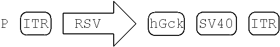

pAAV-RS-hGck-SV40 plasmid sequence
(SEQ ID NO: 26)

| | |
|---|---|
| 1 | GTGATTTAAA TGCGGCCGCA GGAACCCCTA GTGATGGAGT TGGCCACTCC |
| 51 | CTCTCTGCGC GCTCGCTCGC TCACTGAGGC CGGGCGACCA AAGGTCGCCC |
| 101 | GACGCCCGGG CTTTGCCCGG GCGGCCTCAG TGAGCGAGCG AGCGCGCAGC |
| 151 | TGCCTGCAGG GGCGCCTGAT GCGGTATTTT CTCCTTACGC ATCTGTGCGG |
| 201 | TATTTCACAC CGCATACGTC AAAGCAACCA TAGTACGCGC CCTGTAGCGG |
| 251 | CGCATTAAGC GCGGCGGGTG TGGTGGTTAC GCGCAGCGTG ACCGCTACAC |
| 301 | TTGCCAGCGC CCTAGCGCCC GCTCCTTTCG CTTTCTTCCC TTCCTTTCTC |
| 351 | GCCACGTTCG CCGGCTTTCC CCGTCAAGCT CTAAATCGGG GGCTCCCTTT |
| 401 | AGGGTTCCGA TTTAGTGCTT TACGGCACCT CGACCCCAAA AAACTTGATT |
| 451 | TGGGTGATGG TTCACGTAGT GGGCCATCGC CCTGATAGAC GGTTTTTCGC |
| 501 | CCTTTGACGT TGGAGTCCAC GTTCTTTAAT AGTGGACTCT TGTTCCAAAC |
| 551 | TGGAACAACA CTCAACCCTA TCTCGGGCTA TTCTTTTGAT TTATAAGGGA |
| 601 | TTTTGCCGAT TTCGGCCTAT TGGTTAAAAA ATGAGCTGAT TTAACAAAAA |
| 651 | TTTAACGCGA ATTTTAACAA AATATTAACG TTTACAATTT TATGGTGCAC |
| 701 | TCTCAGTACA ATCTGCTCTG ATGCCGCATA GTTAAGCCAG CCCCGACACC |
| 751 | CGCCAACACC CGCTGACGCG CCCTGACGGG CTTGTCTGCT CCCGGCATCC |
| 801 | GCTTACAGAC AAGCTGTGAC CGTCTCCGGG AGCTGCATGT GTCAGAGGTT |
| 851 | TTCACCGTCA TCACCGAAAC GCGCGAGACG AAAGGGCCTC GTGATACGCC |
| 901 | TATTTTTATA GGTTAATGTC ATGATAATAA TGGTTTCTTA GACGTCAGGT |
| 951 | GGCACTTTTC GGGGAAATGT GCGCGGAACC CCTATTTGTT TATTTTTCTA |
| 1001 | AATACATTCA AATATGTATC CGCTCATGAG ACAATAACCC TGATAAATGC |
| 1051 | TTCAATAATA TTGAAAAAGG AAGAGTATGA GTATTCAACA TTTCCGTGTC |
| 1101 | GCCCTTATTC CCTTTTTTGC GGCATTTTGC CTTCCTGTTT TTGCTCACCC |
| 1151 | AGAAACGCTG GTGAAAGTAA AAGATGCTGA AGATCAGTTG GGTGCACGAG |
| 1201 | TGGGTTACAT CGAACTGGAT CTCAACAGCG GTAAGATCCT TGAGAGTTTT |
| 1251 | CGCCCCGAAG AACGTTTTCC AATGATGAGC ACTTTTAAAG TTCTGCTATG |
| 1301 | TGGCGCGGTA TTATCCCGTA TTGACGCCGG GCAAGAGCAA CTCGGTCGCC |
| 1351 | GCATACACTA TTCTCAGAAT GACTTGGTTG AGTACTCACC AGTCACAGAA |
| 1401 | AAGCATCTTA CGGATGGCAT GACAGTAAGA GAATTATGCA GTGCTGCCAT |
| 1451 | AACCATGAGT GATAACACTG CGGCCAACTT ACTTCTGACA ACGATCGGAG |
| 1501 | GACCGAAGGA GCTAACCGCT TTTTTGCACA ACATGGGGGA TCATGTAACT |
| 1551 | CGCCTTGATC GTTGGGAACC GGAGCTGAAT GAAGCCATAC CAAACGACGA |
| 1601 | GCGTGACACC ACGATGCCTG TAGCAATGGC AACAACGTTG CGCAAACTAT |
| 1651 | TAACTGGCGA ACTACTTACT CTAGCTTCCC GGCAACAATT AATAGACTGG |
| 1701 | ATGGAGGCGG ATAAAGTTGC AGGACCACTT CTGCGCTCGG CCCTTCCGGC |
| 1751 | TGGCTGGTTT ATTGCTGATA AATCTGGAGC CGGTGAGCGT GGGTCTCGCG |
| 1801 | GTATCATTGC AGCACTGGGG CCAGATGGTA AGCCCTCCCG TATCGTAGTT |
| 1851 | ATCTACACGA CGGGGAGTCA GGCAACTATG GATGAACGAA ATAGACAGAT |
| 1901 | CGCTGAGATA GGTGCCTCAC TGATTAAGCA TTGGTAACTG TCAGACCAAG |
| 1951 | TTTACTCATA TATACTTTAG ATTGATTTAA AACTTCATTT TTAATTTAAA |
| 2001 | AGGATCTAGG TGAAGATCCT TTTTGATAAT CTCATGACCA AAATCCCTTA |
| 2051 | ACGTGAGTTT TCGTTCCACT GAGCGTCAGA CCCCGTAGAA AAGATCAAAG |
| 2101 | GATCTTCTTG AGATCCTTTT TTTCTGCGCG TAATCTGCTG CTTGCAAACA |
| 2151 | AAAAAACCAC CGCTACCAGC GGTGGTTTGT TTGCCGGATC AAGAGCTACC |
| 2201 | AACTCTTTTT CCGAAGGTAA CTGGCTTCAG CAGAGCGCAG ATACCAAATA |
| 2251 | CTGTCCTTCT AGTGTAGCCG TAGTTAGGCC ACCACTTCAA GAACTCTGTA |
| 2301 | GCACCGCCTA CATACCTCGC TCTGCTAATC CTGTTACCAG TGGCTGCTGC |
| 2351 | CAGTGGCGAT AAGTCGTGTC TTACCGGGTT GGACTCAAGA CGATAGTTAC |
| 2401 | CGGATAAGGC GCAGCGGTCG GGCTGAACGG GGGGTTCGTG CACACAGCCC |
| 2451 | AGCTTGGAGC GAACGACCTA CACCGAACTG AGATACCTAC AGCGTGAGCT |
| 2501 | ATGAGAAAGC GCCACGCTTC CCGAAGGGAG AAAGGCGGAC AGGTATCCGG |
| 2551 | TAAGCGGCAG GGTCGGAACA GGAGAGCGCA CGAGGGAGCT TCCAGGGGGA |
| 2601 | AACGCCTGGT ATCTTTATAG TCCTGTCGGG TTTCGCCACC TCTGACTTGA |
| 2651 | GCGTCGATTT TTGTGATGCT CGTCAGGGGG GCGGAGCCTA TGGAAAAACG |
| 2701 | CCAGCAACGC GGCCTTTTTA CGGTTCCTGG CCTTTTGCTG GCCTTTTGCT |
| 2751 | CACATGTCCT GCAGGCAGCT GCGCGCTCGC TCGCTCACTG AGGCCGCCCG |
| 2801 | GGCAAAGCCC GGGCGTCGGG CGACCTTTGG TCGCCCGGCC TCAGTGAGCG |
| 2851 | AGCGAGCGCG CAGAGAGGGA GTGGCCAACT CCATCACTAG GGGTTCCTGC |
| 2901 | GGCCGCGATA TCCATGTTTG ACAGCTTATC ATCGCAGATC CGTATGGTGC |
| 2951 | ACTCTCAGTA CAATCTGCTC TGATGCCGCA TAGTTAAGCC AGTATCTGCT |
| 3001 | CCCTGCTTGT GTGTTGGAGG TCGCTGAGTA GTGCGCGAGC AAAATTTAAG |
| 3051 | CTACAACAAG GCAAGGCTTG ACCGACAATT GCATGAAGAA TCTGCTTAGG |
| 3101 | GTTAGGCGTT TTGCGCTGCT TCGCGATGTA CGGGCCAGAT ATTCGCGTAT |
| 3151 | CTGAGGGGAC TAGGGTGTGT TTAGGCGAAA AGCGGGGCTT CGGTTGTACG |
| 3201 | CGGTTAGGAG TCCCCTCAGG ATATAGTAGT TTCGCTTTTG CATAGGGAGG |
| 3251 | GGGAAATGTA GTCTTATGCA ATACTCTTGT AGTCTTGCAA CATGGTAACG |
| 3301 | ATGAGTTAGC AACATGCCTT ACAAGGAGAG AAAAAGCACC GTGCATGCCG |
| 3351 | ATTGGTGGAA GTAAGGTGGT ACGATCGTGC CTTATTAGGA AGGCAACAGA |
| 3401 | CGGGTCTGAC ATGGATTGGA CGAACCACTA AATTCCGCAT TGCAGAGATA |
| 3451 | TTGTATTTAA GTGCCTAGCT CGATACAATA AACGCCATTT GACCATTCAC |
| 3501 | CACATTGGTG TGCACCTCCA AGCTGGGTAC CAGCTTCTAG AGAGATCTGC |
| 3551 | TTCAGCTGGA GGCACTGGGC AGGTAAGTAT CAAGGTTACA AGACAGGTTT |
| 3601 | AAGGAGACCA ATAGAAACTG GGCTTGTCGA GACAGAGAAG ACTCTTGCGT |
| 3651 | TTCTGATAGG CACCTATTGG TCTTACTGAC ATCCACTTTG CCTTTCTCTC |
| 3701 | CACAGGTGCA GCTGCTGCAG CGGTCTAGAA CTCGAGTCGA GACCATGGCG |
| 3751 | ATGGATGTCA CAAGGAGCCA GGCCCAGACA GCCTTGACTC TGGTAGAGCA |
| 3801 | GATCCTGGCA GAGTTCCAGC TGCAGGAGGA GGACCTGAAG AAGGTGATGA |
| 3851 | GACGGATGCA GAAGGAGATG GACCGCGGCC TGAGGCTGGA GACCCATGAA |
| 3901 | GAGGCCAGTG TGAAGATGCT GCCCACCTAC GTGCGCTCCA CCCCAGAAGG |
| 3951 | CTCAGAAGTC GGGGACTTCC TCTCCCTGGA CCTGGGTGGC ACTAACTTCA |
| 4001 | GGGTGATGCT GGTGAAGGTG GGAGAAGGTG AGGAGGGGCA GTGGAGCGTG |
| 4051 | AAGACCAAAC ACCAGATGTA CTCCATCCCC GAGGACGCCA TGACCGGCAC |
| 4101 | TGCTGAGATG CTCTTCGACT ACATCTCTGA GTGCATCTCC GACTTCCTGG |
| 4151 | ACAAGCATCA GATGAAACAC AAGAAGCTGC CCCTGGGCTT CACCTTCTCC |
| 4201 | TTTCCTGTGA GGCACGAAGA CATCGATAAG GGCATCCTTC TCAACTGGAC |
| 4251 | CAAGGGCTTC AAGGCCTCAG GAGCAGAAGG GAACAATGTC GTGGGGCTTC |
| 4301 | TGCGAGACGC TATCAAACGG AGAGGGGACT TTGAAATGGA TGTGGTGGCA |
| 4351 | ATGGTGAATG ACACGGTGGC CACGATGATC TCCTGCTACT ACGAAGACCA |
| 4401 | TCAGTGCGAG GTCGGCATGA TCGTGGGCAC GGGCTGCAAT GCCTGCTACA |
| 4451 | TGGAGGAGAT GCAGAATGTG GAGCTGGTGG AGGGGGACGA GGGCCGCATG |
| 4501 | TGCGTCAATA CCGAGTGGGG CGCCTTCGGG GACTCCGGCG AGCTGGACGA |
| 4551 | GTTCCTGCTG GAGTATGACC GCCTGGTGGA CGAGAGCTCT GCAAACCCCG |
| 4601 | GTCAGCAGCT GTATGAGAAG CTCATAGGTG GCAAGTACAT GGGCGAGCTG |
| 4651 | GTGCGGCTTG TGCTGCTCAG GCTCGTGGAC GAAAACCTGC TCTTCCACGG |
| 4701 | GGAGGCCTCC GAGCAGCTGC GCACACGCGG AGCCTTCGAG ACGCGCTTCG |
| 4751 | TGTCGCAGGT GGAGAGCGAC ACGGGCGACC GCAAGCAGAT CTACAACATC |
| 4801 | CTGAGCACGC TGGGGCTGCG ACCCTCGACC ACCGACTGCG ACATCGTGCG |
| 4851 | CCGCGCCTGC GAGAGCGTGT CTACGCGCGC TGCGCACATG TGCTCGGCGG |
| 4901 | GGCTGGCGGG CGTCATCAAC CGCATGCGCG AGAGCCGCAG CGAGGACGTA |
| 4951 | ATGCGCATCA CTGTGGGCGT GGATGGCTCC GTGTACAAGC TGCACCCCAG |
| 5001 | CTTCAAGGAG CGGTTCCATG CCAGCGTGCG CAGGCTGACG CCCAGCTGCG |
| 5051 | AGATCACCTT CATCGAGTCG GAGGAGGGCA GTGGCCGGGG CGCGGCCCTG |
| 5101 | GTCTCGGCGG TGGCCTGTAA GAAGGCCTGT ATGCTGGGCC AGTGACTCGA |
| 5151 | GCACGCTGTC GAGGCCGCTT CGAGCAGACA TGATAAGATA CATTGATGAG |
| 5201 | TTTGGACAAA CCACAACTAG AATGCAGTGA AAAAAATGCT TTATTTGTGA |
| 5251 | AATTTGTGAT GCTATTGCTT TATTTGTAAC CATTATAAGC TGCAATAAAC |
| 5301 | AAGTTAACAA CAACAATTGC ATTCATTTTA TGTTTCAGGT TCAGGGGGAG |
| 5351 | ATGTGGGAGG TTTTTTAAAG CAAGTAAAAC CTCTACAAAT GTGGTAAAAT |
| 5401 | CGATTAGGAT CTTCCTAGAG CATGGCTACC TAGACATGGC TCGACAGATC |
| 5451 | AGC |

ITR 5′: 2758-2898 bp
RSV promoter: 2996-3705 bp
hGck: 3730-5144 bp
SV40 polyA: 5155-5450 bp
ITR 3′: 20-160 bp
Q: miniCMV-hIns-bGH(rev)-RSV-hGck-SV40

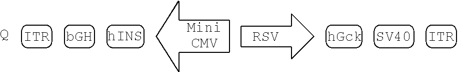

pAAV-miniCMV-hIns-bGH(rev)-RSV-hGck-SV40 plasmid sequence
(SEQ ID NO: 27)

| | |
|---|---|
| 1 | ATCCATGTTT GACAGCTTAT CATCGCAGAT CCGTATGGTG CACTCTCAGT |
| 51 | ACAATCTGCT CTGATGCCGC ATAGTTAAGC CAGTATCTGC TCCCTGCTTG |
| 101 | TGTGTTGGAG GTCGCTGAGT AGTGCGCGAG CAAAATTTAA GCTACAACAA |
| 151 | GGCAAGGCTT GACCGACAAT TGCATGAAGA ATCTGCTTAG GGTTAGGCGT |
| 201 | TTTGCGCTGC TTCGCGATGT ACGGGCCAGA TATTCGCGTA TCTGAGGGGA |
| 251 | CTAGGGTGTG TTTAGGCGAA AAGCGGGGCT TCGGTTGTAC GCGGTTAGGA |
| 301 | GTCCCCTCAG GATATAGTAG TTTCGCTTTT GCATAGGGAG GGGGAAATGT |
| 351 | AGTCTTATGC AATACTCTTG TAGTCTTGCA ACATGGTAAC GATGAGTTAG |
| 401 | CAACATGCCT TACAAGGAGA GAAAAAGCAC CGTGCATGCC GATTGGTGGA |
| 451 | AGTAAGGTGG TACGATCGTG CCTTATTAGG AAGGCAACAG ACGGGTCTGA |
| 501 | CATGGATTGG ACGAACCACT AAATTCCGCA TTGCAGAGAT ATTGTATTTA |
| 551 | AGTGCCTAGC TCGATACAAT AAACGCCATT TGACCATTCA CCACATTGGT |
| 601 | GTGCACCTCC AAGCTGGGTA CCAGCTTCTA GAGAGATCTG CTTCAGCTGG |
| 651 | AGGCACTGGG CAGGTAAGTA TCAAGGTTAC AAGACAGGTT TAAGGAGACC |
| 701 | AATAGAAACT GGGCTTGTCG AGACAGAGAA GACTCTTGCG TTTCTGATAG |
| 751 | GCACCTATTG GTCTTACTGA CATCCACTTT GCCTTTCTCT CCACAGGTGC |
| 801 | AGCTGCTGCA GCGGTCTAGA ACTCGAGTCG AGACCATGGC GATGGATGTC |
| 851 | ACAAGGAGCC AGGCCCAGAC AGCCTTGACT CTGGTAGAGC AGATCCTGGC |
| 901 | AGAGTTCCAG CTGCAGGAGG AGGACCTGAA GAAGGTGATG AGACGGATGC |
| 951 | AGAAGGAGAT GGACCGCGGC CTGAGGCTGG AGACCCATGA AGAGGCCAGT |
| 1001 | GTGAAGATGC TGCCCACCTA CGTGCGCTCC ACCCCAGAAG GCTCAGAAGT |
| 1051 | CGGGGACTTC CTCTCCCTGG ACCTGGGTGG CACTAACTTC AGGGTGATGC |
| 1101 | TGGTGAAGGT GGGAGAAGGT GAGGAGGGGC AGTGGAGCGT GAAGACCAAA |
| 1151 | CACCAGATGT ACTCCATCCC CGAGGACGCC ATGACCGGCA CTGCTGAGAT |
| 1201 | GCTCTTCGAC TACATCTCTG AGTGCATCTC CGACTTCCTG GACAAGCATC |
| 1251 | AGATGAAACA CAAGAAGCTG CCCCTGGGCT TCACCTTCTC CTTTCCTGTG |
| 1301 | AGGCACGAAG ACATCGATAA GGGCATCCTT CTCAACTGGA CCAAGGGCTT |
| 1351 | CAAGGCCTCA GGAGCAGAAG GGAACAATGT CGTGGGGCTT CTGCGAGACG |
| 1401 | CTATCAAACG GAGAGGGGAC TTTGAAATGG ATGTGGTGGC AATGGTGAAT |
| 1451 | GACACGGTGG CCACGATGAT CTCCTGCTAC TACGAAGACC ATCAGTGCGA |
| 1501 | GGTCGGCATG ATCGTGGGCA CGGGCTGCAA TGCCTGCTAC ATGGAGGAGA |
| 1551 | TGCAGAATGT GGAGCTGGTG GAGGGGGACG AGGGCCGCAT GTGCGTCAAT |
| 1601 | ACCGAGTGGG GCGCCTTCGG GGACTCCGGC GAGCTGGACG AGTTCCTGCT |
| 1651 | GGAGTATGAC CGCCTGGTGG ACGAGAGCTC TGCAAACCCC GGTCAGCAGC |
| 1701 | TGTATGAGAA GCTCATAGGT GGCAAGTACA TGGGCGAGCT GGTGCGGCTT |
| 1751 | GTGCTGCTCA GGCTCGTGGA CGAAAACCTG CTCTTCCACG GGGAGGCCTC |
| 1801 | CGAGCAGCTG CGCACACGCG GAGCCTTCGA GACGCGCTTC GTGTCGCAGG |
| 1851 | TGGAGAGCGA CACGGGCGAC CGCAAGCAGA TCTACAACAT CCTGAGCACG |
| 1901 | CTGGGGCTGC GACCCTCGAC CACCGACTGC GACATCGTGC GCCGCGCCTG |
| 1951 | CGAGAGCGTG TCTACGCGCG CTGCGCACAT GTGCTCGGCG GGGCTGGCGG |
| 2001 | GCGTCATCAA CCGCATGCGC GAGAGCCGCA GCGAGGACGT AATGCGCATC |
| 2051 | ACTGTGGGCG TGGATGGCTC CGTGTACAAG CTGCACCCCA GCTTCAAGGA |
| 2101 | GCGGTTCCAT GCCAGCGTGC GCAGGCTGAC GCCCAGCTGC GAGATCACCT |
| 2151 | TCATCGAGTC GGAGGAGGGC AGTGGCCGGG GCGCGGCCCT GGTCTCGGCG |
| 2201 | GTGGCCTGTA AGAAGGCCTG TATGCTGGGC CAGTGACTCG AGCACGCTGT |
| 2251 | CGAGGCCGCT TCGAGCAGAC ATGATAAGAT ACATTGATGA GTTTGGACAA |
| 2301 | ACCACAACTA GAATGCAGTG AAAAAAATGC TTTATTTGTG AAATTTGTGA |
| 2351 | TGCTATTGCT TTATTTGTAA CCATTATAAG CTGCAATAAA CAAGTTAACA |
| 2401 | ACAACAATTG CATTCATTTT ATGTTTCAGG TTCAGGGGGA GATGTGGGAG |
| 2451 | GTTTTTTAAA GCAAGTAAAA CCTCTACAAA TGTGGTAAAA TCGATTAGGA |
| 2501 | TCTTCCTAGA GCATGGCTAC CTAGACATGG CTCGACAGAT CAGCGTGATT |
| 2551 | TAAATGCGGC CGCAGGAACC CCTAGTGATG GAGTTGGCCA CTCCCTCTCT |
| 2601 | GCGCGCTCGC TCGCTCACTG AGGCCGGGCG ACCAAAGGTC GCCCGACGCC |
| 2651 | CGGGCTTTGC CCGGGCGGCC TCAGTGAGCG AGCGAGCGCG CAGCTGCCTG |
| 2701 | CAGGGGCGCC TGATGCGGTA TTTTCTCCTT ACGCATCTGT GCGGTATTTC |
| 2751 | ACACCGCATA CGTCAAAGCA ACCATAGTAC GCGCCCTGTA GCGGCGCATT |
| 2801 | AAGCGCGGCG GGTGTGGTGG TTACGCGCAG CGTGACCGCT ACACTTGCCA |
| 2851 | GCGCCCTAGC GCCCGCTCCT TTCGCTTTCT TCCCTTCCTT TCTCGCCACG |
| 2901 | TTCGCCGGCT TTCCCCGTCA AGCTCTAAAT CGGGGGCTCC CTTTAGGGTT |
| 2951 | CCGATTTAGT GCTTTACGGC ACCTCGACCC CAAAAAACTT GATTTGGGTG |
| 3001 | ATGGTTCACG TAGTGGGCCA TCGCCCTGAT AGACGGTTTT TCGCCCTTTG |
| 3051 | ACGTTGGAGT CCACGTTCTT TAATAGTGGA CTCTTGTTCC AAACTGGAAC |
| 3101 | AACACTCAAC CCTATCTCGG GCTATTCTTT TGATTTATAA GGGATTTTGC |
| 3151 | CGATTTCGGC CTATTGGTTA AAAAATGAGC TGATTTAACA AAAATTTAAC |
| 3201 | GCGAATTTTA ACAAAATATT AACGTTTACA ATTTTATGGT GCACTCTCAG |
| 3251 | TACAATCTGC TCTGATGCCG CATAGTTAAG CCAGCCCCGA CACCCGCCAA |
| 3301 | CACCCGCTGA CGCGCCCTGA CGGGCTTGTC TGCTCCCGGC ATCCGCTTAC |
| 3351 | AGACAAGCTG TGACCGTCTC CGGGAGCTGC ATGTGTCAGA GGTTTTCACC |
| 3401 | GTCATCACCG AAACGCGCGA GACGAAAGGG CCTCGTGATA CGCCTATTTT |
| 3451 | TATAGGTTAA TGTCATGATA ATAATGGTTT CTTAGACGTC AGGTGGCACT |
| 3501 | TTTCGGGGAA ATGTGCGCGG AACCCCTATT TGTTTATTTT TCTAAATACA |
| 3551 | TTCAAATATG TATCCGCTCA TGAGACAATA ACCCTGATAA ATGCTTCAAT |
| 3601 | AATATTGAAA AAGGAAGAGT ATGAGTATTC AACATTTCCG TGTCGCCCTT |
| 3651 | ATTCCCTTTT TTGCGGCATT TTGCCTTCCT GTTTTTGCTC ACCCAGAAAC |
| 3701 | GCTGGTGAAA GTAAAAGATG CTGAAGATCA GTTGGGTGCA CGAGTGGGTT |
| 3751 | ACATCGAACT GGATCTCAAC AGCGGTAAGA TCCTTGAGAG TTTTCGCCCC |
| 3801 | GAAGAACGTT TTCCAATGAT GAGCACTTTT AAAGTTCTGC TATGTGGCGC |
| 3851 | GGTATTATCC CGTATTGACG CCGGGCAAGA GCAACTCGGT CGCCGCATAC |
| 3901 | ACTATTCTCA GAATGACTTG GTTGAGTACT CACCAGTCAC AGAAAAGCAT |
| 3951 | CTTACGGATG GCATGACAGT AAGAGAATTA TGCAGTGCTG CCATAACCAT |
| 4001 | GAGTGATAAC ACTGCGGCCA ACTTACTTCT GACAACGATC GGAGGACCGA |
| 4051 | AGGAGCTAAC CGCTTTTTTG CACAACATGG GGGATCATGT AACTCGCCTT |
| 4101 | GATCGTTGGG AACCGGAGCT GAATGAAGCC ATACCAAACG ACGAGCGTGA |
| 4151 | CACCACGATG CCTGTAGCAA TGGCAACAAC GTTGCGCAAA CTATTAACTG |
| 4201 | GCGAACTACT TACTCTAGCT TCCCGGCAAC AATTAATAGA CTGGATGGAG |
| 4251 | GCGGATAAAG TTGCAGGACC ACTTCTGCGC TCGGCCCTTC CGGCTGGCTG |
| 4301 | GTTTATTGCT GATAAATCTG GAGCCGGTGA GCGTGGGTCT CGCGGTATCA |
| 4351 | TTGCAGCACT GGGGCCAGAT GGTAAGCCCT CCCGTATCGT AGTTATCTAC |
| 4401 | ACGACGGGGA GTCAGGCAAC TATGGATGAA CGAAATAGAC AGATCGCTGA |
| 4451 | GATAGGTGCC TCACTGATTA AGCATTGGTA ACTGTCAGAC CAAGTTTACT |
| 4501 | CATATATACT TTAGATTGAT TTAAAACTTC ATTTTTAATT TAAAAGGATC |
| 4551 | TAGGTGAAGA TCCTTTTTGA TAATCTCATG ACCAAAATCC CTTAACGTGA |
| 4601 | GTTTTCGTTC CACTGAGCGT CAGACCCCGT AGAAAAGATC AAAGGATCTT |
| 4651 | CTTGAGATCC TTTTTTTCTG CGCGTAATCT GCTGCTTGCA AACAAAAAAA |
| 4701 | CCACCGCTAC CAGCGGTGGT TTGTTTGCCG GATCAAGAGC TACCAACTCT |
| 4751 | TTTTCCGAAG GTAACTGGCT TCAGCAGAGC GCAGATACCA AATACTGTCC |
| 4801 | TTCTAGTGTA GCCGTAGTTA GGCCACCACT TCAAGAACTC TGTAGCACCG |
| 4851 | CCTACATACC TCGCTCTGCT AATCCTGTTA CCAGTGGCTG CTGCCAGTGG |
| 4901 | CGATAAGTCG TGTCTTACCG GGTTGGACTC AAGACGATAG TTACCGGATA |
| 4951 | AGGCGCAGCG GTCGGGCTGA ACGGGGGGTT CGTGCACACA GCCCAGCTTG |
| 5001 | GAGCGAACGA CCTACACCGA ACTGAGATAC CTACAGCGTG AGCTATGAGA |
| 5051 | AAGCGCCACG CTTCCCGAAG GGAGAAAGGC GGACAGGTAT CCGGTAAGCG |
| 5101 | GCAGGGTCGG AACAGGAGAG CGCACGAGGG AGCTTCCAGG GGGAAACGCC |
| 5151 | TGGTATCTTT ATAGTCCTGT CGGGTTTCGC CACCTCTGAC TTGAGCGTCG |
| 5201 | ATTTTTGTGA TGCTCGTCAG GGGGGCGGAG CCTATGGAAA AACGCCAGCA |
| 5251 | ACGCGGCCTT TTTACGGTTC CTGGCCTTTT GCTGGCCTTT TGCTCACATG |
| 5301 | TCCTGCAGGC AGCTGCGCGC TCGCTCGCTC ACTGAGGCCG CCCGGGCAAA |
| 5351 | GCCCGGGCGT CGGGCGACCT TTGGTCGCCC GGCCTCAGTG AGCGAGCGAG |
| 5401 | CGCGCAGAGA GGGAGTGGCC AACTCCATCA CTAGGGGTTC CTGCGGCCGC |
| 5451 | GATAAATCGA GATCTTCCAG AGCATGAGCG TGGCCATAGA GCCCACCGCA |
| 5501 | TCCCCAGCAT GCCTGCTATT GTCTTCCCAA TCCTCCCCCT TGCTGTCCTG |
| 5551 | CCCCACCCCA CCCCCCAGAA TAGAATGACA CCTACTCAGA CAATGCGATG |
| 5601 | CAATTTCCTC ATTTTATTAG GAAAGGACAG TGGGAGTGGC ACCTTCCAGG |
| 5651 | GTCAAGGAAG GCACGGGGGA GGGGCAAACA ACAGATGGCT GGCAACTAGA |
| 5701 | AGGCACAGTC GAGGCTGATC AGCGAGCTCC ACGCTGGTAC CGTCGACGGC |
| 5751 | TGCGTCTAGT TGCAGTAGTT CTCCAGCTGG TAGAGGGAGC AGATGCTGGT |
| 5801 | ACAGCATTGT TCCACAATGC CACGCTTCTG TCGCGACCCC TCCAGGGCCA |
| 5851 | AGGGCTGCAG GCTGCCTGCA CCAGGGCCCC CGCCCAGCTC CACCTGCCCC |
| 5901 | ACCTGCAGGT CCTCTGCCTC CCGCTTGGTC CTGGGTGTGT AGAAGAAGCC |
| 5951 | TCGTTCCCCG CACACTAGGT AGAGAGCTTC CACCAGATCT GAGCCGCACA |
| 6001 | GGTGTTGGTT CACAAAGGCT GCGGCTGGGT CAGGTCCCCA GAGGGCCAGC |
| 6051 | AGCGCCAGCA GGGGCAGGAG GCGCATCCAC AGGGCCATGG CAGAAGGTCG |
| 6101 | ACGCTAGCAA GCTTGGGTCT CCCTATAGTG AGTCGTATTA ATTTCGATAA |
| 6151 | GCCAGTTAAG CAGTGGGTTC TCTAGTTAGC CAGAGAGCTC TGCTTATATA |
| 6201 | GACCTCCCAC CGTACACGCC TACCGCCCAT TTGCGTCAAT GGGGCGGAGT |
| 6251 | TGTTACGACA TTTTGGAAAG TCCCGTTGAT TTTGGTGCCA AAACAAACTC |
| 6301 | CCATTGACGT CAATGGGGTG GAGACTTGGA AATCCCCGTG AGTCAAACCG |
| 6351 | CTATCCACGC CCATTGATGT ACTGCCAAAA CCGCATCACC ATGGTAATAG |
| 6401 | CGATGACTAA TACGTAGATG TACTGCCAAG TAGGAAAGTC CCATAAGGTC |
| 6451 | ATGTACTGGG CATAATGCCA GGCGGGCCAT TTACCGTCAT TGACGTCAAT |
| 6501 | AGGGGGCGTA CTTGGCATAG AT |

ITR 5′: 5302-5442 bp
miniCMV promoter: 6109-6519 bp
hIns: 5749-6095 bp
bGH polyA: 5480-5732 bp
RSV promoter: 87-796 bp
hGck: 821-2235 bp
SV40 polyA: 2246-2541 bp
ITR 3′: 2564-2704 bp
R: miniCMV-hIns-SV40 enhancer

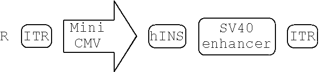

pAAV-miniCMV-hIns-SV40 enhancer plasmid sequence
(SEQ ID NO: 28)

| | |
|---|---|
| 1 | GCTCATGCTC TGGAAGATCT CGATTTAAAT GCGGCCGCAG GAACCCCTAG |
| 51 | TGATGGAGTT GGCCACTCCC TCTCTGCGCG CTCGCTCGCT CACTGAGGCC |
| 101 | GGGCGACCAA AGGTCGCCCG ACGCCCGGGC TTTGCCCGGG CGGCCTCAGT |
| 151 | GAGCGAGCGA GCGCGCAGCT GCCTGCAGGG GCGCCTGATG CGGTATTTTC |
| 201 | TCCTTACGCA TCTGTGCGGT ATTTCACACC GCATACGTCA AAGCAACCAT |
| 251 | AGTACGCGCC CTGTAGCGGC GCATTAAGCG CGGCGGGTGT GGTGGTTACG |
| 301 | CGCAGCGTGA CCGCTACACT TGCCAGCGCC CTAGCGCCCG CTCCTTTCGC |
| 351 | TTTCTTCCCT TCCTTTCTCG CCACGTTCGC CGGCTTTCCC CGTCAAGCTC |
| 401 | TAAATCGGGG GCTCCCTTTA GGGTTCCGAT TTAGTGCTTT ACGGCACCTC |
| 451 | GACCCCAAAA AACTTGATTT GGGTGATGGT TCACGTAGTG GGCCATCGCC |
| 501 | CTGATAGACG GTTTTTCGCC CTTTGACGTT GGAGTCCACG TTCTTTAATA |
| 551 | GTGGACTCTT GTTCCAAACT GGAACAACAC TCAACCCTAT CTCGGGCTAT |
| 601 | TCTTTTGATT TATAAGGGAT TTTGCCGATT TCGGCCTATT GGTTAAAAAA |
| 651 | TGAGCTGATT TAACAAAAAT TTAACGCGAA TTTTAACAAA ATATTAACGT |
| 701 | TTACAATTTT ATGGTGCACT CTCAGTACAA TCTGCTCTGA TGCCGCATAG |
| 751 | TTAAGCCAGC CCCGACACCC GCCAACACCC GCTGACGCGC CCTGACGGGC |
| 801 | TTGTCTGCTC CCGGCATCCG CTTACAGACA AGCTGTGACC GTCTCCGGGA |
| 851 | GCTGCATGTG TCAGAGGTTT TCACCGTCAT CACCGAAACG CGCGAGACGA |
| 901 | AAGGGCCTCG TGATACGCCT ATTTTTATAG GTTAATGTCA TGATAATAAT |
| 951 | GGTTTCTTAG ACGTCAGGTG GCACTTTTCG GGGAAATGTG CGCGGAACCC |
| 1001 | CTATTTGTTT ATTTTTCTAA ATACATTCAA ATATGTATCC GCTCATGAGA |
| 1051 | CAATAACCCT GATAAATGCT TCAATAATAT TGAAAAAGGA AGAGTATGAG |
| 1101 | TATTCAACAT TTCCGTGTCG CCCTTATTCC CTTTTTTGCG GCATTTTGCC |
| 1151 | TTCCTGTTTT TGCTCACCCA GAAACGCTGG TGAAAGTAAA AGATGCTGAA |
| 1201 | GATCAGTTGG GTGCACGAGT GGGTTACATC GAACTGGATC TCAACAGCGG |
| 1251 | TAAGATCCTT GAGAGTTTTC GCCCCGAAGA ACGTTTTCCA ATGATGAGCA |
| 1301 | CTTTTAAAGT TCTGCTATGT GGCGCGGTAT TATCCCGTAT TGACGCCGGG |
| 1351 | CAAGAGCAAC TCGGTCGCCG CATACACTAT TCTCAGAATG ACTTGGTTGA |
| 1401 | GTACTCACCA GTCACAGAAA AGCATCTTAC GGATGGCATG ACAGTAAGAG |
| 1451 | AATTATGCAG TGCTGCCATA ACCATGAGTG ATAACACTGC GGCCAACTTA |
| 1501 | CTTCTGACAA CGATCGGAGG ACCGAAGGAG CTAACCGCTT TTTTGCACAA |
| 1551 | CATGGGGGAT CATGTAACTC GCCTTGATCG TTGGGAACCG GAGCTGAATG |
| 1601 | AAGCCATACC AAACGACGAG CGTGACACCA CGATGCCTGT AGCAATGGCA |
| 1601 | ACAACGTTGC GCAAACTATT AACTGGCGAA CTACTTACTC TAGCTTCCCG |
| 1701 | GCAACAATTA ATAGACTGGA TGGAGGCGGA TAAAGTTGCA GGACCACTTC |
| 1751 | TGCGCTCGGC CCTTCCGGCT GGCTGGTTTA TTGCTGATAA ATCTGGAGCC |
| 1801 | GGTGAGCGTG GGTCTCGCGG TATCATTGCA GCACTGGGGC CAGATGGTAA |
| 1851 | GCCCTCCCGT ATCGTAGTTA TCTACACGAC GGGGAGTCAG GCAACTATGG |
| 1901 | ATGAACGAAA TAGACAGATC GCTGAGATAG GTGCCTCACT GATTAAGCAT |
| 1951 | TGGTAACTGT CAGACCAAGT TTACTCATAT ATACTTTAGA TTGATTTAAA |
| 2001 | ACTTCATTTT TAATTTAAAA GGATCTAGGT GAAGATCCTT TTTGATAATC |
| 2051 | TCATGACCAA AATCCCTTAA CGTGAGTTTT CGTTCCACTG AGCGTCAGAC |
| 2101 | CCCGTAGAAA AGATCAAAGG ATCTTCTTGA GATCCTTTTT TTCTGCGCGT |
| 2151 | AATCTGCTGC TTGCAAACAA AAAAACCACC GCTACCAGCG GTGGTTTGTT |
| 2201 | TGCCGGATCA AGAGCTACCA ACTCTTTTTC CGAAGGTAAC TGGCTTCAGC |
| 2251 | AGAGCGCAGA TACCAAATAC TGTCCTTCTA GTGTAGCCGT AGTTAGGCCA |
| 2301 | CCACTTCAAG AACTCTGTAG CACCGCCTAC ATACCTCGCT CTGCTAATCC |
| 2351 | TGTTACCAGT GGCTGCTGCC AGTGGCGATA AGTCGTGTCT TACCGGGTTG |
| 2401 | GACTCAAGAC GATAGTTACC GGATAAGGCG CAGCGGTCGG GCTGAACGGG |
| 2451 | GGGTTCGTGC ACACAGCCCA GCTTGGAGCG AACGACCTAC ACCGAACTGA |
| 2501 | GATACCTACA GCGTGAGCTA TGAGAAAGCG CCACGCTTCC CGAAGGGAGA |
| 2551 | AAGGCGGACA GGTATCCGGT AAGCGGCAGG GTCGGAACAG GAGAGCGCAC |
| 2601 | GAGGGAGCTT CCAGGGGGAA ACGCCTGGTA TCTTTATAGT CCTGTCGGGT |
| 2651 | TTCGCCACCT CTGACTTGAG CGTCGATTTT TGTGATGCTC GTCAGGGGGG |
| 2701 | CGGAGCCTAT GGAAAAACGC CAGCAACGCG GCCTTTTTAC GGTTCCTGGC |
| 2731 | CTTTTGCTGG CCTTTTGCTC ACATGTCCTG CAGGCAGCTG CGCGCTCGCT |
| 2801 | CGCTCACTGA GGCCGCCCGG GCAAAGCCCG GGCGTCGGGC GACCTTTGGT |
| 2851 | CGCCCGGCCT CAGTGAGCGA GCGAGCGCGC AGAGAGGGAG TGGCCAACTC |
| 2901 | CATCACTAGG GGTTCCTGCG GCCGCGATAT CTATGCCAAG TACGCCCCCT |
| 2951 | ATTGACGTCA ATGACGGTAA ATGGCCCGCC TGGCATTATG CCCAGTACAT |
| 3001 | GACCTTATGG GACTTTCCTA CTTGGCAGTA CATCTACGTA TTAGTCATCG |
| 3051 | CTATTACCAT GGTGATGCGG TTTTGGCAGT ACATCAATGG GCGTGGATAG |
| 3101 | CGGTTTGACT CACGGGGATT TCCAAGTCTC CACCCCATTG ACGTCAATGG |
| 3151 | GAGTTTGTTT TGGCACCAAA ATCAACGGGA CTTTCCAAAA TGTCGTAACA |
| 3201 | ACTCCGCCCC ATTGACGCAA ATGGGCGGTA GGCGTGTACG GTGGGAGGTC |
| 3251 | TATATAAGCA GAGCTCTCTG GCTAACTAGA GAACCCACTG CTTAACTGGC |
| 3301 | TTATCGAAAT TAATACGACT CACTATAGGG AGACCCAAGC TTGCTAGCGT |
| 3351 | CGACCTTCTG CCATGGCCCT GTGGATGCGC CTCCTGCCCC TGCTGGCGCT |
| 3401 | GCTGGCCCTC TGGGGACCTG ACCCAGCCGC AGCCTTTGTG AACCAACACC |
| 3451 | TGTGCGGCTC AGATCTGGTG GAAGCTCTCT ACCTAGTGTG CGGGGAACGA |
| 3501 | GGCTTCTTCT ACACACCCAG GACCAAGCGG GAGGCAGAGG ACCTGCAGGT |
| 3551 | GGGGCAGGTG GAGCTGGGCG GGGGCCCTGG TGCAGGCAGC CTGCAGCCCT |
| 3601 | TGGCCCTGGA GGGGTCGCGA CAGAAGCGTG GCATTGTGGA ACAATGCTGT |
| 3651 | ACCAGCATCT GCTCCCTCTA CCAGCTGGAG AACTACTGCA ACTAGACGCA |
| 3701 | GCCGTCGACG GTACCAGCGC TGAGTCGGGG CGGCCGGCCG CTTCGAGCAG |
| 3751 | ACATGATAAG ATACATTGAT GAGTTTGGAC AAACCACAAC TAGAATGCAG |
| 3801 | TGAAAAAAAT GCTTTATTTG TGAAATTTGT GATGCTATTG CTTTATTTGT |
| 3851 | AACCATTATA AGCTGCAATA AACAAGTTAA CAACAACAAT TGCATTCATT |
| 3901 | TTATGTTTCA GGTTCAGGGG GAGGTGTGGG AGGTTTTTTA AAGCAAGTAA |
| 3951 | AACCTCTACA AATTTGGTAA AATCGATAAG GATCTGAACG ATGGAGCGGA |
| 4001 | GAATGGGCGG AACTGGGCGG AGTTAGGGGC GGGATGGGCG GAGTTAGGGG |
| 4051 | CGGGACTATG GTTGCTGACT AATTGAGATG CATGCTTTGC ATACTTCTGC |
| 4101 | CTGCTGGGGA GCCTGGGGAC TTTCCACACC TGGTTGCTGA CTAATTGAGA |
| 4151 | TGCATGCTTT GCATACTTCT GCCTGCTGGG GAGCCTGGGG ACTTTCCACA |
| 4201 | CCCTAACTGA CACACATTCC ACAGCGGCAA ATTTGAGC |

ITR 5′: 2777-2917 bp
miniCMV promoter: 2932-3342 bp
hIns: 3356-3702 bp
SV40 enhancer and SV40 polyA: 3719-4238 bp
ITR 3′: 39-179 bp
S. miniCMV-hIns-SV40enhancer(rev)-RSV-hGck-bGH

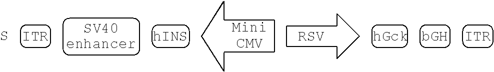

pAAV-miniCMV-hIns-SV40enhancer(rev)-RSV-hGck-bGH plasmid
sequence
ITR 5′: 5256-5396 bp
miniCMV promoter: 6330-6740 bp
hIns: 5970-6316 bp
SV40 enhancer and SV40 polyA: 5434-5953 bp
RSV promoter: 87-796 bp
hGck: 821-2235 bp
bGH polyA: 2243-2501 bp
ITR 3′: 2518-2658 bp
(SEQ ID NO: 29)

| | |
|---|---|
| 1 | ATCCATGTTT GACAGCTTAT CATCGCAGAT CCGTATGGTG CACTCTCAGT |
| 51 | ACAATCTGCT CTGATGCCGC ATAGTTAAGC CAGTATCTGC TCCCTGCTTG |
| 101 | TGTGTTGGAG GTCGCTGAGT AGTGCGCGAG CAAAATTTAA GCTACAACAA |
| 151 | GGCAAGGCTT GACCGACAAT TGCATGAAGA ATCTGCTTAG GGTTAGGCGT |
| 201 | TTTGCGCTGC TTCGCGATGT ACGGGCCAGA TATTCGCGTA TCTGAGGGGA |
| 251 | CTAGGGTGTG TTTAGGCGAA AAGCGGGGCT TCGGTTGTAC GCGGTTAGGA |
| 301 | GTCCCCTCAG GATATAGTAG TTTCGCTTTT GCATAGGGAG GGGGAAATGT |
| 351 | AGTCTTATGC AATACTCTTG TAGTCTTGCA ACATGGTAAC GATGAGTTAG |
| 401 | CAACATGCCT TACAAGGAGA GAAAAAGCAC CGTGCATGCC GATTGGTGGA |
| 451 | AGTAAGGTGG TACGATCGTG CCTTATTAGG AAGGCAACAG ACGGGTCTGA |
| 501 | CATGGATTGG ACGAACCACT AAATTCCGCA TTGCAGAGAT ATTGTATTTA |
| 551 | AGTGCCTAGC TCGATACAAT AAACGCCATT TGACCATTCA CCACATTGGT |
| 601 | GTGCACCTCC AAGCTGGGTA CCAGCTTCTA GAGAGATCTG CTTCAGCTGG |
| 651 | AGGCACTGGG CAGGTAAGTA TCAAGGTTAC AAGACAGGTT TAAGGAGACC |
| 701 | AATAGAAACT GGGCTTGTCG AGACAGAGAA GACTCTTGCG TTTCTGATAG |
| 751 | GCACCTATTG GTCTTACTGA CATCCACTTT GCCTTTCTCT CCACAGGTGC |
| 801 | AGCTGCTGCA GCGGTCTAGA ACTCGAGTCG AGACCATGGC GATGGATGTC |
| 851 | ACAAGGAGCC AGGCCCAGAC AGCCTTGACT CTGGTAGAGC AGATCCTGGC |
| 901 | AGAGTTCCAG CTGCAGGAGG AGGACCTGAA GAAGGTGATG AGACGGATGC |
| 951 | AGAAGGAGAT GGACCGCGGC CTGAGGCTGG AGACCCATGA AGAGGCCAGT |
| 1001 | GTGAAGATGC TGCCCACCTA CGTGCGCTCC ACCCCAGAAG GCTCAGAAGT |
| 1051 | CGGGGACTTC CTCTCCCTGG ACCTGGGTGG CACTAACTTC AGGGTGATGC |
| 1101 | TGGTGAAGGT GGGAGAAGGT GAGGAGGGGC AGTGGAGCGT GAAGACCAAA |
| 1151 | CACCAGATGT ACTCCATCCC CGAGGACGCC ATGACCGGCA CTGCTGAGAT |
| 1201 | GCTCTTCGAC TACATCTCTG AGTGCATCTC CGACTTCCTG GACAAGCATC |
| 1251 | AGATGAAACA CAAGAAGCTG CCCCTGGGCT TCACCTTCTC CTTTCCTGTG |
| 1301 | AGGCACGAAG ACATCGATAA GGGCATCCTT CTCAACTGGA CCAAGGGCTT |
| 1351 | CAAGGCCTCA GGAGCAGAAG GGAACAATGT CGTGGGGCTT CTGCGAGACG |
| 1401 | CTATCAAACG GAGAGGGGAC TTTGAAATGG ATGTGGTGGC AATGGTGAAT |
| 1451 | GACACGGTGG CCACGATGAT CTCCTGCTAC TACGAAGACC ATCAGTGCGA |
| 1501 | GGTCGGCATG ATCGTGGGCA CGGGCTGCAA TGCCTGCTAC ATGGAGGAGA |
| 1551 | TGCAGAATGT GGAGCTGGTG GAGGGGGACG AGGGCCGCAT GTGCGTCAAT |
| 1601 | ACCGAGTGGG GCGCCTTCGG GGACTCCGGC GAGCTGGACG AGTTCCTGCT |
| 1651 | GGAGTATGAC CGCCTGGTGG ACGAGAGCTC TGCAAACCCC GGTCAGCAGC |
| 1701 | TGTATGAGAA GCTCATAGGT GGCAAGTACA TGGGCGAGCT GGTGCGGCTT |
| 1751 | GTGCTGCTCA GGCTCGTGGA CGAAAACCTG CTCTTCCACG GGGAGGCCTC |
| 1801 | CGAGCAGCTG CGCACACGCG GAGCCTTCGA GACGCGCTTC GTGTCGCAGG |
| 1851 | TGGAGAGCGA CACGGGCGAC CGCAAGCAGA TCTACAACAT CCTGAGCACG |
| 1901 | CTGGGGCTGC GACCCTCGAC CACCGACTGC GACATCGTGC GCCGCGCCTG |
| 1951 | CGAGAGCGTG TCTACGCGCG CTGCGCACAT GTGCTCGGCG GGGCTGGCGG |
| 2001 | GCGTCATCAA CCGCATGCGC GAGAGCCGCA GCGAGGACGT AATGCGCATC |
| 2051 | ACTGTGGGCG TGGATGGCTC CGTGTACAAG CTGCACCCCA GCTTCAAGGA |
| 2101 | GCGGTTCCAT GCCAGCGTGC GCAGGCTGAC GCCCAGCTGC GAGATCACCT |
| 2151 | TCATCGAGTC GGAGGAGGGC AGTGGCCGGG GCGCGGCCCT GGTCTCGGCG |
| 2201 | GTGGCCTGTA AGAAGGCCTG TATGCTGGGC CAGTGACTCG AGCACGTGGA |
| 2251 | GCTCGCTGAT CAGCCTCGAC TGTGCCTTCT AGTTGCCAGC CATCTGTTGT |
| 2301 | TTGCCCCTCC CCCGTGCCTT CCTTGACCCT GGAAGGTGCC ACTCCCACTG |
| 2351 | TCCTTTCCTA ATAAAATGAG GAAATTGCAT CGCATTGTCT GAGTAGGTGT |
| 2401 | CATTCTATTC TGGGGGGTGG GGTGGGGCAG GACAGCAAGG GGGAGGATTG |
| 2451 | GGAAGACAAT AGCAGGCATG CTGGGGATGC GGTGGGCTCT ATGGCCACGT |
| 2501 | GATTTAAATG CGGCCGCAGG AACCCCTAGT GATGGAGTTG GCCACTCCCT |
| 2551 | CTCTGCGCGC TCGCTCGCTC ACTGAGGCCG GGCGACCAAA GGTCGCCCGA |
| 2601 | CGCCCGGGCT TTGCCCGGGC GGCCTCAGTG AGCGAGCGAG CGCGCAGCTG |
| 2651 | CCTGCAGGGG CGCCTGATGC GGTATTTTCT CCTTACGCAT CTGTGCGGTA |
| 2701 | TTTCACACCG CATACGTCAA AGCAACCATA GTACGCGCCC TGTAGCGGCG |
| 2751 | CATTAAGCGC GGCGGGTGTG GTGGTTACGC GCAGCGTGAC CGCTACACTT |
| 2801 | GCCAGCGCCC TAGCGCCCGC TCCTTTCGCT TTCTTCCCTT CCTTTCTCGC |
| 2851 | CACGTTCGCC GGCTTTCCCC GTCAAGCTCT AAATCGGGGG CTCCCTTTAG |
| 2901 | GGTTCCGATT TAGTGCTTTA CGGCACCTCG ACCCCAAAAA ACTTGATTTG |
| 2951 | GGTGATGGTT CACGTAGTGG GCCATCGCCC TGATAGACGG TTTTTCGCCC |
| 3001 | TTTGACGTTG GAGTCCACGT TCTTTAATAG TGGACTCTTG TTCCAAACTG |
| 3051 | GAACAACACT CAACCCTATC TCGGGCTATT CTTTTGATTT ATAAGGGATT |
| 3101 | TTGCCGATTT CGGCCTATTG GTTAAAAAAT GAGCTGATTT AACAAAAATT |
| 3151 | TAACGCGAAT TTTAACAAAA TATTAACGTT TACAATTTTA TGGTGCACTC |
| 3201 | TCAGTACAAT CTGCTCTGAT GCCGCATAGT TAAGCCAGCC CCGACACCCG |
| 3251 | CCAACACCCG CTGACGCGCC CTGACGGGCT TGTCTGCTCC CGGCATCCGC |
| 3301 | TTACAGACAA GCTGTGACCG TCTCCGGGAG CTGCATGTGT CAGAGGTTTT |
| 3351 | CACCGTCATC ACCGAAACGC GCGAGACGAA AGGGCCTCGT GATACGCCTA |
| 3401 | TTTTTATAGG TTAATGTCAT GATAATAATG GTTTCTTAGA CGTCAGGTGG |
| 3451 | CACTTTTCGG GGAAATGTGC GCGGAACCCC TATTTGTTTA TTTTTCTAAA |
| 3501 | TACATTCAAA TATGTATCCG CTCATGAGAC AATAACCCTG ATAAATGCTT |
| 3551 | CAATAATATT GAAAAAGGAA GAGTATGAGT ATTCAACATT TCCGTGTCGC |
| 3601 | CCTTATTCCC TTTTTTGCGG CATTTTGCCT TCCTGTTTTT GCTCACCCAG |
| 3651 | AAACGCTGGT GAAAGTAAAA GATGCTGAAG ATCAGTTGGG TGCACGAGTG |
| 3701 | GGTTACATCG AACTGGATCT CAACAGCGGT AAGATCCTTG AGAGTTTTCG |
| 3751 | CCCCGAAGAA CGTTTTCCAA TGATGAGCAC TTTTAAAGTT CTGCTATGTG |
| 3851 | GCGCGGTATT ATCCCGTATT GACGCCGGGC AAGAGCAACT CGGTCGCCGC |
| 3851 | ATACACTATT CTCAGAATGA CTTGGTTGAG TACTCACCAG TCACAGAAAA |
| 3901 | GCATCTTACG GATGGCATGA CAGTAAGAGA ATTATGCAGT GCTGCCATAA |
| 3951 | CCATGAGTGA TAACACTGCG GCCAACTTAC TTCTGACAAC GATCGGAGGA |
| 4001 | CCGAAGGAGC TAACCGCTTT TTTGCACAAC ATGGGGGATC ATGTAACTCG |
| 4051 | CCTTGATCGT TGGGAACCGG AGCTGAATGA AGCCATACCA AACGACGAGC |
| 4101 | GTGACACCAC GATGCCTGTA GCAATGGCAA CAACGTTGCG CAAACTATTA |
| 4151 | ACTGGCGAAC TACTTACTCT AGCTTCCCGG CAACAATTAA TAGACTGGAT |
| 4201 | GGAGGCGGAT AAAGTTGCAG GACCACTTCT GCGCTCGGCC CTTCCGGCTG |
| 4251 | GCTGGTTTAT TGCTGATAAA TCTGGAGCCG GTGAGCGTGG GTCTCGCGGT |
| 4351 | ATCATTGCAG CACTGGGGCC AGATGGTAAG CCCTCCCGTA TCGTAGTTAT |
| 4351 | CTACACGACG GGGAGTCAGG CAACTATGGA TGAACGAAAT AGACAGATCG |
| 4401 | CTGAGATAGG TGCCTCACTG ATTAAGCATT GGTAACTGTC AGACCAAGTT |
| 4451 | TACTCATATA TACTTTAGAT TGATTTAAAA CTTCATTTTT AATTTAAAAG |
| 4501 | GATCTAGGTG AAGATCCTTT TTGATAATCT CATGACCAAA ATCCCTTAAC |
| 4551 | GTGAGTTTTC GTTCCACTGA GCGTCAGACC CCGTAGAAAA GATCAAAGGA |
| 4601 | TCTTCTTGAG ATCCTTTTTT TCTGCGCGTA ATCTGCTGCT TGCAAACAAA |
| 4651 | AAAACCACCG CTACCAGCGG TGGTTTGTTT GCCGGATCAA GAGCTACCAA |
| 4701 | CTCTTTTTCC GAAGGTAACT GGCTTCAGCA GAGCGCAGAT ACCAAATACT |
| 4751 | GTCCTTCTAG TGTAGCCGTA GTTAGGCCAC CACTTCAAGA ACTCTGTAGC |
| 4801 | ACCGCCTACA TACCTCGCTC TGCTAATCCT GTTACCAGTG GCTGCTGCCA |
| 4851 | GTGGCGATAA GTCGTGTCTT ACCGGGTTGG ACTCAAGACG ATAGTTACCG |
| 4901 | GATAAGGCGC AGCGGTCGGG CTGAACGGGG GGTTCGTGCA CACAGCCCAG |
| 4951 | CTTGGAGCGA ACGACCTACA CCGAACTGAG ATACCTACAG CGTGAGCTAT |
| 5001 | GAGAAAGCGC CACGCTTCCC GAAGGGAGAA AGGCGGACAG GTATCCGGTA |
| 5051 | AGCGGCAGGG TCGGAACAGG AGAGCGCACG AGGGAGCTTC CAGGGGGAAA |
| 5101 | CGCCTGGTAT CTTTATAGTC CTGTCGGGTT TCGCCACCTC TGACTTGAGC |
| 5151 | GTCGATTTTT GTGATGCTCG TCAGGGGGGC GGAGCCTATG GAAAAACGCC |
| 5201 | AGCAACGCGG CCTTTTTACG GTTCCTGGCC TTTTGCTGGC CTTTTGCTCA |
| 5251 | CATGTCCTGC AGGCAGCTGC GCGCTCGCTC GCTCACTGAG GCCGCCCGGG |
| 5301 | CAAAGCCCGG GCGTCGGGCG ACCTTTGGTC GCCCGGCCTC AGTGAGCGAG |
| 5351 | CGAGCGCGCA GAGAGGGAGT GGCCAACTCC ATCACTAGGG GTTCCTGCGG |
| 5401 | CCGCGATAAA TCGAGATCTT CCAGAGCATG AGCGCTCAAA TTTGCCGCTG |
| 5451 | TGGAATGTGT GTCAGTTAGG GTGTGGAAAG TCCCCAGGCT CCCCAGCAGG |
| 5501 | CAGAAGTATG CAAAGCATGC ATCTCAATTA GTCAGCAACC AGGTGTGGAA |
| 5551 | AGTCCCCAGG CTCCCCAGCA GGCAGAAGTA TGCAAAGCAT GCATCTCAAT |
| 5601 | TAGTCAGCAA CCATAGTCCC GCCCCTAACT CCGCCCATCC CGCCCCTAAC |
| 5651 | TCCGCCCAGT TCCGCCCATT CTCCGCTCCA TCGTTCAGAT CCTTATCGAT |
| 5701 | TTTACCAAAT TTGTAGAGGT TTTACTTGCT TTAAAAAACC TCCCACACCT |
| 5751 | CCCCCTGAAC CTGAAACATA AAATGAATGC AATTGTTGTT GTTAACTTGT |
| 5801 | TTATTGCAGC TTATAATGGT TACAAATAAA GCAATAGCAT CACAAATTTC |
| 5851 | ACAAATAAAG CATTTTTTTC ACTGCATTCT AGTTGTGGTT TGTCCAAACT |
| 5901 | CATCAATGTA TCTTATCATG TCTGCTCGAA GCGGCCGGCC GCCCCGACTC |
| 5951 | AGCGCTGGTA CCGTCGACGG CTGCGTCTAG TTGCAGTAGT TCTCCAGCTG |
| 6001 | GTAGAGGGAG CAGATGCTGG TACAGCATTG TTCCACAATG CCACGCTTCT |
| 6051 | GTCGCGACCC CTCCAGGGCC AAGGGCTGCA GGCTGCCTGC ACCAGGGCCC |
| 6101 | CCGCCCAGCT CCACCTGCCC CACCTGCAGG TCCTCTGCCT CCCGCTTGGT |
| 6151 | CCTGGGTGTG TAGAAGAAGC CTCGTTCCCC GCACACTAGG TAGAGAGCTT |
| 6201 | CCACCAGATC TGAGCCGCAC AGGTGTTGGT TCACAAAGGC TGCGGCTGGG |
| 6251 | TCAGGTCCCC AGAGGGCCAG CAGCGCCAGC AGGGGCAGGA GGCGCATCCA |
| 6301 | CAGGGCCATG GCAGAAGGTC GACGCTAGCA AGCTTGGGTC TCCCTATAGT |
| 6351 | GAGTCGTATT AATTTCGATA AGCCAGTTAA GCAGTGGGTT CTCTAGTTAG |
| 6401 | CCAGAGAGCT CTGCTTATAT AGACCTCCCA CCGTACACGC CTACCGCCCA |
| 6451 | TTTGCGTCAA TGGGGCGGAG TTGTTACGAC ATTTTGGAAA GTCCCGTTGA |
| 6501 | TTTTGGTGCC AAAACAAACT CCCATTGACG TCAATGGGGT GGAGACTTGG |
| 6551 | AAATCCCCGT GAGTCAAACC GCTATCCACG CCCATTGATG TACTGCCAAA |
| 6601 | ACCGCATCAC CATGGTAATA GCGATGACTA ATACGTAGAT GTACTGCCAA |
| 6651 | GTAGGAAAGT CCCATAAGGT CATGTACTGG GCATAATGCC AGGCGGGCCA |
| 6701 | TTTACCGTCA TTGACGTCAA TAGGGGGCGT ACTTGGCATA GAT |

1. A method for preventing, delaying, reverting, curing and/or treating a diabetes using a viral expression construct, wherein said viral expression construct comprises the elements a) and b):
a) a nucleotide sequence encoding an insulin operably linked to a first promoter,
b) a nucleotide sequence encoding a glucokinase operably linked to a second promoter and said viral expression construct comprising at least one of elements c), d) and e):
c) the first and the second promoters are positioned in reverse orientation within the expression construct,
d) the first and the second promoters are positioned in reversed orientation within the expression construct and are adjacent to each other, and
e) the first promoter is a CMV promoter.

2. The method of claim 1, wherein said CMV promoter is a mini CMV promoter.

3. The method of claim 1, wherein said viral expression construct comprises elements a), b) and d) or wherein said construct comprises elements a), b) and e) wherein the first promoter is a mini CMV promoter.

4. The method of claim 1, wherein said viral expression construct is such that, the first promoter is a CMV promoter, a murine moloney leukaemia virus (MMLV) promoter, a rous sarcoma virus (RSV) promoter, a HTLV-1 promoter, an SV40 early promoter, or a herpes simplex virus thymidine kinase promoter, and/or the second promoter is a CMV promoter, a MMLV promoter, a RSV promoter, a HTLV-1 promoter, an SV40 early promoter, or a herpes simplex virus thymidine kinase promoter.

5. The method of claim 1, wherein said viral expression construct comprises an additional sequence selected from the group consisting of: Inverted Terminal Repeats (ITRs), SV40 polyadenylation signal, SV40 enhancer sequence, bovine growth hormone (bGH) polyadenylation signal and SV40 polyadenylation signal and enhancer sequence.

6. The method of claim 1, wherein the viral expression construct is represented by a nucleotide sequence comprising SEQ ID NO: 8, 9, 10, 11, 12, 13, 14, 15, 16, 27 or 29 or a sequence having at least 60% identity with SEQ ID NO: 8, 9, 10, 11, 12, 13, 14, 15, 16, 27 or 29.

7. The method of claim 1, wherein said viral expression construct is comprised in a viral vector and said viral vector is a retrovirus vector, an adenovirus vector, an adeno-associated virus vector, a herpesvirus vector, a polyoma virus vector or a vaccinia virus vector.

8. The method of claim 7, wherein said viral vector is an adeno-associated virus vector.

9. The method of claim 8, wherein the adeno-associated virus vector is an AAV1 vector, an AAV2 vector, an AAV3 vector, an AAV4 vector, or an AAV5 vector.

10. The method of claim 1, wherein the viral expression construct is comprised in a composition.

11. The method of claim 10, wherein said composition is a pharmaceutical composition.

12. The method of claim 11, wherein the pharmaceutical composition is delivered by intravenous, subcutaneous, intramuscular, intrathecal, intraarticular and/or intraventricular administration.

13. The method of claim 1, wherein said viral expression construct comprises elements a), b) and d).

14. The method of claim 13, wherein the nucleotide sequence encoding an insulin is at least 80% identical to the sequence set forth as SEQ ID NO: 1, wherein the first promoter is a CMV promoter.

15. The method of claim 14, wherein the nucleotide sequence encoding a glucokinase is at least 80% identical to the sequence set forth as SEQ ID NO: 2, wherein the second promoter is a RSV promoter.

16. A viral expression construct comprising the elements a) and b):
a) a nucleotide sequence encoding an insulin operably linked to a first promoter,
b) a nucleotide sequence encoding a glucokinase operably linked to a second promoter, and
wherein said viral expression construct comprising at least one of elements c), d) and e):
c) the first and the second promoters are positioned in reverse orientation within the expression construct,
d) the first and the second promoters are positioned in reversed orientation within the expression construct and are adjacent to each other, and
e) the first promoter is a CMV promoter.

17. The viral expression construct of claim 16, wherein an additional sequence is present which is selected from the group consisting of: Inverted Terminal Repeats (ITRs), SV40 polyadenylation signal, SV40 enhancer sequence, bovine growth hormone (bGH) polyadenylation signal, SV40 polyadenylation signal and enhancer sequence.

18. The viral expression construct of claim 16, wherein said viral expression construct is comprised in a viral vector which is a retrovirus vector, an adenovirus vector, an adeno-associated virus vector, a herpesvirus vector, a polyoma virus vector or a vaccinia virus vector.

19. The viral expression construct of claim 18, wherein said viral vector is an adeno-associated virus vector, wherein the adeno-associated virus vector is an AAV1 vector, an AAV2 vector, an AAV3 vector, an AAV4 vector, or an AAV5 vector.

20. A method of producing an adeno-associated virus (AAV) vector comprising:
a) introducing an AAV genome into a host cell, wherein the AAV genome comprises i) a nucleotide sequence encoding an insulin operably linked to a first promoter and ii) a nucleotide sequence encoding a glucokinase operably linked to a second promoter,
b) introducing an AAV helper construct into the host cell, wherein the helper construct comprises the viral functions missing from the rAAV genome,
c) introducing a helper virus into the host cell,
d) culturing the host cells to produce the rAAV vectors, and
e) purifying the rAAV vectors.


SEQUENCE LISTING (33 sequences)

SEQ ID NO: 1 - DNA, 349 bp, Homo sapiens
  misc_feature 1..349: note = Human Insulin(A718) Cdna
  source 1..349: mol_type = genomic DNA
cttctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagcc

SEQ ID NO: 2 - DNA, 1409 bp, Homo sapiens
  misc_feature 1..1409: note = cDNA human glucokinase
  source 1..1409: mol_type = genomic DNA
tcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtga

SEQ ID NO: 3 - DNA, 797 bp, synthetic construct
  source 1..797: mol_type = other DNA; note = CMV promoter
tgtagttaatgattaacccgccatgctacttatctacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactag

SEQ ID NO: 4 - DNA, 346 bp, synthetic construct
  source 1..346: mol_type = other DNA
  misc_feature 1..346: note = intronic sequence associated with CMV promoter
tattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactatagaatacgactcactatagggagac

SEQ ID NO: 5 - DNA, 411 bp, synthetic construct
  misc_feature 1..411: note = Mini CMV promoter
  source 1..411: mol_type = other DNA
tatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagctt

SEQ ID NO: 6 - DNA, 623 bp, synthetic construct
  misc_feature 1..623: note = RSV promoter
  source 1..623: mol_type = other DNA
catgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagct

SEQ ID NO: 7 - DNA, 259 bp, synthetic construct
  misc_feature 1..259: note = bGH polyadenylation signal
  source 1..259: mol_type = other DNA
cacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccacgtg

SEQ ID NO: 8 - DNA, 8520 bp, synthetic construct
  misc_feature 1..8520: note = Construct A
  source 1..8520: mol_type = other DNA
ctagacatggctcgacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagctgcaagcttatcgataccgtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtagccatgctctggaagatctcgacgcgtcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagctgctagcaagcttgagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcgggaattcaacaggtggcctcaggagtcaggaacatctctacttccccaacgacccctgggttgtcctctcagagatggctatggatactacaaggtgtggagcccagttgttgactctggtcgagcagatcctggcagagttccagctgcaggaggaagacctgaagaaggtgatgagccggatgcagaaggagatggaccgtggcctgaggctggagacccacgaggaggccagtgtaaagatgttacccacctacgtgcgttccaccccagaaggctcagaagtcggagactttctctccttagacctgggaggaaccaacttcagagtgatgctggtcaaagtgggagagggggaggcagggcagtggagcgtgaagacaaaacaccagatgtactccatccccgaggacgccatgacgggcactgccgagatgctctttgactacatctctgaatgcatctctgacttccttgacaagcatcagatgaagcacaagaaactgcccctgggcttcaccttctccttccctgtgaggcacgaagacctagacaagggcatcctcctcaattggaccaagggcttcaaggcctctggagcagaagggaacaacatcgtaggacttctccgagatgctatcaagaggagaggggactttgagatggatgtggtggcaatggtgaacgacacagtggccacaatgatctcctgctactatgaagaccgccaatgtgaggtcggcatgattgtgggcactggctgcaatgcctgctacatggaggaaatgcagaatgtggagctggtggaaggggatgagggacgcatgtgcgtcaacacggagtggggcgccttcggggactcgggcgagctggatgagttcctactggagtatgaccggatggtggatgaaagctcagcgaaccccggtcagcagctgtacgagaagatcatcggtgggaagtatatgggcgagctggtacgacttgtgctgcttaagctggtggacgagaaccttctgttccacggagaggcctcggagcagctgcgcacgcgtggtgcttttgagacccgtttcgtgtcacaagtggagagcgactccggggaccgaaagcagatccacaacatcctaagcactctggggcttcgaccctctgtcaccgactgcgacattgtgcgccgtgcctgtgaaagcgtgtccactcgcgccgcccatatgtgctccgcaggactagctggggtcataaatcgcatgcgcgaaagccgcagtgaggacgtgatgcgcatcactgtgggcgtggatggctccgtgtacaagctgcacccgagcttcaaggagcggtttcacgccagtgtgcgcaggctgacacccaactgcgaaatcaccttcatcgaatcagaggagggcagcggcaggggagccgcactggtctctgcggtggcctgcaagaaggcttgcatgctggcccagtgaaatccaggtcatatggaccgggacctgggttccacggggactccacacaccacaaatgctcccagcccaccggggcaggagacctattctgctgctacccctggaaaatggggagaggcccctgcaagccgagtcggccagtgggacagccctaggctggatcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctac

SEQ ID NO: 9 - DNA, 7260 bp, synthetic construct
  misc_feature 1..7260: note = Construct D
  source 1..7260: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatctgtagttaatgattaacccgccatgctacttatctacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactaggctagctattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactatagaatacgactcactatagggagacgctagcgtcgaccttctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagccgtcgacggtaccagcgctgtcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctacctagacatggctcgacagatcagcgctcatgctctggaagatctcgatttatccatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagcttctagagagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcggtctagaactcgagtcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgactcgagcacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccacgtgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 10 - DNA, 7260 bp, synthetic construct
  misc_feature 1..7260: note = Construct E
  source 1..7260: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatccatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagcttctagagagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcggtctagaactcgagtcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgactcgagcacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccacgtgatttatctgtagttaatgattaacccgccatgctacttatctacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactaggctagctattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactatagaatacgactcactatagggagacgctagcgtcgaccttctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagccgtcgacggtaccagcgctgtcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctacctagacatggctcgacagatcagcgctcatgctctggaagatctcgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - DNA, 7260 bp, synthetic construct
  misc_feature 1..7260: note = Construct F
  source 1..7260: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgataaatcgagatcttccagagcatgagcgctgatctgtcgagccatgtctaggtagccatgctctaggaagatcctaatcgattttaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggcctcgacagcgctggtaccgtcgacggctgcgtctagttgcagtagttctccagctggtagagggagcagatgctggtacagcattgttccacaatgccacgcttctgtcgcgacccctccagggccaagggctgcaggctgcctgcaccagggcccccgcccagctccacctgccccacctgcaggtcctctgcctcccgcttggtcctgggtgtgtagaagaagcctcgttccccgcacactaggtagagagcttccaccagatctgagccgcacaggtgttggttcacaaaggctgcggctgggtcaggtccccagagggccagcagcgccagcaggggcaggaggcgcatccacagggccatggcagaaggtcgacgctagcgtctccctatagtgagtcgtattctatagtgagtcgtattaagtactctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttctgcaaggctaccttaagagagtcactgcagcttaagttcgagactgttgtgtcagaagcactgactgcgttagcaatttaactgtgataaactaccgcaatagctagcctagtgatctgacggttcactaaacgagctctgcttatatagacctcccaccgtacacgcctaccgcccatttgcgtcaacggggcgggcgatcgcagttgttacgacattttggaaagtcccgttgattttggtgccaaaacaaactcccattgacgtcaatggggtggagacttggaaatccccgtgagtcaaaccgctatccacgcccattggtgtactgccaaaaccgcatcaccatggtaatagcgatgactaatacgtagatgtactgccaagtaggaaagtcccgtaaggtcatgtactgggcataatgccaggcgggccatttaccgtcattgacgtcaatagggggcggacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgccaacatggcggtcatattggacatgagccaatataaatgtacatattatgatatagatacaacgtatgcaatggccaatagccaatattgatttatgctatataaccaatgaataatatggctaatggccaatattgagatctgtagataagtagcatggcgggttaatcattaactacagatatccatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagcttctagagagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcggtctagaactcgagtcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgactcgagcacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccacgtgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 12 - DNA, 7260 bp, synthetic construct
  misc_feature 1..7260: note = Construct G
  source 1..7260: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatccatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagcttctagagagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcggtctagaactcgagtcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgactcgagcacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccacgtgatttaaatcgagatcttccagagcatgagcgctgatctgtcgagccatgtctaggtagccatgctctaggaagatcctaatcgattttaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggcctcgacagcgctggtaccgtcgacggctgcgtctagttgcagtagttctccagctggtagagggagcagatgctggtacagcattgttccacaatgccacgcttctgtcgcgacccctccagggccaagggctgcaggctgcctgcaccagggcccccgcccagctccacctgccccacctgcaggtcctctgcctcccgcttggtcctgggtgtgtagaagaagcctcgttccccgcacactaggtagagagcttccaccagatctgagccgcacaggtgttggttcacaaaggctgcggctgggtcaggtccccagagggccagcagcgccagcaggggcaggaggcgcatccacagggccatggcagaaggtcgacgctagcgtctccctatagtgagtcgtattctatagtgagtcgtattaagtactctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttctgcaaggctaccttaagagagtcactgcagcttaagttcgagactgttgtgtcagaagcactgactgcgttagcaatttaactgtgataaactaccgcaatagctagcctagtgatctgacggttcactaaacgagctctgcttatatagacctcccaccgtacacgcctaccgcccatttgcgtcaacggggcgggcgatcgcagttgttacgacattttggaaagtcccgttgattttggtgccaaaacaaactcccattgacgtcaatggggtggagacttggaaatccccgtgagtcaaaccgctatccacgcccattggtgtactgccaaaaccgcatcaccatggtaatagcgatgactaatacgtagatgtactgccaagtaggaaagtcccgtaaggtcatgtactgggcataatgccaggcgggccatttaccgtcattgacgtcaatagggggcggacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgccaacatggcggtcatattggacatgagccaatataaatgtacatattatgatatagatacaacgtatgcaatggccaatagccaatattgatttatgctatataaccaatgaataatatggctaatggccaatattgagatctgtagataagtagcatggcgggttaatcattaactacagataaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 13 - DNA, 6522 bp, synthetic construct
  misc_feature 1..6522: note = Construct J
  source 1..6522: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatctatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagcttgctagcgtcgaccttctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagccgtcgacggtaccagcgctgtcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctacctagacatggctcgacagatcagcgctcatgctctggaagatctcgatttatccatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagcttctagagagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcggtctagaactcgagtcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgactcgagcacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccacgtgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 14 - DNA, 6522 bp, synthetic construct
  misc_feature 1..6522: note = Construct K
  misc_feature 1..6522: note = Construct L
  source 1..6522: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatccatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagcttctagagagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcggtctagaactcgagtcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgactcgagcacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccacgtgatttatctatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagcttgctagcgtcgaccttctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagccgtcgacggtaccagcgctgtcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctacctagacatggctcgacagatcagcgctcatgctctggaagatctcgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 15 - DNA, 6522 bp, synthetic construct
  misc_feature 1..6522: note = Construct L
  source 1..6522: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgataaatcgagatcttccagagcatgagcgctgatctgtcgagccatgtctaggtagccatgctctaggaagatcctaatcgattttaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggcctcgacagcgctggtaccgtcgacggctgcgtctagttgcagtagttctccagctggtagagggagcagatgctggtacagcattgttccacaatgccacgcttctgtcgcgacccctccagggccaagggctgcaggctgcctgcaccagggcccccgcccagctccacctgccccacctgcaggtcctctgcctcccgcttggtcctgggtgtgtagaagaagcctcgttccccgcacactaggtagagagcttccaccagatctgagccgcacaggtgttggttcacaaaggctgcggctgggtcaggtccccagagggccagcagcgccagcaggggcaggaggcgcatccacagggccatggcagaaggtcgacgctagcaagcttgggtctccctatagtgagtcgtattaatttcgataagccagttaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatagatatccatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagcttctagagagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcggtctagaactcgagtcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgactcgagcacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccacgtgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 16 - DNA, 6522 bp, synthetic construct
  misc_feature 1..6522: note = Construct M
  source 1..6522: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatccatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagcttctagagagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcggtctagaactcgagtcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgactcgagcacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccacgtgatttaaatcgagatcttccagagcatgagcgctgatctgtcgagccatgtctaggtagccatgctctaggaagatcctaatcgattttaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggcctcgacagcgctggtaccgtcgacggctgcgtctagttgcagtagttctccagctggtagagggagcagatgctggtacagcattgttccacaatgccacgcttctgtcgcgacccctccagggccaagggctgcaggctgcctgcaccagggcccccgcccagctccacctgccccacctgcaggtcctctgcctcccgcttggtcctgggtgtgtagaagaagcctcgttccccgcacactaggtagagagcttccaccagatctgagccgcacaggtgttggttcacaaaggctgcggctgggtcaggtccccagagggccagcagcgccagcaggggcaggaggcgcatccacagggccatggcagaaggtcgacgctagcaagcttgggtctccctatagtgagtcgtattaatttcgataagccagttaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatagataaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 17 - DNA, 5804 bp, synthetic construct
  misc_feature 1..5804: note = Construct B
  source 1..5804: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagctgcaagcttatcgataccgtcgacctcgaggaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 18 - DNA, 6722 bp, synthetic construct
  misc_feature 1..6722: note = Construct C
  source 1..6722: mol_type = other DNA
gtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctggaagatctcgacgcgtcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagctgctagcaagcttgagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcgggaattcaacaggtggcctcaggagtcaggaacatctctacttccccaacgacccctgggttgtcctctcagagatggctatggatactacaaggtgtggagcccagttgttgactctggtcgagcagatcctggcagagttccagctgcaggaggaagacctgaagaaggtgatgagccggatgcagaaggagatggaccgtggcctgaggctggagacccacgaggaggccagtgtaaagatgttacccacctacgtgcgttccaccccagaaggctcagaagtcggagactttctctccttagacctgggaggaaccaacttcagagtgatgctggtcaaagtgggagagggggaggcagggcagtggagcgtgaagacaaaacaccagatgtactccatccccgaggacgccatgacgggcactgccgagatgctctttgactacatctctgaatgcatctctgacttccttgacaagcatcagatgaagcacaagaaactgcccctgggcttcaccttctccttccctgtgaggcacgaagacctagacaagggcatcctcctcaattggaccaagggcttcaaggcctctggagcagaagggaacaacatcgtaggacttctccgagatgctatcaagaggagaggggactttgagatggatgtggtggcaatggtgaacgacacagtggccacaatgatctcctgctactatgaagaccgccaatgtgaggtcggcatgattgtgggcactggctgcaatgcctgctacatggaggaaatgcagaatgtggagctggtggaaggggatgagggacgcatgtgcgtcaacacggagtggggcgccttcggggactcgggcgagctggatgagttcctactggagtatgaccggatggtggatgaaagctcagcgaaccccggtcagcagctgtacgagaagatcatcggtgggaagtatatgggcgagctggtacgacttgtgctgcttaagctggtggacgagaaccttctgttccacggagaggcctcggagcagctgcgcacgcgtggtgcttttgagacccgtttcgtgtcacaagtggagagcgactccggggaccgaaagcagatccacaacatcctaagcactctggggcttcgaccctctgtcaccgactgcgacattgtgcgccgtgcctgtgaaagcgtgtccactcgcgccgcccatatgtgctccgcaggactagctggggtcataaatcgcatgcgcgaaagccgcagtgaggacgtgatgcgcatcactgtgggcgtggatggctccgtgtacaagctgcacccgagcttcaaggagcggtttcacgccagtgtgcgcaggctgacacccaactgcgaaatcaccttcatcgaatcagaggagggcagcggcaggggagccgcactggtctctgcggtggcctgcaagaaggcttgcatgctggcccagtgaaatccaggtcatatggaccgggacctgggttccacggggactccacacaccacaaatgctcccagcccaccggggcaggagacctattctgctgctacccctggaaaatggggagaggcccctgcaagccgagtcggccagtgggacagccctaggctggatcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctac

SEQ ID NO: 19 - DNA, 4755 bp, synthetic construct
  misc_feature 1..4755: note = Construct H
  source 1..4755: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatctgtagttaatgattaacccgccatgctacttatctacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactaggctagctattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactatagaatacgactcactatagggagacgctagcgtcgaccttctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagccgtcgacggtaccagcgctgtcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctacctagacatggctcgacagatcagcgctcatgctctggaagatctcgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 20 - DNA, 5417 bp, synthetic construct
  misc_feature 1..5417: note = Construct I
  source 1..5417: mol_type = other DNA
artcaartcacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatccatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagcttctagagagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcggtctagaactcgagtcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgactcgagcacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccacgtgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 21 - DNA, 4017 bp, synthetic construct
  misc_feature 1..4017: note = Construct N
  source 1..4017: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatctatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagcttgctagcgtcgaccttctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagccgtcgacggtaccagcgctgtcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctacctagacatggctcgacagatcagcgctcatgctctggaagatctcgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 22 - DNA, 330 bp, synthetic construct
  misc_feature 1..330: note = SV40
  source 1..330: mol_type = other DNA; note = SV40 polyadenylation signal
ggtaccagcgctgtcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctacctagacatggctcgacagatcagcgctcatgctctggaagatctcg

SEQ ID NO: 23 - DNA, 182 bp, synthetic construct
  misc_feature 1..182: note = Intronic sequence associated with RSV promoter
  source 1..182: mol_type = other DNA
gagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcgg

SEQ ID NO: 24 - DNA, 94 bp, synthetic construct
  misc_feature 1..94: note = Equivalent mini CMV promoter
  source 1..94: mol_type = other DNA
taggcgtgtacggtgggaggtctatataagcagagctctctggctaactagagaacccactgcttaactggcttatcgaaattaatacgactca

SEQ ID NO: 25 - DNA, 3971 bp, synthetic construct
  source 1..3971: mol_type = other DNA; note = Construct O
gctcatgctctggaagatctcgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatctatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagcttgctagcgtcgaccttctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagccgtcgacggtaccagcgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccac

SEQ ID NO: 26 - DNA, 5453 bp, synthetic construct
  source 1..5453: mol_type = other DNA; note = Construct P
gtgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatccatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagcttctagagagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcggtctagaactcgagtcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgactcgagcacgctgtcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctacctagacatggctcgacagatcagc

SEQ ID NO: 27 - DNA, 6522 bp, synthetic construct
  source 1..6522: mol_type = other DNA; note = Construct Q
atccatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagcttctagagagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcggtctagaactcgagtcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgactcgagcacgctgtcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctacctagacatggctcgacagatcagcgtgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgataaatcgagatcttccagagcatgagcgtggccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctccacgctggtaccgtcgacggctgcgtctagttgcagtagttctccagctggtagagggagcagatgctggtacagcattgttccacaatgccacgcttctgtcgcgacccctccagggccaagggctgcaggctgcctgcaccagggcccccgcccagctccacctgccccacctgcaggtcctctgcctcccgcttggtcctgggtgtgtagaagaagcctcgttccccgcacactaggtagagagcttccaccagatctgagccgcacaggtgttggttcacaaaggctgcggctgggtcaggtccccagagggccagcagcgccagcaggggcaggaggcgcatccacagggccatggcagaaggtcgacgctagcaagcttgggtctccctatagtgagtcgtattaatttcgataagccagttaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatagat

SEQ ID NO: 28 - DNA, 4238 bp, synthetic construct
  source 1..4238: mol_type = other DNA; note = Construct R
gctcatgctctggaagatctcgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatctatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagcttgctagcgtcgaccttctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagccgtcgacggtaccagcgctgagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatttggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggcaaatttgagc

SEQ ID NO: 29 - DNA, 6743 bp, synthetic construct
  source 1..6743: mol_type = other DNA; note = Construct S
atccatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagcttctagagagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcggtctagaactcgagtcgagaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgactcgagcacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccacgtgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgataaatcgagatcttccagagcatgagcgctcaaatttgccgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgctccatcgttcagatccttatcgattttaccaaatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccggccgccccgactcagcgctggtaccgtcgacggctgcgtctagttgcagtagttctccagctggtagagggagcagatgctggtacagcattgttccacaatgccacgcttctgtcgcgacccctccagggccaagggctgcaggctgcctgcaccagggcccccgcccagctccacctgccccacctgcaggtcctctgcctcccgcttggtcctgggtgtgtagaagaagcctcgttccccgcacactaggtagagagcttccaccagatctgagccgcacaggtgttggttcacaaaggctgcggctgggtcaggtccccagagggccagcagcgccagcaggggcaggaggcgcatccacagggccatggcagaaggtcgacgctagcaagcttgggtctccctatagtgagtcgtattaatttcgataagccagttaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatagat

SEQ ID NO: 30 - DNA, 520 bp, synthetic construct
  source 1..520: mol_type = other DNA; note = SV40 polyadenylation signal and enhancer sequence
gctgagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatttggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggcaaatttgagc

SEQ ID NO: 31 - DNA, 141 bp, synthetic construct
  source 1..141: mol_type = other DNA; note = 5' ITR AAV2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 32 - DNA, 141 bp, synthetic construct
  source 1..141: mol_type = other DNA; note = 3' ITR AAV2
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 33 - DNA, 230 bp, synthetic construct
  source 1..230: mol_type = other DNA; note = SV40 enhancer sequence
gcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagc